SEQUENCE LISTING (786 sequences)

SEQ ID NO: 1 - protein, 16 aa, Artificial Sequence
  Description of Artificial Sequence Synthetic peptide
RQIKIWFQNRRMKWKK

SEQ ID NO: 2 - protein, 14 aa, Artificial Sequence
  Description of Artificial Sequence Synthetic peptide
YGRKKRRQRRRPPQ

SEQ ID NO: 3 - protein, 12 aa, Artificial Sequence
  Description of Artificial Sequence Synthetic peptide
PLSSIFSRIGDP

SEQ ID NO: 4 - protein, 9 aa, Artificial Sequence
  Description of Artificial Sequence Synthetic peptide
RRRRRRRRR

SEQ ID NO: 5 - protein, 16 aa, Artificial Sequence
  Description of Artificial Sequence Synthetic peptide
AAVALLPAVLLALLAP

SEQ ID NO: 6 - protein, 18 aa, Artificial Sequence
  Description of Artificial Sequence Synthetic peptide
RGGRLSYSRRRFSTSTGR

SEQ ID NO: 7 - protein, 18 aa, Artificial Sequence
  Description of Artificial Sequence Synthetic peptide
LLIILRRRIRKQAHAHSK

SEQ ID NO: 8 - protein, 7 aa, Artificial Sequence
  Description of Artificial Sequence Synthetic peptide
PKKKRKV

SEQ ID NO: 9 - protein, 9 aa, Artificial Sequence
  Description of Artificial Sequence Synthetic peptide
RKKRRQRRR

SEQ ID NO: 10 - protein, 13 aa, Artificial Sequence
  Description of Artificial Sequence Synthetic peptide
GRKKRRQRRRPPQ

SEQ ID NO: 11 - protein, 24 aa, Artificial Sequence
  Description of Artificial Sequence Synthetic peptide
LGTYTQDFNKFHTFPQTAIGVGAP

SEQ ID NO: 12 - protein, 10 aa, Artificial Sequence
  Description of Artificial Sequence Synthetic peptide
VQRKRQKLMP

SEQ ID NO: 13 - protein, 8 aa, Artificial Sequence
  Description of Artificial Sequence Synthetic peptide
SKKKKTKV

SEQ ID NO: 14 - protein, 8 aa, Artificial Sequence
  Description of Artificial Sequence Synthetic peptide
GRKRKKRT

SEQ ID NO: 15 - protein, 9 aa, Artificial Sequence
  Description of Artificial Sequence Synthetic peptide
RRRERRAEK

SEQ ID NO: 16 - protein, 9 aa, Artificial Sequence
  Description of Artificial Sequence Synthetic peptide
KCPSRRPKR

SEQ ID NO: 17 - protein, 6 aa, Artificial Sequence
  Description of Artificial Sequence Synthetic peptide
VRLPPP

SEQ ID NO: 18 - protein, 6 aa, Artificial Sequence
  Description of Artificial Sequence Synthetic peptide
VHLPPP

SEQ ID NO: 19 - protein, 6 aa, Artificial Sequence
  Description of Artificial Sequence Synthetic peptide
VKLPPP

SEQ ID NO: 20 - protein, 17 aa, Artificial Sequence
  Description of Artificial Sequence Synthetic peptide
AVGAIGALFLGFLGAAG

SEQ ID NO: 21 - protein, 15 aa, Artificial Sequence
  Description of Artificial Sequence Synthetic peptide
VTVLALGALAGVGVG

SEQ ID NO: 22 - protein, 18 aa, Artificial Sequence
  Description of Artificial Sequence Synthetic peptide
IAARIKLRSRQHIKLRHL

SEQ ID NO: 23 - protein, 27 aa, Artificial Sequence
  Description of Artificial Sequence Synthetic peptide
DTWPGVEALIRILQQLLFIHFRIGCQH

SEQ ID NO: 24 - protein, 34 aa, Artificial Sequence
  Description of Artificial Sequence Synthetic polypeptide
DAATATRGRSAASRPTERPRAPARSASRPRRPVD

SEQ ID NO: 25 - protein, 17 aa, Artificial Sequence
  Description of Artificial Sequence Synthetic peptide
GALFLGWLGAAGSTMGA

SEQ ID NO: 26 - protein, 17 aa, Artificial Sequence
  Description of Artificial Sequence Synthetic peptide
MGLGLHLLVLAAALQGA

SEQ ID NO: 27 - protein, 27 aa, Artificial Sequence
  Description of Artificial Sequence Synthetic peptide
RQGAARVTSWLGRQLRIAGKRLEGRSK

SEQ ID NO: 28 - protein, 28 aa, Artificial Sequence
  Description of Artificial Sequence Synthetic peptide
MANLGYWLLALFVTMWTDVGLCKKRPKP

SEQ ID NO: 29 - protein, 16 aa, Artificial Sequence
  Description of Artificial Sequence Synthetic peptide
TRRNKRNRIQEQLNRK

SEQ ID NO: 30 - protein, 18 aa, Artificial Sequence
  Description of Artificial Sequence Synthetic peptide
TAKTRYKARRAELIAERR

SEQ ID NO: 31 - protein, 22 aa, Artificial Sequence
  Description of Artificial Sequence Synthetic peptide
MDAQTRRRERRAEKQAQWKAAN

SEQ ID NO: 32 - protein, 15 aa, Artificial Sequence
  Description of Artificial Sequence Synthetic peptide
RRRRNRTRRNRRRVR

SEQ ID NO: 33 - protein, 19 aa, Artificial Sequence
  Description of Artificial Sequence Synthetic peptide
KMTRAQRRAAARRNRWTAR

SEQ ID NO: 34 - protein, 13 aa, Artificial Sequence
  Description of Artificial Sequence Synthetic peptide
TRRQRTRRARRNR

SEQ ID NO: 35 - protein, 12 aa, Artificial Sequence
  Description of Artificial Sequence Synthetic peptide
RQARRNRRRRWR

SEQ ID NO: 36 - protein, 22 aa, Artificial Sequence
  Description of Artificial Sequence Synthetic peptide
DRRRRGSRPSGAERRRRRAAAA

SEQ ID NO: 37 - protein, 15 aa, Artificial Sequence
  Description of Artificial Sequence Synthetic peptide
QTRRRERRAEKQAQW

SEQ ID NO: 38 - protein, 16 aa, Artificial Sequence
  Description of Artificial Sequence Synthetic peptide
KRPAAIKKAGQAKKKK

SEQ ID NO: 39 - protein, 12 aa, Artificial Sequence
  Description of Artificial Sequence Synthetic peptide
TRRSKRRSHRKF

SEQ ID NO: 40 - protein, 17 aa, Artificial Sequence
  Description of Artificial Sequence Synthetic peptide
RAGLQFPVGRVHRLLRK

SEQ ID NO: 41 - protein, 21 aa, Artificial Sequence
  Description of Artificial Sequence Synthetic peptide
TRSSRAGLQFPVGRVHRLLRK

SEQ ID NO: 42 - protein, 16 aa, Artificial Sequence
  Description of Artificial Sequence Synthetic peptide
RHIKIWFQNRRMKWKK

SEQ ID NO: 43 - protein, 42 aa, Artificial Sequence
  Description of Artificial Sequence Synthetic polypeptide
YKQCHKKGGHCFPKEKICLPPSSDFGKMDCRWRWKCCKKGSG

SEQ ID NO: 44 - protein, 16 aa, Artificial Sequence
  Description of Artificial Sequence Synthetic peptide
RVIRVWFQNKRCKDKK

SEQ ID NO: 45 - protein, 60 aa, Artificial Sequence
  Description of Artificial Sequence Synthetic polypeptide
SKRTRQTYTRYQTLELEKEFHFNRYITRRRRIDIANALSLSERQIKIWFQNRRMKSKKDR

SEQ ID NO: 46 - protein, 60 aa, Artificial Sequence
  Description of Artificial Sequence Synthetic polypeptide
EKRPRTAFSSEQLARLKREFNENRYLTERRRQQLSSELGLNEAQIKIWFQNKRAKIKKST

SEQ ID NO: 47 - protein, 27 aa, Artificial Sequence
  Description of Artificial Sequence Synthetic peptide
GWTLNSAGYLLGKINLKALAALAKKIL

SEQ ID NO: 48 - protein, 8 aa, Artificial Sequence
  Description of Artificial Sequence Synthetic peptide
RRRRRRRR

SEQ ID NO: 49 - protein, 9 aa, Artificial Sequence
  Description of Artificial Sequence Synthetic peptide
  MOD_RES (1)..(9): D-amino acid
RRRRRRRRR

SEQ ID NO: 50 - protein, 18 aa, Artificial Sequence
  Description of Artificial Sequence Synthetic peptide
KLALKLALKALKAALKLA

SEQ ID NO: 51 - protein, 30 aa, Artificial Sequence
  Description of Artificial Sequence Synthetic polypeptide
WEAKLAKALAKALAKHLAKALAKALKACEA

SEQ ID NO: 52 - protein, 11 aa, Artificial Sequence
  Description of Artificial Sequence Synthetic peptide
YARLAARQARA

SEQ ID NO: 53 - protein, 27 aa, Artificial Sequence
  Description of Artificial Sequence Synthetic peptide
DPKGDPPKGVTVTVTVTVTGKGDPKPD

SEQ ID NO: 54 - protein, 16 aa, Artificial Sequence
  Description of Artificial Sequence Synthetic peptide
KKWKMRRNQFWVRVQR

SEQ ID NO: 55 - protein, 17 aa, Artificial Sequence
  Description of Artificial Sequence Synthetic peptide
RRWRRWWWRRWWRRWRR

SEQ ID NO: 56 - protein, 28 aa, Artificial Sequence
  Description of Artificial Sequence Synthetic peptide
GALFLGFLGAAGSTMGAWSQPKSKRKVC

SEQ ID NO: 57 - protein, 21 aa, Artificial Sequence
  Description of Artificial Sequence Synthetic peptide
KETWWETWWTEWSQPKKKRKV

SEQ ID NO: 58 - protein, 27 aa, Artificial Sequence
  Description of Artificial Sequence Synthetic peptide
GALFLGWLGAAGSTMGAWSQPKKKRKV

SEQ ID NO: 59 - protein, 12 aa, Artificial Sequence
  Description of Artificial Sequence Synthetic peptide
RRQRRTSKLMKR

SEQ ID NO: 60 - protein, 10 aa, Artificial Sequence
  Description of Artificial Sequence Synthetic peptide
RRIPNRRPRR

SEQ ID NO: 61 - protein, 11 aa, Artificial Sequence
  Description of Artificial Sequence Synthetic peptide
YGRRARRRRRR

SEQ ID NO: 62 - protein, 12 aa, Artificial Sequence
  Description of Artificial Sequence Synthetic peptide
SQMTRQARRLYV

SEQ ID NO: 63 - protein, 16 aa, Artificial Sequence
  Description of Artificial Sequence Synthetic peptide
SIPPEVKFNKPFVYLI

SEQ ID NO: 64 - protein, 17 aa, Artificial Sequence
  Description of Artificial Sequence Synthetic peptide
KKWKMRRNQFWVKVQRG

SEQ ID NO: 65 - protein, 43 aa, Artificial Sequence
  Description of Artificial Sequence Synthetic polypeptide
AAVALLPAVLLALLAVTDQLGEDFFAVDLEAFLQEFGLLPEKE

SEQ ID NO: 66 - protein, 15 aa, Artificial Sequence
  Description of Artificial Sequence Synthetic peptide
KKAAAVLLPVLLAAP

SEQ ID NO: 67 - protein, 14 aa, Artificial Sequence
  Description of Artificial Sequence Synthetic peptide
INLKALAALAKKIL

SEQ ID NO: 68 - protein, 13 aa, Artificial Sequence
  Description of Artificial Sequence Synthetic peptide
MPKKKPTPIQLNP

SEQ ID NO: 69 - protein, 16 aa, Artificial Sequence
  Description of Artificial Sequence Synthetic peptide
AAVALLPAVLLALLAK

SEQ ID NO: 70 - protein, 29 aa, Artificial Sequence
  Description of Artificial Sequence Synthetic peptide
MNLLRKIVKNRRDEDTQKSSPASAPLDDG

SEQ ID NO: 71 - protein, 11 aa, Artificial Sequence
  Description of Artificial Sequence Synthetic peptide
ACSSSPSKHCG

SEQ ID NO: 72 - protein, 10 aa, Artificial Sequence
  Description of Artificial Sequence Synthetic peptide
RRLSYSRRRF

SEQ ID NO: 73 - protein, 12 aa, Artificial Sequence
  Description of Artificial Sequence Synthetic peptide
PIRRRKKLRRLK

SEQ ID NO: 74 - protein, 27 aa, Artificial Sequence
  Description of Artificial Sequence Synthetic peptide
MGLGLHLLVLAAALQGAWSQPKKKRKV

SEQ ID NO: 75 - protein, 21 aa, Artificial Sequence
  Description of Artificial Sequence Synthetic peptide
KETWEETWFTEWSQPKKKRKV

SEQ ID NO: 76 - protein, 3 aa, Artificial Sequence
  Description of Artificial Sequence Synthetic peptide
  See specification as filed for detailed description of substitutions and preferred embodiments
RRR

SEQ ID NO: 77 - protein, 3 aa, Artificial Sequence
  Description of Artificial Sequence Synthetic peptide
  See specification as filed for detailed description of substitutions and preferred embodiments
KKK

SEQ ID NO: 78 - protein, 18 aa, Artificial Sequence
  Description of Artificial Sequence Synthetic peptide
KIAAKSIAKIWKSILKIA

SEQ ID NO: 79 - protein, 18 aa, Artificial Sequence
  Description of Artificial Sequence Synthetic peptide
KALAKALAKLWKALAKAA

SEQ ID NO: 80 - protein, 18 aa, Artificial Sequence
  Description of Artificial Sequence Synthetic peptide
KLALKLALKWAKLALKAA

SEQ ID NO: 81 - protein, 18 aa, Artificial Sequence
  Description of Artificial Sequence Synthetic peptide
KLLAKAAKKWLLLALKAA

SEQ ID NO: 82 - protein, 18 aa, Artificial Sequence
  Description of Artificial Sequence Synthetic peptide
KLLAKAALKWLLKALKAA

SEQ ID NO: 83 - protein, 18 aa, Artificial Sequence
  Description of Artificial Sequence Synthetic peptide
KALKKLLAKWLAAAKALL

SEQ ID NO: 84 - protein, 18 aa, Artificial Sequence
  Description of Artificial Sequence Synthetic peptide
KLAAALLKKWKKLAAALL

SEQ ID NO: 85 - protein, 18 aa, Artificial Sequence
  Description of Artificial Sequence Synthetic peptide
KALAALLKKWAKLLAALK

SEQ ID NO: 86 - protein, 18 aa, Artificial Sequence
  Description of Artificial Sequence Synthetic peptide
KALAALLKKLAKLLAALK

SEQ ID NO: 87 - protein, 16 aa, Artificial Sequence
  Description of Artificial Sequence Synthetic peptide
KLALKLALKALKLALK

SEQ ID NO: 88 - protein, 14 aa, Artificial Sequence
  Description of Artificial Sequence Synthetic peptide
KLALKALKAALKLA

SEQ ID NO: 89 - protein, 14 aa, Artificial Sequence
  Description of Artificial Sequence Synthetic peptide
KLALKLALKALKAA

SEQ ID NO: 90 - protein, 18 aa, Artificial Sequence
  Description of Artificial Sequence Synthetic peptide
KLGLKLGLKGLKGGLKLG

SEQ ID NO: 91 - protein, 18 aa, Artificial Sequence
  Description of Artificial Sequence Synthetic peptide
KLALKLALKALQAALQLA

SEQ ID NO: 92 - protein, 18 aa, Artificial Sequence
  Description of Artificial Sequence Synthetic peptide
KLALQLALQALQAALQLA

SEQ ID NO: 93 - protein, 18 aa, Artificial Sequence
  Description of Artificial Sequence Synthetic peptide
QLALQLALQALQAALQLA

SEQ ID NO: 94 - protein, 18 aa, Artificial Sequence
  Description of Artificial Sequence Synthetic peptide
ELALELALEALEAALELA

SEQ ID NO: 95 - protein, 18 aa, Artificial Sequence
  Description of Artificial Sequence Synthetic peptide
LKTLATALTKLAKTLTTL

SEQ ID NO: 96 - protein, 18 aa, Artificial Sequence
  Description of Artificial Sequence Synthetic peptide
LLKTTALLKTTALLKTTA

SEQ ID NO: 97 - protein, 18 aa, Artificial Sequence
  Description of Artificial Sequence Synthetic peptide
LKTLTETLKELTKTLTEL

SEQ ID NO: 98 - protein, 18 aa, Artificial Sequence
  Description of Artificial Sequence Synthetic peptide
LLKTTELLKTTELLKTTE

SEQ ID NO: 99 - protein, 18 aa, Artificial Sequence
  Description of Artificial Sequence Synthetic peptide
  MOD_RES (1)..(18): D-amino acid
KLALKLALKALKAALKLA

SEQ ID NO: 100 - protein, 18 aa, Artificial Sequence
  Description of Artificial Sequence Synthetic peptide
KALKLKLALALLAKLKLA

SEQ ID NO: 101 - protein, 16 aa, Artificial Sequence
  Description of Artificial Sequence Synthetic peptide
KKWKMRRNQFWIKIQR

SEQ ID NO: 102 - protein, 16 aa, Artificial Sequence
  Description of Artificial Sequence Synthetic peptide
  MOD_RES (1)..(16): D-amino acid
RQIKIWFQNRRMKWKK

SEQ ID NO: 103 - protein, 16 aa, Artificial Sequence
  Description of Artificial Sequence Synthetic peptide
RQIKIWFPNRRMKWKK

SEQ ID NO: 104 - protein, 16 aa, Artificial Sequence
  Description of Artificial Sequence Synthetic peptide
RQPKIWFPNRRKPWKK

SEQ ID NO: 105 - protein, 15 aa, Artificial Sequence
  Description of Artificial Sequence Synthetic peptide
RQIKIWFQNRRMKWK

SEQ ID NO: 106 - protein, 14 aa, Artificial Sequence
  Description of Artificial Sequence Synthetic peptide
RQIKIWFQNRRMKW

SEQ ID NO: 107 - protein, 13 aa, Artificial Sequence
  Description of Artificial Sequence Synthetic peptide
RQIKIWFQNRRMK

SEQ ID NO: 108 - protein, 12 aa, Artificial Sequence
  Description of Artificial Sequence Synthetic peptide
RQIKIWFQNRRM

SEQ ID NO: 109 - protein, 11 aa, Artificial Sequence
  Description of Artificial Sequence Synthetic peptide
RQIKTWFQNRR

SEQ ID NO: 110 - protein, 10 aa, Artificial Sequence
  Description of Artificial Sequence Synthetic peptide
RQIKIWFQNR

SEQ ID NO: 111 - protein, 9 aa, Artificial Sequence
  Description of Artificial Sequence Synthetic peptide
RQIKIWFQN

SEQ ID NO: 112 - protein, 8 aa, Artificial Sequence
  Description of Artificial Sequence Synthetic peptide
RQIKIWFQ

SEQ ID NO: 113 - protein, 6 aa, Artificial Sequence
  Description of Artificial Sequence Synthetic peptide
RQIKIW

SEQ ID NO: 114 - protein, 15 aa, Artificial Sequence
  Description of Artificial Sequence Synthetic peptide
QIKIWFQNRRMKWKK

SEQ ID NO: 115 - protein, 14 aa, Artificial Sequence
  Description of Artificial Sequence Synthetic peptide
IKIWFQNRRMKWKK

SEQ ID NO: 116 - protein, 13 aa, Artificial Sequence
  Description of Artificial Sequence Synthetic peptide
KIWFQNRRMKWKK

SEQ ID NO: 117 - protein, 12 aa, Artificial Sequence
  Description of Artificial Sequence Synthetic peptide
IWFQNRRMKWKK

SEQ ID NO: 118 - protein, 11 aa, Artificial Sequence
  Description of Artificial Sequence Synthetic peptide
WFQNRRMKWKK

SEQ ID NO: 119 - protein, 10 aa, Artificial Sequence
  Description of Artificial Sequence Synthetic peptide
FQNRRMKWKK

SEQ ID NO: 120 - protein, 9 aa, Artificial Sequence
  Description of Artificial Sequence Synthetic peptide
QNRRMKWKK

SEQ ID NO: 121 - protein, 8 aa, Artificial Sequence
  Description of Artificial Sequence Synthetic peptide
NRRMKWKK

SEQ ID NO: 122 - protein, 7 aa, Artificial Sequence
  Description of Artificial Sequence Synthetic peptide
RRMKWKK

SEQ ID NO: 123 - protein, 6 aa, Artificial Sequence
  Description of Artificial Sequence Synthetic peptide
RMKWKK

SEQ ID NO: 124 - protein, 16 aa, Artificial Sequence
  Description of Artificial Sequence Synthetic peptide
AQIKIWFQNRRMKWKK

SEQ ID NO: 125 - protein, 16 aa, Artificial Sequence
  Description of Artificial Sequence Synthetic peptide
RAIKIWFQNRRMKWKK

SEQ ID NO: 126 - protein, 16 aa, Artificial Sequence
  Description of Artificial Sequence Synthetic peptide
RQAKIWFQNRRMKWKK

SEQ ID NO: 127 - protein, 16 aa, Artificial Sequence
  Description of Artificial Sequence Synthetic peptide
RQIAIWFQNRRMKWKK

SEQ ID NO: 128 - protein, 16 aa, Artificial Sequence
  Description of Artificial Sequence Synthetic peptide
RQIKAWFQNRRMKWKK

SEQ ID NO: 129 - protein, 16 aa, Artificial Sequence
  Description of Artificial Sequence Synthetic peptide
RQIKIWAQNRRMKWKK

SEQ ID NO: 130 - protein, 16 aa, Artificial Sequence
  Description of Artificial Sequence Synthetic peptide
RQIKIWAQNRRMKWKK

SEQ ID NO: 131 - protein, 16 aa, Artificial Sequence
  Description of Artificial Sequence Synthetic peptide
RQIKIWFANRRMKWKK

SEQ ID NO: 132 - protein, 16 aa, Artificial Sequence
  Description of Artificial Sequence Synthetic peptide
RQIKIWFQARRMKWKK

SEQ ID NO: 133 - protein, 16 aa, Artificial Sequence
  Description of Artificial Sequence Synthetic peptide
RQIKIWFQNARMKWKK

SEQ ID NO: 134 - protein, 15 aa, Artificial Sequence
  Description of Artificial Sequence Synthetic peptide
KIKIWFQNRAMKWKK

SEQ ID NO: 135 - protein, 16 aa, Artificial Sequence
  Description of Artificial Sequence Synthetic peptide
RQIKIWFQNRRAKWKK

SEQ ID NO: 136 - protein, 16 aa, Artificial Sequence
  Description of Artificial Sequence Synthetic peptide
RQTKIWFQNRRMAWKK

SEQ ID NO: 137 - protein, 16 aa, Artificial Sequence
  Description of Artificial Sequence Synthetic peptide
RQIKIWFQNRRMKAKK

SEQ ID NO: 138 - protein, 16 aa, Artificial Sequence
  Description of Artificial Sequence Synthetic peptide
RQIKIWFQNRRMKWAK

SEQ ID NO: 139 - protein, 16 aa, Artificial Sequence
  Description of Artificial Sequence Synthetic peptide
RQIKIWFQNRRMKWKA

SEQ ID NO: 140 - protein, 18 aa, Artificial Sequence
  Description of Artificial Sequence Synthetic peptide
CRQIKIWFPNRRMKWKKC

SEQ ID NO: 141 - protein, 16 aa, Artificial Sequence
  Description of Artificial Sequence Synthetic peptide
RQIKIWFPNRRMKWKK

SEQ ID NO: 142 - protein, 16 aa, Artificial Sequence
  Description of Artificial Sequence Synthetic peptide
RQIKIWFQNRRMKAKK

SEQ ID NO: 143 - protein, 16 aa, Artificial Sequence
  Description of Artificial Sequence Synthetic peptide
RQIRIWFQNRRMRWRR

SEQ ID NO: 144 - protein, 8 aa, Artificial Sequence
  Description of Artificial Sequence Synthetic peptide
RRRRRRRW

SEQ ID NO: 145 - protein, 13 aa, Artificial Sequence
  Description of Artificial Sequence Synthetic peptide
GRKKRRQRRRPWQ

SEQ ID NO: 146 - protein, 13 aa, Artificial Sequence
  Description of Artificial Sequence Synthetic peptide
GRKKRRQRRRPWQ

SEQ ID NO: 147 - protein, 16 aa, Artificial Sequence
  Description of Artificial Sequence Synthetic peptide
RQIRIWFQNRRMRWRR

SEQ ID NO: 148 - protein, 16 aa, Artificial Sequence
  Description of Artificial Sequence Synthetic peptide
RRWRRWWRRWWRRWRR

SEQ ID NO: 149 - protein, 16 aa, Artificial Sequence
  Description of Artificial Sequence Synthetic peptide
RQIKIWFQNMRRKWKK

SEQ ID NO: 150 - protein, 13 aa, Artificial Sequence
  Description of Artificial Sequence Synthetic peptide
KMDCRWRWKCCKK

SEQ ID NO: 151 - protein, 12 aa, Artificial Sequence
  Description of Artificial Sequence Synthetic peptide
MDCRWRWKCCKK

SEQ ID NO: 152 - protein, 11 aa, Artificial Sequence
  Description of Artificial Sequence Synthetic peptide
DCRWRWKCCKK

SEQ ID NO: 153 - protein, 10 aa, Artificial Sequence
  Description of Artificial Sequence Synthetic peptide
CRWRWKCCKK

SEQ ID NO: 154 - protein, 9 aa, Artificial Sequence
  Description of Artificial Sequence Synthetic peptide
RWRWKCCKK

SEQ ID NO: 155 - protein, 12 aa, Artificial Sequence
  Description of Artificial Sequence Synthetic peptide
KMDCRWRWKCKK

SEQ ID NO: 156 - protein, 11 aa, Artificial Sequence
  Description of Artificial Sequence Synthetic peptide
KMDCRWRWKKK

SEQ ID NO: 157 - protein, 10 aa, Artificial Sequence
  Description of Artificial Sequence Synthetic peptide
KMDRWRWKKK

SEQ ID NO: 158 - protein, 12 aa, Artificial Sequence
  Description of Artificial Sequence Synthetic peptide
KDCRWRWKCCKK

SEQ ID NO: 159 - protein, 11 aa, Artificial Sequence
  Description of Artificial Sequence Synthetic peptide
KCRWRWKCCKK

SEQ ID NO: 160 - protein, 10 aa, Artificial Sequence
  Description of Artificial Sequence Synthetic peptide
KRWRWKCCKK

SEQ ID NO: 161 - protein, 12 aa, Artificial Sequence
  Description of Artificial Sequence Synthetic peptide
  MOD_RES (9)..(9): Any amino acid
MDCRWRWKXCKK

SEQ ID NO: 162 - protein, 11 aa, Artificial Sequence
  Description of Artificial Sequence Synthetic peptide
  MOD_RES (8)..(8): Any amino acid
DCRWRWKXCKK

SEQ ID NO: 163 - protein, 11 aa, Artificial Sequence
  Description of Artificial Sequence Synthetic peptide
  MOD_RES (9)..(9): Any amino acid
DCRWRWKCXKK

SEQ ID NO: 164 - protein, 10 aa, Artificial Sequence
  Description of Artificial Sequence Synthetic peptide
  MOD_RES (7)..(7): Any amino acid
CRWRWKXCKK

SEQ ID NO: 165 - protein, 10 aa, Artificial Sequence
  Description of Artificial Sequence Synthetic peptide
  MOD_RES (8)..(8): Any amino acid
CRWRWKCXKK

SEQ ID NO: 166 - protein, 9 aa, Artificial Sequence
  Description of Artificial Sequence Synthetic peptide
  MOD_RES (6)..(6): Any amino acid
RWRWKXCKK

SEQ ID NO: 167 - protein, 12 aa, Artificial Sequence
  Description of Artificial Sequence Synthetic peptide
  MOD_RES (9)..(10): Any amino acid
MDCRWRWKXXKK

SEQ ID NO: 168 - protein, 11 aa, Artificial Sequence
  Description of Artificial Sequence Synthetic peptide
  MOD_RES (8)..(9): Any amino acid
DCRWRWKXXKK

SEQ ID NO: 169 - protein, 10 aa, Artificial Sequence
  Description of Artificial Sequence Synthetic peptide
  MOD_RES (7)..(8): Any amino acid
CRWRWKXXKK

SEQ ID NO: 170 - protein, 9 aa, Artificial Sequence
  Description of Artificial Sequence Synthetic peptide
  MOD_RES (6)..(7): Any amino acid
RWRWKXXKK

SEQ ID NO: 171 - protein, 10 aa, Artificial Sequence
  Description of Artificial Sequence Synthetic peptide
CRWRWKCSKK

SEQ ID NO: 172 - protein, 10 aa, Artificial Sequence
  Description of Artificial Sequence Synthetic peptide
SRWRWKCCKK

SEQ ID NO: 173 - protein, 10 aa, Artificial Sequence
  Description of Artificial Sequence Synthetic peptide
SRWRWKCSKK

SEQ ID NO: 174 - protein, 10 aa, Artificial Sequence
  Description of Artificial Sequence Synthetic peptide
SRWRWKSCKK

SEQ ID NO: 175 - protein, 10 aa, Artificial Sequence
  Description of Artificial Sequence Synthetic peptide
CRWRWKSSKK

SEQ ID NO: 176 - protein, 10 aa, Artificial Sequence
  Description of Artificial Sequence Synthetic peptide
SRWRWKSSKK

SEQ ID NO: 177 - protein, 10 aa, Artificial Sequence
  Description of Artificial Sequence Synthetic peptide
CRFRWKCCKK

SEQ ID NO: 178 - protein, 10 aa, Artificial Sequence
  Description of Artificial Sequence Synthetic peptide
CRWRFKCCKK

SEQ ID NO: 179 - protein, 10 aa, Artificial Sequence
  Description of Artificial Sequence Synthetic peptide
CRFRFKCCKK

SEQ ID NO: 180 - protein, 10 aa, Artificial Sequence
  Description of Artificial Sequence Synthetic peptide
  MOD_RES (1)..(10): D-amino acid
CRWRWKCCKK

SEQ ID NO: 181 - protein, 10 aa, Artificial Sequence
  Description of Artificial Sequence Synthetic peptide
KCCKWRWRCK

SEQ ID NO: 182 - protein, 10 aa, Artificial Sequence
  Description of Artificial Sequence Synthetic peptide
  MOD_RES (1)..(10): D-amino acid
KCCKWRWRCK

SEQ ID NO: 183 - protein, 10 aa, Artificial Sequence
  Description of Artificial Sequence Synthetic peptide
  MOD_RES (2)..(2): D-amino acid
CRWRWKCCKK

SEQ ID NO: 184 - protein, 10 aa, Artificial Sequence
  Description of Artificial Sequence Synthetic peptide
  MOD_RES (3)..(3): D-amino acid
CRWRWKCCKK

SEQ ID NO: 185 - protein, 10 aa, Artificial Sequence
  Description of Artificial Sequence Synthetic peptide
  MOD_RES (4)..(4): D-amino acid
CRWRWKCCKK

SEQ ID NO: 186 - protein, 10 aa, Artificial Sequence
  Description of Artificial Sequence Synthetic peptide
  MOD_RES (5)..(5): D-amino acid
CRWRWKCCKK

SEQ ID NO: 187 - protein, 10 aa, Artificial Sequence
  Description of Artificial Sequence Synthetic peptide
  MOD_RES (2)..(5): D-amino acid
CRWRWKCCKK

SEQ ID NO: 188 - protein, 11 aa, Artificial Sequence
  Description of Artificial Sequence Synthetic peptide
CRWRWKCGCKK

SEQ ID NO: 189 - protein, 14 aa, Artificial Sequence
  Description of Artificial Sequence Synthetic peptide
KCGCRWRWKCGCKK

SEQ ID NO: 190 - protein, 8 aa, Artificial Sequence
  Description of Artificial Sequence Synthetic peptide
CRWRWKCG

SEQ ID NO: 191 - protein, 13 aa, Artificial Sequence
  Description of Artificial Sequence Synthetic peptide
  MOD_RES (4)..(4): Any amino acid
KMDXRWRWKCCKK

SEQ ID NO: 192 - protein, 13 aa, Artificial Sequence
  Description of Artificial Sequence Synthetic peptide
  MOD_RES (4)..(4): Any amino acid
  MOD_RES (10)..(10): Any amino acid
KMDXRWRWKXCKK

SEQ ID NO: 193 - protein, 13 aa, Artificial Sequence
  Description of Artificial Sequence Synthetic peptide
  MOD_RES (4)..(4): Any amino acid
  MOD_RES (10)..(11): Any amino acid
KMDXRWRWKXXKK

SEQ ID NO: 194 - protein, 13 aa, Artificial Sequence
  Description of Artificial Sequence Synthetic peptide
  MOD_RES (4)..(4): Any amino acid
  MOD_RES (11)..(11): Any amino acid
KMDXRWRWKCXKK

SEQ ID NO: 195 - protein, 12 aa, Artificial Sequence
  Description of Artificial Sequence Synthetic peptide
  MOD_RES (10)..(10): Any amino acid
MDCRWRWKCXKK

SEQ ID NO: 196 - protein, 13 aa, Artificial Sequence
  Description of Artificial Sequence Synthetic peptide
KMDCRWRWKCSKK

SEQ ID NO: 197 - protein, 13 aa, Artificial Sequence
  Description of Artificial Sequence Synthetic peptide
KMDCRWRWKSCKK

SEQ ID NO: 198 - protein, 13 aa, Artificial Sequence
  Description of Artificial Sequence Synthetic peptide
  MOD_RES (4)..(4): Any amino acid
KMDXRWRWKCCKK

SEQ ID NO: 199 - protein, 13 aa, Artificial Sequence
  Description of Artificial Sequence Synthetic peptide
KMDCRWRWKSSKK

SEQ ID NO: 200 - protein, 13 aa, Artificial Sequence
  Description of Artificial Sequence Synthetic peptide
KMDSRWRWKSSKK

SEQ ID NO: 201 - protein, 13 aa, Artificial Sequence
  Description of Artificial Sequence Synthetic peptide
KMDSRWRWKSCKK

SEQ ID NO: 202 - protein, 13 aa, Artificial Sequence
  Description of Artificial Sequence Synthetic peptide
KMDSRWRWKCSKK

SEQ ID NO: 203 - protein, 13 aa, Artificial Sequence
  Description of Artificial Sequence Synthetic peptide
KMDCRWRPKCCKK

SEQ ID NO: 204 - protein, 13 aa, Artificial Sequence
  Description of Artificial Sequence Synthetic peptide
KMDCRPRPKCCKK

SEQ ID NO: 205 - protein, 13 aa, Artificial Sequence
  Description of Artificial Sequence Synthetic peptide
  MOD_RES (4)..(4): Any amino acid
KMDXRPRPKCCKK

SEQ ID NO: 206 - protein, 13 aa, Artificial Sequence
  Description of Artificial Sequence Synthetic peptide
  MOD_RES (4)..(4): Any amino acid
  MOD_RES (10)..(10): Any amino acid
KMDXRPRPKXCKK

SEQ ID NO: 207 - protein, 13 aa, Artificial Sequence
  Description of Artificial Sequence Synthetic peptide
  MOD_RES (4)..(4): Any amino acid
  MOD_RES (11)..(11): Any amino acid
KMDXRPRPKCXKK

SEQ ID NO: 208 - protein, 13 aa, Artificial Sequence
  Description of Artificial Sequence Synthetic peptide
  MOD_RES (10)..(10): Any amino acid
KMDCRPRPKXCKK

SEQ ID NO: 209 - protein, 13 aa, Artificial Sequence
  Description of Artificial Sequence Synthetic peptide
  MOD_RES (11)..(11): Any amino acid
KMDCRPRPKCXKK

SEQ ID NO: 210 - protein, 9 aa, Artificial Sequence
  Description of Artificial Sequence Synthetic peptide
  MOD_RES (1)..(9): D-amino acid
RKKRRQRRR

SEQ ID NO: 211 - protein, 9 aa, Artificial Sequence
  Description of Artificial Sequence Synthetic peptide
  MOD_RES (1)..(9): D-amino acid
RRRCRRKKR

SEQ ID NO: 212 - protein, 16 aa, Artificial Sequence
  Description of Artificial Sequence Synthetic peptide
RKKRRRESRKKRRRES

SEQ ID NO: 213 - protein, 17 aa, Artificial Sequence
  Description of Artificial Sequence Synthetic peptide
GRPRESGKKRKRKRLKP

SEQ ID NO: 214 - protein, 15 aa, Artificial Sequence
  Description of Artificial Sequence Synthetic peptide
GKRKKKGKLGKKRDP

SEQ ID NO: 215 - protein, 17 aa, Artificial Sequence
  Description of Artificial Sequence Synthetic peptide
GKRKKKGKLGKKRPRSR

SEQ ID NO: 216 - protein, 18 aa, Artificial Sequence
  Description of Artificial Sequence Synthetic peptide
RKKRRRESRRARRSPRHL

SEQ ID NO: 217 - protein, 19 aa, Artificial Sequence
  Description of Artificial Sequence Synthetic peptide
SRRARRSPRESGKKRKRKR

SEQ ID NO: 218 - protein, 19 aa, Artificial Sequence
  Description of Artificial Sequence Synthetic peptide
VKRGLKLRHVRPRVTRMDV

SEQ ID NO: 219 - protein, 14 aa, Artificial Sequence
  Description of Artificial Sequence Synthetic peptide
SRRARRSPRHLGSG

SEQ ID NO: 220 - protein, 16 aa, Artificial Sequence
  Description of Artificial Sequence Synthetic peptide
LRRERQSRLRRERQSR

SEQ ID NO: 221 - protein, 22 aa, Artificial Sequence
  Description of Artificial Sequence Synthetic peptide
GAYDLRRRERQSRLRRRERQSR

SEQ ID NO: 222 - protein, 5 aa, Artificial Sequence
  Description of Artificial Sequence Synthetic peptide
VPMLK

SEQ ID NO: 223 - protein, 5 aa, Artificial Sequence
  Description of Artificial Sequence Synthetic peptide
VPMLK

SEQ ID NO: 224 - protein, 5 aa, Artificial Sequence
  Description of Artificial Sequence Synthetic peptide
VPALR

SEQ ID NO: 225 - protein, 5 aa, Artificial Sequence
  Description of Artificial Sequence Synthetic peptide
VSALK

SEQ ID NO: 226 - protein, 5 aa, Artificial Sequence
  Description of Artificial Sequence Synthetic peptide
PMLKE

SEQ ID NO: 227 - protein, 5 aa, Artificial Sequence
  Description of Artificial Sequence Synthetic peptide
VPALK

SEQ ID NO: 228 - protein, 5 aa, Artificial Sequence
  Description of Artificial Sequence Synthetic peptide
VSLKK

SEQ ID NO: 229 - protein, 5 aa, Artificial Sequence
  Description of Artificial Sequence Synthetic peptide
VSGKK

SEQ ID NO: 230 - protein, 5 aa, Artificial Sequence
  Description of Artificial Sequence Synthetic peptide
KLPVM

SEQ ID NO: 231 - protein, 5 aa, Artificial Sequence
  Description of Artificial Sequence Synthetic peptide
IPMIK

SEQ ID NO: 232 - protein, 5 aa, Artificial Sequence
  Description of Artificial Sequence Synthetic peptide
KLGVM

SEQ ID NO: 233 - protein, 5 aa, Artificial Sequence
  Description of Artificial Sequence Synthetic peptide
KLPVT

SEQ ID NO: 234 - protein, 5 aa, Artificial Sequence
  Description of Artificial Sequence Synthetic peptide
VPMIK

SEQ ID NO: 235 - protein, 5 aa, Artificial Sequence
  Description of Artificial Sequence Synthetic peptide
IPALK

SEQ ID NO: 236 - protein, 5 aa, Artificial Sequence
  Description of Artificial Sequence Synthetic peptide
IPMLK

SEQ ID NO: 237 - protein, 5 aa, Artificial Sequence
  Description of Artificial Sequence Synthetic peptide
VPTLQ

SEQ ID NO: 238 - protein, 5 aa, Artificial Sequence
  Description of Artificial Sequence Synthetic peptide
QLPVM

SEQ ID NO: 239 - protein, 5 aa, Artificial Sequence
  Description of Artificial Sequence Synthetic peptide
ELPVM

SEQ ID NO: 240 - protein, 5 aa, Artificial Sequence
  Description of Artificial Sequence Synthetic peptide
VPTLE

SEQ ID NO: 241 - protein, 5 aa, Artificial Sequence
  Description of Artificial Sequence Synthetic peptide
  MOD_RES (1)..(5): D-amino acid
VPTLK

SEQ ID NO: 242 - protein, 20 aa, Artificial Sequence
  Description of Artificial Sequence Synthetic peptide
AYRIKPTFRRLKWKYKGKFW

SEQ ID NO: 243 - protein, 20 aa, Artificial Sequence
  Description of Artificial Sequence Synthetic peptide
HARIKPTFRRLKWKYKGKFW

SEQ ID NO: 244 - protein, 20 aa, Artificial Sequence
  Description of Artificial Sequence Synthetic peptide
HYRIKPTARRLKWKYKGKFW

SEQ ID NO: 245 - protein, 20 aa, Artificial Sequence
  Description of Artificial Sequence Synthetic peptide
HYRIKPTFRRLAWKYKGKFW

SEQ ID NO: 246 - protein, 20 aa, Artificial Sequence
  Description of Artificial Sequence Synthetic peptide
HYRIKPTFRRLKWKYKGKFA

SEQ ID NO: 247 - protein, 21 aa, Artificial Sequence
  Description of Artificial Sequence Synthetic peptide
VNADIKATTVFGGKYVSLTTP

SEQ ID NO: 248 - protein, 22 aa, Artificial Sequence
  Description of Artificial Sequence Synthetic peptide
GKYVSLTTPKNPTKRRITPKDV

SEQ ID NO: 249 - protein, 22 aa, Artificial Sequence
  Description of Artificial Sequence Synthetic peptide
TKRRITPKDVIDVRSVTTEINT

SEQ ID NO: 250 - protein, 23 aa, Artificial Sequence
  Description of Artificial Sequence Synthetic peptide
RSVTTEINTLFQTLTSIAEKVDP

SEQ ID NO: 251 - protein, 26 aa, Artificial Sequence
  Description of Artificial Sequence Synthetic peptide
AEKVDPVKLNLTLSAAAEALTGLGDK

SEQ ID NO: 252 - protein, 34 aa, Artificial Sequence
  Description of Artificial Sequence Synthetic polypeptide
GLGDKFGESIVNANTVLDDLNSRMPQSRHDIQQL

SEQ ID NO: 253 - protein, 27 aa, Artificial Sequence
  Description of Artificial Sequence Synthetic peptide
GDVYADAAPDLFDFLDSSVTTARTINA

SEQ ID NO: 254 - protein, 32 aa, Artificial Sequence
  Description of Artificial Sequence Synthetic polypeptide
ARTINAQQAELDSALLAAAGFGNTTADVFDRG

SEQ ID NO: 255 - protein, 30 aa, Artificial Sequence
  Description of Artificial Sequence Synthetic polypeptide
ADVFDRGGPYLQRGVADLVPTATLLDTYSP

SEQ ID NO: 256 - protein, 27 aa, Artificial Sequence
  Description of Artificial Sequence Synthetic peptide
LDTYSPELFCTIRNFYDADRPDRGAAA

SEQ ID NO: 257 - protein, 22 aa, Artificial Sequence
  Description of Artificial Sequence Synthetic peptide
TKRRITPKDVIDVRSVTTEINT

SEQ ID NO: 258 - protein, 22 aa, Artificial Sequence
  Description of Artificial Sequence Synthetic peptide
TKRRITPDDVIDVRSVTTEINT

SEQ ID NO: 259 - protein, 22 aa, Artificial Sequence
  Description of Artificial Sequence Synthetic peptide
TKRRITPKKVIDVRSVTTEINT

SEQ ID NO: 260 - protein, 22 aa, Artificial Sequence
  Description of Artificial Sequence Synthetic peptide
TKRRITPKDVIDVRSVTTKINT

SEQ ID NO: 261 - protein, 13 aa, Artificial Sequence
  Description of Artificial Sequence Synthetic peptide
TKRRITPKDVIDV

SEQ ID NO: 262 - protein, 22 aa, Artificial Sequence
  Description of Artificial Sequence Synthetic peptide
TKRRITPKDVIDVESVTTEINT

SEQ ID NO: 263 - protein, 22 aa, Artificial Sequence
  Description of Artificial Sequence Synthetic peptide
TARRITPKDVIDVRSVTTEINT

SEQ ID NO: 264 - protein, 23 aa, Artificial Sequence
  Description of Artificial Sequence Synthetic peptide
TKAARITPKDVIDVRSVTTEINT

SEQ ID NO: 265 - protein, 28 aa, Artificial Sequence
  Description of Artificial Sequence Synthetic peptide
HHHHHHTKRRITPKDVIDVRSVTTEINT

SEQ ID NO: 266 - protein, 15 aa, Artificial Sequence
  Description of Artificial Sequence Synthetic peptide
KLWMRWYSPTTRRYG

SEQ ID NO: 267 - protein, 15 aa, Artificial Sequence
  Description of Artificial Sequence Synthetic peptide
DSLKSYWYLQKFSWR

SEQ ID NO: 268 - protein, 15 aa, Artificial Sequence
  Description of Artificial Sequence Synthetic peptide
RTLVNEYKNTLKFSK

SEQ ID NO: 269 - protein, 15 aa, Artificial Sequence
  Description of Artificial Sequence Synthetic peptide
IPSRWKDQFWKRWHY

SEQ ID NO: 270 - protein, 15 aa, Artificial Sequence
  Description of Artificial Sequence Synthetic peptide
GYGNCRHFKQKPRRD

SEQ ID NO: 271 - protein, 15 aa, Artificial Sequence
  Description of Artificial Sequence Synthetic peptide
KNAWKHSSCHHRHQI

SEQ ID NO: 272 - protein, 15 aa, Artificial Sequence
  Description of Artificial Sequence Synthetic peptide
RVREWWYTITLKQES

SEQ ID NO: 273 - protein, 15 aa, Artificial Sequence
  Description of Artificial Sequence Synthetic peptide
QQHLLIAINGYPRYN

SEQ ID NO: 274 - protein, 15 aa, Artificial Sequence
  Description of Artificial Sequence Synthetic peptide
WKCRRQCFRVLHHWN

SEQ ID NO: 275 - protein, 15 aa, Artificial Sequence
  Description of Artificial Sequence Synthetic peptide
RLWMRWYSPTTRRYG

SEQ ID NO: 276 - protein, 15 aa, Artificial Sequence
  Description of Artificial Sequence Synthetic peptide
KLWMRWYSATTRRYG

SEQ ID NO: 277 - protein, 15 aa, Artificial Sequence
  Description of Artificial Sequence Synthetic peptide
KLWMRWYSPWTRRYG

SEQ ID NO: 278 - protein, 15 aa, Artificial Sequence
  Description of Artificial Sequence Synthetic peptide
RLWMRWYSPWTRRYG

SEQ ID NO: 279 - protein, 15 aa, Artificial Sequence
  Description of Artificial Sequence Synthetic peptide
RLWMRWYSPWTRRWG

SEQ ID NO: 280 - protein, 15 aa, Artificial Sequence
  Description of Artificial Sequence Synthetic peptide
ALWMRWYSPTTRRYG

SEQ ID NO: 281 - protein, 15 aa, Artificial Sequence
  Description of Artificial Sequence Synthetic peptide
RAWMRWYSPTTRRYG

SEQ ID NO: 282 - protein, 15 aa, Artificial Sequence
  Description of Artificial Sequence Synthetic peptide
RLWMRWYSPTTRRYG

SEQ ID NO: 283 - protein, 15 aa, Artificial Sequence
  Description of Artificial Sequence Synthetic peptide
RLWARWYSPTTRRYG

SEQ ID NO: 284 - protein, 15 aa, Artificial Sequence
  Description of Artificial Sequence Synthetic peptide
RLWMAWYSPTIRRYG

SEQ ID NO: 285 - protein, 15 aa, Artificial Sequence
  Description of Artificial Sequence Synthetic peptide
RLWMRWYAPTTRRYG

SEQ ID NO: 286 - protein, 15 aa, Artificial Sequence
  Description of Artificial Sequence Synthetic peptide
RLWMRWYAPTTRRYG

SEQ ID NO: 287 - protein, 15 aa, Artificial Sequence
  Description of Artificial Sequence Synthetic peptide
RLWMRWYAPTTRRYG

SEQ ID NO: 288 - protein, 15 aa, Artificial Sequence
  Description of Artificial Sequence Synthetic peptide
RLWMRWYSPATRRYG

SEQ ID NO: 289 - protein, 15 aa, Artificial Sequence
  Description of Artificial Sequence Synthetic peptide
RLWMRWYSPTARRYG

SEQ ID NO: 290 - protein, 15 aa, Artificial Sequence
  Description of Artificial Sequence Synthetic peptide
RLWMRWYSPTARRYG

SEQ ID NO: 291 - protein, 15 aa, Artificial Sequence
  Description of Artificial Sequence Synthetic peptide
RLWMRWYSPTTRAYG

SEQ ID NO: 292 - protein, 15 aa, Artificial Sequence
  Description of Artificial Sequence Synthetic peptide
RLWMRWYSPTTRRAG

SEQ ID NO: 293 - protein, 15 aa, Artificial Sequence
  Description of Artificial Sequence Synthetic peptide
RLWMRWYSPTTRRYA

SEQ ID NO: 294 - protein, 15 aa, Artificial Sequence
  Description of Artificial Sequence Synthetic peptide
RLIMRIYSPTTRRYG

SEQ ID NO: 295 - protein, 15 aa, Artificial Sequence
  Description of Artificial Sequence Synthetic peptide
RLIMRIYSPTTRRYG

SEQ ID NO: 296 - protein, 15 aa, Artificial Sequence
  Description of Artificial Sequence Synthetic peptide
RLIMRIYSPTTRRYG

SEQ ID NO: 297 - protein, 15 aa, Artificial Sequence
  Description of Artificial Sequence Synthetic peptide
RLVMRVYSPTTRRYG

SEQ ID NO: 298 - protein, 15 aa, Artificial Sequence
  Description of Artificial Sequence Synthetic peptide
RLYMRYYSPTTRRYG

SEQ ID NO: 299 - protein, 12 aa, Artificial Sequence
  Description of Artificial Sequence Synthetic peptide
YGRKKKRRQRRR

SEQ ID NO: 300 - protein, 18 aa, Artificial Sequence
  Description of Artificial Sequence Synthetic peptide
ALIILRRRIRKQAHAHSK

SEQ ID NO: 301 - protein, 18 aa, Artificial Sequence
  Description of Artificial Sequence Synthetic peptide
LAIILRRRIRKQAHAHSK

SEQ ID NO: 302 - protein, 18 aa, Artificial Sequence
  Description of Artificial Sequence Synthetic peptide
LLAILRRRIRKQAHAHSK

SEQ ID NO: 303 - protein, 18 aa, Artificial Sequence
  Description of Artificial Sequence Synthetic peptide
LLIALRRRIRKQAHAHSK

SEQ ID NO: 304 - protein, 18 aa, Artificial Sequence
  Description of Artificial Sequence Synthetic peptide
LLIIARRRIRKQAHAHSK

SEQ ID NO: 305 - protein, 18 aa, Artificial Sequence
  Description of Artificial Sequence Synthetic peptide
LLIILARRIRKQAHAHSK

SEQ ID NO: 306 - protein, 18 aa, Artificial Sequence
  Description of Artificial Sequence Synthetic peptide
LLIILRARIRKQAHAHSK

SEQ ID NO: 307 - protein, 18 aa, Artificial Sequence
  Description of Artificial Sequence Synthetic peptide
LLIILRRAIRKQAHAHSK

SEQ ID NO: 308 - protein, 18 aa, Artificial Sequence
  Description of Artificial Sequence Synthetic peptide
LLIILRRRARKQAHAHSK

SEQ ID NO: 309 - protein, 19 aa, Artificial Sequence
  Description of Artificial Sequence Synthetic peptide
LLIILRRRIARKQAHAHSK

SEQ ID NO: 310 - protein, 18 aa, Artificial Sequence
  Description of Artificial Sequence Synthetic peptide
LLIILRRRIRAQAHAHSK

SEQ ID NO: 311 - protein, 18 aa, Artificial Sequence
  Description of Artificial Sequence Synthetic peptide
LLIILRRRIRKAAHAHSK

SEQ ID NO: 312 - protein, 18 aa, Artificial Sequence
  Description of Artificial Sequence Synthetic peptide
  MOD_RES (13)..(13): D-amino acid
LLIILRRRIRKQAHAHSK

SEQ ID NO: 313 - protein, 18 aa, Artificial Sequence
  Description of Artificial Sequence Synthetic peptide
LLIILRRRIRKQAAAHSK

SEQ ID NO: 314 - protein, 18 aa, Artificial Sequence
  Description of Artificial Sequence Synthetic peptide
  MOD_RES (15)..(15): D-amino acid
LLIILRRRIRKQAHAHSK

SEQ ID NO: 315 - protein, 18 aa, Artificial Sequence
  Description of Artificial Sequence Synthetic peptide
LLIILRRRIRKQAHAASK

SEQ ID NO: 316 - protein, 18 aa, Artificial Sequence
  Description of Artificial Sequence Synthetic peptide
LLIILRRRIRKQAHAHAK

SEQ ID NO: 317 - protein, 18 aa, Artificial Sequence
  Description of Artificial Sequence Synthetic peptide
LLIILRRRIRKQAHAHSA

SEQ ID NO: 318 - protein, 18 aa, Artificial Sequence
  Description of Artificial Sequence Synthetic peptide
KSHAHAQKRIRRRLIILL

SEQ ID NO: 319 - protein, 18 aa, Artificial Sequence
  Description of Artificial Sequence Synthetic peptide
  MOD_RES (1)..(18): D-amino acid
LLIILRRRIRKQAHAHSK

SEQ ID NO: 320 - protein, 7 aa, Artificial Sequence
  Description of Artificial Sequence Synthetic peptide
RRIRPRP

SEQ ID NO: 321 - protein, 15 aa, Artificial Sequence
  Description of Artificial Sequence Synthetic peptide
RRIRPRPPRLPRPRP

SEQ ID NO: 322 - protein, 24 aa, Artificial Sequence
  Description of Artificial Sequence Synthetic peptide
RRIRPRPPRLPRPRPRPLPFPRPG

SEQ ID NO: 323 - protein, 17 aa, Artificial Sequence
  Description of Artificial Sequence Synthetic peptide
RRIRPRPPRLPRPRPRP

SEQ ID NO: 324 - protein, 20 aa, Artificial Sequence
  Description of Artificial Sequence Synthetic peptide
PRPPRLPRPRPRPLPFPRPG

SEQ ID NO: 325 - protein, 18 aa, Artificial Sequence
  Description of Artificial Sequence Synthetic peptide
PPRLPRPRPRPLPFPRPG

SEQ ID NO: 326 - protein, 16 aa, Artificial Sequence
  Description of Artificial Sequence Synthetic peptide
RLPRPRPRPLPFPRPG

SEQ ID NO: 327 - protein, 14 aa, Artificial Sequence
  Description of Artificial Sequence Synthetic peptide
PRPRPRPLPFPRPG

SEQ ID NO: 328 - protein, 12 aa, Artificial Sequence
  Description of Artificial Sequence Synthetic peptide
PRPRPLPFPRPG

SEQ ID NO: 329 - protein, 10 aa, Artificial Sequence
  Description of Artificial Sequence Synthetic peptide
PRPLPFPRPG

SEQ ID NO: 330 - protein, 22 aa, Artificial Sequence
  Description of Artificial Sequence Synthetic peptide
RVTSWLGRQLRIAGKRLEGRSK

SEQ ID NO: 331 - protein, 16 aa, Artificial Sequence
  Description of Artificial Sequence Synthetic peptide
GRQLRIAGKRLEGRSK

SEQ ID NO: 332 - protein, 23 aa, Artificial Sequence
  Description of Artificial Sequence Synthetic peptide
RRVTSWLGRQLRIAGKRLEGRSK

SEQ ID NO: 333 - protein, 22 aa, Artificial Sequence
  Description of Artificial Sequence Synthetic peptide
RVRSWLGRQLRIAGKRLEGRSK

SEQ ID NO: 334 - protein, 16 aa, Artificial Sequence
  Description of Artificial Sequence Synthetic peptide
GRQLRIAGKRLRGRSK

SEQ ID NO: 335 - protein, 16 aa, Artificial Sequence
  Description of Artificial Sequence Synthetic peptide
GRQLRIAGRRLRGRSR

SEQ ID NO: 336 - protein, 16 aa, Artificial Sequence
  Description of Artificial Sequence Synthetic peptide
GRQLRRAGRRLRGRSR

SEQ ID NO: 337 - protein, 16 aa, Artificial Sequence
  Description of Artificial Sequence Synthetic peptide
GRQLRIAGRRLRRRSR

SEQ ID NO: 338 - protein, 16 aa, Artificial Sequence
  Description of Artificial Sequence Synthetic peptide
GRQLRRAGRRLRRRSR

SEQ ID NO: 339 - protein, 15 aa, Artificial Sequence
  Description of Artificial Sequence Synthetic peptide
RQLRIAGRRLRGRSR

SEQ ID NO: 340 - protein, 16 aa, Artificial Sequence
  Description of Artificial Sequence Synthetic peptide
  MOD_RES (1)..(16): D-amino acid
RSRGRLRRGAIRLQRG

SEQ ID NO: 341 - protein, 29 aa, Artificial Sequence
  Description of Artificial Sequence Synthetic peptide
KLIKGRTPIKFGKADCDRPPKHSQNGMGK

SEQ ID NO: 342 - protein, 27 aa, Artificial Sequence
  Description of Artificial Sequence Synthetic peptide
KLIKGRTPIKFGKADCDRPPKHSQNGM

SEQ ID NO: 343 - protein, 27 aa, Artificial Sequence
  Description of Artificial Sequence Synthetic peptide
KLIKGRTPIKFGKADCDRPPKHSQNGK

SEQ ID NO: 344 - protein, 26 aa, Artificial Sequence
  Description of Artificial Sequence Synthetic peptide
KGRTPIKFGKADCDRPPKHSQNGMGK

SEQ ID NO: 345 - protein, 25 aa, Artificial Sequence
  Description of Artificial Sequence Synthetic peptide
KLIKGRTPIKFGKADCDRPPKHSGK

SEQ ID NO: 346 - protein, 25 aa, Artificial Sequence
  Description of Artificial Sequence Synthetic peptide
KLIKGRTPIKFGKARCRRPPKHSGK

SEQ ID NO: 347 - protein, 13 aa, Artificial Sequence
  Description of Artificial Sequence Synthetic peptide
KLIKGRTPIKFGK

SEQ ID NO: 348 - protein, 37 aa, Artificial Sequence
  Description of Artificial Sequence Synthetic polypeptide
KRIPNKKPGKKTTTKPTKKPTIKTTKKDLKPQTTKPK

SEQ ID NO: 349 - protein, 30 aa, Artificial Sequence
  Description of Artificial Sequence Synthetic polypeptide
KRIPNKKPGKKTTTKPTKKPTIKTTKKDLK

SEQ ID NO: 350 - protein, 27 aa, Artificial Sequence
  Description of Artificial Sequence Synthetic peptide
KRTPNKKPGKKTTTKPTKKPTIKTTKK

SEQ ID NO: 351 - protein, 23 aa, Artificial Sequence
  Description of Artificial Sequence Synthetic peptide
KRIPNKKPGKKTTTKPTKKPTIK

SEQ ID NO: 352 - protein, 19 aa, Artificial Sequence
  Description of Artificial Sequence Synthetic peptide
KRIPNKKPGKKTTTKPTKK

SEQ ID NO: 353 - protein, 12 aa, Artificial Sequence
  Description of Artificial Sequence Synthetic peptide
KRIPNKKPGKKT

SEQ ID NO: 354 - protein, 11 aa, Artificial Sequence
  Description of Artificial Sequence Synthetic peptide
KRIPNKKPGKK

SEQ ID NO: 355 - protein, 10 aa, Artificial Sequence
  Description of Artificial Sequence Synthetic peptide
KRIPNKKPKK

SEQ ID NO: 356 - protein, 22 aa, Artificial Sequence
  Description of Artificial Sequence Synthetic peptide
KKPGKKTTTKPTKKPTIKTTKK

SEQ ID NO: 357 - protein, 14 aa, Artificial Sequence
  Description of Artificial Sequence Synthetic peptide
KKPGKKTTTKPTKK

SEQ ID NO: 358 - protein, 10 aa, Artificial Sequence
  Description of Artificial Sequence Synthetic peptide
KKPTIKTTKK

SEQ ID NO: 359 - protein, 10 aa, Artificial Sequence
  Description of Artificial Sequence Synthetic peptide
KKTTTKPTKK

SEQ ID NO: 360 - protein, 15 aa, Artificial Sequence
  Description of Artificial Sequence Synthetic peptide
KSICKTIPSNKPKKK

SEQ ID NO: 361 - protein, 11 aa, Artificial Sequence
  Description of Artificial Sequence Synthetic peptide
KTIPSNKPKKK

SEQ ID NO: 362 - protein, 12 aa, Artificial Sequence
  Description of Artificial Sequence Synthetic peptide
KPRSKNPPKKPK

SEQ ID NO: 363 - protein, 17 aa, Artificial Sequence
  Description of Artificial Sequence Synthetic peptide
DRDDRDDRDDRDDRDDR

SEQ ID NO: 364 - protein, 14 aa, Artificial Sequence
  Description of Artificial Sequence Synthetic peptide
ERERERERERERER

SEQ ID NO: 365 - protein, 14 aa, Artificial Sequence
  Description of Artificial Sequence Synthetic peptide
WRWRWRWRWRWRWR

SEQ ID NO: 366 - protein, 10 aa, Artificial Sequence
  Description of Artificial Sequence Synthetic peptide
DRDRDRDRDR

SEQ ID NO: 367 - protein, 27 aa, Artificial Sequence
  Description of Artificial Sequence Synthetic peptide
GALFLGFLGAAGSTMGAWSQPKKKRKV

SEQ ID NO: 368 - protein, 17 aa, Artificial Sequence
  Description of Artificial Sequence Synthetic peptide
DRRRRGSRPSGAERRRR

SEQ ID NO: 369 - protein, 11 aa, Artificial Sequence
  Description of Artificial Sequence Synthetic peptide
NRARRNRRRVR

SEQ ID NO: 370 - protein, 10 aa, Artificial Sequence
  Description of Artificial Sequence Synthetic peptide
RTRRNRRRVR

SEQ ID NO: 371 - protein, 8 aa, Artificial Sequence
  Description of Artificial Sequence Synthetic peptide
RNRSRHRR

SEQ ID NO: 372 - protein, 22 aa, Artificial Sequence
  Description of Artificial Sequence Synthetic peptide
MVRRFLVTLRIRRACGPPRVRV

SEQ ID NO: 373 - protein, 22 aa, Artificial Sequence
  Description of Artificial Sequence Synthetic peptide
FVTRGCPRRLVARLIRVMVPRR

SEQ ID NO: 374 - protein, 13 aa, Artificial Sequence
  Description of Artificial Sequence Synthetic peptide
VRRFLVTLRIRRA

SEQ ID NO: 375 - protein, 13 aa, Artificial Sequence
  Description of Artificial Sequence Synthetic peptide
RVRILARFLRTRV

SEQ ID NO: 376 - protein, 14 aa, Artificial Sequence
  Description of Artificial Sequence Synthetic peptide
RVRVIEVVHIPRLT

SEQ ID NO: 377 - protein, 13 aa, Artificial Sequence
  Description of Artificial Sequence Synthetic peptide
VIRVHFRLPVRTV

SEQ ID NO: 378 - protein, 37 aa, Artificial Sequence
  Description of Artificial Sequence Synthetic polypeptide
MVRRELVTLRIRRACGPPRVRVFVVHIPRLTGEWAAP

SEQ ID NO: 379 - protein, 37 aa, Artificial Sequence
  Description of Artificial Sequence Synthetic polypeptide
FRVPLRIRPCVVAPRLVMVRHTFGRIARWVAGPLETR

SEQ ID NO: 380 - protein, 21 aa, Artificial Sequence
  Description of Artificial Sequence Synthetic peptide
AGYLLGKINLKALAALAKKIL

SEQ ID NO: 381 - protein, 25 aa, Artificial Sequence
  Description of Artificial Sequence Synthetic peptide
GTKMIFVGIKKKEERADLIAYLKKA

SEQ ID NO: 382 - protein, 16 aa, Artificial Sequence
  Description of Artificial Sequence Synthetic peptide
KKKEERADLIAYLKKA

SEQ ID NO: 383 - protein, 14 aa, Artificial Sequence
  Description of Artificial Sequence Synthetic peptide
KMIFVGIKKKEERA

SEQ ID NO: 384 - protein, 10 aa, Artificial Sequence
  Description of Artificial Sequence Synthetic peptide
KMIFVGIKKK

SEQ ID NO: 385 - protein, 10 aa, Artificial Sequence
  Description of Artificial Sequence Synthetic peptide
EKGKKIFIMK

SEQ ID NO: 386 - protein, 9 aa, Artificial Sequence
  Description of Artificial Sequence Synthetic peptide
KGKKIFIMK

SEQ ID NO: 387 - protein, 17 aa, Artificial Sequence
  Description of Artificial Sequence Synthetic peptide
RRRRNRTRRNRRRVRGC

SEQ ID NO: 388 - protein, 15 aa, Artificial Sequence
  Description of Artificial Sequence Synthetic peptide
TRRQRTRRARRNRGC

SEQ ID NO: 389 - protein, 21 aa, Artificial Sequence
  Description of Artificial Sequence Synthetic peptide
KMTRAQRRAAARRNRWTARGC

SEQ ID NO: 390 - protein, 21 aa, Artificial Sequence
  Description of Artificial Sequence Synthetic peptide
KLTRAQRRAAARKNKRNTRGC

SEQ ID NO: 391 - protein, 19 aa, Artificial Sequence
  Description of Artificial Sequence Synthetic peptide
NAKTRRHERRRKLAIERGC

SEQ ID NO: 392 - protein, 24 aa, Artificial Sequence
  Description of Artificial Sequence Synthetic peptide
MDAQTRRRERRAEKQAQWKAANGC

SEQ ID NO: 393 - protein, 20 aa, Artificial Sequence
  Description of Artificial Sequence Synthetic peptide
TAKTRYKARRAELIAERRGC

SEQ ID NO: 394 - protein, 14 aa, Artificial Sequence
  Description of Artificial Sequence Synthetic peptide
SQMTRQARRLYBGC

SEQ ID NO: 395 - protein, 28 aa, Artificial Sequence
  Description of Artificial Sequence Synthetic peptide
KRRIRRERNKMAAAKSRNRRRELTDTGC

SEQ ID NO: 396 - protein, 30 aa, Artificial Sequence
  Description of Artificial Sequence Synthetic polypeptide
RIKAERKRMRNRIAASKSRKRKLERIARGC

SEQ ID NO: 397 - protein, 24 aa, Artificial Sequence
  Description of Artificial Sequence Synthetic peptide
KRARNTEAARRSRARKLQRMKQGC

SEQ ID NO: 398 - protein, 22 aa, Artificial Sequence
  Description of Artificial Sequence Synthetic peptide
KCFQWQRNMRKVRGPPVSCIKR

SEQ ID NO: 399 - protein, 19 aa, Artificial Sequence
  Description of Artificial Sequence Synthetic peptide
KCFQWQRNMRKVRGPPVSC

SEQ ID NO: 400 - protein, 22 aa, Artificial Sequence
  Description of Artificial Sequence Synthetic peptide
KCFQWQRNMRKVRGPPVSSIKR

SEQ ID NO: 401 - protein, 13 aa, Artificial Sequence
  Description of Artificial Sequence Synthetic peptide
KCFQWQRNMRKVR

SEQ ID NO: 402 - protein, 16 aa, Artificial Sequence
  Description of Artificial Sequence Synthetic peptide
FQWQRNMRKVRGPPVS

SEQ ID NO: 403 - protein, 19 aa, Artificial Sequence
  Description of Artificial Sequence Synthetic peptide
QWQRNMRKVRGPPVSCIKR

SEQ ID NO: 404 - protein, 10 aa, Artificial Sequence
  Description of Artificial Sequence Synthetic peptide
QWQRNMRKVR

SEQ ID NO: 405 - protein, 22 aa, Artificial Sequence
  Description of Artificial Sequence Synthetic peptide
KCFMWQEMLNKAGVPKLRCARK

SEQ ID NO: 406 - protein, 15 aa, Artificial Sequence
  Description of Artificial Sequence Synthetic peptide
KWFETWFTEWPKKRK

SEQ ID NO: 407 - protein, 20 aa, Artificial Sequence
  Description of Artificial Sequence Synthetic peptide
GLWRALWRLLRSLWRLLWRA

SEQ ID NO: 408 - protein, 20 aa, Artificial Sequence
  Description of Artificial Sequence Synthetic peptide
GLWWRLWWRLRSWFRLWFRA

SEQ ID NO: 409 - protein, 34 aa, Artificial Sequence
  Description of Artificial Sequence Synthetic polypeptide
DAATATRGRSAASRPTQRPRAPARSASRPRRPVE

SEQ ID NO: 410 - protein, 27 aa, Artificial Sequence
  Description of Artificial Sequence Synthetic peptide
GALFLGFLGAAGSTMGAWSQPKKKRKV

SEQ ID NO: 411 - protein, 27 aa, Artificial Sequence
  Description of Artificial Sequence Synthetic peptide
GALFLGFLGAAGSTMGAWSQPKSKRKV

SEQ ID NO: 412 - protein, 34 aa, Artificial Sequence
  Description of Artificial Sequence Synthetic polypeptide
AKVKDEPQRRSARLSAKPAPPKPEPKPKKAPAKK

SEQ ID NO: 413 - protein, 34 aa, Artificial Sequence
  Description of Artificial Sequence Synthetic polypeptide
  MOD_RES (1)..(34): D-amino acid
AKYKDEPGRRSARLSAKPAPPKPEPKPKKAPAKK

SEQ ID NO: 414 - protein, 12 aa, Artificial Sequence
  Description of Artificial Sequence Synthetic peptide
PSSSSSSRIGDP

SEQ ID NO: 415 - protein, 18 aa, Artificial Sequence
  Description of Artificial Sequence Synthetic peptide
  MOD_RES (1)..(18): D-amino acid
VRLPPPYRIPPPVRLPPP

SEQ ID NO: 416 - protein, 18 aa, Artificial Sequence
  Description of Artificial Sequence Synthetic peptide
VELPPPVELPPPVELPPP

SEQ ID NO: 417 - protein, 28 aa, Artificial Sequence
  Description of Artificial Sequence Synthetic peptide
ALWMTLLKKVLKAAAKAALNAVLVGANA

SEQ ID NO: 418 - protein, 13 aa, Artificial Sequence
  Description of Artificial Sequence Synthetic peptide
ALWKTLLKKVLKA

SEQ ID NO: 419 - protein, 20 aa, Artificial Sequence
  Description of Artificial Sequence Synthetic peptide
ALWKTLLKKVLKAPKKKRKV

SEQ ID NO: 420 - protein, 20 aa, Artificial Sequence
  Description of Artificial Sequence Synthetic peptide
PKKKRKVALWKTLLKKVLKA

SEQ ID NO: 421 - protein, 20 aa, Artificial Sequence
  Description of Artificial Sequence Synthetic peptide
VKRKKKPALWKTLLKKVLKA

SEQ ID NO: 422 - protein, 22 aa, Artificial Sequence
  Description of Artificial Sequence Synthetic peptide
RQARRNRRRALWKTLLKKVLKA

SEQ ID NO: 423 - protein, 10 aa, Artificial Sequence
  Description of Artificial Sequence Synthetic peptide
RQARRNRRRC

SEQ ID NO: 424 - protein, 13 aa, Artificial Sequence
  Description of Artificial Sequence Synthetic peptide
EEEAAGRKRKKRT

SEQ ID NO: 425 - protein, 3 aa, Artificial Sequence
  Description of Artificial Sequence Synthetic peptide
EEE

SEQ ID NO: 426 - protein, 5 aa, Artificial Sequence
  Description of Artificial Sequence Synthetic peptide
EEEAA

SEQ ID NO: 427 - protein, 8 aa, Artificial Sequence
  Description of Artificial Sequence Synthetic peptide
EEEAAKKK

SEQ ID NO: 428 - protein, 13 aa, Artificial Sequence
  Description of Artificial Sequence Synthetic peptide
FFFAAGRKRKKRT

SEQ ID NO: 429 - protein, 10 aa, Artificial Sequence
  Description of Artificial Sequence Synthetic peptide
AAGRKRKKRT

SEQ ID NO: 430 - protein, 13 aa, Artificial Sequence
  Description of Artificial Sequence Synthetic peptide
YYYAAGRKRKKRT

SEQ ID NO: 431 - protein, 22 aa, Artificial Sequence
  Description of Artificial Sequence Synthetic peptide
MVTVLFRRLRIRRACGPPRVRV

SEQ ID NO: 432 - protein, 21 aa, Artificial Sequence
  Description of Artificial Sequence Synthetic peptide
AGYLLGKINLKALAALAKKIL

SEQ ID NO: 433 - protein, 11 aa, Artificial Sequence
  Description of Artificial Sequence Synthetic peptide
GKKKKRKREKL

SEQ ID NO: 434 - protein, 8 aa, Artificial Sequence
  Description of Artificial Sequence Synthetic peptide
ERKKRRRE

SEQ ID NO: 435 - protein, 7 aa, Artificial Sequence
  Description of Artificial Sequence Synthetic peptide
FKKFRKF

SEQ ID NO: 436 - protein, 21 aa, Artificial Sequence
  Description of Artificial Sequence Synthetic peptide
YTQDFNKFHTFPQTAIGVGAP

SEQ ID NO: 437 - protein, 18 aa, Artificial Sequence
  Description of Artificial Sequence Synthetic peptide
DFNKFHTFPQTAIGVGAP

SEQ ID NO: 438 - protein, 15 aa, Artificial Sequence
  Description of Artificial Sequence Synthetic peptide
KFHTFPQTAIGVGAP

SEQ ID NO: 439 - protein, 12 aa, Artificial Sequence
  Description of Artificial Sequence Synthetic peptide
TFPQTAIGVGAP

SEQ ID NO: 440 - protein, 13 aa, Artificial Sequence
  Description of Artificial Sequence Synthetic peptide
GYGRKKRRQRRRG

SEQ ID NO: 441 - protein, 15 aa, Artificial Sequence
  Description of Artificial Sequence Synthetic peptide
FLGKKFKKYFLQLLK

SEQ ID NO: 442 - protein, 23 aa, Artificial Sequence
  Description of Artificial Sequence Synthetic peptide
FLIFIRVICIVIAKLKANLMCKT

SEQ ID NO: 443 - protein, 17 aa, Artificial Sequence
  Description of Artificial Sequence Synthetic peptide
KKAAQIRSQVMTHLRVI

SEQ ID NO: 444 - protein, 16 aa, Artificial Sequence
  Description of Artificial Sequence Synthetic peptide
YIVLRRRRKRVNTKRS

SEQ ID NO: 445 - protein, 11 aa, Artificial Sequence
  Description of Artificial Sequence Synthetic peptide
RRKLSQQKEKK

SEQ ID NO: 446 - protein, 15 aa, Artificial Sequence
  Description of Artificial Sequence Synthetic peptide
VQAILRRNWNQYKIQ

SEQ ID NO: 447 - protein, 16 aa, Artificial Sequence
  Description of Artificial Sequence Synthetic peptide
KTVLLRKLLKLLVRKI

SEQ ID NO: 448 - protein, 16 aa, Artificial Sequence
  Description of Artificial Sequence Synthetic peptide
LLKKRKVVRLIKFLLK

SEQ ID NO: 449 - protein, 19 aa, Artificial Sequence
  Description of Artificial Sequence Synthetic peptide
KLPCRSNTFLNIFRRKKPG

SEQ ID NO: 450 - protein, 16 aa, Artificial Sequence
  Description of Artificial Sequence Synthetic peptide
KKICTRKPRFMSAWAQ

SEQ ID NO: 451 - protein, 18 aa, Artificial Sequence
  Description of Artificial Sequence Synthetic peptide
  MOD_RES (1)..(18): D-amino acid
RGGRLSYSRRRFSTSTGR

SEQ ID NO: 452 - protein, 10 aa, Artificial Sequence
  Description of Artificial Sequence Synthetic peptide
  MOD_RES (1)..(10): D-amino acid
RRLSYSRRRF

SEQ ID NO: 453 - protein, 17 aa, Artificial Sequence
  Description of Artificial Sequence Synthetic peptide
RGGRLAYLRRRWAVLGR

SEQ ID NO: 454 - protein, 28 aa, Artificial Sequence
  Description of Artificial Sequence Synthetic peptide
MANLGCWMLVLEVATWSDLGLCKKRPKP

SEQ ID NO: 455 - protein, 30 aa, Artificial Sequence
  Description of Artificial Sequence Synthetic polypeptide
MVKSKIGSWILVLFVAMWSDVGLCKKRPKP

SEQ ID NO: 456 - protein, 38 aa, Artificial Sequence
  Description of Artificial Sequence Synthetic polypeptide
LLGDFFRKSKEKIGKEFKRIVQRIKDFLRNLVPRTESC

SEQ ID NO: 457 - protein, 23 aa, Artificial Sequence
  Description of Artificial Sequence Synthetic peptide
GIGKFLHSAKKWGKAFVGQIMNC

SEQ ID NO: 458 - protein, 24 aa, Artificial Sequence
  Description of Artificial Sequence Synthetic peptide
TRSSRAGLQWPVGRVHRLLRKGGC

SEQ ID NO: 459 - protein, 11 aa, Artificial Sequence
  Description of Artificial Sequence Synthetic peptide
YGRKKRRQRRR

SEQ ID NO: 460 - protein, 61 aa, Artificial Sequence
  Description of Artificial Sequence Synthetic polypeptide
EKRPRTAFSSEQLARLKREFNENRYLTTERRRQQLSSELGLNEAQIKIWFQNKRAKIKKST

SEQ ID NO: 461 - protein, 13 aa, Artificial Sequence
  Description of Artificial Sequence Synthetic peptide
GRRRRRRRRRPPQ

SEQ ID NO: 462 - protein, 27 aa, Artificial Sequence
  Description of Artificial Sequence Synthetic peptide
GALFLGFLGAAGSTMGAWSQPKKKRKV

SEQ ID NO: 463 - protein, 27 aa, Artificial Sequence
  Description of Artificial Sequence Synthetic peptide
GALFLAFLAAALSLMGLWSQPKKKRRV

SEQ ID NO: 464 - protein, 10 aa, Artificial Sequence
  Description of Artificial Sequence Synthetic peptide
MLLLTRRRST

SEQ ID NO: 465 - protein, 9 aa, Artificial Sequence
  Description of Artificial Sequence Synthetic peptide
CGNKRTRGC

SEQ ID NO: 466 - protein, 12 aa, Artificial Sequence
  Description of Artificial Sequence Synthetic peptide
TSPLNIHNGQKL

SEQ ID NO: 467 - protein, 16 aa, Artificial Sequence
  Description of Artificial Sequence Synthetic peptide
GLRKRLRKFRNKIKEK

SEQ ID NO: 468 - protein, 27 aa, Artificial Sequence
  Description of Artificial Sequence Synthetic peptide
GLLEALAELLEGLRKRLRKFRNKIKEK

SEQ ID NO: 469 - protein, 13 aa, Artificial Sequence
  Description of Artificial Sequence Synthetic peptide
CVQWSLLRGYQPC

SEQ ID NO: 470 - protein, 16 aa, Artificial Sequence
  Description of Artificial Sequence Synthetic peptide
RQIKIFFQNRRMKWKK

SEQ ID NO: 471 - protein, 21 aa, Artificial Sequence
  Description of Artificial Sequence Synthetic peptide
ASMWERVKSIIKSSLAAASNI

SEQ ID NO: 472 - protein, 21 aa, Artificial Sequence
  Description of Artificial Sequence Synthetic peptide
ASMWERVKSIIKSSLAAASNI

SEQ ID NO: 473 - protein, 26 aa, Artificial Sequence
  Description of Artificial Sequence Synthetic peptide
DPKGDPKGVTVTVTVIVTGKGDPKPD

SEQ ID NO: 474 - protein, 16 aa, Artificial Sequence
  Description of Artificial Sequence Synthetic peptide
CSIPPEVKFNPFVYLI

SEQ ID NO: 475 - protein, 16 aa, Artificial Sequence
  Description of Artificial Sequence Synthetic peptide
  MOD_RES (1)..(16): D-amino acid
CSIPPEVKFNPFVYLI

SEQ ID NO: 476 - protein, 6 aa, Artificial Sequence
  Description of Artificial Sequence Synthetic peptide
PFVYLI

SEQ ID NO: 477 - protein, 8 aa, Artificial Sequence
  Description of Artificial Sequence Synthetic peptide
NKPILVFY

SEQ ID NO: 478 - protein, 14 aa, Artificial Sequence
  Description of Artificial Sequence Synthetic peptide
YKQCHKKGGKKGSG

SEQ ID NO: 479 - protein, 15 aa, Artificial Sequence
  Description of Artificial Sequence Synthetic peptide
  MOD_RES (10)..(10): Any amino acid
YKQCHKKGGXKKGSG

SEQ ID NO: 480 - protein, 14 aa, Artificial Sequence
  Description of Artificial Sequence Synthetic peptide
GSGKKGGKKHCQKY

SEQ ID NO: 481 - protein, 14 aa, Artificial Sequence
  Description of Artificial Sequence Synthetic peptide
GSGKKGGKKICQKY

SEQ ID NO: 482 - protein, 16 aa, Artificial Sequence
  Description of Artificial Sequence Synthetic peptide
YTAIAWVKAFIRKLRK

SEQ ID NO: 483 - protein, 17 aa, Artificial Sequence
  Description of Artificial Sequence Synthetic peptide
IAWVKAFIRKLRKGPLG

SEQ ID NO: 484 - protein, 23 aa, Artificial Sequence
  Description of Artificial Sequence Synthetic peptide
LIRLWSHLIHIWFQNRRLKWKKK

SEQ ID NO: 485 - protein, 34 aa, Artificial Sequence
  Description of Artificial Sequence Synthetic polypeptide
KKKKKKGGFLGEWRGENGRKTRSAYERMCILKGK

SEQ ID NO: 486 - protein, 14 aa, Artificial Sequence
  Description of Artificial Sequence Synthetic peptide
RLSGMNEVLSFRWL

SEQ ID NO: 487 - protein, 13 aa, Artificial Sequence
  Description of Artificial Sequence Synthetic peptide
GPFHEYQFLEPPV

SEQ ID NO: 488 - protein, 13 aa, Artificial Sequence
  Description of Artificial Sequence Synthetic peptide
GSPWGLQHHPPRT

SEQ ID NO: 489 - protein, 41 aa, Artificial Sequence
  Description of Artificial Sequence Synthetic polypeptide
AAVALLPAVLLALLAPEILLPNNYNAYESYKYPGMFIALSK

SEQ ID NO: 490 - protein, 26 aa, Artificial Sequence
  Description of Artificial Sequence Synthetic peptide
AAVALLPAVLLALLAPVQRKRQKLMP

SEQ ID NO: 491 - protein, 23 aa, Artificial Sequence
  Description of Artificial Sequence Synthetic peptide
MGLGLHLLVLAAALQGAKKKRKV

SEQ ID NO: 492 - protein, 30 aa, Artificial Sequence
  Description of Artificial Sequence Synthetic polypeptide
WEAALAEALAEALAEHLAEALAEALEALAA

SEQ ID NO: 493 - protein, 20 aa, Artificial Sequence
  Description of Artificial Sequence Synthetic peptide
GLFEALLELLESLWELLLEA

SEQ ID NO: 494 - protein, 20 aa, Artificial Sequence
  Description of Artificial Sequence Synthetic peptide
GLFKALLKLLKSLWKLLLKA

SEQ ID NO: 495 - protein, 20 aa, Artificial Sequence
  Description of Artificial Sequence Synthetic peptide
GLFRALLRLLRSLWRLLLRA

SEQ ID NO: 496 - protein, 23 aa, Artificial Sequence
  Description of Artificial Sequence Synthetic peptide
CGAYDLRRRERQSRLRRRERQSR

SEQ ID NO: 497 - protein, 17 aa, Artificial Sequence
  Description of Artificial Sequence Synthetic peptide
RKKRRRESRKKRRRESC

SEQ ID NO: 498 - protein, 19 aa, Artificial Sequence
  Description of Artificial Sequence Synthetic peptide
CVKRGLKLRHVRPRVTRDV

SEQ ID NO: 499 - protein, 17 aa, Artificial Sequence
  Description of Artificial Sequence Synthetic peptide
CRQIKIWFQNRRMKWKK

SEQ ID NO: 500 - protein, 15 aa, Artificial Sequence
  Description of Artificial Sequence Synthetic peptide
PPKKSAQCLRYKKPE

SEQ ID NO: 501 - protein, 15 aa, Artificial Sequence
  Description of Artificial Sequence Synthetic peptide
DPVDTPNPTRRKPGK

SEQ ID NO: 502 - protein, 13 aa, Artificial Sequence
  Description of Artificial Sequence Synthetic peptide
KRVSRNKSEKKRR

SEQ ID NO: 503 - protein, 16 aa, Artificial Sequence
  Description of Artificial Sequence Synthetic peptide
GRRHHCRSKAKRSRHH

SEQ ID NO: 504 - protein, 16 aa, Artificial Sequence
  Description of Artificial Sequence Synthetic peptide
SARHHCRSKAKRSRHH

SEQ ID NO: 505 - protein, 16 aa, Artificial Sequence
  Description of Artificial Sequence Synthetic peptide
SRAHHCRSKAKRSRHH

SEQ ID NO: 506 - protein, 16 aa, Artificial Sequence
  Description of Artificial Sequence Synthetic peptide
SRRAHCRSKAKRSRHH

SEQ ID NO: 507 - protein, 16 aa, Artificial Sequence
  Description of Artificial Sequence Synthetic peptide
SRRHACRSKAKRSRHH

SEQ ID NO: 508 - protein, 16 aa, Artificial Sequence
  Description of Artificial Sequence Synthetic peptide
SRRHHARSKAKRSRHH

SEQ ID NO: 509 - protein, 16 aa, Artificial Sequence
  Description of Artificial Sequence Synthetic peptide
SRRHHCRAKAKRSRHH

SEQ ID NO: 510 - protein, 16 aa, Artificial Sequence
  Description of Artificial Sequence Synthetic peptide
SRRHHCRSAAKRSRHH

SEQ ID NO: 511 - protein, 16 aa, Artificial Sequence
  Description of Artificial Sequence Synthetic peptide
SRRHHCRSKAARSRRH

SEQ ID NO: 512 - protein, 16 aa, Artificial Sequence
  Description of Artificial Sequence Synthetic peptide
SRRHHCRSKAKASRHH

SEQ ID NO: 513 - protein, 16 aa, Artificial Sequence
  Description of Artificial Sequence Synthetic peptide
SRRHHCRSKAKRARHH

SEQ ID NO: 514 - protein, 16 aa, Artificial Sequence
  Description of Artificial Sequence Synthetic peptide
SRRHHCRSKAKRSAHH

SEQ ID NO: 515 - protein, 13 aa, Artificial Sequence
  Description of Artificial Sequence Synthetic peptide
RRHHCRSKAKRSR

SEQ ID NO: 516 - protein, 12 aa, Artificial Sequence
  Description of Artificial Sequence Synthetic peptide
GRKGKHKRKKLP

SEQ ID NO: 517 - protein, 10 aa, Artificial Sequence
  Description of Artificial Sequence Synthetic peptide
GKKKKKKKKK

SEQ ID NO: 518 - protein, 18 aa, Artificial Sequence
  Description of Artificial Sequence Synthetic peptide
GKRVAKRKLIEQNRERRR

SEQ ID NO: 519 - protein, 18 aa, Artificial Sequence
  Description of Artificial Sequence Synthetic peptide
GRKLKKKKNEKEDKRPRT

SEQ ID NO: 520 - protein, 17 aa, Artificial Sequence
  Description of Artificial Sequence Synthetic peptide
GKKTNLFSALIKKKKTA

SEQ ID NO: 521 - protein, 18 aa, Artificial Sequence
  Description of Artificial Sequence Synthetic peptide
GRRERNKMAAAKCRNRRR

SEQ ID NO: 522 - protein, 18 aa, Artificial Sequence
  Description of Artificial Sequence Synthetic peptide
GKRARNTEAARRSRARKL

SEQ ID NO: 523 - protein, 14 aa, Artificial Sequence
  Description of Artificial Sequence Synthetic peptide
GRRRRATAKYRTAH

SEQ ID NO: 524 - protein, 15 aa, Artificial Sequence
  Description of Artificial Sequence Synthetic peptide
GKRRRRATAKYRSAH

SEQ ID NO: 525 - protein, 12 aa, Artificial Sequence
  Description of Artificial Sequence Synthetic peptide
GRRRRKRLSHRT

SEQ ID NO: 526 - protein, 10 aa, Artificial Sequence
  Description of Artificial Sequence Synthetic peptide
GRRRRRERNK

SEQ ID NO: 527 - protein, 16 aa, Artificial Sequence
  Description of Artificial Sequence Synthetic peptide
GKHRHERGHHRDRRER

SEQ ID NO: 528 - protein, 17 aa, Artificial Sequence
  Description of Artificial Sequence Synthetic peptide
GKKKRKLSNRESAKRSR

SEQ ID NO: 529 - protein, 10 aa, Artificial Sequence
  Description of Artificial Sequence Synthetic peptide
MIIYRDLISH

SEQ ID NO: 530 - protein, 9 aa, Artificial Sequence
  Description of Artificial Sequence Synthetic peptide
MIIYRDLIS

SEQ ID NO: 531 - protein, 8 aa, Artificial Sequence
  Description of Artificial Sequence Synthetic peptide
MIIYRDLI

SEQ ID NO: 532 - protein, 9 aa, Artificial Sequence
  Description of Artificial Sequence Synthetic peptide
IIYRDLISH

SEQ ID NO: 533 - protein, 7 aa, Artificial Sequence
  Description of Artificial Sequence Synthetic peptide
MIIYRDL

SEQ ID NO: 534 - protein, 6 aa, Artificial Sequence
  Description of Artificial Sequence Synthetic peptide
MIIYRD

SEQ ID NO: 535 - protein, 8 aa, Artificial Sequence
  Description of Artificial Sequence Synthetic peptide
IYRDLISH

SEQ ID NO: 536 - protein, 9 aa, Artificial Sequence
  Description of Artificial Sequence Synthetic peptide
AIIYRDLIS

SEQ ID NO: 537 - protein, 9 aa, Artificial Sequence
  Description of Artificial Sequence Synthetic peptide
MAIYRDLIS

SEQ ID NO: 538 - protein, 9 aa, Artificial Sequence
  Description of Artificial Sequence Synthetic peptide
MIAYRDLIS

SEQ ID NO: 539 - protein, 9 aa, Artificial Sequence
  Description of Artificial Sequence Synthetic peptide
MIIARDLIS

SEQ ID NO: 540 - protein, 9 aa, Artificial Sequence
  Description of Artificial Sequence Synthetic peptide
MIIYADLIS

SEQ ID NO: 541 - protein, 9 aa, Artificial Sequence
  Description of Artificial Sequence Synthetic peptide
MIIYRDLIS

SEQ ID NO: 542 - protein, 9 aa, Artificial Sequence
  Description of Artificial Sequence Synthetic peptide
MIIYRDAIS

SEQ ID NO: 543 - protein, 9 aa, Artificial Sequence
  Description of Artificial Sequence Synthetic peptide
MIIYRDLAS

SEQ ID NO: 544 - protein, 9 aa, Artificial Sequence
  Description of Artificial Sequence Synthetic peptide
MIIYRDLIA

SEQ ID NO: 545 - protein, 11 aa, Artificial Sequence
  Description of Artificial Sequence Synthetic peptide
MIIYRDLISKK

SEQ ID NO: 546 - protein, 10 aa, Artificial Sequence
  Description of Artificial Sequence Synthetic peptide
MITYRDKKSH

SEQ ID NO: 547 - protein, 10 aa, Artificial Sequence
  Description of Artificial Sequence Synthetic peptide
MIIFRDLISH

SEQ ID NO: 548 - protein, 10 aa, Artificial Sequence
  Description of Artificial Sequence Synthetic peptide
MIISRDLISH

SEQ ID NO: 549 - protein, 10 aa, Artificial Sequence
  Description of Artificial Sequence Synthetic peptide
QIISRDLISH

SEQ ID NO: 550 - protein, 10 aa, Artificial Sequence
  Description of Artificial Sequence Synthetic peptide
CIISRDLISH

SEQ ID NO: 551 - protein, 12 aa, Artificial Sequence
  Description of Artificial Sequence Synthetic peptide
MIIYRALISHKK

SEQ ID NO: 552 - protein, 12 aa, Artificial Sequence
  Description of Artificial Sequence Synthetic peptide
MIIYRIAASHKK

SEQ ID NO: 553 - protein, 10 aa, Artificial Sequence
  Description of Artificial Sequence Synthetic peptide
MIIRRDLISE

SEQ ID NO: 554 - protein, 10 aa, Artificial Sequence
  Description of Artificial Sequence Synthetic peptide
MIIYRAEISH

SEQ ID NO: 555 - protein, 10 aa, Artificial Sequence
  Description of Artificial Sequence Synthetic peptide
MIIYARRAEE

SEQ ID NO: 556 - protein, 12 aa, Artificial Sequence
  Description of Artificial Sequence Synthetic peptide
MIIFRIAASHKK

SEQ ID NO: 557 - protein, 12 aa, Artificial Sequence
  Description of Artificial Sequence Synthetic peptide
MIIFRALISHKK

SEQ ID NO: 558 - protein, 12 aa, Artificial Sequence
  Description of Artificial Sequence Synthetic peptide
MIIFRAAASHKK

SEQ ID NO: 559 - protein, 12 aa, Artificial Sequence
  Description of Artificial Sequence Synthetic peptide
FIIFRIAASHKK

SEQ ID NO: 560 - protein, 12 aa, Artificial Sequence
  Description of Artificial Sequence Synthetic peptide
LIIFRIAASHKK

SEQ ID NO: 561 - protein, 12 aa, Artificial Sequence
  Description of Artificial Sequence Synthetic peptide
WIIFRIAASHKK

SEQ ID NO: 562 - protein, 12 aa, Artificial Sequence
  Description of Artificial Sequence Synthetic peptide
WIIFRAAASHKK

SEQ ID NO: 563 - protein, 12 aa, Artificial Sequence
  Description of Artificial Sequence Synthetic peptide
WIIFRALISHKK

SEQ ID NO: 564 - protein, 12 aa, Artificial Sequence
  Description of Artificial Sequence Synthetic peptide
MIIFRIAAYHKK

SEQ ID NO: 565 - protein, 12 aa, Artificial Sequence
  Description of Artificial Sequence Synthetic peptide
WIIFRIAAYHKK

SEQ ID NO: 566 - protein, 12 aa, Artificial Sequence
  Description of Artificial Sequence Synthetic peptide
MIIFRIAATHKK

SEQ ID NO: 567 - protein, 12 aa, Artificial Sequence
  Description of Artificial Sequence Synthetic peptide
WIIFRIAATHKK

SEQ ID NO: 568 - protein, 12 aa, Artificial Sequence
  Description of Artificial Sequence Synthetic peptide
MIIFKIAASHKK

SEQ ID NO: 569 - protein, 12 aa, Artificial Sequence
  Description of Artificial Sequence Synthetic peptide
WIIFKIAASHKK

SEQ ID NO: 570 - protein, 12 aa, Artificial Sequence
  Description of Artificial Sequence Synthetic peptide
MIIFRIAASHKK

SEQ ID NO: 571 - protein, 12 aa, Artificial Sequence
  Description of Artificial Sequence Synthetic peptide
LIIFRILISHKK

SEQ ID NO: 572 - protein, 12 aa, Artificial Sequence
  Description of Artificial Sequence Synthetic peptide
MIIFRILISHKK

SEQ ID NO: 573 - protein, 12 aa, Artificial Sequence
  Description of Artificial Sequence Synthetic peptide
LIIFRILISHRR

SEQ ID NO: 574 - protein, 12 aa, Artificial Sequence
  Description of Artificial Sequence Synthetic peptide
LIIFRILISHHH

SEQ ID NO: 575 - protein, 11 aa, Artificial Sequence
  Description of Artificial Sequence Synthetic peptide
LIIFRILISHK

SEQ ID NO: 576 - protein, 11 aa, Artificial Sequence
  Description of Artificial Sequence Synthetic peptide
LIIFRILISHR

SEQ ID NO: 577 - protein, 10 aa, Artificial Sequence
  Description of Artificial Sequence Synthetic peptide
LIIFRILISH

SEQ ID NO: 578 - protein, 12 aa, Artificial Sequence
  Description of Artificial Sequence Synthetic peptide
LIIFAIAASHKK

SEQ ID NO: 579 - protein, 12 aa, Artificial Sequence
  Description of Artificial Sequence Synthetic peptide
LIIFAILISHKK

SEQ ID NO: 580 - protein, 20 aa, Artificial Sequence
  Description of Artificial Sequence Synthetic peptide
RILQQLLFIHFRIGCRHSRI

SEQ ID NO: 581 - protein, 17 aa, Artificial Sequence
  Description of Artificial Sequence Synthetic peptide
RILQQLLFIHFRIGCRH

SEQ ID NO: 582 - protein, 15 aa, Artificial Sequence
  Description of Artificial Sequence Synthetic peptide
RILQQLLFIHFRIGC

SEQ ID NO: 583 - protein, 10 aa, Artificial Sequence
  Description of Artificial Sequence Synthetic peptide
RIFIHFRIGC

SEQ ID NO: 584 - protein, 8 aa, Artificial Sequence
  Description of Artificial Sequence Synthetic peptide
RIFIRIGC

SEQ ID NO: 585 - protein, 11 aa, Artificial Sequence
  Description of Artificial Sequence Synthetic peptide
RILQQLLFTHF

SEQ ID NO: 586 - protein, 6 aa, Artificial Sequence
  Description of Artificial Sequence Synthetic peptide
RIFIGC

SEQ ID NO: 587 - protein, 6 aa, Artificial Sequence
  Description of Artificial Sequence Synthetic peptide
FIRIGC

SEQ ID NO: 588 - protein, 22 aa, Artificial Sequence
  Description of Artificial Sequence Synthetic peptide
DTWAGVEATIRILQQLLFTHFR

SEQ ID NO: 589 - protein, 5 aa, Artificial Sequence
  Description of Artificial Sequence Synthetic peptide
ICCRH

SEQ ID NO: 590 - protein, 23 aa, Artificial Sequence
  Description of Artificial Sequence Synthetic peptide
GYGRKKRRGRRRTHRLPRRRRRR

SEQ ID NO: 591 - protein, 12 aa, Artificial Sequence
  Description of Artificial Sequence Synthetic peptide
KRIIQRILSRNS

SEQ ID NO: 592 - protein, 12 aa, Artificial Sequence
  Description of Artificial Sequence Synthetic peptide
KRIHPRLTRSIR

SEQ ID NO: 593 - protein, 12 aa, Artificial Sequence
  Description of Artificial Sequence Synthetic peptide
PPRLRKRRQLNM

SEQ ID NO: 594 - protein, 12 aa, Artificial Sequence
  Description of Artificial Sequence Synthetic peptide
MHKRPTTPSRKM

SEQ ID NO: 595 - protein, 12 aa, Artificial Sequence
  Description of Artificial Sequence Synthetic peptide
RQRSRRRPLNIR

SEQ ID NO: 596 - protein, 12 aa, Artificial Sequence
  Description of Artificial Sequence Synthetic peptide
RIRMIQNLIKKT

SEQ ID NO: 597 - protein, 12 aa, Artificial Sequence
  Description of Artificial Sequence Synthetic peptide
SRRKRQRSNMRI

SEQ ID NO: 598 - protein, 12 aa, Artificial Sequence
  Description of Artificial Sequence Synthetic peptide
QRIRKSKISRTL

SEQ ID NO: 599 - protein, 12 aa, Artificial Sequence
  Description of Artificial Sequence Synthetic peptide
PSKRLLHNNLRR

SEQ ID NO: 600 - protein, 12 aa, Artificial Sequence
  Description of Artificial Sequence Synthetic peptide
HRHIRRQSLIML

SEQ ID NO: 601 - protein, 12 aa, Artificial Sequence
  Description of Artificial Sequence Synthetic peptide
PQNRLQIRRHSK

SEQ ID NO: 602 - protein, 12 aa, Artificial Sequence
  Description of Artificial Sequence Synthetic peptide
PPHNRIQRRLNM

SEQ ID NO: 603 - protein, 12 aa, Artificial Sequence
  Description of Artificial Sequence Synthetic peptide
SMLKRNHSTSNR

SEQ ID NO: 604 - protein, 12 aa, Artificial Sequence
  Description of Artificial Sequence Synthetic peptide
GSRHPSLIIPRQ

SEQ ID NO: 605 - protein, 12 aa, Artificial Sequence
  Description of Artificial Sequence Synthetic peptide
SPMQKTMNLPPM

SEQ ID NO: 606 - protein, 12 aa, Artificial Sequence
  Description of Artificial Sequence Synthetic peptide
NKRILIRIMTRP

SEQ ID NO: 607 - protein, 12 aa, Artificial Sequence
  Description of Artificial Sequence Synthetic peptide
HGWZIHGLLHRA

SEQ ID NO: 608 - protein, 11 aa, Artificial Sequence
  Description of Artificial Sequence Synthetic peptide
AVPAKKRZKSV

SEQ ID NO: 609 - protein, 11 aa, Artificial Sequence
  Description of Artificial Sequence Synthetic peptide
PNTRVRPDVSF

SEQ ID NO: 610 - protein, 12 aa, Artificial Sequence
  Description of Artificial Sequence Synthetic peptide
LTRNYEAWVPTP

SEQ ID NO: 611 - protein, 13 aa, Artificial Sequence
  Description of Artificial Sequence Synthetic peptide
SAETVESCLAKSH

SEQ ID NO: 612 - protein, 12 aa, Artificial Sequence
  Description of Artificial Sequence Synthetic peptide
YSHIATLPFTPT

SEQ ID NO: 613 - protein, 12 aa, Artificial Sequence
  Description of Artificial Sequence Synthetic peptide
SYIQRTPSTTLP

SEQ ID NO: 614 - protein, 12 aa, Artificial Sequence
  Description of Artificial Sequence Synthetic peptide
AVPAENALNNPF

SEQ ID NO: 615 - protein, 12 aa, Artificial Sequence
  Description of Artificial Sequence Synthetic peptide
SFHQFARATLAS

SEQ ID NO: 616 - protein, 12 aa, Artificial Sequence
  Description of Artificial Sequence Synthetic peptide
QSPTDFTFPNPL

SEQ ID NO: 617 - protein, 12 aa, Artificial Sequence
  Description of Artificial Sequence Synthetic peptide
HFAAWGGWSLVH

SEQ ID NO: 618 - protein, 12 aa, Artificial Sequence
  Description of Artificial Sequence Synthetic peptide
HIQLSPFSQSWR

SEQ ID NO: 619 - protein, 12 aa, Artificial Sequence
  Description of Artificial Sequence Synthetic peptide
LTMPSDLQPVLW

SEQ ID NO: 620 - protein, 12 aa, Artificial Sequence
  Description of Artificial Sequence Synthetic peptide
FQPYDHPAEVSY

SEQ ID NO: 621 - protein, 12 aa, Artificial Sequence
  Description of Artificial Sequence Synthetic peptide
FDPFFWKYSPRD

SEQ ID NO: 622 - protein, 12 aa, Artificial Sequence
  Description of Artificial Sequence Synthetic peptide
FAPWDTASFMLG

SEQ ID NO: 623 - protein, 12 aa, Artificial Sequence
  Description of Artificial Sequence Synthetic peptide
FTYKNFFWLPEL

SEQ ID NO: 624 - protein, 12 aa, Artificial Sequence
  Description of Artificial Sequence Synthetic peptide
SATGAPWKMWVR

SEQ ID NO: 625 - protein, 12 aa, Artificial Sequence
  Description of Artificial Sequence Synthetic peptide
SLGWMLPFSPPF

SEQ ID NO: 626 - protein, 12 aa, Artificial Sequence
  Description of Artificial Sequence Synthetic peptide
SHAFTWPTYLQL

SEQ ID NO: 627 - protein, 12 aa, Artificial Sequence
  Description of Artificial Sequence Synthetic peptide
SHNWLPLWPLRP

SEQ ID NO: 628 - protein, 12 aa, Artificial Sequence
  Description of Artificial Sequence Synthetic peptide
SWLPYPWHVPSS

SEQ ID NO: 629 - protein, 12 aa, Artificial Sequence
  Description of Artificial Sequence Synthetic peptide
SWWTPWHVHSES

SEQ ID NO: 630 - protein, 12 aa, Artificial Sequence
  Description of Artificial Sequence Synthetic peptide
SWAQHLSLPPVL

SEQ ID NO: 631 - protein, 12 aa, Artificial Sequence
  Description of Artificial Sequence Synthetic peptide
SSSIFPPWLSFF

SEQ ID NO: 632 - protein, 12 aa, Artificial Sequence
  Description of Artificial Sequence Synthetic peptide
LNVPPSWFLSQR

SEQ ID NO: 633 - protein, 12 aa, Artificial Sequence
  Description of Artificial Sequence Synthetic peptide
LDITPFLSLTLP

SEQ ID NO: 634 - protein, 12 aa, Artificial Sequence
  Description of Artificial Sequence Synthetic peptide
LPHPVLHMGPLR

SEQ ID NO: 635 - protein, 12 aa, Artificial Sequence
  Description of Artificial Sequence Synthetic peptide
VSKQPYYMWNGN

SEQ ID NO: 636 - protein, 12 aa, Artificial Sequence
  Description of Artificial Sequence Synthetic peptide
NYTTYKSHFQDR

SEQ ID NO: 637 - protein, 12 aa, Artificial Sequence
  Description of Artificial Sequence Synthetic peptide
AIPNNQLGFPFK

SEQ ID NO: 638 - protein, 12 aa, Artificial Sequence
  Description of Artificial Sequence Synthetic peptide
NIENSTLATPLS

SEQ ID NO: 639 - protein, 12 aa, Artificial Sequence
  Description of Artificial Sequence Synthetic peptide
YPYDANHTRSPT

SEQ ID NO: 640 - protein, 12 aa, Artificial Sequence
  Description of Artificial Sequence Synthetic peptide
DPATNPGPHFPR

SEQ ID NO: 641 - protein, 12 aa, Artificial Sequence
  Description of Artificial Sequence Synthetic peptide
TLPSPLALLTVH

SEQ ID NO: 642 - protein, 12 aa, Artificial Sequence
  Description of Artificial Sequence Synthetic peptide
HPGSPFPPEHRP

SEQ ID NO: 643 - protein, 12 aa, Artificial Sequence
  Description of Artificial Sequence Synthetic peptide
TSHTDAPPARSP

SEQ ID NO: 644 - protein, 12 aa, Artificial Sequence
  Description of Artificial Sequence Synthetic peptide
MTPSSLSTLPWP

SEQ ID NO: 645 - protein, 12 aa, Artificial Sequence
  Description of Artificial Sequence Synthetic peptide
VLGQSGYLMPMR

SEQ ID NO: 646 - protein, 12 aa, Artificial Sequence
  Description of Artificial Sequence Synthetic peptide
QPIIITSPYLPS

SEQ ID NO: 647 - protein, 12 aa, Artificial Sequence
  Description of Artificial Sequence Synthetic peptide
TPKTMTQTYDFS

SEQ ID NO: 648 - protein, 12 aa, Artificial Sequence
  Description of Artificial Sequence Synthetic peptide
NSGTMQSASRAT

SEQ ID NO: 649 - protein, 12 aa, Artificial Sequence
  Description of Artificial Sequence Synthetic peptide
QAASRVENYMHR

SEQ ID NO: 650 - protein, 12 aa, Artificial Sequence
  Description of Artificial Sequence Synthetic peptide
HQHKPPPLTNNW

SEQ ID NO: 651 - protein, 12 aa, Artificial Sequence
  Description of Artificial Sequence Synthetic peptide
SNPWDSLLSVST

SEQ ID NO: 652 - protein, 12 aa, Artificial Sequence
  Description of Artificial Sequence Synthetic peptide
KTIEAHPPYYAS

SEQ ID NO: 653 - protein, 12 aa, Artificial Sequence
  Description of Artificial Sequence Synthetic peptide
EPDNWSLDFPRR

SEQ ID NO: 654 - protein, 12 aa, Artificial Sequence
  Description of Artificial Sequence Synthetic peptide
HQHKPPPLTNNW

SEQ ID NO: 655 - protein, 20 aa, Artificial Sequence
  Description of Artificial Sequence Synthetic peptide
GLWRALWRLLRSLWRLLWKA

SEQ ID NO: 656 - protein, 21 aa, Artificial Sequence
  Description of Artificial Sequence Synthetic peptide
GLWRALWRALWRSLWKLKRKV

SEQ ID NO: 657 - protein, 20 aa, Artificial Sequence
  Description of Artificial Sequence Synthetic peptide
GLWRALWRALRSLWKLKRKV

SEQ ID NO: 658 - protein, 20 aa, Artificial Sequence
  Description of Artificial Sequence Synthetic peptide
GLWRALWRGLRSLWKLKRKV

SEQ ID NO: 659 - protein, 20 aa, Artificial Sequence
  Description of Artificial Sequence Synthetic peptide
GLWRALWRGLRSLWKKKRKV

SEQ ID NO: 660 - protein, 21 aa, Artificial Sequence
  Description of Artificial Sequence Synthetic peptide
GLWRALWRALWRSLWKLKWKV

SEQ ID NO: 661 - protein, 21 aa, Artificial Sequence
  Description of Artificial Sequence Synthetic peptide
GLWRALWRALWRSLWKSKRKV

SEQ ID NO: 662 - protein, 21 aa, Artificial Sequence
  Description of Artificial Sequence Synthetic peptide
GLWRALWRALWRSLWKKKRKV

SEQ ID NO: 663 - protein, 21 aa, Artificial Sequence
  Description of Artificial Sequence Synthetic peptide
GLWRALWRALWRSLWKLKRKV

SEQ ID NO: 664 - protein, 27 aa, Artificial Sequence
  Description of Artificial Sequence Synthetic peptide
GLWRALWRLLRSLWRLLWSQPKKKRKV

SEQ ID NO: 665 - protein, 11 aa, Artificial Sequence
  Description of Artificial Sequence Synthetic peptide
YARAARRAARR

SEQ ID NO: 666 - protein, 11 aa, Artificial Sequence
  Description of Artificial Sequence Synthetic peptide
PARAARRAARR

SEQ ID NO: 667 - protein, 11 aa, Artificial Sequence
  Description of Artificial Sequence Synthetic peptide
YPRAARRAARR

SEQ ID NO: 668 - protein, 11 aa, Artificial Sequence
  Description of Artificial Sequence Synthetic peptide
YRRAARRAARA

SEQ ID NO: 669 - protein, 11 aa, Artificial Sequence
  Description of Artificial Sequence Synthetic peptide
YGRRARRAARR

SEQ ID NO: 670 - protein, 11 aa, Artificial Sequence
  Description of Artificial Sequence Synthetic peptide
YAREARRAARR

SEQ ID NO: 671 - protein, 11 aa, Artificial Sequence
  Description of Artificial Sequence Synthetic peptide
YGRRARRAARR

SEQ ID NO: 672 - protein, 11 aa, Artificial Sequence
  Description of Artificial Sequence Synthetic peptide
YKRAARRAARR

SEQ ID NO: 673 - protein, 11 aa, Artificial Sequence
  Description of Artificial Sequence Synthetic peptide
YARKARRAARR

SEQ ID NO: 674 - protein, 11 aa, Artificial Sequence
  Description of Artificial Sequence Synthetic peptide
YKRKARRAARR

SEQ ID NO: 675 - protein, 11 aa, Artificial Sequence
  Description of Artificial Sequence Synthetic peptide
YGRRARRAARR

SEQ ID NO: 676 - protein, 11 aa, Artificial Sequence
  Description of Artificial Sequence Synthetic peptide
YGRRARRRARR

SEQ ID NO: 677 - protein, 11 aa, Artificial Sequence
  Description of Artificial Sequence Synthetic peptide
YGRRRRRRRRR

SEQ ID NO: 678 - protein, 11 aa, Artificial Sequence
  Description of Artificial Sequence Synthetic peptide
YRRRRRRRRRR

SEQ ID NO: 679 - protein, 16 aa, Artificial Sequence
  Description of Artificial Sequence Synthetic peptide
GKINLKALAALAKKIL

SEQ ID NO: 680 - protein, 39 aa, Artificial Sequence
  Description of Artificial Sequence Synthetic polypeptide
GRKKRRQRRRPPQGRKKRRQRRRPPQGRKKRRQRRRPPQ

SEQ ID NO: 681 - protein, 22 aa, Artificial Sequence
  Description of Artificial Sequence Synthetic peptide
GEQIAQLIAGYIDIILKKKKSK

SEQ ID NO: 682 - protein, 28 aa, Artificial Sequence
  Description of Artificial Sequence Synthetic peptide
AAVALLPAVLLALLAPRKKRRQRRRPPQ

SEQ ID NO: 683 - protein, 29 aa, Artificial Sequence
  Description of Artificial Sequence Synthetic peptide
AAVALLPAVLLALLAPRKKRRQRRRPPQC

SEQ ID NO: 684 - protein, 29 aa, Artificial Sequence
  Description of Artificial Sequence Synthetic peptide
RKKRRQRRRPPQCAAVALLPAVLLALLAP

SEQ ID NO: 685 - protein, 29 aa, Artificial Sequence
  Description of Artificial Sequence Synthetic peptide
RRRQRRKRGGDIMGEWGNEIFGAIAGFLG

SEQ ID NO: 686 - protein, 29 aa, Artificial Sequence
  Description of Artificial Sequence Synthetic peptide
RRRQRRKRGGDIMGEWGNEIFGAIAGFLG

SEQ ID NO: 687 - protein, 25 aa, Artificial Sequence
  Description of Artificial Sequence Synthetic peptide
YGRKKRRQRRRGCYGRKKRRQRRRG

SEQ ID NO: 688 - protein, 22 aa, Artificial Sequence
  Description of Artificial Sequence Synthetic peptide
AAVALLPAVLLALLAPRRRRRR

SEQ ID NO: 689 - protein, 16 aa, Artificial Sequence
  Description of Artificial Sequence Synthetic peptide
RLWRALPRVLRRLLRP

SEQ ID NO: 690 - protein, 28 aa, Artificial Sequence
  Description of Artificial Sequence Synthetic peptide
AAVALLPAVLLALLAPSGASGLDKRDYV

SEQ ID NO: 691 - protein, 33 aa, Artificial Sequence
  Description of Artificial Sequence Synthetic polypeptide
LLETLLKPFQCRICMRNFSTRQARRNHRRRHRR

SEQ ID NO: 692 - protein, 27 aa, Artificial Sequence
  Description of Artificial Sequence Synthetic peptide
AAVACRICMRNFSTRQARRNHRRRHRR

SEQ ID NO: 693 - protein, 35 aa, Artificial Sequence
  Description of Artificial Sequence Synthetic polypeptide
RQIKIWFQNRRMKWKKDIMGEWGNEIFGAIAGFLG

SEQ ID NO: 694 - protein, 37 aa, Artificial Sequence
  Description of Artificial Sequence Synthetic polypeptide
SGRGKQGGKARAKAKTRSSRAGLQFPVGRVHRLLRKG

SEQ ID NO: 695 - protein, 38 aa, Artificial Sequence
  Description of Artificial Sequence Synthetic polypeptide
SGRGKQGGKARAKAKTRSSRAGLQFPVGRVHRLLRKGC

SEQ ID NO: 696 - protein, 25 aa, Artificial Sequence
  Description of Artificial Sequence Synthetic peptide
KKDGKKRKRSRKESYSVYVYKVLKQ

SEQ ID NO: 697 - protein, 36 aa, Artificial Sequence
  Description of Artificial Sequence Synthetic polypeptide
KGSKKAVTKAQKKDGKKRKRSRKESYSVYVYKVLKQ

SEQ ID NO: 698 - protein, 28 aa, Artificial Sequence
  Description of Artificial Sequence Synthetic peptide
KETWWETWWTEWSQPGRKKRRQRRRPPQ

SEQ ID NO: 699 - protein, 15 aa, Artificial Sequence
  Description of Artificial Sequence Synthetic peptide
RVIRWFQNKRCKDKK

SEQ ID NO: 700 - protein, 18 aa, Artificial Sequence
  Description of Artificial Sequence Synthetic peptide
LGLLLRHLRHHSNLLANI

SEQ ID NO: 701 - protein, 16 aa, Artificial Sequence
  Description of Artificial Sequence Synthetic peptide
KLWSAWPSLWSSLWKP

SEQ ID NO: 702 - protein, 22 aa, Artificial Sequence
  Description of Artificial Sequence Synthetic peptide
GLGSLLKKAGKKLKQPKSKRKV

SEQ ID NO: 703 - protein, 12 aa, Artificial Sequence
  Description of Artificial Sequence Synthetic peptide
  MOD_RES (4)..(4): D-amino acid
  MOD_RES (6)..(6): D-amino acid
  MOD_RES (8)..(8): D-amino acid
  MOD_RES (10)..(10): D-amino acid
  MOD_RES (12)..(12): D-amino acid
FKQQQQQQQQQQ

SEQ ID NO: 704 - protein, 4 aa, Artificial Sequence
  Description of Artificial Sequence Synthetic peptide
YRFK

SEQ ID NO: 705 - protein, 13 aa, Artificial Sequence
  Description of Artificial Sequence Synthetic peptide
YRFKYRFKYRLFK

SEQ ID NO: 706 - protein, 10 aa, Artificial Sequence
  Description of Artificial Sequence Synthetic peptide
WRFKKSKRKV

SEQ ID NO: 707 - protein, 20 aa, Artificial Sequence
  Description of Artificial Sequence Synthetic peptide
WRFKAAVALLPAVLLALLAP

SEQ ID NO: 708 - protein, 8 aa, Artificial Sequence
  Description of Artificial Sequence Synthetic peptide
WRFKWRFK

SEQ ID NO: 709 - protein, 12 aa, Artificial Sequence
  Description of Artificial Sequence Synthetic peptide
WRFKWRFKWRFK

SEQ ID NO: 710 - protein, 36 aa, Artificial Sequence; identical to SEQ ID NO: 697
  Description of Artificial Sequence Synthetic polypeptide

SEQ ID NO: 711 - protein, 36 aa, Artificial Sequence
  Description of Artificial Sequence Synthetic polypeptide
RGSRRAVTRAQRRDGRRRRRSRRESYSVYVYRVLRQ

SEQ ID NO: 712 - protein, 15 aa, Artificial Sequence
  Description of Artificial Sequence Synthetic peptide
RVIRWFQNKRSKDKK

SEQ ID NO: 713 - protein, 28 aa, Artificial Sequence
  Description of Artificial Sequence Synthetic peptide
AAVALLPAVLLALLAPRKKRRQRRRPPQ

SEQ ID NO: 714 - protein, 5 aa, Artificial Sequence
  Description of Artificial Sequence Synthetic peptide
CWKKK

SEQ ID NO: 715 - protein, 10 aa, Artificial Sequence
  Description of Artificial Sequence Synthetic peptide
CWKKKKKKKK

SEQ ID NO: 716 - protein, 15 aa, Artificial Sequence
  Description of Artificial Sequence Synthetic peptide
CWKKKKKKKKKKKKK

SEQ ID NO: 717 - protein, 20 aa, Artificial Sequence
  Description of Artificial Sequence Synthetic peptide
CWKKKKKKKKKKKKKKKKKK

SEQ ID NO: 718 - protein, 19 aa, Artificial Sequence
  Description of Artificial Sequence Synthetic peptide
KKKKKKKKKKKKKKKKKKK

SEQ ID NO: 719 - protein, 19 aa, Artificial Sequence
  Description of Artificial Sequence Synthetic peptide
  MOD_RES (1)..(7): D-amino acid
  MOD_RES (9)..(9): D-amino acid
  MOD_RES (11)..(19): D-amino acid
KKWKMRRGAGRRRRRRRRR

SEQ ID NO: 720 - protein, 12 aa, Artificial Sequence
  Description of Artificial Sequence Synthetic peptide
APWHLSSQYSRT

SEQ ID NO: 721 - protein, 26 aa, Artificial Sequence
  Description of Artificial Sequence Synthetic peptide
AAVALLPAVLLALLAKKNNLKDCGLF

SEQ ID NO: 722 - protein, 26 aa, Artificial Sequence
  Description of Artificial Sequence Synthetic peptide
AAVALLPAVLLALLAKKNNLKECGLY

SEQ ID NO: 723 - protein, 26 aa, Artificial Sequence
  Description of Artificial Sequence Synthetic peptide
AHALCLTERQIKIWFQNRRMKWKKEN

SEQ ID NO: 724 - protein, 26 aa, Artificial Sequence
  Description of Artificial Sequence Synthetic peptide
AHALCPPERQIKIWFQNRRMKWKKEN

SEQ ID NO: 725 - protein, 26 aa, Artificial Sequence
  Description of Artificial Sequence Synthetic peptide
AYALCLTERQIKIWFANRRMKWKKEN

SEQ ID NO: 726 - protein, 34 aa, Artificial Sequence
  Description of Artificial Sequence Synthetic polypeptide
GGVCPKILKKGRRDSDCPGACICRGNGYCGSGSD

SEQ ID NO: 727 - protein, 34 aa, Artificial Sequence
  Description of Artificial Sequence Synthetic polypeptide
GGVCPKILAACRRDSDCPGACICRGNGYCGSGSD

SEQ ID NO: 728 - protein, 34 aa, Artificial Sequence
  Description of Artificial Sequence Synthetic polypeptide
GGVCPAILKKCRRDSDCPGACICRGNGYCGSGSD

SEQ ID NO: 729 - protein, 34 aa, Artificial Sequence
  Description of Artificial Sequence Synthetic polypeptide
GGVCPKILAKCRRDSDCPGACICRGNGYCGSGSD

SEQ ID NO: 730 - protein, 34 aa, Artificial Sequence
  Description of Artificial Sequence Synthetic polypeptide
GGVCPKILKACRRDSDCPGACICRGNGYCGSGSD

SEQ ID NO: 731 - protein, 29 aa, Artificial Sequence
  Description of Artificial Sequence Synthetic peptide
GLPVCGETCVGGICNIPGCKCSWPVCIRN

SEQ ID NO: 732 - protein, 29 aa, Artificial Sequence
  Description of Artificial Sequence Synthetic peptide
GLPVCGETCVGGTCNTPGCTCSWPKCTRN

SEQ ID NO: 733 - protein, 14 aa, Artificial Sequence
  Description of Artificial Sequence Synthetic peptide
GRCTKSIPPICFPD

SEQ ID NO: 734 - protein, 33 aa, Artificial Sequence
  Description of Artificial Sequence Synthetic polypeptide
RQIKIWFQNRRMKWKKTYADFIASGRTGRRNAI

SEQ ID NO: 735 - protein, 30 aa, Artificial Sequence
  Description of Artificial Sequence Synthetic polypeptide
GRKKRRQRRRPPQTYADFIASGRTGRRNAI

SEQ ID NO: 736 - protein, 21 aa, Artificial Sequence
  Description of Artificial Sequence Synthetic peptide
AGYLLGKINLKALAALAKKIL

SEQ ID NO: 737 - protein, 38 aa, Artificial Sequence
  Description of Artificial Sequence Synthetic polypeptide
AGYLLGKINLKALAALAKKILTYADFIASGRTGRRNAI

SEQ ID NO: 738 - protein, 11 aa, Artificial Sequence
  Description of Artificial Sequence Synthetic peptide
RRRRRRRRRRR

SEQ ID NO: 739 - protein, 28 aa, Artificial Sequence
  Description of Artificial Sequence Synthetic peptide
RRRRRRRRRRRTYADFIASGRTGRRNAI

SEQ ID NO: 740 - protein, 10 aa, Artificial Sequence
  Description of Artificial Sequence Synthetic peptide
  MOD_RES (1)..(10): D-amino acid
RRRRRRRRRK

SEQ ID NO: 741 - protein, 9 aa, Artificial Sequence
  Description of Artificial Sequence Synthetic peptide
  MOD_RES (1)..(1): D-amino acid
  MOD_RES (9)..(9): D-amino acid
RRRRRRRRR

SEQ ID NO: 742 - protein, 9 aa, Artificial Sequence
  Description of Artificial Sequence Synthetic peptide
  MOD_RES (1)..(1): D-amino acid
  MOD_RES (3)..(3): D-amino acid
  MOD_RES (5)..(5): D-amino acid
  MOD_RES (7)..(7): D-amino acid
  MOD_RES (9)..(9): D-amino acid
RRRRRRRRR

SEQ ID NO: 743 - protein, 22 aa, Artificial Sequence
  Description of Artificial Sequence Synthetic peptide
KCFQWQRNMRKVRGPPVSCIKR

SEQ ID NO: 744 - protein, 22 aa, Artificial Sequence
  Description of Artificial Sequence Synthetic peptide
  MOD_RES (1)..(22): D-amino acid
KCFQWQRNMRKVRQPPYSCIKR

SEQ ID NO: 745 - protein, 20 aa, Artificial Sequence
  Description of Artificial Sequence Synthetic peptide
KLALKLALKALKAALKLAGC

SEQ ID NO: 746 - protein, 20 aa, Artificial Sequence
  Description of Artificial Sequence Synthetic peptide
  MOD_RES (3)..(3): Aib
  MOD_RES (7)..(7): Aib
  MOD_RES (10)..(10): Aib
  MOD_RES (14)..(14): Aib
  MOD_RES (18)..(18): Aib
KLXLKLXLKXLKAXLKLXGC

SEQ ID NO: 747 - protein, 35 aa, Artificial Sequence
  Description of Artificial Sequence Synthetic polypeptide
GGGARKKAAKAARKKAAKAARKKAAKAARKKAAKA

SEQ ID NO: 748 - protein, 14 aa, Artificial Sequence
  Description of Artificial Sequence Synthetic peptide
GRKKRRQRRRPPQC

SEQ ID NO: 749 - protein, 19 aa, Artificial Sequence
  Description of Artificial Sequence Synthetic peptide
TRQARRNRRRRWRERQRGC

SEQ ID NO: 750 - protein, 17 aa, Artificial Sequence
  Description of Artificial Sequence Synthetic peptide
RRRRNRTRRNRRRVRGC

SEQ ID NO: 751 - protein, 21 aa, Artificial Sequence
  Description of Artificial Sequence Synthetic peptide
KMTRAQRRAAARRNRWTARGC

SEQ ID NO: 752 - protein, 15 aa, Artificial Sequence
  Description of Artificial Sequence Synthetic peptide
TRRQRTRRARRNRGC

SEQ ID NO: 753 - protein, 30 aa, Artificial Sequence; identical to SEQ ID NO: 396
  Description of Artificial Sequence Synthetic polypeptide

SEQ ID NO: 754 - protein, 28 aa, Artificial Sequence
  Description of Artificial Sequence Synthetic peptide
KRRIRRERNKMAAAKSRNRRRELTDTGC

SEQ ID NO: 755 - protein, 23 aa, Artificial Sequence
  Description of Artificial Sequence Synthetic peptide
WLRRIKAWLRRIKALNRQLGVAA

SEQ ID NO: 756 - protein, 10 aa, Artificial Sequence
  Description of Artificial Sequence Synthetic peptide
  MOD_RES (1)..(10): D-amino acid
CRKKRRQRRR

SEQ ID NO: 757 - protein, 10 aa, Artificial Sequence
  Description of Artificial Sequence Synthetic peptide
  MOD_RES (1)..(10): D-amino acid
CRRRRRRRRR

SEQ ID NO: 758 - protein, 10 aa, Artificial Sequence
  Description of Artificial Sequence Synthetic peptide
  MOD_RES (1)..(10): D-amino acid
CKKKKKKKKK

SEQ ID NO: 759 - protein, 12 aa, Artificial Sequence
  Description of Artificial Sequence Synthetic peptide
GRKKRRQRRRPP

SEQ ID NO: 760 - protein, 8 aa, Artificial Sequence
  Description of Artificial Sequence Synthetic peptide
  MOD_RES (1)..(8): D-amino acid
RRRRRRRR

SEQ ID NO: 761 - DNA, 8972 bp, Homo sapiens
gtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcacccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgt

SEQ ID NO: 762 - DNA, 2779 bp, Homo sapiens
cacagcggggcggggcgcggggctgagcaggcgcgagggctggctgctgggccgacgaggggcggtgccaggccgtgggtccttagtcaagtgacgcgaagcggccggcctgggcgccgactgcagagccgggaggctggtggtcatgccggggttcctggttcgcatcctccctctgttgctggttctgctgcttctgggccctacgcgcggcttgcgctctcgattccttccttgggcttttcatctgccacggcaagcgcctaagtctcagctgccgcttcacaaaagaggtaccaagactgcaccgaatgagcacgcaagcagcaacaggagtgggagaagaaggaggcggcagcagtggaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgatgatgaaagcctgtgtctttgagggattctaccctgaacatacctcacagatcctccctgtcatgccacatttcactgattggaatgtggaaatggaaaaggaatttaggatgtgcattttcacctgaggtttccctgcatccctgcagtgccaaagccccaccttcagggaccacctggaatgtgtgaggggctgacagcacagtaacgtgcatacatatctgcagggctggaatggaagctttaaaggtggtagtgatttttattttggaagaatcatgttacctttttgttaaataaaatttgtactcaaatgatgatgtcactgtttttaatgtgcaggtattgaattatatggtctgacttaaatcataactagacttgagtgggctgaataaaccacttcactaacttgaagttcaaaaggatggaaaataaaaaaaaaaaaaaaaaaaa

SEQ ID NO: 763 - DNA, 468 bp, Homo sapiens
acatatccactcctgctctccctcctgcaggtgaccccagccatgaggaccatcgccatccttgctgccattctcctggtggccctgcaggcccaggctgagtcactccaggaaagagctgatgaggctacaacccagaagcagtctggggaagacaaccaggaccttgctatctcctttgcaggaaatggactctctgctcttagaacctcaggttctcaggcaagagccacctgctattgccgaaccggccgttgtgctacccgtgagtccctctccggggtgtgtgaaatcagtggccgcctctacagactctgctgtcgctgagcttcctagatagaaaccaaagcagtgcaagattcagttcaaggtcctgaaaaaagaaaaacattttactctgtgtaccttgtgtctttctaaatttctctctccaaaataaagttcaagcattaaacttaaaaaaaaaaa

SEQ ID NO: 764 - DNA, 3060 bp, Homo sapiens
attcagcacaattcctatcaaaatccccatggcattctttacagaaataaaaaaaaaatcctaaaatttgtatgaaatggtgaaagaacccaaatagacaaaacaattctgagaaaaaaaaacaaagttgaagacatcacaattcctggttttaaattatattacaaagctacggtaatcaaaacaatatggtattggcataaaaccagacacataccagtgaaataaaattgagagtccagaaacacatcgaaacacatatggtcaagtaacttttgacaatggcatgaagaagacacaatagggacaggagagtctcttcaataaatggtggtgggaatactatttccatatgcaaaacaataaaactggacccttctcttttggcatatacaaaacttaactcaaaatggataaaatacctgatagaagaccagaaacaagagactatgaaaataatggtagagttctatatgttttgagctacaaagtattcaaattacagcattagggagaaaaaacaaagcacaaaccaatgtttaatttttaagtggagatttcctggagcagagctgcagggctggggcaaggatggaggctcattactccttttaccctttgaatggtccttgtgggcaattcaacattttctaaaattaaaaataaaataaaatttcagagggggattgtgggcatgtcaattgtttcgatatttctcaaagtggagagaaatacctagtacatattttacttgatacattcagatcacatgggtttcttgaaataaatcttttggctttagttttactaactctttacctagtatcccactgagttcttttcccttataggctgataaggtcattatcttctccacactgtgccccacaagagcctattcaccccataatctcaaagcctccttcatgagggcagaaatgccccctgaatcctgcaatgaattaactctctactctagcgggatccagctctggcctcaaggtctacacctccagagagtggccagccccaccttcagaaaataagaggcatttgattcctgaaattattcattgaaagcactgttcttttcttttttgaatattaacaagtaaatattccagcagatggaaaacaggacaatgtaacactgttcttatcatcactatcagctgggaccagaacagacactcaataaacagcctcacactacaatgaagcttggagaacaaaggagcatcaaagggacatggagggcaagggtagctcttctgctccccaatcacatgcactccccgtctcaccgcaacatctgtccctgagccttctccaagcagacctataaatccaggctggctcctcactccccacacatctgctcctgctctctctcctccagcgaccctagccatgagaaccctcaccatcctcactgctgttctcctcgtggccctccaggccaaggctgagccactccaagctgaggatgatccactgcaggcaaaagcttatgaggctgatgcccaggagcagcgtggggcaaatgaccaggactttgccgtctcctttgcagaggatgcaagctcaagtcttagagctttgggtaagagacaccagcattgcagagctaggagtgtagagaggaaaaccaagcacttctagaattagatccaacagctggctctttctcttaggtgatcacctccccaggcctcaattactttatgtttgtactgaaaaggaagaatcagtgatattcaaggcatgacttttctctaaagattttttaattctatgataagtctatgaaattttctaattttttgccatgtagaaatgtattgaggagtctctacttcaaggaagagagcctaattttaaaggaatgttttgttttgctttgttttgttttgttttgtttgatggagtctcgctctgtcacccaggctggagtgcagtggcactatctcagatcactgcaacctctgcctcctgggttcaagcaattctctccctcagcctcccaagtcactaggattacaggcaccagacaccaagcctggctaaattttgtattttttttttttcagtagagacggggtttcaccatcttggccaggctagtcttgaactcctgaccttgtgataaacccaccttggcctcccaaagtgctgggattacaggcgtaagccaccacatccagctgctaaaggaatgttttttaatctgacttttataggaaccgttggaaactggagacagtcatatgggtgcattcagatgtgtgtgtgacagggaaggagcagataagtacagcatatcagaatgggtctctaatcctgtgtgtgaccaacactgctctgcgtatttattcctattgatggtgtgatcatgctattggctgtaatgcagccagcattacatgtcagcaagcatgcaacttcctgaagattctctttactgcccgctgctgaccctggtgctcaatttctgatgctctctctctctgtccccaggctcaacaagggctttcacttgccattgcagaaggtcctgttattcaacagaatattcctatgggacctgcactgtcatgggtattaaccacagattctgctgcctctgagggatgagaacagagagaaatatattcataatttactttatgacctagaaggaaactgtcgtgtgtcctatacattgccatcaactttgtttcctcatctcaaataaagtcctttcagcaagttcttttgtgtttgtgcttttctggtgtttgataattcaggattcttcagatgcaaaaacaaaaacccaagtcgtatctcagaacactagctcttcgaaagagttttctatgtagaccagagaagtggtagagaggatgttgagagaagagaggatttgggttttttgtttttttgttttttgctttctgagatggagtctcgctctgttgcccaggctgcagtgcagtggcacaatcttggctcactgaaacctttacctcctgggttcaagtgattctcctgcctcagcctcccaagtagctgggatta

SEQ ID NO: 765 - DNA, 10211 bp, Artificial Sequence
  Description of Artificial Sequence Synthetic polynucleotide
ccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacgccgccatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtatgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcgagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggggcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcttcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggtcttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccggtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaggatccagatctttttccctctgccaaaaattatggggacatcatgaagccccttgagcatctgacttctggctaataaaggaaatttattttcattgcaatagtgtgttggaattttttgtgtctctcactcggaaggacataagggcggccgctagc

SEQ ID NO: 766 - protein, 7 aa, Simian virus 40
PKKKRKV

SEQ ID NO: 767 - protein, 16 aa, Unknown
  Description of Unknown Nucleoplasmin bipartite NLS sequence
KRPAATKKAGQAKKKK

SEQ ID NO: 768 - protein, 9 aa, Unknown
  Description of Unknown C-myc NLS sequence
PAAKRVKLD

SEQ ID NO: 769 - protein, 11 aa, Unknown
  Description of Unknown C-myc NLS sequence
RQRRNELKRSP

SEQ ID NO: 770 - protein, 38 aa, Homo sapiens
NQSSNFGPMKGGNFGGRSSGPYGGGGQYFAKPRNQGGY

SEQ ID NO: 771 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 772 - protein, 8 aa, Unknown
  Description of Unknown Myoma T protein sequence
VSRKRPRP

SEQ ID NO: 773 - protein, 8 aa, Unknown
  Description of Unknown Myoma T protein sequence
PPKKARED

SEQ ID NO: 774 - protein, 8 aa, Homo sapiens
PQPKKKPL

SEQ ID NO: 775 - protein, 12 aa, Mus musculus
SALIKKKKKMAP

SEQ ID NO: 776 - protein, 5 aa, Influenza virus
DRLRR

SEQ ID NO: 777 - protein, 7 aa, Influenza virus
PKQKKRK

SEQ ID NO: 778 - protein, 10 aa, Hepatitis delta virus
RKLKKKIKKL

SEQ ID NO: 779 - protein, 10 aa, Mus musculus
REKKKFLKRR

SEQ ID NO: 780 - protein, 20 aa, Homo sapiens
KRKGDEVDGVDEVAKKKSKK

SEQ ID NO: 781 - protein, 17 aa, Homo sapiens
RKCLQAGMNLEARKTKK

SEQ ID NO: 782 - DNA, 18 bp, Artificial Sequence
  Description of Artificial Sequence Synthetic oligonucleotide
gatctcgagctccctgca

SEQ ID NO: 783 - DNA, 10 bp, Artificial Sequence
  Description of Artificial Sequence Synthetic oligonucleotide
gggagctcga

SEQ ID NO: 784 - DNA, 34 bp, Artificial Sequence
  Description of Artificial Sequence Synthetic oligonucleotide
tcgacgccgccatggctgcagaaaaaaggatcca

SEQ ID NO: 785 - DNA, 34 bp, Artificial Sequence
  Description of Artificial Sequence Synthetic oligonucleotide
gatctggatccttttttctgcagccatggcggcg

SEQ ID NO: 786 - DNA, 10 bp, Artificial Sequence
  Description of Artificial Sequence Synthetic oligonucleotide
gccgccatgg

(19) United States
(12) Patent Application Publication
(10) Pub. No.: US 2019/0351071 A1
(43) Pub. Date: Nov. 21, 2019

(54) STRUCTURES AND METHODS FOR GENE THERAPY

(71) Applicant: DNALITE THERAPEUTICS, INC., San Francisco, CA (US)

(72) Inventors: Mubhij AHMAD, San Francisco, CA (US); Timothy Paul DAY, San Francisco, CA (US)

(21) Appl. No.: 16/348,405

(22) PCT Filed: Nov. 10, 2017
(86) PCT No.: PCT/US17/61111

§ 371 Date: May 8, 2019

Related U.S. Application Data
(60) Provisional application No. 62/421,103, filed on Nov. 11, 2016.
(60) Provisional application No. 62/485,493, filed on Apr. 14, 2017.

(51) Int. Cl. A61K 48/00; A61K 9/127; A61K 9/51; A61K 9/00; A61K 47/69; A61K 47/68; C12N 15/113; A61K 9/19; A61P 35/00; A61K 45/06; A61K 31/635; A61P 1/00

(52) U.S. Cl. CPC A61K 48/0033; C12N 2310/14; A61K 9/5138; A61K 9/0053; A61K 47/6911; A61K 47/6913; A61K 47/6849; C12N 15/113; A61K 9/19; A61P 35/00; A61K 45/06; A61K 31/635; A61K 9/1277; A61P 1/00; A61K 9/1271

(57) ABSTRACT

Provided are liposomal structures comprising polynucleic acids. Also disclosed are polypeptides encoded by polynucleic acids, pharmaceutical compositions of liposomal structures, and methods of making and using the same.

### CROSS REFERENCE

This application claims the benefit of U.S. Provisional Application No. 62/421,103, filed Nov. 11, 2016 and U.S. Provisional Application No. 62/485,493, filed Apr. 14, 2017; both of which are herein incorporated by reference in their entirety.

### BACKGROUND

Despite advances in gene therapy over the last 50 years, there remain many diseases that are recalcitrant to conventional methods, particularly in cases where a target location for gene therapy may provide challenges for delivery.

The disclosed compositions and methods herein can be used for the generation of nanoparticles for gene therapy. Nanoparticles can non-invasively supply therapeutic genes to sites of disease.

### INCORPORATION BY REFERENCE

All publications, patents, and patent applications herein are incorporated by reference in their entirety to the same extent as if each individual publication, patent, or patent application was specifically and individually indicated to be incorporated by reference. In the event of a conflict between a term herein and a term in an incorporated reference, the term herein controls.

### SUMMARY

Disclosed herein are liposomal structures. A liposomal structure can comprise a polynucleic acid. A polynucleic acid can be isolated. A polynucleic acid can be purified. A polynucleic acid can be isolated and purified. A liposomal structure can comprise a surface modification. The surface modification can enhance an average rate at which a liposomal structure moves in mucus compared to a comparable liposomal structure. A comparable liposomal structure can be surface modified with a polymer. A polymer can be a polyethylene glycol (PEG). A PEG can have an average molecular weight ranging from about 2000 to about 3000 Daltons (Da). In some cases, a liposomal structure can be a liposome, a lipoplex, or a lipopolyplex. In some cases, a liposomal structure can be a liposome. A liposomal structure can be a lipoplex. A liposomal structure can be a lipopolyplex. In some cases, a surface modification can comprise a polymer of Formula I:

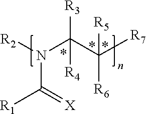

wherein R₁ can be independently selected from a group consisting of hydrogen; deuterium; C₁₋₆ alkyl; C₂₋₆ alkynyl; C₂₋₆ alkynyl, C₃₋₈ cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be selected from a group consisting of a coupling group capable of coupling to a linker, or a substrate; hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R3 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R4 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R5 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R6 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R7 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, wherein when R₃ and R₄ are not the same * can independent be R or S; wherein when R₅, R₆ and R₇ are not the same ** can be R or S; wherein X can be independently selected from oxygen or sulfur; Y can be independently selected from deuterium or hydrogen; A is hydrogen, deuterium, aryl, or heteroaryl and n is 1-100. In some cases, R₁ can be a C₁₋₆ alkyl. In some cases, any one of R₃, R₄, R₅, or R₆ can be selected from the group consisting of deuterium and hydrogen. In some cases, X can be oxygen. Formula I can have an average molecular weight from about 1000 Da to about 8000 Da. A surface modification can comprise poly (2-methyl-2-oxazoline), poly (2-ethyl-2-oxazoline), a salt thereof, a di block polymer thereof, a tri block polymer thereof, or a combination thereof. A surface modification can be at a density from about 0.05 ug/nm² to about 0.25 ug/nm². An average rate at which a liposomal structure moves in mucus can be from about 2 fold to about 5 fold greater than an average rate of a comparable liposomal structure as measured by a transwell migration assay. In some cases, a polynucleic acid can comprises DNA. A polynucleic acid can be minicircle DNA or closed-linear DNA. In some cases, a polynucleic acid can comprise minicircle DNA. In some cases, a polynucleic acid can comprises RNA. A polynucleic acid can be at least partially enclosed within a liposomal structure. A liposomal structure can comprise at least two polynucleic acids. A liposomal structure can further comprises a peptide, antibody or fragment thereof, carbohydrate, single chain variable fragment (scFv), cellular receptor, or any combination thereof. In some cases, a liposomal structure can comprise a peptide, antibody or fragment thereof, single chain variable fragment (scFv), or cellular receptor in contact with a surface modification. In some cases, a liposomal structure can comprise a peptide. A peptide can be a cell-penetrating peptide. A liposomal structure can comprise an antibody or fragment thereof. A liposomal structure can comprise an antibody or fragment thereof that can target a leucine-rich repeat-containing G-protein coupled receptor 5 (LGR5). A liposomal structure can further comprise an exterior coating. An exterior coating can be cationic. An exterior coating can be anionic. An exterior coating can be neutral. An exterior coating can comprise ethyl acrylate in polymerized form. An exterior coating can have a near-neutral zeta potential as measured by laser doppler anemometry. In some cases, a near-neutral zeta potential can be from about −100 mV to about 100 mV. A liposomal structure can comprise a lipid bilayer. A lipid bilayer that can comprise one or more of cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt of any of these, or any combination thereof. In some cases, a material can be a lipid with a net positive charge or a lipid with a neutral charge. A lipid bilayer can comprise MVL5. A lipid bilayer can comprise MVL5 and GMO. In some cases, a molar ratio of MVL5 to GMO ranges from about 10:1 to about 1:25. A molar ratio of MVL5 and GMO can range from about 10:1 to about 1:10. A lipid bilayer can comprise DOGS and DOPE. A polynucleic acid can be fully encapsulated in a lipid bilayer. A polynucleic acid can be in contact with a lipid bilayer. In some cases, a polynucleic acid may not be in contact with a lipid bilayer. In some cases, a liposomal structure can further comprise a second lipid bilayer. In some cases, a liposomal structure can further comprise a linker. A linker can be an acid sensitive linker. A linker associates with the surface modification of the liposomal structure. A linker can directly associate with a surface modification of a liposomal structure. In some cases, a linker can indirectly associate with a surface modification of a liposomal structure. A polynucleic acid can encode for at least a fragment of a protein. At least a fragment of a protein can be active in a gastrointestinal (GI) tract. In some cases, at least a fragment of a protein can be active in a bodily area comprising a mucosal membrane. A polynucleic acid can encode for at least a fragment of adenomatous polyposis coli (APC), defensin alpha 5 (HD-5), defensin alpha 6 (HD-6), or any combination thereof. In some cases, a liposomal structure can have a diameter selected from the group consisting of: from about 10 nm to about 100 nm, from about 100 nm to about 200 nm, from about 200 nm to about 300 nm, from about 300 nm to about 400 nm, and from about 400 nm to about 500 nm as measured by dynamic light scattering.

Disclosed herein are liposomal structures. A liposomal structure can comprise a polynucleic acid. A polynucleic acid can be isolated. A polynucleic acid can be purified. A polynucleic acid can be isolated and purified. A polynucleic acid can be free of a bacterial origin of replication. A liposomal structure can be surface modified with a polymer. A liposomal structure can comprise a polymer comprising Formula I

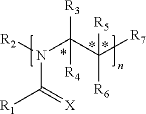

wherein R₁ can be independently selected from a group consisting of hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be independently selected from a group consisting of a coupling group capable of coupling to a linker, or a substrate; hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₃ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R₄ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R₅ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R₆ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R₇ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, wherein * can independently be R, S or achiral; wherein ** can independently be R, S or achiral; wherein X can be independently selected from oxygen or sulfur; Y can be independently selected from deuterium or hydrogen; A can be hydrogen, deuterium, aryl, or heteroaryl and n can be from about 1 to about 100. In some cases, R₁ can be a C₁₋₆ alkyl. R₃, R₄, R₅, or R₆ can be selected from the group consisting of deuterium and hydrogen. X can be an oxygen. In some cases, Formula I can have an average molecular weight from about 1000 Da to about 8000 Da. In some cases, a polymer can comprise poly (2-methyl-2-oxazoline), poly (2-ethyl-2-oxazoline), a salt thereof, a di block polymer thereof, a tri block polymer thereof, or a combination thereof. A liposomal structure can be a liposome, a lipoplex, or a lipopolyplex. A liposomal structure can be a liposome. A liposomal structure can be a lipoplex. A liposomal structure can be a lipopolyplex. In some cases, a polymer can have a density of from about 0.05 ug/nm² to about 0.25 ug/nm². A polymer can enhance an average rate at which a liposomal structure moves in mucus compared to an otherwise comparable liposomal structure, wherein the comparable liposomal structure is surface modified with polyethylene glycol (PEG). In some cases, PEG can have an average molecular weight from 2000 Da to 3000 Da. An average rate at which a liposomal structure moves in mucus can be from about 2 fold to about 5 fold greater than the average rate of the comparable liposomal structure as measured by a transwell migration assay. A liposomal structure can have increased hydrophilicity compared to a comparable liposomal structure. A polynucleic acid can comprise DNA. A polynucleic acid can be minicircle DNA or closed-linear DNA. A polynucleic acid can comprise minicircle DNA. A polynucleic acid can comprise ribonucleic acid (RNA). A polynucleic acid can be at least partially water soluble. A polynucleic acid can be at least partially enclosed within a liposomal structure. A liposomal structure can comprise at least two polynucleic acids. A polynucleic acid can comprise at least one promoter. At least one promoter can be selected from cytomegalovirus (CMV) derived promoter, chicken 3-actin (CBM) derived promoter, adenomatous polyposis coli (APC) derived promoter, leucine-rich repeat containing G protein-coupled receptor 5 (LGR5), CAG promoter, Beta actin promoter, elongation factor-1 (EF1) promoter, early growth response 1 (EGR-1) promoter, eukaryotic initiation factor 4A (EIF4A1) promoter, or any combination thereof. A liposomal structure can further comprise a peptide, antibody or fragment thereof, carbohydrate, single chain variable fragment (scFv), cellular receptor, or any combination thereof. A liposomal structure can comprise a peptide, antibody or fragment thereof, single chain variable fragment (scFv), or cellular receptor in contact with a polymer. A liposomal structure can comprise a peptide, wherein a peptide can be a cell-penetrating peptide. In some cases, a peptide can be in contact with a polynucleic acid. A peptide may not be in contact with a polynucleic acid. A liposomal structure can comprise an antibody or fragment thereof, wherein an antibody or fragment thereof can target a leucine-rich repeat-containing G-protein coupled receptor 5 (LGR5). In some cases, a liposomal structure can further comprise a nuclease inhibitor. A nuclease inhibitor can be selected from the group consisting of aurintricarboxylic acid (ATA), Zn²⁺, DMI-2, salts thereof, or a combination thereof. A liposomal structure can further comprise an effector of RNA interference (RNAi). An effector of RNA interference (RNAi) can be CEQ508 or salt thereof. A liposomal structure can further comprise an exterior coating. An exterior coating can be cationic. An exterior coating can be anionic. An exterior coating can be neutral. An exterior coating can comprise ethyl acrylate in polymerized form. In some cases, an exterior coating can have a near-neutral zeta potential as measured by laser doppler anemometry. A near-neutral zeta potential can be from about −100 mV to about 100 mV. A liposomal structure can comprise a lipid bilayer. In some cases, a polynucleic acid can be present in an aqueous solution enclosed in a lipid bilayer. A lipid bilayer can comprise one or more of cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt of any of these, or any combination thereof. In some cases, a material can be a lipid with a net positive charge or a lipid with a neutral charge. A lipid bilayer can comprise MVL5. A lipid bilayer can comprise MVL5 and GMO. A molar ratio of MVL5 to GMO can range from about 10:1 to about 1:25. A molar ratio of MVL5 and GMO can range from about 10:1 to about 1:10. A lipid bilayer can comprise DOGS and DOPE. A polynucleic acid can be fully encapsulated in a lipid bilayer. A polynucleic acid can be in contact with a lipid bilayer. A polynucleic acid may not be in contact with a lipid bilayer. A liposomal structure can further comprise a second lipid bilayer. In some cases, a liposomal structure can further comprise a linker. A linker can be an acid sensitive linker. A linker can associate with a polymer. A linker can directly associate with a polymer. A linker can indirectly associate with a polymer. A polynucleic acid can encode for at least a fragment of a protein. In some cases, at least a fragment of a protein can be active in a gastrointestinal (GI) tract. At least a fragment of a protein can be active in a bodily area comprising a mucosal membrane. A polynucleic acid can encode for at least a fragment of adenomatous polyposis coli (APC), defensin alpha 5 (HD-5), defensin alpha 6 (HD-6), or any combination thereof. A liposomal structure can have a diameter selected from the group consisting of: from about 10 nm to about 100 nm, from about 100 nm to about 200 nm, from about 200 nm to about 300 nm, from about 300 nm to about 400 nm, and from about 400 nm to about 500 nm as measured by dynamic light scattering. A liposomal structure can have a diameter from about 100 nm to about 200 nm as measured by dynamic light scattering.

Disclosed herein are liposomal structures. A liposomal structure can comprise a polynucleic acid. A polynucleic acid can be isolated. A polynucleic acid can be purified. A polynucleic acid can be isolated and purified. A liposomal structure can be surface modified with the polymer of Formula I

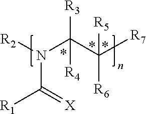

wherein R₁ can be independently selected from a group consisting of hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be independently selected from a group consisting of a coupling group capable of coupling to a linker, or a substrate; hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₃ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₄ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₅ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₆ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₇ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; wherein * can independently be R, S or achiral; wherein ** can independently be R, S or achiral; wherein X can be independently selected from oxygen or sulfur; Y can be independently selected from deuterium or hydrogen; A can be hydrogen, deuterium, aryl, or heteroaryl and n can be from about 1 to about 100. In some cases, R₁ can be a C₁₋₆ alkyl. R₃, R₄, R₅, or R₆ can be selected from the group consisting of deuterium and hydrogen. X can be an oxygen. In some cases, Formula I can have an average molecular weight from about 1000 Da to about 8000 Da. In some cases, a polymer can comprise poly (2-methyl-2-oxazoline), poly (2-ethyl-2-oxazoline), a salt thereof, a di block polymer thereof, a tri block polymer thereof, or a combination thereof. A liposomal structure can be a liposome, a lipoplex, or a lipopolyplex. A liposomal structure can be a liposome. A liposomal structure can be a lipoplex. A liposomal structure can be a lipopolyplex. In some cases, a polymer can have a density of from about 0.05 ug/nm² to about 0.25 ug/nm². A polymer can enhance an average rate at which a liposomal structure moves in mucus compared to an otherwise comparable liposomal structure, wherein the comparable liposomal structure is surface modified with polyethylene glycol (PEG). In some cases, PEG can have an average molecular weight from 2000 Da to 3000 Da. An average rate at which a liposomal structure moves in mucus can be from about 2 fold to about 5 fold greater than the average rate of the comparable liposomal structure as measured by a transwell migration assay. A liposomal structure can have increased hydrophilicity compared to a comparable liposomal structure. A polynucleic acid can comprise DNA. A polynucleic acid can be minicircle DNA or closed-linear DNA. A polynucleic acid can comprise minicircle DNA. A polynucleic acid can comprise ribonucleic acid (RNA). A polynucleic acid can be at least partially water soluble. A polynucleic acid can be at least partially enclosed within a liposomal structure. A liposomal structure can comprise at least two polynucleic acids. A polynucleic acid can comprise at least one promoter. At least one promoter can be selected from cytomegalovirus (CMV) derived promoter, chicken 3-actin (CBM) derived promoter, adenomatous polyposis coli (APC) derived promoter, leucine-rich repeat containing G protein-coupled receptor 5 (LGR5), CAG promoter, Beta actin promoter, elongation factor-1 (EF1) promoter, early growth response 1 (EGR-1) promoter, eukaryotic initiation factor 4A (EIF4A1) promoter, or any combination thereof. A liposomal structure can further comprise a peptide, antibody or fragment thereof, carbohydrate, single chain variable fragment (scFv), cellular receptor, or any combination thereof. A liposomal structure can comprise a peptide, antibody or fragment thereof, single chain variable fragment (scFv), or cellular receptor in contact with a polymer. A liposomal structure can comprise a peptide, wherein a peptide can be a cell-penetrating peptide. In some cases, a peptide can be in contact with a polynucleic acid. A peptide may not be in contact with a polynucleic acid. A liposomal structure can comprise an antibody or fragment thereof, wherein an antibody or fragment thereof can target a leucine-rich repeat-containing G-protein coupled receptor 5 (LGR5). In some cases, a liposomal structure can further comprise a nuclease inhibitor. A nuclease inhibitor can be selected from the group consisting of aurintricarboxylic acid (ATA), Zn²⁺, DMI-2, salts thereof, or a combination thereof. A liposomal structure can further comprise an effector of RNA interference (RNAi). An effector of RNA interference (RNAi) can be CEQ508 or salt thereof. A liposomal structure can further comprise an exterior coating. An exterior coating can be cationic. An exterior coating can be anionic. An exterior coating can be neutral. An exterior coating can comprise ethyl acrylate in polymerized form. In some cases, an exterior coating can have a near-neutral zeta potential as measured by laser doppler anemometry. A near-neutral zeta potential can be from about −20 mV to about 20 mV. A near-neutral zeta potential can also be from about −100 mV to about 100 mV. A liposomal structure can comprise a lipid bilayer. In some cases, a polynucleic acid can be present in an aqueous solution enclosed in a lipid bilayer. A lipid bilayer can comprise one or more of cholesterol, N-[1-(2,3-dioleyloxy)propyl]-N,N,N-trimethylammonium chloride (DOTMA), [1,2-bis(oleoyloxy)-3 (trimethylammonio)propane](DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt of any of these, or any combination thereof. In some cases, a material can be a lipid with a net positive charge or a lipid with a neutral charge. A lipid bilayer can comprise MVL5. A lipid bilayer can comprise MVL5 and GMO. A molar ratio of MVL5 to GMO can range from about 10:1 to about 1:25. A molar ratio of MVL5 and GMO can range from about 10:1 to about 1:10. A lipid bilayer can comprise DOGS and DOPE. A polynucleic acid can be fully encapsulated in a lipid bilayer. A polynucleic acid can be in contact with a lipid bilayer. A polynucleic acid may not be in contact with a lipid bilayer. A liposomal structure can further comprise a second lipid bilayer. In some cases, a liposomal structure can further comprise a linker. A linker can be an acid sensitive linker. A linker can associate with a polymer. A linker can directly associate with a polymer. A linker can indirectly associate with a polymer. A polynucleic acid can encode for at least a fragment of a protein. In some cases, at least a fragment of a protein can be active in a gastrointestinal (GI) tract. At least a fragment of a protein can be active in a bodily area comprising a mucosal membrane. A polynucleic acid can encode for at least a fragment of adenomatous polyposis coli (APC), defensin alpha 5 (HD-5), defensin alpha 6 (HD-6), or any combination thereof. A liposomal structure can have a diameter selected from the group consisting of: from about 10 nm to about 100 nm, from about 100 nm to about 200 nm, from about 200 nm to about 300 nm, from about 300 nm to about 400 nm, and from about 400 nm to about 500 nm as measured by dynamic light scattering. A liposomal structure can have a diameter from about 100 nm to about 200 nm as measured by dynamic light scattering.

Disclosed herein are pharmaceutical compositions. A pharmaceutical composition can comprise a liposomal structure. A pharmaceutical composition can comprise an excipient. A pharmaceutical composition can comprise a diluent. A pharmaceutical composition can comprise a carrier. A pharmaceutical composition can be in unit dosage form. A pharmaceutical composition can be in the form of a tablet. A pharmaceutical composition can be in the form of a liquid. A pharmaceutical composition can be in the form of a syrup. A pharmaceutical composition can be in the form of an oral formulation. A pharmaceutical composition can be in the form of an intravenous formulation. A pharmaceutical composition can be in the form of an intranasal formulation. A pharmaceutical composition can be in the form of a subcutaneous formulation. A pharmaceutical composition can be in the form of an inhalable respiratory formulation. A pharmaceutical composition can be in the form of a suppository. A pharmaceutical composition can be in the form of a tablet, a liquid, a syrup, an oral formulation, an intravenous formulation, an intranasal formulation, a subcutaneous formulation, an inhalable respiratory formulation, a suppository, and any combination thereof.

Disclosed herein are methods of treating a subject in need thereof. In some cases, a method of treating a subject can comprise administering to a subject in need thereof a therapeutically effective amount of a liposomal structure. In some cases, a method of treating a subject can comprise administering to a subject in need thereof a pharmaceutical composition. In some cases, administration of a liposomal structure or a pharmaceutical composition can at least partially ameliorate a disease or condition in a subject in need thereof. A disease or condition can comprise familial adenomatous polyposis (FAP), attenuated FAP, cancer, chronic inflammatory bowel disease, chronic inflammatory bowel disease, ileal Crohn's or any combination thereof. A disease or condition can be FAP. A subject can have a polyp in a gastrointestinal tract. A subject in need thereof can have a polyp surgically removed prior to, after, or concurrent with administration of a liposomal structure or a pharmaceutical composition. A liposomal structure or a pharmaceutical composition can be administered orally, rectally, or orally and rectally. A liposomal structure or a pharmaceutical composition can be administered routinely. A liposomal structure or a pharmaceutical composition can be administered prophylactically. A liposomal structure or a pharmaceutical composition can be administered 1 time per day, 2 times per day, 3 times per day, daily, weekly, yearly or any combination thereof. A subject can be administered an additional therapy in a therapeutically effective amount. An additional therapy can comprise a non-steroidal anti-inflammatory drug (NSAID) or a salt thereof, a miRNA against β-catenin, a mucus disrupting agent or a salt a salt thereof, or any combination thereof. A non-steroidal anti-inflammatory drug (NSAID) can comprise Celecoxib. A mucus disrupting agent can comprise guaifenesin. A subject in need thereof can be genetically screened for a disease or condition.

A liposomal structure or a pharmaceutical composition described herein can be comprised in a kit. A kit can comprise a pharmaceutical composition described herein, and instructions for use thereof. A kit can further comprise a container. Also disclosed herein are methods of making a kit. A method of making a kit can comprise placing a liposomal structure described herein or a pharmaceutical composition described herein into a container. A kit or method of making a kit can further comprise instructions for use.

Disclosed herein are methods of making a liposomal structure. Also disclosed herein are methods of making a pharmaceutical composition. A method of making a liposomal structure or a pharmaceutical composition can comprise forming a liposome around a polynucleic acid. A liposomal structure can be surface modified with a polymer. A polynucleic acid can encode for a protein or portion thereof that can be active in a gastrointestinal tract or a tumor suppressor protein or portion thereof. A liposomal structure can be a liposome, a lipoplex, or a lipopolyplex. A liposomal structure can be a liposome. A polymer can comprise Formula I:

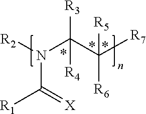

wherein R₁ can be independently selected from a group consisting of hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be independently selected from a group consisting of a coupling group capable of coupling to a linker, or a substrate; hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₃ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₄ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₅ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₆ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₇ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; wherein * can independently be R, S or achiral; wherein ** can independently be R, S or achiral; wherein X can be independently selected from oxygen or sulfur; Y can be independently selected from deuterium or hydrogen; A can be hydrogen, deuterium, aryl, or heteroaryl and n can be from about 1 to about 100. R₁ can be C₁₋₆ alkyl. Any one of R₃, R₄, R₅, or R₆ can be selected from the group consisting of deuterium and hydrogen. X can be oxygen. In some cases, Formula I can have an average molecular weight from about 1000 Da to about 8000 Da. A polymer can comprise poly (2-methyl-2-oxazoline), poly (2-ethyl-2-oxazoline), a salt thereof, a di block polymer thereof, a tri block polymer thereof, or a combination thereof. A method can further comprise introducing a solvent. A solvent can comprise chloroform. A method can further comprise drying a solvent. Drying can comprise exposing a solvent to dry nitrogen, argon stream, rotary evaporation, vacuum, or any combination thereof. Drying can comprise exposing a solvent to dry nitrogen. Drying can comprise exposing a drying to a vacuum. In some cases, drying can comprise exposing a solvent to a dry nitrogen stream followed by a vacuum. Drying can form a lipid film that can be hydrated by addition of an aqueous solution. A method can further comprise an aqueous solution. A method can comprise a polynucleic acid comprising DNA, RNA, or any combination thereof. A polynucleic acid can comprise DNA. A polynucleic acid can comprise mini-circle DNA.

Disclosed herein are liposomal structures. A liposomal structure can comprise a polynucleic acid. A polynucleic acid can be isolated. A polynucleic acid can be purified. A polynucleic acid can be isolated and purified. A purified polynucleic acid can be at least partially enclosed within a liposomal structure. A liposomal structure can have increased hydrophilicity compared to a comparable liposomal structure A comparable liposomal structure can comprise a polyethylene glycol (PEG) surface modification. In some cases, increased hydrophilicity can be caused by a non-PEG surface modification. A non-PEG surface modification can comprise a polymer of Formula I:

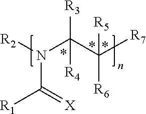

wherein R₁ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be independently selected from a group consisting of a coupling group capable of coupling to a linker, or a substrate; hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R3 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R4 can independently be selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R5 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R6 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R7 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, wherein when R₃ and R₄ are not the same * can independent be R or S; wherein when R₅, R₆ and R₇ are not the same ** can independent be R or S; wherein X can be independently selected from oxygen or sulfur; Y can be independently selected from deuterium or hydrogen; A can be hydrogen, deuterium, aryl, or heteroaryl and n can be from about 1 to about 100. R₁ can be a C₁₋₆ alkyl. Any one of R₃, R₄, R₅, or R₆ can be selected from the group consisting of deuterium and hydrogen. X can be oxygen. Formula I can have an average molecular weight from about 1000 Da to about 8000 Da. A surface modification can comprise poly (2-methyl-2-oxazoline), poly (2-ethyl-2-oxazoline), a salt thereof, a di block polymer thereof, a tri block polymer thereof, or a combination thereof. A polynucleic acid can comprise minicircle DNA. A liposomal structure can comprise a lipid bilayer. A lipid bilayer can comprise one or more of cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt of any of these, or any combination thereof. In some cases, a material can be a lipid with a net positive charge or a lipid with a neutral charge. A lipid bilayer can comprise MVL5 and GMO.

Disclosed herein are nanostructures. In some embodiments a nanostructure can comprise: at least one polynucleic acid encoding at least one protein or portion thereof, at least one lipid bilayer contacting at least one polymer; and at least one external coating; wherein a polynucleic acid can be at least partially encapsulated within a lipid bilayer and an external coating can be at least partially coating a nanostructure and wherein at least one of the following can be comprised: an external coating can be an enteric coating at least one polymer comprises a polyglycol polymer, or any combination thereof. In some cases, a polynucleic acid can be isolated. In some cases, a polynucleic acid can be purified. In some cases, a polynucleic acid can be isolated and purified. In some cases, a polynucleic acid can be circular. In some cases, a nanostructure can have a diameter from about 500 nm or less. In other cases, a nanostructure can have a diameter selected from a list comprising: from about 10 nm to from about 100 nm, from about 100 nm to from about 200 nm, from about 200 nm to from about 300 nm, from about 300 nm to from about 400 nm, or from about 400 nm to from about 500 nm. At least one external coating can be partially coating. At least one external coating can be fully coating. In some cases, a nanostructure can comprise at least one external coating that can be selected from a list comprising: Cellulose acetate phthalate, Polyvinyl acetate phthalate, Hydroxypropylmethylcellulose acetate succinate, Poly(methacylic acid-co-ethyl acrylate) 1:1, Poly(methacrylic acid-co-ethyl acrylate) 1:1, Poly(methacylic acid-co-methyl methacrylate) 1:1, Poly(methacylic acid-co-methyl methacrylate) 1:1, Poly(methacylic acid-co-methyl methacrylate) 1:2, Poly(methacylic acid-co-methyl methacrylate) 1:2, or Poly(methyl acrylate-co-methyl methacrylate-co-methacrylic acid) 7:3:1. At least one external coating can be Poly (methacylic acid-co-ethyl acrylate) 1:1. At least one external coating can be a mucoadhesive hydrogel. A mucoadhesive hydrogel can be selected from a group consisting of Hydroxyethyl Cellulose (HEC), polyacrylates (carbomer), alginates, chitosan, and cellulosic derivatives (hydroxyethylcellulose, hydroxypropylmethylcellulose, or carboxymethylcellulose. In some cases, at least one external coating can be pH sensitive. A pH sensitive coating can dissolve above a pH of 5.5 as measured at 37 degrees Celsius with a pH meter when dissolved in 1 L of water with a stirring rod rotating at 200 revolutions per minute. A pH sensitive coating can dissolve above a pH of 7 as measured at 37 degrees Celsius with a pH meter when dissolved in 1 L of water with a stirring rod rotating at 200 revolutions per minute. In other cases, a pH sensitive coating can dissolve above a pH of 6 as measured at 37 degrees Celsius with a pH meter when dissolved in 1 L of water with a stirring rod rotating at 200 revolutions per minute. Dissolution can occur enterically. In other cases, dissolution can occur in an organ selected from a group consisting of a duodenum, jejunum, ilium, and colon. In some cases, dissolution can occur in proximity to an intestinal crypt cell. In proximity can refer to adjacent to an intestinal crypt cell. For example, adjacent can mean immediately next to. In other cases, adjacent can mean within the same section of an intestine. For example, if dissolution occurs in a duodenum of a small intestine it may be considered as adjacent to an intestinal crypt cell found in a duodenum. In some embodiments, at least one polymer can be attached to a lipid bilayer directly, covalently, non-covalently, via a linker, or any combination thereof. A polymer can be attached covalently to a lipid bilayer. At least one polymer can be polyethylene glycol (PEG), a triblock copolymer of PEG-polypropyelene oxide, poly(2-methyl-2-oxazoline), poly(vinyl alcohol), poly(vinyl ethers), poly(N-[2-hydroxypropyl)methylacrylamide), polyethyleneimine (PEI), poly(2-dimethylaminoethyl methacrylate) (pDMAEMA), and poly-L-lysine (pLL) a modified version, or derivative thereof. In some cases, a polymer comprises poly(2-methyl-2-oxazoline) or PEG. In some cases, at least one polymer may not interact with mucus as measured by an increase in the distance of mucus transversed by a nanostructure comprising at least one polymer that may not interact with mucus compared to a nanostructure that may not contain a polymer. In some cases, PEG can be PEG 2000 comprising a molecular weight average from 1900 g/mol to 2200 g/mol. PEG can be attached to a lipid bilayer from about 10 to 20 chains per 100 nm². PEG surface density can be estimated using an actual molar ratio of lipid-PEG in the liposome and a calculated weighted average surface area of a liposome. The actual molar ratio of lipid-PEG that can be determined can be 1HNMR prepared in D2O with 1% w/w DSS as reference. 500 MHz, 10 s relaxation time and ZG pulse set at 90 degrees. PEG peaks occur at 3.3-4.1 ppm and their integral can be compared to standards. In some cases, PEG can be in a mushroom configuration. In other cases, PEG can be in a brush configuration. In other cases, PEG can be in a pancake configuration. A lipid bilayer can be in a form of a liposome. In some cases, a lipid bilayer can be generated from a list of lipids selected from a group consisting of: cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), Dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO) derivatives thereof, and combinations thereof. A lipid bilayer can be comprised of DOGS and DOPE. In some cases, DOGS and DOPE can be at an 80 Mol to 20 Mol ratios. In some cases, PEG can be combined with at least one lipid at a concentration of 5 Mol to 10 Mol. In some cases, a PEG concentration can be selected from a list comprising: DOGS/DOPE/PEG at 80 mol/20 mol/5 mol, 80 mol/20 mol/6 mol, 80 mol/20 mol/7 mol, 80 mol/20 mol/8 mol, 80 mol/20 mol/9 mol, or 80 mol/20 mol/10 mol. In some cases, MVL5 and GMO can be used as lipids in a liposome structure. Molar concentrations of MVL5 and GMO can range from about 10:1 to 1:25. A molar ratio of MVL5 and GMO can range from about 10:1 to about 1:10. In other cases, a molar ratio of MVL5 and GMO can range from about 50:1 to 1:1. For example a molar ratio can be from about 50:1 to 1:1, 40:1 to 1:1, 30:1 to 1:1, 20:1 to 1:1, 10:1 to 1:1 or about 5:1 to 1:1. A molar ratio of MVL5 and GMO can range from about 10:1 to about 1:10. In some cases, a molar ratio of MVL5/GMO/lipid-HPEG can be from about 50 mol/45 mol/5 mol, 50 mol/44 mol/6 mol, 50 mol/43 mol/7 mol, 50 mol/42 mol/8 mol, 50 mol/41 mol/9 mol, to about 50 mol/40 mol/10 mol. In some cases, a lipid, such as MVL5, can hydrogen bond a polynucleic acid such as a minicircle polynucleic acid. In some cases, a polymer can further comprise a peptide, antibody, carbohydrate, or a combination thereof. A peptide or antibody can be selected from a list comprising antibodies, single chain variable fragments (scFv), cellular receptors, barcodes, linkers, or any combination thereof. A peptide can be a cell-penetrating peptide. In some cases, an antibody can be leucine-rich repeat-containing G-protein coupled receptor 5 (LGR5). A nanostructure can be cationic, anionic, neutral, or any combination thereof. A nanostructure can be neutral. In some cases, a nanostructure can have a near-neutral zeta potential as measured by laser doppler anemometry. In some cases, charge can be from −20 mV to 20 mV for nanostructures at a DNA charge ratio of 10 in 1 mL of high-resistivity water are measured by a Malvern Nanosizer ZS. In some cases, charge can be from −100 mV to about 20 mV for nanostructures at a DNA charge ratio of 10 in 1 mL of high-resistivity water are measured by a Malvern Nanosizer ZS. A DNA charge ratio can be from 0 to 20. In some cases, polymers that can form a nanoparticle can comprise a “charge ratio”. The charge ratio can refer to a ratio of the number of positive charges (cationic) on the cationic monomers that comprise the second block of the polymers (N) to the number of negative charges (anionic) on the polynucleotides that are incorporated into the nanostructure (P). In some embodiments the cationic charges are cationic amines of the cationic monomers. In some embodiments the anionic charges are anionic charges of the phosphate groups on the backbone the polynucleic acid (e.g., minicircle DNA). In some embodiments the ratio can be calculated at physiological pH, neutral pH, or a combination thereof. In further embodiments, for the purpose of calculating a N:P ratio, the cationic monomers are assumed to have about half (50%) of their cationic species (e.g., amines) charged at the neutral and/or physiological pH. Exemplary nanostructures can be formed with a particular N:P ratio so as to determine or estimate the dosage of polynucleotides in the nanoparticles. Exemplary nanoparticles can also be made at an N:P ratio that achieves the desired characteristics, including size, stability, surface charge, and the like, for the nanoparticles. In some embodiments the N:P ratio can be a value between about 0.5 and about 20. In some embodiments the N:P ratio can be a value between about 1.0 and about 30. In some embodiments the N:P ratio can be a value between about 5.0 and about 15, or a value between about 10 and 10. In further embodiments the N:P ratio can be a value from about 1, 2, 3, 4, 5, 6, 7, 8, 9, 10, 11, 12, 13, 14, 15, 16, 17, 18, 19, or up to about 20. In some cases, a polynucleic acid can be DNA, RNA, or any combination thereof. Polynucleic acid can be DNA. DNA can be mini-circle DNA. In some cases, a polynucleic acid can be water soluble. A polynucleic acid can be suspended in aqueous solution within a lipid bilayer. In some cases, at least one protein or portion thereof can be adenomatous polyposis coli (APC), B-galactosidase, (B-Gal), or any combination thereof. In some cases, at least one protein or portion thereof can be adenomatous polyposis coli (APC). In some cases, a polynucleic acid can comprise at least one promoter. A promoter can be selected from a list comprising cytomegalovirus (CMV) derived promoter, chicken (3-actin (CBM) derived promoter, adenomatous polyposis coli (APC) derived promoter, leucine-rich repeat containing G protein-coupled receptor 5 (LGR5), CAG promoter, Beta actin promoter, elongation factor-1 (EF1) promoter, early growth response 1 (EGR-1) promoter, eukaryotic initiation factor 4A (EIF4A1) promoter, or any combination thereof. Disclosed herein can be a nanostructure further comprising a protein or peptide. A protein or peptide can comprise a nuclear localization signal. In some cases, a protein or peptide can bind a polynucleic acid. In some cases, a nanostructure can further comprise a DNase inhibitor. A nanostructure can be a mucus penetrating particle (MPP). An MPP can be able to penetrate mucus from 1 to 200 micrometers in thickness. An MPP can have a near neutral zeta potential from about −20 mV to about 20 mV. A nanostructure can be at least partially biodegradable. A nanostructure can be freeze-dried. A nanostructure can be administered as a pill, as a hydrogel, or any combination thereof. Disclosed herein can be a pharmaceutical composition comprising a nanostructure disclosed herein. A pharmaceutical composition can comprise a pharmaceutically acceptable excipient.

In some instance, a pharmaceutical composition disclosed herein can be administered to a patient in unit dosage form. In some cases a method disclosed herein can treat or prevent at least one condition in a patient. In some cases, a nanostructure can be used to treat familial adenomatous polyposis (FAP), attenuated FAP, colorectal cancer, chronic inflammatory bowel disease, chronic inflammatory bowel disease or any combination thereof. A nanostructure can be used to treat FAP. A nanostructure can be administered orally or rectally. A nanostructure can be administered routinely. A nanostructure can be administered preventively. In some cases, a patient can be administered at least one additional therapy. One additional therapy can be a non-steroidal anti-inflammatory drug, NSAID, a miRNA against B-catenin or any agent that disrupts mucus. In some cases, a non-steroidal anti-inflammatory drug can be Celecoxib. Disclosed herein can be a nanostructure comprising the polynucleic acid of SEQ ID 5.

Disclosed herein are methods of making a nanostructure for gene therapy. A method of making a nanostructure can comprise contacting at least one lipid with at least one polymer in the presence of at least one solvent to form a mixture; re-suspending the mixture in an aqueous solution; incubating a mixture to form at least one liposome; contacting a liposome with at least one polynucleic acid encoding at least one protein or portion thereof; and applying at least a partial coating comprising at least one polymer. In some cases, a polynucleic acid can be isolated. In some cases, a polynucleic acid can be purified. In some cases, a polynucleic acid can be isolated and purified. In some cases, at least one lipid can be DOGS (dioctadecylamidoglycylspermine), DOPE (dioleoylphosphatidylethanolamine), or a combination thereof. In some cases, DOGS and DOPE can be mixed at an 80/20 Mol/Mol ratio. In some cases, at least one polymer can be mixed with DOGS and DOPE. In some cases, a polymer can be mixed at a concentration of 5 to 10 Mol ratio. A polymer can be polyethylene glycol (PEG). PEG can be PEG 2000. In some cases, DOGs, DOPE, PEG2000 can be mixed at 80/20/8 Mol/Mol/mol. In some cases, a method can further comprise at least one modification of a lipid. A modification can be selected from a list comprising additions of peptides, antibodies, single chain variable fragments (scFv), cellular receptors, barcodes, linkers, or any combination thereof. A solvent can be an organic solvent. A solvent can be chloroform. A method can further comprise drying of a solvent. In some cases, drying of a solvent comprises dry nitrogen, argon stream, rotary evaporation, vacuum, or any combination thereof. Drying can be dry nitrogen drying. In other cases, drying can be vacuuming. In some cases, drying can be a dry nitrogen stream followed by a vacuuming. Drying can form a lipid film that can be hydrated by addition of an aqueous solution. An aqueous solution can be high-resistivity water. In some cases, a method can have an incubation that occurs at 37 degrees Celsius. A polynucleic acid can be DNA, RNA, or any combination thereof. A polynucleic acid can be DNA. Polynucleic acid can be mini-circle DNA. In some cases, mini-circle DNA can be mixed with a liposome at a ratio of 4 to 1. In some cases, at least one coating can be pH sensitive. A pH sensitive coating can dissolve at a pH above 5.5. A pH sensitive coating can be poly (methacylic acid-co-ethyl acrylate) 1:1. In some cases, at least one protein can be adenomatous polyposis coli (APC), B-galactosidase, (B-Gal), or any combination thereof. In some cases, at least one protein can be APC. In some cases, a nanostructure can be a mucus penetrating particle (MPP). A MPP can be able to penetrate mucus from 1 to 200 micrometers in thickness. An MPP can have a near neutral zeta potential from about −20 mV to about 20 mV.

Disclosed herein are liposomal structures. A liposomal structure can comprise a polynucleic acid. A polynucleic acid can be isolated. A polynucleic acid can be purified. A polynucleic acid can be isolated and purified. A liposomal structure can comprise a surface modification. The surface modification can enhance an average rate at which a liposomal structure moves in mucus compared to a comparable liposomal structure. A comparable liposomal structure can be surface modified with a polymer. A polymer can be a polyethylene glycol (PEG). A PEG can have an average molecular weight ranging from about 2000 to about 3000 Daltons (Da). In some cases, a liposomal structure can be a liposome, a lipoplex, or a lipopolyplex. In some cases, a liposomal structure can be a liposome. A liposomal structure can be a lipoplex. A liposomal structure can be a lipopolyplex. In some cases, a surface modification can comprise a polymer of Formula I:

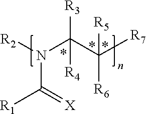

wherein R₁ can be independently selected from a group consisting of hydrogen; deuterium; C₁₋₆ alkyl; C₂₋₆ alkynyl; C₂₋₆ alkynyl, C₃₋₈ cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be selected from a group consisting of a coupling group capable of coupling to a linker, or a substrate; hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R3 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R4 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R5 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R6 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R7 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, wherein when R₃ and R₄ are not the same * can independent be R or S; wherein when R₅, R₆ and R₇ are not the same ** can be R or S; wherein X can be independently selected from oxygen or sulfur; Y can be independently selected from deuterium or hydrogen; A is hydrogen, deuterium, aryl, or heteroaryl and n is 1-100. In some cases, R₁ can be a C₁₋₆ alkyl. In some cases, any one of R₃, R4, R₅, or R₆ can be selected from the group consisting of deuterium and hydrogen. In some cases, X can be oxygen. Formula I can have an average molecular weight from about 1000 Da to about 8000 Da. A surface modification can comprise poly (2-methyl-2-oxazoline), poly (2-ethyl-2-oxazoline), a salt thereof, a di block polymer thereof, a tri block polymer thereof, or a combination thereof. A surface modification can be at a density from about 0.05 μg/nm² to about 0.25 μg/nm². An average rate at which a liposomal structure moves in mucus can be from about 2 fold to about 5 fold greater than an average rate of a comparable liposomal structure as measured by a transwell migration assay. In some cases, a polynucleic acid can comprises DNA. A polynucleic acid can be minicircle DNA or closed-linear DNA. In some cases, a polynucleic acid can comprise minicircle DNA. In some cases, a polynucleic acid can comprises RNA. A polynucleic acid can be at least partially enclosed within a liposomal structure. A liposomal structure can comprise at least two polynucleic acids. A liposomal structure can further comprises a peptide, antibody or fragment thereof, carbohydrate, single chain variable fragment (scFv), cellular receptor, or any combination thereof. In some cases, a liposomal structure can comprise a peptide, antibody or fragment thereof, single chain variable fragment (scFv), or cellular receptor in contact with a surface modification. In some cases, a liposomal structure can comprise a peptide. A peptide can be a cell-penetrating peptide. A liposomal structure can comprise an antibody or fragment thereof. A liposomal structure can comprise an antibody or fragment thereof that can target a leucine-rich repeat-containing G-protein coupled receptor 5 (LGR5). A liposomal structure can further comprise an exterior coating. An exterior coating can be cationic. An exterior coating can be anionic. An exterior coating can be neutral. An exterior coating can comprise ethyl acrylate in polymerized form. An exterior coating can have a near-neutral zeta potential as measured by laser doppler anemometry. In some cases, a near-neutral zeta potential can be from about −20 mV to about 20 mV. In some cases, a near-neutral zeta potential can be from about −100 mV to about 100 mV. A liposomal structure can comprise a lipid bilayer. A lipid bilayer that can comprise one or more of cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt of any of these, or any combination thereof. In some cases, a material can be a lipid with a net positive charge or a lipid with a neutral charge. A lipid bilayer can comprise MVL5. A lipid bilayer can comprise MVL5 and GMO. In some cases, a molar ratio of MVL5 to GMO ranges from about 10:1 to about 1:25. A lipid bilayer can comprise DOGS and DOPE. A polynucleic acid can be fully encapsulated in a lipid bilayer. A polynucleic acid can be in contact with a lipid bilayer. In some cases, a polynucleic acid may not be in contact with a lipid bilayer. In some cases, a liposomal structure can further comprise a second lipid bilayer. In some cases, a liposomal structure can further comprise a linker. A linker can be an acid sensitive linker. A linker associates with the surface modification of the liposomal structure. A linker can directly associate with a surface modification of a liposomal structure. In some cases, a linker can indirectly associate with a surface modification of a liposomal structure. A polynucleic acid can encode for at least a fragment of a protein. At least a fragment of a protein can be active in a gastrointestinal (GI) tract. In some cases, at least a fragment of a protein can be active in a bodily area comprising a mucosal membrane. A polynucleic acid can encode for at least a fragment of adenomatous polyposis coli (APC), defensin alpha 5 (HD-5), defensin alpha 6 (HD-6), or any combination thereof. In some cases, a liposomal structure can have a diameter selected from the group consisting of: from about 10 nm to about 100 nm, from about 100 nm to about 200 nm, from about 200 nm to about 300 nm, from about 300 nm to about 400 nm, and from about 400 nm to about 500 nm as measured by dynamic light scattering.

Disclosed herein are liposomal structures. A liposomal structure can comprise a polynucleic acid. A polynucleic acid can be isolated. A polynucleic acid can be purified. A polynucleic acid can be isolated and purified. A polynucleic acid can be free of a bacterial origin of replication. A liposomal structure can be surface modified with a polymer. A liposomal structure can comprise a polymer comprising Formula I

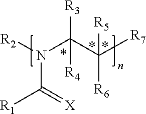

wherein R₁ can be independently selected from a group consisting of hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be independently selected from a group consisting of a coupling group capable of coupling to a linker, or a substrate; hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₃ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₄ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₅ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₆ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₇ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; wherein * can independently be R, S or achiral; wherein ** can independently be R, S or achiral; wherein X can be independently selected from oxygen or sulfur; Y can be independently selected from deuterium or hydrogen; A can be hydrogen, deuterium, aryl, or heteroaryl and n can be from about 1 to about 100. In some cases, R₁ can be a C₁₋₆ alkyl. R₃, R₄, R₅, or R₆ can be selected from the group consisting of deuterium and hydrogen. X can be an oxygen. In some cases, Formula I can have an average molecular weight from about 1000 Da to about 8000 Da. In some cases, a polymer can comprise poly (2-methyl-2-oxazoline), poly (2-ethyl-2-oxazoline), a salt thereof, a di block polymer thereof, a tri block polymer thereof, or a combination thereof. A liposomal structure can be a liposome, a lipoplex, or a lipopolyplex. A liposomal structure can be a liposome. A liposomal structure can be a lipoplex. A liposomal structure can be a lipopolyplex. In some cases, a polymer can have a density of from about 0.05 μg/nm² to about 0.25 μg/nm². A polymer can enhance an average rate at which a liposomal structure moves in mucus compared to an otherwise comparable liposomal structure, wherein the comparable liposomal structure is surface modified with polyethylene glycol (PEG). In some cases, PEG can have an average molecular weight from 2000 Da to 3000 Da. An average rate at which a liposomal structure moves in mucus can be from about 2 fold to about 5 fold greater than the average rate of the comparable liposomal structure as measured by a transwell migration assay. A liposomal structure can have increased hydrophilicity compared to a comparable liposomal structure. A polynucleic acid can comprise DNA. A polynucleic acid can be minicircle DNA or closed-linear DNA. A polynucleic acid can comprise minicircle DNA. A polynucleic acid can comprise ribonucleic acid (RNA). A polynucleic acid can be at least partially water soluble. A polynucleic acid can be at least partially enclosed within a liposomal structure. A liposomal structure can comprise at least two polynucleic acids. A polynucleic acid can comprise at least one promoter. At least one promoter can be selected from cytomegalovirus (CMV) derived promoter, chicken β-actin (CBM) derived promoter, adenomatous polyposis coli (APC) derived promoter, leucine-rich repeat containing G protein-coupled receptor 5 (LGR5), CAG promoter, Beta actin promoter, elongation factor-1 (EF1) promoter, early growth response 1 (EGR-1) promoter, eukaryotic initiation factor 4A (EIF4A1) promoter, or any combination thereof. A liposomal structure can further comprise a peptide, antibody or fragment thereof, carbohydrate, single chain variable fragment (scFv), cellular receptor, or any combination thereof. A liposomal structure can comprise a peptide, antibody or fragment thereof, single chain variable fragment (scFv), or cellular receptor in contact with a polymer. A liposomal structure can comprise a peptide, wherein a peptide can be a cell-penetrating peptide. In some cases, a peptide can be in contact with a polynucleic acid. A peptide may not be in contact with a polynucleic acid. A liposomal structure can comprise an antibody or fragment thereof, wherein an antibody or fragment thereof can target a leucine-rich repeat-containing G-protein coupled receptor 5 (LGR5). In some cases, a liposomal structure can further comprise a nuclease inhibitor. A nuclease inhibitor can be selected from the group consisting of aurintricarboxylic acid (ATA), Zn²⁺, DMI-2, salts thereof, or a combination thereof. A liposomal structure can further comprise an effector of RNA interference (RNAi). An effector of RNA interference (RNAi) can be CEQ508 or salt thereof. A liposomal structure can further comprise an exterior coating. An exterior coating can be cationic. An exterior coating can be anionic. An exterior coating can be neutral. An exterior coating can comprise ethyl acrylate in polymerized form. In some cases, an exterior coating can have a near-neutral zeta potential as measured by laser doppler anemometry. A near-neutral zeta potential can be from about −20 mV to about 20 mV. A near-neutral zeta potential can be from about −100 mV to about 100 mV. A liposomal structure can comprise a lipid bilayer. In some cases, a polynucleic acid can be present in an aqueous solution enclosed in a lipid bilayer. A lipid bilayer can comprise one or more of cholesterol, N-[1-(2,3-dioleyloxy)propyl]-N,N,N-trimethylammonium chloride (DOTMA), [1,2-bis(oleoyloxy)-3 (trimethylammonio)propane](DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt of any of these, or any combination thereof. In some cases, a material can be a lipid with a net positive charge or a lipid with a neutral charge. A lipid bilayer can comprise MVL5. A lipid bilayer can comprise MVL5 and GMO. A molar ratio of MVL5 to GMO can range from about 10:1 to about 1:25. A molar ratio of MVL5 and GMO can range from about 10:1 to about 1:10. A lipid bilayer can comprise DOGS and DOPE. A polynucleic acid can be fully encapsulated in a lipid bilayer. A polynucleic acid can be in contact with a lipid bilayer. A polynucleic acid may not be in contact with a lipid bilayer. A liposomal structure can further comprise a second lipid bilayer. In some cases, a liposomal structure can further comprise a linker. A linker can be an acid sensitive linker. A linker can associate with a polymer. A linker can directly associate with a polymer. A linker can indirectly associate with a polymer. A polynucleic acid can encode for at least a fragment of a protein. In some cases, at least a fragment of a protein can be active in a gastrointestinal (GI) tract. At least a fragment of a protein can be active in a bodily area comprising a mucosal membrane. A polynucleic acid can encode for at least a fragment of adenomatous polyposis coli (APC), defensin alpha 5 (HD-5), defensin alpha 6 (HD-6), or any combination thereof. A liposomal structure can have a diameter selected from the group consisting of: from about 10 nm to about 100 nm, from about 100 nm to about 200 nm, from about 200 nm to about 300 nm, from about 300 nm to about 400 nm, and from about 400 nm to about 500 nm as measured by dynamic light scattering. A liposomal structure can have a diameter from about 100 nm to about 200 nm as measured by dynamic light scattering.

Disclosed herein are liposomal structures. A liposomal structure can comprise a polynucleic acid. A polynucleic acid can be isolated. A polynucleic acid can be purified. A polynucleic acid can be isolated and purified. A liposomal structure can be surface modified with the polymer of Formula I

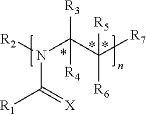

wherein R₁ can be independently selected from a group consisting of hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be independently selected from a group consisting of a coupling group capable of coupling to a linker, or a substrate; hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₃ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₄ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₅ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₆ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₇ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; wherein * can independently be R, S or achiral; wherein ** can independently be R, S or achiral; wherein X can be independently selected from oxygen or sulfur; Y can be independently selected from deuterium or hydrogen; A can be hydrogen, deuterium, aryl, or heteroaryl and n can be from about 1 to about 100. In some cases, R₁ can be a C₁₋₆ alkyl. R₃, R₄, R₅, or R₆ can be selected from the group consisting of deuterium and hydrogen. X can be an oxygen. In some cases, Formula I can have an average molecular weight from about 1000 Da to about 8000 Da. In some cases, a polymer can comprise poly (2-methyl-2-oxazoline), poly (2-ethyl-2-oxazoline), a salt thereof, a di block polymer thereof, a tri block polymer thereof, or a combination thereof. A liposomal structure can be a liposome, a lipoplex, or a lipopolyplex. A liposomal structure can be a liposome. A liposomal structure can be a lipoplex. A liposomal structure can be a lipopolyplex. In some cases, a polymer can have a density of from about 0.05 μg/nm² to about 0.25 μg/nm². A polymer can enhance an average rate at which a liposomal structure moves in mucus compared to an otherwise comparable liposomal structure, wherein the comparable liposomal structure is surface modified with polyethylene glycol (PEG). In some cases, PEG can have an average molecular weight from 2000 Da to 3000 Da. An average rate at which a liposomal structure moves in mucus can be from about 2 fold to about 5 fold greater than the average rate of the comparable liposomal structure as measured by a transwell migration assay. A liposomal structure can have increased hydrophilicity compared to a comparable liposomal structure. A polynucleic acid can comprise DNA. A polynucleic acid can be minicircle DNA or closed-linear DNA. A polynucleic acid can comprise minicircle DNA. A polynucleic acid can comprise ribonucleic acid (RNA). A polynucleic acid can be at least partially water soluble. A polynucleic acid can be at least partially enclosed within a liposomal structure. A liposomal structure can comprise at least two polynucleic acids. A polynucleic acid can comprise at least one promoter. At least one promoter can be selected from cytomegalovirus (CMV) derived promoter, chicken β-actin (CBM) derived promoter, adenomatous polyposis coli (APC) derived promoter, leucine-rich repeat containing G protein-coupled receptor 5 (LGR5), CAG promoter, Beta actin promoter, elongation factor-1 (EF1) promoter, early growth response 1 (EGR-1) promoter, eukaryotic initiation factor 4A (EIF4A1) promoter, or any combination thereof. A liposomal structure can further comprise a peptide, antibody or fragment thereof, carbohydrate, single chain variable fragment (scFv), cellular receptor, or any combination thereof. A liposomal structure can comprise a peptide, antibody or fragment thereof, single chain variable fragment (scFv), or cellular receptor in contact with a polymer. A liposomal structure can comprise a peptide, wherein a peptide can be a cell-penetrating peptide. In some cases, a peptide can be in contact with a polynucleic acid. A peptide may not be in contact with a polynucleic acid. A liposomal structure can comprise an antibody or fragment thereof, wherein an antibody or fragment thereof can target a leucine-rich repeat-containing G-protein coupled receptor 5 (LGR5). In some cases, a liposomal structure can further comprise a nuclease inhibitor. A nuclease inhibitor can be selected from the group consisting of aurintricarboxylic acid (ATA), Zn²⁺, DMI-2, salts thereof, or a combination thereof. A liposomal structure can further comprise an effector of RNA interference (RNAi). An effector of RNA interference (RNAi) can be CEQ508 or salt thereof. A liposomal structure can further comprise an exterior coating. An exterior coating can be cationic. An exterior coating can be anionic. An exterior coating can be neutral. An exterior coating can comprise ethyl acrylate in polymerized form. In some cases, an exterior coating can have a near-neutral zeta potential as measured by laser doppler anemometry. A near-neutral zeta potential can be from about −20 mV to about 20 mV. A near-neutral zeta potential can be from about −100 mV to about 100 mV. A liposomal structure can comprise a lipid bilayer. In some cases, a polynucleic acid can be present in an aqueous solution enclosed in a lipid bilayer. A lipid bilayer can comprise one or more of cholesterol, N-[1-(2,3-dioleyloxy)propyl]-N,N,N-trimethylammonium chloride (DOTMA), [1,2-bis(oleoyloxy)-3 (trimethylammonio)propane](DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt of any of these, or any combination thereof. In some cases, a material can be a lipid with a net positive charge or a lipid with a neutral charge. A lipid bilayer can comprise MVL5. A lipid bilayer can comprise MVL5 and GMO. A molar ratio of MVL5 to GMO can range from about 10:1 to about 1:25. A molar ratio of MVL5 and GMO can range from about 10:1 to about 1:10. A lipid bilayer can comprise DOGS and DOPE. A polynucleic acid can be fully encapsulated in a lipid bilayer. A polynucleic acid can be in contact with a lipid bilayer. A polynucleic acid may not be in contact with a lipid bilayer. A liposomal structure can further comprise a second lipid bilayer. In some cases, a liposomal structure can further comprise a linker. A linker can be an acid sensitive linker. A linker can associate with a polymer. A linker can directly associate with a polymer. A linker can indirectly associate with a polymer. A polynucleic acid can encode for at least a fragment of a protein. In some cases, at least a fragment of a protein can be active in a gastrointestinal (GI) tract. At least a fragment of a protein can be active in a bodily area comprising a mucosal membrane. A polynucleic acid can encode for at least a fragment of adenomatous polyposis coli (APC), defensin alpha 5 (HD-5), defensin alpha 6 (HD-6), or any combination thereof. A liposomal structure can have a diameter selected from the group consisting of: from about 10 nm to about 100 nm, from about 100 nm to about 200 nm, from about 200 nm to about 300 nm, from about 300 nm to about 400 nm, and from about 400 nm to about 500 nm as measured by dynamic light scattering. A liposomal structure can have a diameter from about 100 nm to about 200 nm as measured by dynamic light scattering.

Disclosed herein are pharmaceutical compositions. A pharmaceutical composition can comprise a liposomal structure. A pharmaceutical composition can comprise an excipient. A pharmaceutical composition can comprise a diluent. A pharmaceutical composition can comprise a carrier. A pharmaceutical composition can be in unit dosage form. A pharmaceutical composition can be in the form of a tablet. A pharmaceutical composition can be in the form of a liquid. A pharmaceutical composition can be in the form of a syrup. A pharmaceutical composition can be in the form of an oral formulation. A pharmaceutical composition can be in the form of an intravenous formulation. A pharmaceutical composition can be in the form of an intranasal formulation. A pharmaceutical composition can be in the form of a subcutaneous formulation. A pharmaceutical composition can be in the form of an inhalable respiratory formulation. A pharmaceutical composition can be in the form of a suppository. A pharmaceutical composition can be in the form of a tablet, a liquid, a syrup, an oral formulation, an intravenous formulation, an intranasal formulation, a subcutaneous formulation, an inhalable respiratory formulation, a suppository, and any combination thereof.

Disclosed herein are methods of treating a subject in need thereof. In some cases, a method of treating a subject can comprise administering to a subject in need thereof a therapeutically effective amount of a liposomal structure. In some cases, a method of treating a subject can comprise administering to a subject in need thereof a pharmaceutical composition. In some cases, administration of a liposomal structure or a pharmaceutical composition can at least partially ameliorate a disease or condition in a subject in need thereof. A disease or condition can comprise familial adenomatous polyposis (FAP), attenuated FAP, cancer, chronic inflammatory bowel disease, chronic inflammatory bowel disease, ileal Crohn's or any combination thereof. A disease or condition can be FAP. A subject can have a polyp in a gastrointestinal tract. A subject in need thereof can have a polyp surgically removed prior to, after, or concurrent with administration of a liposomal structure or a pharmaceutical composition. A liposomal structure or a pharmaceutical composition can be administered orally, rectally, or orally and rectally. A liposomal structure or a pharmaceutical composition can be administered routinely. A liposomal structure or a pharmaceutical composition can be administered prophylactically. A liposomal structure or a pharmaceutical composition can be administered 1 time per day, 2 times per day, 3 times per day, daily, weekly, yearly or any combination thereof. A subject can be administered an additional therapy in a therapeutically effective amount. An additional therapy can comprise a non-steroidal anti-inflammatory drug (NSAID) or a salt thereof, a miRNA against (3-catenin, a mucus disrupting agent or a salt a salt thereof, or any combination thereof. A non-steroidal anti-inflammatory drug (NSAID) can comprise Celecoxib. A mucus disrupting agent can comprise guaifenesin. A subject in need thereof can be genetically screened for a disease or condition.

A liposomal structure or a pharmaceutical composition described herein can be comprised in a kit. A kit can comprise a pharmaceutical composition described herein, and instructions for use thereof. A kit can further comprise a container. Also disclosed herein are methods of making a kit. A method of making a kit can comprise placing a liposomal structure described herein or a pharmaceutical composition described herein into a container. A kit or method of making a kit can further comprise instructions for use.

Disclosed herein are methods of making a liposomal structure. Also disclosed herein are methods of making a pharmaceutical composition. A method of making a liposomal structure or a pharmaceutical composition can comprise forming a liposome around a polynucleic acid. A liposomal structure can be surface modified with a polymer. A polynucleic acid can encode for a protein or portion thereof that can be active in a gastrointestinal tract or a tumor suppressor protein or portion thereof. A liposomal structure can be a liposome, a lipoplex, or a lipopolyplex. A liposomal structure can be a liposome. A polymer can comprise Formula I:

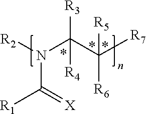

wherein R₁ can be independently selected from a group consisting of hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₂ can be independently selected from a group consisting of a coupling group capable of coupling to a linker, or a substrate; hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof; R₃ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₄ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₅ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₆ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; R₇ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; wherein * can independently be R, S or achiral; wherein ** can independently be R, S or achiral; wherein X can be independently selected from oxygen or sulfur; Y can be independently selected from deuterium or hydrogen; A can be hydrogen, deuterium, aryl, or heteroaryl and n can be from about 1 to about 100. R₁ can be C₁₋₆ alkyl. Any one of R₃, R₄, R₅, or R₆ can be selected from the group consisting of deuterium and hydrogen. X can be oxygen. In some cases, Formula I can have an average molecular weight from about 1000 Da to about 8000 Da. A polymer can comprise poly (2-methyl-2-oxazoline), poly (2-ethyl-2-oxazoline), a salt thereof, a di block polymer thereof, a tri block polymer thereof, or a combination thereof. A method can further comprise introducing a solvent. A solvent can comprise chloroform. A method can further comprise drying a solvent. Drying can comprise exposing a solvent to dry nitrogen, argon stream, rotary evaporation, vacuum, or any combination thereof. Drying can comprise exposing a solvent to dry nitrogen. Drying can comprise exposing a drying to a vacuum. In some cases, drying can comprise exposing a solvent to a dry nitrogen stream followed by a vacuum. Drying can form a lipid film that can be hydrated by addition of an aqueous solution. A method can further comprise an aqueous solution. A method can comprise a polynucleic acid comprising DNA, RNA, or any combination thereof. A polynucleic acid can comprise DNA. A polynucleic acid can comprise mini-circle DNA.

Disclosed herein are liposomal structures. A liposomal structure can comprise a polynucleic acid. A polynucleic acid can be isolated. A polynucleic acid can be purified. A polynucleic acid can be isolated and purified. A purified polynucleic acid can be at least partially enclosed within a liposomal structure. A liposomal structure can have increased hydrophilicity compared to a comparable liposomal structure A comparable liposomal structure can comprise a polyethylene glycol (PEG) surface modification. In some cases, increased hydrophilicity can be caused by a non-PEG surface modification. A non-PEG surface modification can comprise a polymer of Formula I:

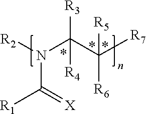

wherein R₁ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof, R₂ can be independently selected from a group consisting of a coupling group capable of coupling to a linker, or a substrate; hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof, R3 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R4 can independently be selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R5 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R6 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, R7 can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof, wherein when R₃ and R₄ are not the same * can independent be R or S; wherein when R₅, R₆ and R₇ are not the same ** can independent be R or S; wherein X can be independently selected from oxygen or sulfur; Y can be independently selected from deuterium or hydrogen; A can be hydrogen, deuterium, aryl, or heteroaryl and n can be from about 1 to about 100. R₁ can be a C₁₋₆ alkyl. Any one of R₃, R₄, R₅, or R₆ can be selected from the group consisting of deuterium and hydrogen. X can be oxygen. Formula I can have an average molecular weight from about 1000 Da to about 8000 Da. A surface modification can comprise poly (2-methyl-2-oxazoline), poly (2-ethyl-2-oxazoline), a salt thereof, a di block polymer thereof, a tri block polymer thereof, or a combination thereof. A polynucleic acid can comprise minicircle DNA. A liposomal structure can comprise a lipid bilayer. A lipid bilayer can comprise one or more of cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt of any of these, or any combination thereof. In some cases, a material can be a lipid with a net positive charge or a lipid with a neutral charge. A lipid bilayer can comprise MVL5 and GMO.

Disclosed herein are nanostructures. In some embodiments a nanostructure can comprise: at least one polynucleic acid encoding at least one protein or portion thereof, at least one lipid bilayer contacting at least one polymer; and at least one external coating; wherein a polynucleic acid can be at least partially encapsulated within a lipid bilayer and an external coating can be at least partially coating a nanostructure and wherein at least one of the following can be comprised: an external coating can be an enteric coating at least one polymer comprises a polyglycol polymer, or any combination thereof. In some cases, a polynucleic acid can be isolated. In some cases, a polynucleic acid can be purified. In some cases, a polynucleic acid can be isolated and purified. In some cases, a polynucleic acid can be circular. In some cases, a nanostructure can have a diameter from about 500 nm or less. In other cases, a nanostructure can have a diameter selected from a list comprising: from about 10 nm to from about 100 nm, from about 100 nm to from about 200 nm, from about 200 nm to from about 300 nm, from about 300 nm to from about 400 nm, or from about 400 nm to from about 500 nm. At least one external coating can be partially coating. At least one external coating can be fully coating. In some cases, a nanostructure can comprise at least one external coating that can be selected from a list comprising: Cellulose acetate phthalate, Polyvinyl acetate phthalate, Hydroxypropylmethylcellulose acetate succinate, Poly(methacylic acid-co-ethyl acrylate) 1:1, Poly(methacrylic acid-co-ethyl acrylate) 1:1, Poly(methacylic acid-co-methyl methacrylate) 1:1, Poly(methacylic acid-co-methyl methacrylate) 1:1, Poly(methacylic acid-co-methyl methacrylate) 1:2, Poly(methacylic acid-co-methyl methacrylate) 1:2, or Poly(methyl acrylate-co-methyl methacrylate-co-methacrylic acid) 7:3:1. At least one external coating can be Poly (methacylic acid-co-ethyl acrylate) 1:1. At least one external coating can be a mucoadhesive hydrogel. A mucoadhesive hydrogel can be selected from a group consisting of Hydroxyethyl Cellulose (HEC), polyacrylates (carbomer), alginates, chitosan, and cellulosic derivatives (hydroxyethylcellulose, hydroxypropylmethylcellulose, or carboxymethylcellulose. In some cases, at least one external coating can be pH sensitive. A pH sensitive coating can dissolve above a pH of 5.5 as measured at 37 degrees Celsius with a pH meter when dissolved in 1 L of water with a stirring rod rotating at 200 revolutions per minute. A pH sensitive coating can dissolve above a pH of 7 as measured at 37 degrees Celsius with a pH meter when dissolved in 1 L of water with a stirring rod rotating at 200 revolutions per minute. In other cases, a pH sensitive coating can dissolve above a pH of 6 as measured at 37 degrees Celsius with a pH meter when dissolved in 1 L of water with a stirring rod rotating at 200 revolutions per minute. Dissolution can occur enterically. In other cases, dissolution can occur in an organ selected from a group consisting of a duodenum, jejunum, ilium, and colon. In some cases, dissolution can occur in proximity to an intestinal crypt cell. In proximity can refer to adjacent to an intestinal crypt cell. For example, adjacent can mean immediately next to. In other cases, adjacent can mean within the same section of an intestine. For example, if dissolution occurs in a duodenum of a small intestine it may be considered as adjacent to an intestinal crypt cell found in a duodenum. In some embodiments, at least one polymer can be attached to a lipid bilayer directly, covalently, non-covalently, via a linker, or any combination thereof. A polymer can be attached covalently to a lipid bilayer. At least one polymer can be polyethylene glycol (PEG), a triblock copolymer of PEG-polypropyelene oxide, poly(2-methyl-2-oxazoline), poly(vinyl alcohol), poly(vinyl ethers), poly(N-[2-hydroxypropyl)methylacrylamide), polyethyleneimine (PEI), poly(2-dimethylaminoethyl methacrylate) (pDMAEMA), and poly-L-lysine (pLL) a modified version, or derivative thereof. In some cases, a polymer comprises poly(2-methyl-2-oxazoline) or PEG. In some cases, at least one polymer may not interact with mucus as measured by an increase in the distance of mucus transversed by a nanostructure comprising at least one polymer that may not interact with mucus compared to a nanostructure that may not contain a polymer. In some cases, PEG can be PEG 2000 comprising a molecular weight average from 1900 g/mol to 2200 g/mol. PEG can be attached to a lipid bilayer from about 10 to 20 chains per 100 nm². PEG surface density can be estimated using an actual molar ratio of lipid-PEG in the liposome and a calculated weighted average surface area of a liposome. The actual molar ratio of lipid-PEG that can be determined can be 1HNMR prepared in D2O with 1% w/w DSS as reference. 500 MHz, 10 s relaxation time and ZG pulse set at 90 degrees. PEG peaks occur at 3.3-4.1 ppm and their integral can be compared to standards. In some cases, PEG can be in a mushroom configuration. In other cases, PEG can be in a brush configuration. In other cases, PEG can be in a pancake configuration. A lipid bilayer can be in a form of a liposome. In some cases, a lipid bilayer can be generated from a list of lipids selected from a group consisting of: cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), Dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO) derivatives thereof, and combinations thereof. A lipid bilayer can be comprised of DOGS and DOPE. In some cases, DOGS and DOPE can be at an 80 Mol to 20 Mol ratios. In some cases, PEG can be combined with at least one lipid at a concentration of 5 Mol to 10 Mol. In some cases, a PEG concentration can be selected from a list comprising: DOGS/DOPE/PEG at 80 mol/20 mol/5 mol, 80 mol/20 mol/6 mol, 80 mol/20 mol/7 mol, 80 mol/20 mol/8 mol, 80 mol/20 mol/9 mol, or 80 mol/20 mol/10 mol. In some cases, MVL5 and GMO can be used as lipids in a liposome structure. Molar concentrations of MVL5 and GMO can range from about 10:1 to 1:25. A molar ratio of MVL5 and GMO can range from about 10:1 to about 1:10. In other cases, a molar ratio of MVL5 and GMO can range from about 50:1 to 1:1. For example a molar ratio can be from about 50:1 to 1:1, 40:1 to 1:1, 30:1 to 1:1, 20:1 to 1:1, 10:1 to 1:1 or about 5:1 to 1:1. In some cases, a molar ratio of MVL5/GMO/lipid-HPEG can be from about 50 mol/45 mol/5 mol, 50 mol/44 mol/6 mol, 50 mol/43 mol/7 mol, 50 mol/42 mol/8 mol, 50 mol/41 mol/9 mol, to about 50 mol/40 mol/10 mol. In some cases, a lipid, such as MVL5, can hydrogen bond a polynucleic acid such as a minicircle polynucleic acid. In some cases, a polymer can further comprise a peptide, antibody, carbohydrate, or a combination thereof. A peptide or antibody can be selected from a list comprising antibodies, single chain variable fragments (scFv), cellular receptors, barcodes, linkers, or any combination thereof. A peptide can be a cell-penetrating peptide. In some cases, an antibody can be leucine-rich repeat-containing G-protein coupled receptor 5 (LGR5). A nanostructure can be cationic, anionic, neutral, or any combination thereof. A nanostructure can be neutral. In some cases, a nanostructure can have a near-neutral zeta potential as measured by laser doppler anemometry. In some cases, a non-neutral charge can be from −100 mV to 100 mV for nanostructures at a DNA charge ratio of 10 in 1 mL of high-resistivity water are measured by a Malvern Nanosizer ZS. A DNA charge ratio can be from 0 to 20. In some cases, polymers that can form a nanoparticle can comprise a “charge ratio”. The charge ratio can refer to a ratio of the number of positive charges (cationic) on the cationic monomers that comprise the second block of the polymers (N) to the number of negative charges (anionic) on the polynucleotides that are incorporated into the nanostructure (P). In some embodiments the cationic charges are cationic amines of the cationic monomers. In some embodiments the anionic charges are anionic charges of the phosphate groups on the backbone the polynucleic acid (e.g., minicircle DNA). In some embodiments the ratio can be calculated at physiological pH, neutral pH, or a combination thereof. In further embodiments, for the purpose of calculating a N:P ratio, the cationic monomers are assumed to have about half (50%) of their cationic species (e.g., amines) charged at the neutral and/or physiological pH. Exemplary nanostructures can be formed with a particular N:P ratio so as to determine or estimate the dosage of polynucleotides in the nanoparticles. Exemplary nanoparticles can also be made at an N:P ratio that achieves the desired characteristics, including size, stability, surface charge, and the like, for the nanoparticles. In some embodiments the N:P ratio can be a value between about 0.5 and about 20. In some embodiments the N:P ratio can be a value between about 1.0 and about 30. In some embodiments the N:P ratio can be a value between about 5.0 and about 15, or a value between about 10 and 10. In further embodiments the N:P ratio can be a value from about 1, 2, 3, 4, 5, 6, 7, 8, 9, 10, 11, 12, 13, 14, 15, 16, 17, 18, 19, or up to about 20. In some cases, a polynucleic acid can be DNA, RNA, or any combination thereof. Polynucleic acid can be DNA. DNA can be mini-circle DNA. In some cases, a polynucleic acid can be water soluble. A polynucleic acid can be suspended in aqueous solution within a lipid bilayer. In some cases, at least one protein or portion thereof can be adenomatous polyposis coli (APC), B-galactosidase, (B-Gal), or any combination thereof. In some cases, at least one protein or portion thereof can be adenomatous polyposis coli (APC). In some cases, a polynucleic acid can comprise at least one promoter. A promoter can be selected from a list comprising cytomegalovirus (CMV) derived promoter, chicken (3-actin (CBM) derived promoter, adenomatous polyposis coli (APC) derived promoter, leucine-rich repeat containing G protein-coupled receptor 5 (LGR5), CAG promoter, Beta actin promoter, elongation factor-1 (EF1) promoter, early growth response 1 (EGR-1) promoter, eukaryotic initiation factor 4A (EIF4A1) promoter, or any combination thereof. Disclosed herein can be a nanostructure further comprising a protein or peptide. A protein or peptide can comprise a nuclear localization signal. In some cases, a protein or peptide can bind a polynucleic acid. In some cases, a nanostructure can further comprise a DNase inhibitor. A nanostructure can be a mucus penetrating particle (MPP). A MPP can be able to penetrate mucus from 1 to 200 micrometers in thickness. A nanostructure can be at least partially biodegradable. A nanostructure can be freeze-dried. A nanostructure can be administered as a pill, as a hydrogel, or any combination thereof. Disclosed herein can be a pharmaceutical composition comprising a nanostructure disclosed herein. A pharmaceutical composition can comprise a pharmaceutically acceptable excipient.

In some instance, a pharmaceutical composition disclosed herein can be administered to a patient in unit dosage form. In some cases a method disclosed herein can treat or prevent at least one condition in a patient. In some cases, a nanostructure can be used to treat familial adenomatous polyposis (FAP), attenuated FAP, colorectal cancer, chronic inflammatory bowel disease, chronic inflammatory bowel disease or any combination thereof. A nanostructure can be used to treat FAP. A nanostructure can be administered orally or rectally. A nanostructure can be administered routinely. A nanostructure can be administered preventively. In some cases, a patient can be administered at least one additional therapy. One additional therapy can be a non-steroidal anti-inflammatory drug, NSAID, a miRNA against B-catenin or any agent that disrupts mucus. In some cases, a non-steroidal anti-inflammatory drug can be Celecoxib. Disclosed herein can be a nanostructure comprising the polynucleic acid of SEQ ID 5.

Disclosed herein are methods of making a nanostructure for gene therapy. A method of making a nanostructure can comprise contacting at least one lipid with at least one polymer in the presence of at least one solvent to form a mixture; re-suspending the mixture in an aqueous solution; incubating a mixture to form at least one liposome; contacting a liposome with at least one polynucleic acid encoding at least one protein or portion thereof, and applying at least a partial coating comprising at least one polymer. In some cases, a polynucleic acid can be isolated. In some cases, a polynucleic acid can be purified. In some cases, a polynucleic acid can be isolated and purified. In some cases, at least one lipid can be DOGS (dioctadecylamidoglycylspermine), DOPE (dioleoylphosphatidylethanolamine), or a combination thereof. In some cases, DOGS and DOPE can be mixed at an 80/20 Mol/Mol ratio. In some cases, at least one polymer can be mixed with DOGS and DOPE. In some cases, a polymer can be mixed at a concentration of 5 to 10 Mol ratio. A polymer can be polyethylene glycol (PEG). PEG can be PEG 2000. In some cases, DOGs, DOPE, PEG2000 can be mixed at 80/20/8 Mol/Mol/mol. In some cases, a method can further comprise at least one modification of a lipid. A modification can be selected from a list comprising additions of peptides, antibodies, single chain variable fragments (scFv), cellular receptors, barcodes, linkers, or any combination thereof. A solvent can be an organic solvent. A solvent can be chloroform. A method can further comprise drying of a solvent. In some cases, drying of a solvent comprises dry nitrogen, argon stream, rotary evaporation, vacuum, or any combination thereof. Drying can be dry nitrogen drying. In other cases, drying can be vacuuming. In some cases, drying can be a dry nitrogen stream followed by a vacuuming. Drying can form a lipid film that can be hydrated by addition of an aqueous solution. An aqueous solution can be high-resistivity water. In some cases, a method can have an incubation that occurs at 37 degrees Celsius. A polynucleic acid can be DNA, RNA, or any combination thereof. A polynucleic acid can be DNA. Polynucleic acid can be mini-circle DNA. In some cases, mini-circle DNA can be mixed with a liposome at a ratio of 4 to 1. In some cases, at least one coating can be pH sensitive. A pH sensitive coating can dissolve at a pH above 5.5. A pH sensitive coating can be poly (methacylic acid-co-ethyl acrylate) 1:1. In some cases, at least one protein can be adenomatous polyposis coli (APC), B-galactosidase, (B-Gal), or any combination thereof. In some cases, at least one protein can be APC. In some cases, a nanostructure can be a mucus penetrating particle (MPP). A MPP can be able to penetrate mucus from 1 to 200 micrometers in thickness.

### BRIEF DESCRIPTION OF THE DRAWINGS

The novel features of the disclosure are set forth with particularity in the appended claims. A better understanding of the features and advantages of the disclosure will be obtained by reference to the following detailed description that sets forth illustrative embodiments, in which the principles of the disclosure can be utilized, and the accompanying drawings of which:

FIG. 1 shows an APC vector.

FIG. 2 shows a minicircle DNA vector encoding for APC protein.

FIG. 3 shows lipids for liposome generation.

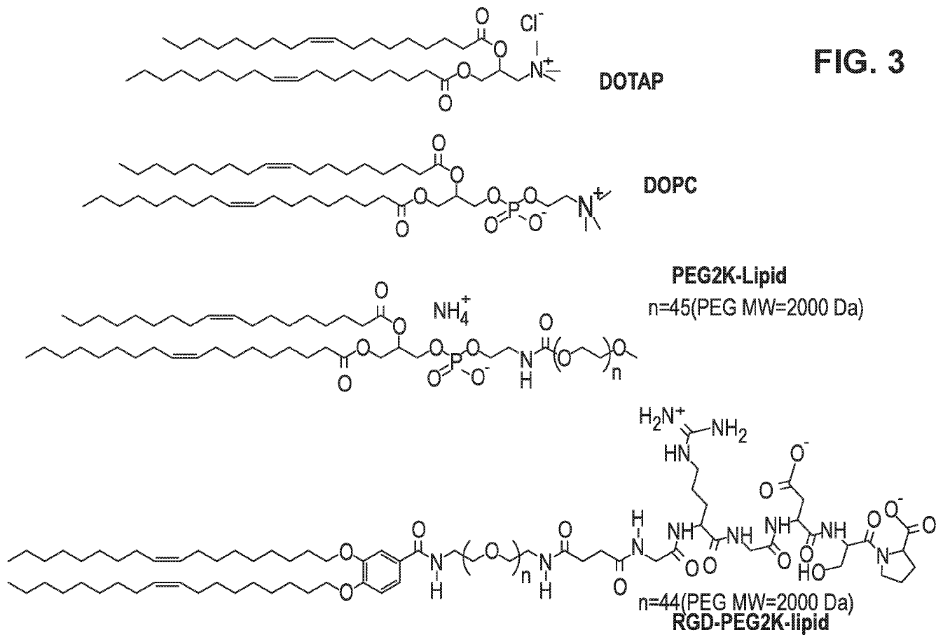

FIG. 4 shows the chemical structure of an exemplary lipid-HPEG.

FIG. 5 shows an exemplary polynucleic acid enclosed in a liposome complex.

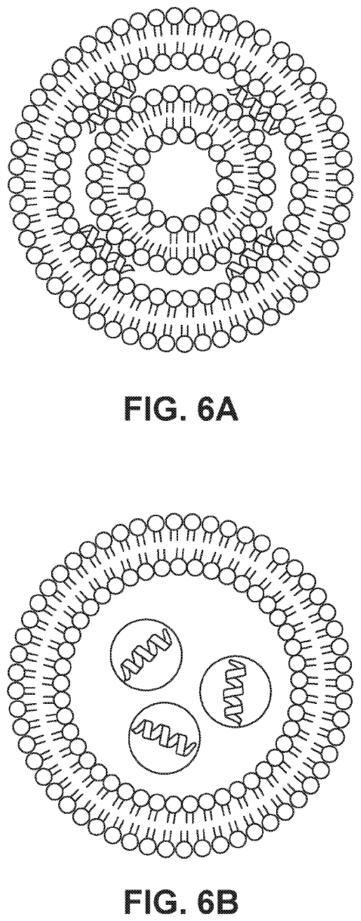

FIG. 6A shows an exemplary lipoplex structure. FIG. 6B shows an exemplary lipopolyplex structure.

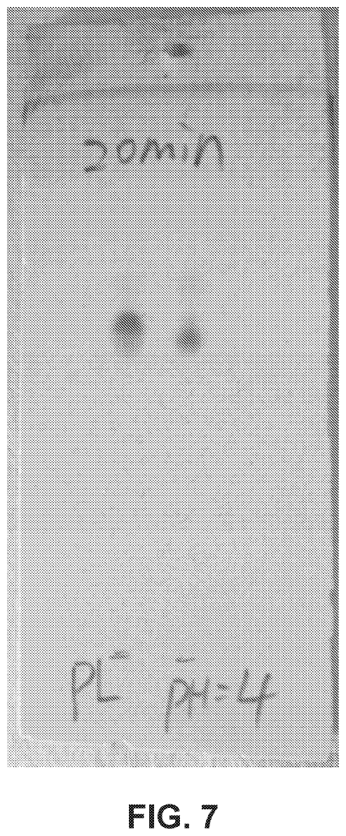

FIG. 7 shows HPEG2K-Lipid analysis using Thin-Layer Chromatography (TLC). Spots were detected using iodine vapor and UV absorption. PL represents HPEG2k-Lipid at neutral pH and was compared to HPEG-2K-lipid incubated at pH4 for 20 min. At pH4, HPEG2k-lipid disintegrated stimulating what would occur to enhance endosomal escape.

FIG. 8A shows a transformed cell line transfected with MVL5/GMO/HPEG liposomes. FIG. 8B shows a transformed cell line transfected with a negative control.

FIG. 9 shows flow cytometric data of percent green fluorescent protein (GFP) positive cells transfected with liposomal structures at low and high charge ratios as compared to positive and negative controls.

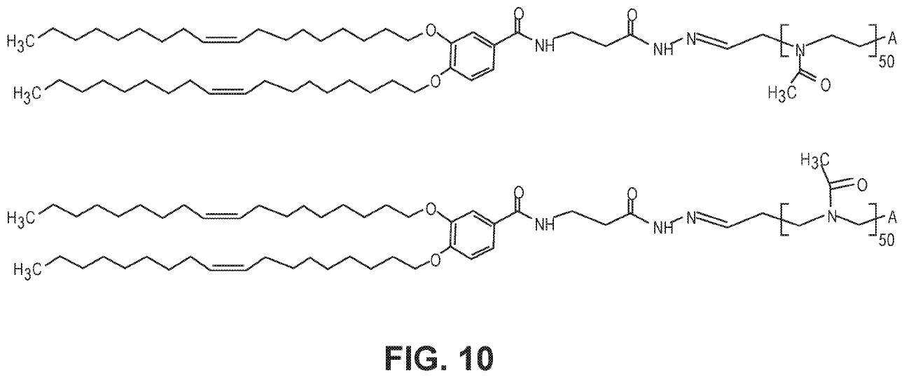

FIG. 10 shows Poly(2-methyl-2-oxazaline) (PMOZ) with a molecular weight of 5 kDa and N=50 was synthesized and attached to a lipid with a hydrazone linker.

FIG. 11 shows MVL5/GMO/lipid-HPMOZ complexes with DNA. From left to right: DNA alone (NTC-eGFP), 0% mol lipid HPMOZ, 2% mol lipid-HPMOZ, 4% mol lipid-HPMOZ, 6% mol lipid-HPMOZ, 8% mol lipid-HPMOZ, 10% mol lipid-HPMOZ. Free DNA was not found in any of the complexes demonstrating that lipid-HPMOZ does not impact DNA complexing efficiencies and little free DNA is present at a charge ratio of 5.

FIG. 12 shows Caco-2 cells transfected with Lipofectamine 2000.

FIG. 13 shows Caco-2 cells transfected with PMOZ 4%.

FIG. 14 shows PMOZ transfection efficiency in Caco-2 cells.

FIG. 15A shows staining of a porcine epithelial layer section. White box indicates the epithelial area selected for pixel intensity for PMOZ 4%. FIG. 15B shows staining of a porcine epithelial layer section. White box indicates the epithelial area selected for pixel intensity for Lipofectamine 2000.

FIG. 16 shows ex vivo mucus penetration at 100 Min.

FIG. 17 shows a western blot of APC present in transfected Rat colorectal cells.

FIG. 18 shows In vivo endoscopic surveillance. The figure shows that at after 4 weeks of dosing, GFP could be observed both in the epithelium and in polyps of RAT cells.

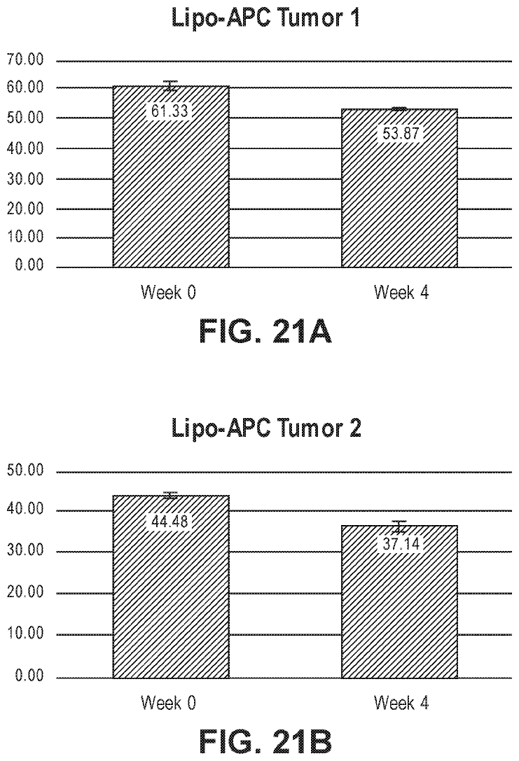

FIG. 19 shows GFP expression observed via endoscopic surveillance only in LiteA1+GFP animals after 4 weeks both in the epithelium and polyps.

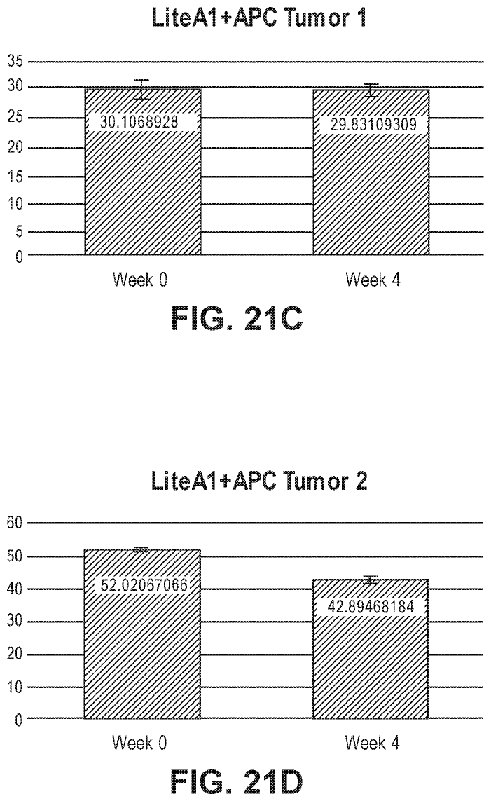

FIG. 20 shows histology of tumors in the LiteA1+GFP control group (7 weeks of dosing) showed that expression was found throughout the tumor as can be observed in the tumor cross-section. Tumor size was measured relative to a probe that was used during endoscopies. Tumor size could then be tracked longitudinally.

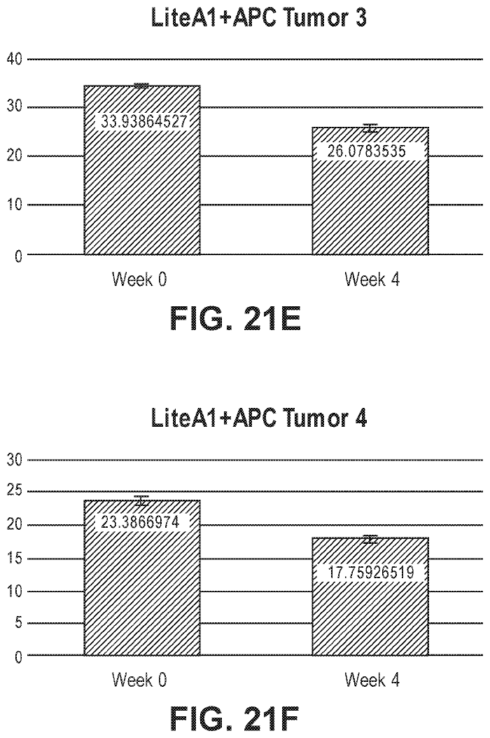

FIG. 21A shows tumor size in Lipofectamine treated animals and FIG. 21B shows tumor size in Lipofectamine treated animals of a second tumor. FIG. 21C shows tumor size in mm in LiteA1-treated animals. FIG. 21D shows tumor size in mm in LiteA1-treated animals of a second tumor.

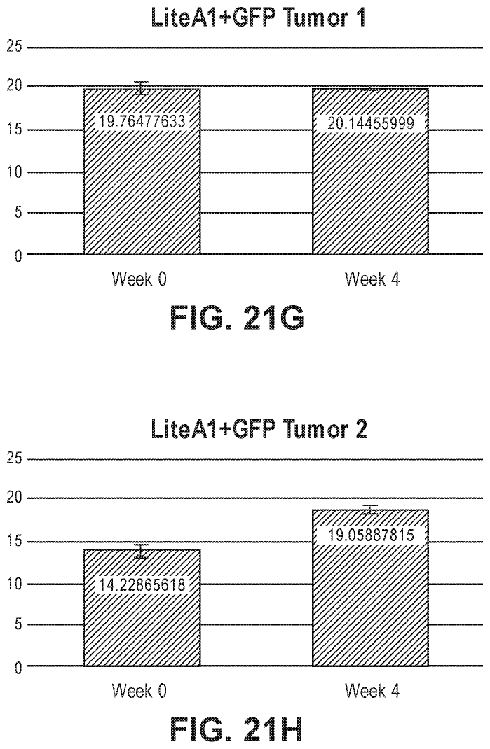

FIG. 21E shows tumor size in mm in LiteA1-treated animals of a third tumor. FIG. 21F shows tumor size in mm in LiteA1-treated animals of a fourth tumor. FIG. 21G shows tumor size in mm in LiteA1-treated+GFP animals of a tumor. FIG. 21H shows tumor size in mm in LiteA1-treated+GFP animals of a second tumor.

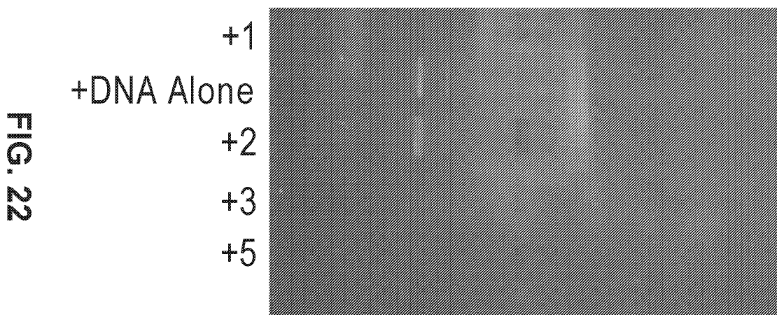

FIG. 22 shows an agarose gel electrophoresis of an in vitro mucus penetration analysis. From right to left (+5, +3, +2, DNA alone and +1).

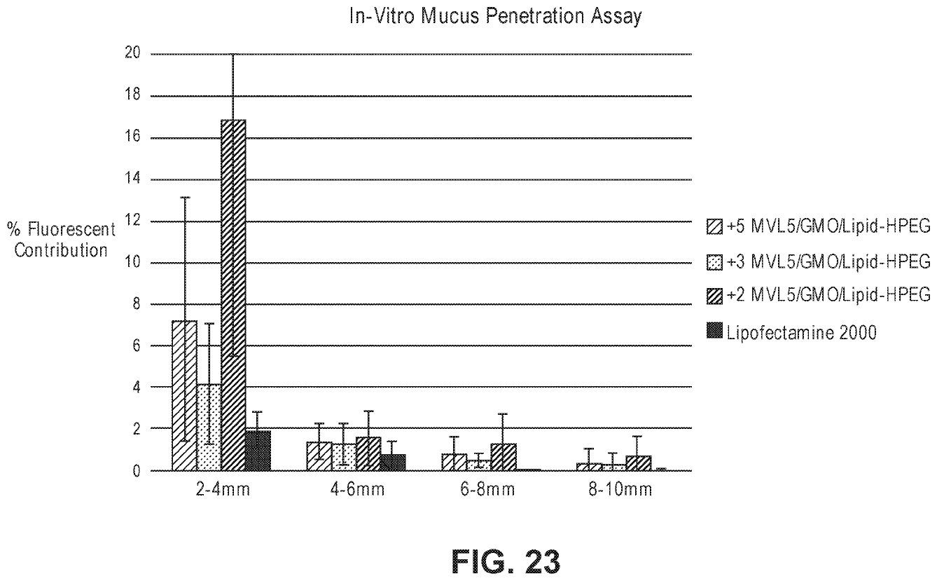

FIG. 23 depicts fluorescent contribution of an in vitro mucus penetration assay for MVL5/GMO/LipidOHPEG at charge ratios of +2, +3, and +5 and Lipofectamine 2000 control.

FIG. 24A depicts transfection of HEK 293T at a charge ratio of 2, FIG. 24B depicts transfection of HEK 293T at a charge ratio of 3, FIG. 24C depicts transfection of HEK 293T at a charge ratio of 5.

FIG. 25A depicts representative images at 5 min of control with Cy5 and FIG. 25B shows bright field at 5 min of the ex vivo porcine mucus experiment. FIG. 25C depicts representative images at 60 min of control with Cy5 and FIG. 25D shows bright field at 60 min of the ex vivo porcine mucus experiment. FIG. 25E depicts representative images at 100 min of control with Cy5 and FIG. 25F shows bright field at 100 min of the ex vivo porcine mucus experiment.

FIG. 26A shows representative images at 5 min of vehicle+60 bp-Cy5 on the left and FIG. 26B shows bright field at 5 min of the ex vivo porcine mucus experiment. FIG. 26C shows representative images at 60 min of vehicle+60 bp-Cy5 and FIG. 26D shows bright field at 60 min of the ex vivo porcine mucus experiment. FIG. 26E shows representative images of the intestinal crypt at 60 min of vehicle+60 bp-Cy5 and FIG. 26F shows bright field at 60 min of the ex vivo porcine mucus experiment.

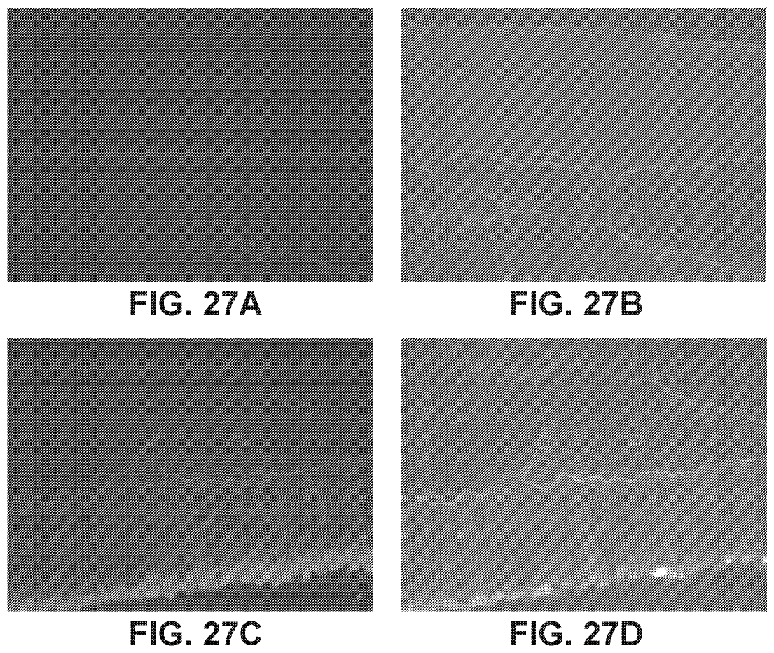

FIG. 27A shows representative images at 100 min of vehicle+60 bp-Cy5 on the left and FIG. 27B shows bright field at 100 min of the ex vivo porcine mucus experiment. FIG. 27C shows representative images of the intestinal crypt at 100 min of vehicle+60 bp-Cy5 and FIG. 27D shows bright field of the intestinal crypt at 100 min of the ex vivo porcine mucus experiment.

FIG. 28A shows intestinal epithelium from liposomal delivery vehicle (Lite)-APC (negative control). FIG. 28B shows intestinal epithelium from Lite and GFP (positive control).

FIG. 29 shows transfection via GFP expression in intestinal crypt cells of Pirc rats treated with liposomal delivery vehicle (Lite).

FIG. 30A shows anti-GFP stained tumor tissue samples of Pirc rats treated with liposomal delivery vehicle (Lite)-APC. FIG. 30B shows anti-GFP tumor tissue samples of Pirc rats treated with liposomal delivery vehicle (Lite)-GFP. FIG. 30C shows a high resolution image of a Pirc rat tumor expressing GFP.

FIG. 31A shows an overlay of 4 different amplitudes of HTLV vs HPRT housekeeping gene of normal colon epethleium of liposomal vehicle treated Pirc rats. FIG. 31B shows an overlay of 4 different amplitudes of HTLV vs HPRT housekeeping gene of colon tumor of liposomal vehicle treated Pirc rats. FIG. 31C shows an overlay of 4 different amplitudes of HTLV vs HPRT housekeeping gene of liver tissue of liposomal vehicle treated Pirc rats. FIG. 31D shows an overlay of 4 different amplitudes of HTLV vs HPRT housekeeping gene of spleen of liposomal vehicle treated Pirc rats. FIG. 31E shows an overlay of 4 different amplitudes of HTLV vs HPRT housekeeping gene of serum (cell free DNA) of liposomal vehicle treated Pirc rats. FIG. 31F shows an overlay of 4 different amplitudes of HTLV vs HPRT housekeeping gene of normal colon epethleium of untreated Pirc rats.

FIG. 32 shows average tumor weight (mg) of Pirc rats treated with (Lipo)-APC, (Lite)-APC, and (Lite)-GFP. Note that the average tumor weight for Lite-GFP is lower than shown as the largest Lite-GFP tumors were fixed for histology rather than weighing.

### DETAILED DESCRIPTION OF THE DISCLOSURE

The following description and examples illustrate embodiments of the disclosure in detail. It is to be understood that this disclosure is not limited to the particular embodiments described herein and as such can vary. Those of skill in the art will recognize that there are numerous variations and modifications of the disclosure, which are encompassed within its scope.

### Definitions

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

The term “administering” and its grammatical equivalents can refer to any method of providing a structure described herein to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a structure disclosed herein can be administered therapeutically. In some instances a structure can be administered to treat an existing disease or condition. In further various aspects, a structure can be administered prophylactically to prevent a disease or condition.

The term “biodegradable” and its grammatical equivalents can refer to polymers, compositions and formulations, such as those described herein that are intended to degrade during use. The term “biodegradable” is intended to cover materials and processes also termed “bioerodible.”

The term “cancer” and its grammatical equivalents as used herein can refer to a hyperproliferation of cells whose unique trait-loss of normal controls-results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

The term “cell” and its grammatical equivalents as used herein can refer to a structural and functional unit of an organism. A cell can be microscopic in size and can consist of a cytoplasm and a nucleus enclosed in a membrane. A cell can refer to an intestinal crypt cell. A crypt cell can refer to the crypts of Lieberkuhn which are pit-like structures that surround the base of the villi in the intestine. A cell can be of human or non-human origin.

A “chemotherapeutic agent” or “Chemotherapeutic compound” and their grammatical equivalents as used herein, can be a chemical compound useful in the treatment of a disease, for example cancer.

The term “function” and its grammatical equivalents as used herein can refer to the capability of operating, having, or serving an intended purpose. Functional can comprise any percent from baseline to 100% of an intended purpose. For example, functional can comprise or comprise about 5, 10, 15, 20, 25, 30, 35, 40, 45, 50, 55, 60, 65, 70, 75, 80, 85, 90, 95, or up to about 100% of an intended purpose. In some cases, the term functional can mean over or over about 100% of normal function, for example, 125, 150, 175, 200, 250, 300%, 400%, 500%, 600%, 700% or up to about 1000% of an intended purpose.

The term “hydrophilic” and its grammatical equivalents as used herein refers to substances or structures that have polar groups that readily interact with water.

The term “hydrophobic” and it's grammatical equivalents as used herein refers to substances or structures that have polar groups that do not readily interact with water.

The term “mucus,” and its grammatical equivalents as used herein, can refer to a viscoelastic natural substance containing primarily mucin glycoproteins and other materials, which protects epithelial surface of various organs/tissues, including but not limited to respiratory, nasal, cervicovaginal, gastrointestinal, rectal, visual and auditory systems.

The term “structure” and its grammatical equivalents as used herein can refer to a nanoparticle or nanostructure. A structure can be a liposomal structure. A structure can also refer to a particle. A structure or particle can be a nanoparticle or nanostructure. A particle or structure can be of any shape having a diameter from about 1 nm up to about 1 micron. A nanoparticle or nanostructure can be or can be about 100 to 200 nm. A nanoparticle or nanostructure can also be up to 500 nm. Nanoparticles or nanostructures having a spherical shape can be referred to as “nanospheres”.

The terms “nucleic acid,” “polynucleotide,” and “oligonucleotide” and their grammatical equivalents can be used interchangeably and can refer to a deoxyribonucleotide and/or ribonucleotide polymer, in linear or circular conformation, and in either single- or double-stranded form. For the purposes of the present disclosure, these terms should not to be construed as limiting with respect to length. The terms can also encompass known analogues of natural nucleotides, as well as nucleotides that are modified in the base, sugar and/or phosphate moieties (e.g., phosphorothioate backbones). In general, an analogue of a particular nucleotide can have the same base-pairing specificity, i.e., an analogue of adenine “A” can base-pair with thymine “T”.

The term “pharmaceutically acceptable carrier” and their grammatical equivalents can refer to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These solutions, dispersions, suspensions or emulsions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides).

The term “predisposed” as used herein can be understood to mean an increased probability (e.g., at least 1%, 5%, 10%, 20%, 30%, 40%, 50%, 60%, 70%, 80%, 90%, 100%, 150%, 200%, or more increase in probability) that a subject will suffer from a disease or condition.

The term “promoter” as used herein can be a region of DNA that initiates transcription of a particular gene or portion thereof.

The term “recipient” and their grammatical equivalents as used herein can refer to a subject. A subject can be a human or non-human animal. The recipient can also be in need thereof, such as needing treatment for a disease such as cancer. In some cases, a recipient may be in need thereof of a preventative therapy. A recipient may not be in need thereof in other cases.

The term “risk” and its grammatical equivalent as used herein can refer to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed.

The term “subject” and its grammatical equivalents as used herein can refer to a human or a non-human. A subject can be a mammal. A subject can be a human mammal of a male or female gender. A subject can be of any age. A subject can be an embryo. A subject can be a newborn or up to about 100 years of age. A subject can be in need thereof. A subject can have a disease such as cancer.

The term “sequence” and its grammatical equivalents as used herein can refer to a nucleotide sequence, which can be DNA and/or RNA; can be linear, circular or branched; and can be either single-stranded or double stranded. A sequence can be of any length, for example, between 2 and 1,000,000 or more nucleotides in length (or any integer value there between or there above), e.g., between about 100 and about 10,000 nucleotides or between about 200 and about 500 nucleotides.

“Surface-alternating agents”, as used herein can refer to an agent or material which modifies one or more properties of a structure's surface, including, but not limited to, hydrophilicity (e.g., can make a surface more or less hydrophilic), surface charge (e.g., makes a surface neutral or near neutral or more negative or positive), and/or enhances transport in or through bodily fluids and/or tissues, such as mucus. A surface-altering agent can be a polymer.

The term “stem cell” as used herein, can refer to an undifferentiated cell of a multicellular organism that is capable of giving rise to indefinitely more cells of the same type. A stem cell can also give rise to other kinds of cells by differentiation. Stem cells can be found in crypts. Stem cells can be progenitors of epithelial cells found on intestinal villi surface. Stem cells can be cancerous. A stem cell can be totipotent, unipotent or pluripotent. A stem cell can be an induced stem cell.

The terms “treatment” or “treating” and their grammatical equivalents can refer to the medical management of a subject with the intent to cure, ameliorate, stabilize, or prevent a disease, condition, or disorder. Treatment can include active treatment, that is, treatment directed specifically toward the improvement of a disease, condition, or disorder. Treatment can include causal treatment, that is, treatment directed toward removal of the cause of the associated disease, condition, or disorder. In addition, this treatment can include palliative treatment, that is, treatment designed for the relief of symptoms rather than the curing of the disease, condition, or disorder. Treatment can include preventative treatment, that is, treatment directed to minimizing or partially or completely inhibiting the development of a disease, condition, or disorder. Treatment can include supportive treatment, that is, treatment employed to supplement another specific therapy directed toward the improvement of the disease, condition, or disorder. In some instances, a condition can be pathological. In some instances, a treatment may not completely cure, ameliorate, stabilize or prevent a disease, condition, or disorder.

When used in the context of a chemical group, “hydrogen” means —H; “hydroxy” means —OH; “halogen” means independently —F, —Cl, —Br or —I;

For the structures provided herein, the following parenthetical subscripts further define the groups as follows: “(Cₙ)” defines the exact number (n) of carbon atoms in the group. For example, “(C₂₋₁₀) alkyl designates those alkyl groups having from 2 to 10 carbon atoms (e.g., 2, 3, 4, 5, 6, 7, 8, 9, or 10, or any range derivable therein (e.g., 3 to 10 carbon atoms).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂(cyclopropyl), —CH₂CH₂CH₂CH₃ (n-Bu), —CH(CH₃)CH₂CH₃ (sec-butyl), —CH₂CH(CH₃)₂(iso-butyl), —C(CH₃)₃(tert-butyl), —CH₂C(CH₃)₃(neo-pentyl), cyclobutyl, cyclopentyl, cyclohexyl, and cyclohexylmethyl are non-limiting examples of alkyl groups. Substituted alkyl refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CH₃, —C≡CC₆H₅ and —CH₂C≡CCH₃, are non-limiting examples of alkynyl groups. Substituted alkynyl refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “aryl” when used without the “substituted” modifier refers to a monovalent group with an aromatic carbon atom as the point of attachment, said carbon atom forming part of one or more six-membered aromatic ring structure(s) wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. Substituted aryl refers to a monovalent group with an aromatic carbon atom as the point of attachment, said carbon atom forming part of one or more six-membered aromatic ring structure(s) wherein the ring atoms are all carbon, and wherein the monovalent group further has at least one atom independently selected from the group consisting of N, O, F, Cl, Br, I, Si, P, and S. Non-limiting examples of substituted aryl groups include the groups: —C₆H₄F, —C₆H₄Cl, —C₆H₄Br, —C₆H₄I, —C₆H₄OH, —C₆H₄OCH₃, C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, C₆H₄C(O)CH₃, —C₆H₄C(O)C₆H₅, —C₆H₄CO₂H, —C₆H₄CO₂CH₃, —C₆H₄CONH₂, C₆H₄CONHCH₃, and —C₆H₄CON(CH₃)₂.

The term “cycloalkyl” refers to a saturated alicyclic moiety having three or more carbon atoms (e.g., from three to six carbon atoms) and which may be optionally benzofused at any available position. Non-limiting examples of cycloalkyl groups include the group cyclopropyl, cyclobutyl, cyclopentyl, cyclohexyl, indanyl and tetrahydronaphthyl.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of heteraryl groups include acridinyl, fliranyl, imidazoimidazolyl, imidazopyrazolyl, imidazopyridinyi, imidazopyrimidivyl, indolyl, indazolinyl, methylpyridyl oxazolyl, phenylimidazolyl, pyridyl, pyrrolyl, pyrimidyl, pyrazinyl, quinolyl, quinazolyl, quinoxalinyl, tetrahvdroquinolinyl, thienyl, triazinyl, pyrrolopyridinyl, pyrrolopyrinidinyl, pyrrolopyrazinyl, pyrrolotriazinyl, pyrroloimidazolyl, chromrenyl (where the point of attachment is one of the aromatic atoms), and chromanyl (where the point of attachment is one of the aromatic atoms). Substituted heteroaryl refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non-aromatic sulfur F, Cl, Br, I, Si, and P.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O— cyclohexyl. Substituted alkoxy” refers to the group —OR, in which R is a substituted alkyl, as that term is defined above. For ex ample, —OCH₂CF₃ is a substituted alkoxy group.

Overview

Disclosed herein are compositions and methods useful for gene therapy. The compositions and methods described throughout can use a structure, for example a nanoparticle to locally deliver a polynucleic acid. Effective gene delivery can be useful to treat diseases, for example, familial adenomatous polyposis (FAP) patients. In some instances, a minicircle DNA encoding for a therapeutic gene can be encapsulated within a nanoparticle for local gene therapy to a site, such as an intestinal crypt cell.

Liposome

A liposome can be a vesicular structure that can form via the accumulation of lipids interacting with one another in an energetically favorable manner. Liposomes can generally be formed by the self-assembly of dissolved lipid molecules, each of which can contain a hydrophilic head group and hydrophobic tails. Liposomes can consist of an aqueous core entrapped by one or more bilayers composed of natural or synthetic lipids. In some cases, liposomes can be highly reactive and immunogenic, or inert and weakly immunogenic. Liposomes composed of natural phospholipids can be biologically inert and weakly immunogenic, and liposomes can possess low intrinsic toxicity. Further, a liposome can carry a cargo. A cargo can be a polynucleic acid, such as a minicircle DNA, or a drug. A drug can be a substance that when administered can cause a physiological change in a subject. A drug can be a medication used to treat a disease, such as cancer. In some instances, drugs can be entrapped completely in a liposomal lipid bilayer, in an aqueous compartment, or in both a liposomal lipid bilayer and an aqueous compartment. Strongly lipophilic drugs can be entrapped almost completely in a lipid bilayer. Strongly hydrophilic drugs can be located exclusively in an aqueous compartment. Drugs with intermediate log P can easily partition between a lipid and aqueous phases, both in a bilayer and in an aqueous core. Liposomes can be classified according to their lamellarity (uni-, oligo-, and multi-lamellar vesicles), size (small, intermediate, or large) and preparation method (such as reverse phase evaporation vesicles, VETs).

A liposomal structure can be a vesicle in some cases. A vesicle can be unilamellar or multilamellar. Unilamellar vesicles can comprise a lipid bilayer and generally have diameters from about 50 nm to about 250 nm. Unilamellar vesicles can comprise a lipid bilayer and generally have diameters from about 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, or up to about 250 nm. Unilamellar vesicles can contain a large aqueous core and can be preferentially used to encapsulate drugs. In some cases, a unilamellar vesicle can partially encapsulate a drug. Multilamellar vesicles can comprise several concentric lipid bilayers in an onion-skin arrangement and have diameters from about 1-5 μm. Onion-skin arrangements can have diameters from about 1 μm, 1.5 μm, 2.0 μm, 2.5 μm, 3 μm, 3.5 μm. 4 μm, 4.5 μm, or up to 5.0 μm or greater. In some cases, a unilamellar vesicle or liposomal structure can have high lipid content. High lipid content can allow a unilamellar vesicle or multilamellar vesicle to passively entrap lipid-soluble drugs. In some instances, unilamellar vesicles can have a diameter of less than one micron, in some cases less than about 500 nm. Vesicle-forming lipids can have at least one hydrocarbon chain. Vesicle-forming lipids can have 1, 2, 3, 4, 5, 6, 7, 8, 9, 10, 11, 12, 13, 14, 15, 16, 17, 18, 19, or up to 20 or greater hydrocarbon chains. A hydrocarbon chain can be an acyl chain. A vesicle-forming lipid can have a head group. A head group can be polar or nonpolar. A hydrocarbon chain may be saturated or unsaturated. A hydrocarbon chain can have varying degrees of saturation. There are a variety of synthetic vesicle-forming lipids and naturally-occurring vesicle-forming lipids, including but not limited to sphingolipids, ether lipids, sterols, phospholipids, particularly the phosphoglycerides, and the glycolipids, such as the cerebrosides and gangliosides.

Liposomes can be biocompatible and biodegradable. For example, in some cases, a liposome may biodegrade after introduction into a subject. Biodegradation can begin immediately after introduction in some cases. Biodegradation can occur within a mucosal tract of a subject that has received an administration of a liposome or liposomal structure. Biodegradation can result release of a liposomal cargo such as a polynucleic acid. In other cases, biodegradation can comprise decomposition of a component of a liposomal structure such as a polymer. Biodegradation can occur under standard bodily conditions such as from about 97.6° F. to about 99° F. In other cases, biodegradation can occur under a temperature from about 95° F. to about 106° F. Biodegradation can occur from about 95° F., 96° F., 97° F., 98° F., 99° F., 100° F., 101° F., 102° F., 103° F., 104° F., 105° F., or up to 106° F. In other aspects, biodegradation can occur from about 50° F. to about 150° F.

In other cases, biodegradation may not occur. When biodegradation occurs it can take from about 1 minute to about 100 years after administration of a liposome or a structure to a subject. Biodegradation can take from about 1 minute, 5 minutes, 30 minutes, 1 hour, 3 hours, 7 hours, 10 hours, 15 hours, 20 hours, 25 hours, 2 days, 4 days, 8 days, 12 days, 20 days, 30 days, 1.5 months, 3 months, 4 months, 5 months, 6 months, 7 months, 8 months, 9 months, 10 months, 11 months, 12 months, 1.5 yrs., 3 years, 5 years, 8 years, 10 years, 15 years, 20 years, 30 years, 40 years, 50 years, 60 years, 70 years, 80 years, 90 years, or at least about 100 years. Lipid of a structure such as a liposome may be or may comprise: fatty acids, glycerolipids, glycerophospholipids, sphingolipids, saccharolipids, polyketides (derived from condensation of ketoacyl subunits); sterol lipids prenol lipids (derived from condensation of isoprene subunits) or any combination thereof.

Glycerolipids can be composed mainly of mono-, di-, or tri-substituted glycerols, the most well-known being the fatty acid triesters of glycerol, called triglycerides. The word “triacylglycerol” can sometimes be used synonymously with “triglyceride”, though the latter lipids contain no hydroxyl group. In glycerolipids, the three hydroxyl groups of glycerol can be esterified, typically by different fatty acids.

Glycerophospholipids, usually referred to as phospholipids can contain a diglyceride, a phosphate group, and a simple organic molecule such as choline. In some cases a glycerophospholipid may not contain a diglyceride, a phosphate group, and a simple organic molecule. A glycerophopholipid that may not contain a diglyceride, a phosphate group, and a simple organic molecule can be sphingomyelin. A sphingomyelin can be derived from sphingosine instead of glycerol.

A structure of a phospholipid molecule generally consists of hydrophobic tails and a hydrophilic head. A hydrophilic head can contain the negatively charged phosphate group, and may contain other polar groups. A hydrophobic tail can consist of long fatty acid hydrocarbon chains. When placed in water, phospholipids can form a variety of structures depending on specific properties of a phospholipid. Lipid bilayers can occur when hydrophobic tails line up against one another, forming a membrane of hydrophilic heads on both sides facing the water. Glycerophospholipids may be subdivided into distinct classes, based on the nature of the polar head group at the −3 position of a glycerol backbone in eukaryotes and eubacteria, or the −1 position in the case of archaebacteria. Examples of glycerophospholipids found in biological membranes are phosphatidylcholine (also known as PC, GPCho or lecithin), phosphatidylethanolamine (PE or GPEtn) and phosphatidylserine (PS or GPSer). In eukaryotes, phospholipids are generally classified into two types: diacylglycerides and phosphingolipids. Examples of diacylglycerides include, but are not limited to, phosphatidic acid (phosphatidate) (PA), phosphatidylethanolamine (cephalin) (PE), phosphatidylcholine (lecithin) (PC), phosphatidylserine (PS), and phosphoinositides, such as phosphatidylinositol (PI), phosphatidylinositol phosphate (PIP), phosphatidylinositol bisphosphate (PIP2) and, phosphatidylinositol triphosphate (PIP3). Examples of phospingolipids include, but are not limited to, ceramide phosphorylcholine (Sphingomyelin) (SPH), ceramide phosphorylethanolamine (Sphingomyelin) (Cer-PE), and Ceramide phosphoryllipid.

Phosphoglycerides include, but are not limited to phospholipids such as phosphatidylcholine, phosphatidylethanolamine, phosphatidic acid, phosphatidylinositol, phosphatidylserine phosphatidylglycerol and diphosphatidylglycerol (cardiolipin). In some instances, two hydrocarbon chains of a phosphoglyceride can be between about 14 to about 22 carbon atoms in length. In some cases, the two hydrocarbon chains of a phosphoglyceride can be between about 1 to about 100 carbon atoms in length. the two hydrocarbon chains of a phosphoglyceride can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or up to about 100 carbon atoms in length. In some instances, the two hydrocarbon chains of a phosphoglyceride can have varying degrees of unsaturation. As used herein, the abbreviation “PC” refers to phosphatidylcholine, and “PS” refers to phosphatidylserine. Lipids containing either saturated and/or unsaturated fatty acids are widely available to those of skill in the art. Fatty acids, or fatty acid residues when part of a lipid, can be a diverse group of molecules. In some instances, fatty acid residues can be prepared synthetically or synthesized naturally. In some instances, fatty acid residues can be synthesized naturally by chain-elongation of an acetyl-CoA primer with malonyl-CoA or methylmalonyl-CoA groups in a process called fatty acid synthesis. Fatty acids can be made of a hydrocarbon chain that can terminate with a carboxylic acid group; this arrangement can confer the molecule with a polar, hydrophilic end, and a nonpolar, hydrophobic end that can be insoluble in water. A carbon chain, typically between 4 and 24 carbons long, may be saturated or unsaturated. In some instances, a carbon chain may be attached to functional groups containing oxygen, halogens, nitrogen, and/or sulfur. Where a double bond exists in a carbon chain, there can be a cis or trans geometric isomerism, which significantly affects the molecule's configuration. Cis-double bonds can cause the fatty acid chain to bend, an effect that is compounded with more double bonds in the chain. Most naturally occurring fatty acids are of the cis configuration, although the trans form does exist in some natural and partially hydrogenated fats and oils. Other lipids in a fatty acid category can be fatty esters and fatty amides. Additionally, the two hydrocarbon chains of a lipid may be symmetrical or asymmetrical. Lipids and phospholipids described herein can contain an acyl chain. In some instances, an acyl chains can have varying lengths and degrees of saturation. Varying lengths and degrees of saturation of acyl chains can be obtained commercially or prepared according to published methods.

In some cases, a liposomal structure can comprise a phosphatidylcholine. Exemplary phosphatidylcholines include but are not limited to dilauroyl phophatidylcholine, dimyristoylphophatidylcholine, dipalmitoylphophatidylcholine, distearoylphophatidyl-choline, diarachidoylphophatidylcholine, dioleoylphophatidylcholine, dilinoleoyl-phophatidylcholine, dierucoylphophatidylcholine, palmitoyl-oleoyl-phophatidylcholine, egg phosphatidylcholine, myristoyl-palmitoylphosphatidylcholine, palmitoyl-myristoyl-phdsphatidylcholine, myristoyl-stearoylphosphatidylcholine, palmitoyl-stearoyl-phosphatidylcholine, stearoyl-palmitoylphosphatidylcholine, stearoyl-oleoyl-phosphatidylcholine, stearoyl-linoleoylphosphatidylcholine and palmitoyl-linoleoyl-phosphatidylcholine. Asymmetric phosphatidylcholines can be referred to as 1-acyl, 2-acyl-sn-glycero-3-phosphocholines, wherein the acyl groups are different from each other. Symmetric phosphatidylcholines can be referred to as 1,2-diacyl-sn-glycero-3-phosphocholines. As used herein, the abbreviation “PC” refers to phosphatidylcholine. The phosphatidylcholine 1,2-dimyristoyl-sn-glycero-3-phosphocholine can be abbreviated herein as “DMPC.” The phosphatidylcholine 1,2-dioleoyl-sn-glycero-3-phosphocholine can be abbreviated herein as “DOPC.” The phosphatidylcholine 1,2-dipalmitoyl-sn-glycero-3-phosphocholine can be abbreviated herein as “DPPC.” In general, saturated acyl groups found in various lipids include groups having the names propionyl, butanoyl, pentanoyl, caproyl, heptanoyl, capryloyl, nonanoyl, capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, behenoyl, trucisanoyl and lignoceroyl. The corresponding IUPAC names for saturated acyl groups are trianoic, tetranoic, pentanoic, hexanoic, heptanoic, octanoic, nonanoic, decanoic, undecanoic, dodecanoic, tridecanoic, tetradecanoic, pentadecanoic, hexadecanoic, 3,7,11,15-tetramethylhexadecanoic, heptadecanoic, octadecanoic, nonadecanoic, eicosanoic, heneicosanoic, docosanoic, trocosanoic and tetracosanoic. Unsaturated acyl groups found in both symmetric and asymmetric phosphatidylcholines include myristoleoyl, palmitoleyl, oleoyl, elaidoyl, linoleoyl, linolenoyl, eicosenoyl and arachidonoyl. The corresponding IUPAC names for unsaturated acyl groups are 9-cis-tetradecanoic, 9-cis-hexadecanoic, 9-cis-octadecanoic, 9-trans-octadecanoic, 9-cis-12-cis-octadecadienoic, 9-cis-12-cis-15-cisoctadecatrienoic, 11-cis-eicosenoic and 5-cis-8-cis-11-cis-14-cis-eicosatetraenoic. Exemplary phosphatidylethanolamines include dimyristoyl-phosphatidylethanolamine, dipalmitoyl-phosphatidylethanolamine, distearoyl phosphatidylethanolamine, dioleoyl-phosphatidylethanolamine and egg phosphatidylethanolamine. Phosphatidylethanolamines may also be referred to under IUPAC naming systems as 1,2-diacyl-sn-glycero-3-phosphoethanolamines or 1-acyl-2-acyl-sn-glycero-3-phosphoethanolamine, depending on whether they are symmetric or asymmetric lipids. Exemplary phosphatidic acids include dimyristoyl phosphatidic acid, dipalmitoyl phosphatidic acid and dioleoyl phosphatidic acid. Phosphatidic acids may also be referred to under IUPAC naming systems as 1,2-diacyl-sn-glycero-3-phosphate or 1-acyl-2-acyl-sn-glycero-3-phosphate, depending on whether they are symmetric or asymmetric lipids. Exemplary phosphatidylserines include dimyristoyl phosphatidylserine, dipalmitoyl phosphatidylserine, dioleoylphosphatidylserine, distearoyl phosphatidylserine, palmitoyl-oleylphosphatidylserine and brain phosphatidylserine. Phosphatidylserines may also be referred to under IUPAC naming systems as 1,2-diacyl-sn-glycero-3-[phospho-L-serine] or 1-acyl-2-acyl-sn-glycero-3-[phospho-L-serine], depending on whether they are symmetric or asymmetric lipids. As used herein, the abbreviation “PS” refers to phosphatidylserine. Exemplary phosphatidylglycerols include dilauryloylphosphatidylglycerol, dipalmitoylphosphatidylglycerol, distearoylphosphatidylglycerol, dioleoyl-phosphatidylglycerol, dimyristoylphosphatidylglycerol, palmitoyl-oleoyl-phosphatidylglycerol and egg phosphatidylglycerol. Phosphatidylglycerols may also be referred to under IUPAC naming systems as 1,2-diacyl-sn-glycero-3-[phospho-rac-(1-glycerol)] or 1-acyl-2-acyl-sn-glycero-3-[phospho-rac-(1-glycerol)], depending on whether they are symmetric or asymmetric lipids. The phosphatidylglycerol 1,2-dimyristoyl-sn-glycero-3-[phospho-rac-(1-glycerol)] is abbreviated herein as “DMPG”. The phosphatidylglycerol 1,2-dipalmitoyl-sn-glycero-3-(phospho-rac-1-glycerol) (sodium salt) is abbreviated herein as “DPPG”. Suitable sphingomyelins might include brain sphingomyelin, egg sphingomyelin, dipalmitoyl sphingomyelin, and distearoyl sphingomyelin. Other suitable lipids include glycolipids, sphingolipids, ether lipids, glycolipids such as the cerebrosides and gangliosides, and sterols, such as cholesterol or ergosterol.

In some cases, a liposomal structure can comprise cholesterol or a derivative thereof, a phospholipid, a mixture of a phospholipid and cholesterol or a derivative thereof, or a combination. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. When a liposomal structure comprises a mixture of a phospholipid and cholesterol or a cholesterol derivative, the liposomal structure may comprise up to about 40, 50, or 60 mol % of the total lipid present in the liposomal structure. One or more phospholipids and/or cholesterol may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the liposomal structure.

Depending on structure, composition, or a combination thereof of bulk solution, liposomes can separate hydrophobic or hydrophilic molecules from solution. In some instances, liposomes can be referred to as vesicles. In some instances, liposomes can be rigid or non-rigid. Liposomes may not be rigid formations but rather fluid entities that can be versatile supramolecular complexes. Liposomes can have a wide array of uses. A liposome may be arranged in many shapes and sizes depending on lipid composition. A liposome can be used to deliver a molecular cargo such as DNA for therapeutic benefit. Lipids used to form liposomes can be cationic, anionic, neutral, or a mixture thereof. A liposome may be a cationic liposome, a neutral liposome or an anionic liposome.

In particular embodiments, a liposomal structure can include one or more of a second amino lipid or cationic lipid, a neutral lipid, a sterol, and a lipid selected to reduce aggregation of lipid particles during formation. Aggregation may result from steric stabilization of liposomal structures which may prevent charge-induced aggregation during formation. Liposomal structures can include two or more cationic lipids. The lipids can be selected to contribute different advantageous properties. For example, cationic lipids that differ in properties such as amine pKₐ, chemical stability, half-life in circulation, half-life in tissue, net accumulation in tissue, or toxicity can be used in a liposomal structure. In particular, cationic lipids can be chosen so that the properties of the mixed-lipid liposomal structure are more desirable than the properties of a single-lipid structure of individual lipids. Net tissue accumulation and long term toxicity (if any) from cationic lipids can be modulated in a favorable way by choosing mixtures of cationic lipids instead of selecting a single cationic lipid in a given formulation. Such mixtures can also provide better encapsulation and/or release of a polynucleic acid encoding at least a portion of a gene, such as APC. A combination of cationic lipids also can affect the systemic stability when compared to single entity in a formulation.

Each lipid of a liposome can have the same or different structural aspects, such as head group size and hydrocarbon tail length. In some cases, a headgroup area can be from about 0.1 nm² to about 10 nm². For example, a headgroup area can be from about 0.1 nm², 0.2 nm², 0.3 nm², 0.4 nm², 0.5 nm² 0.6 nm², 0.6 nm², 0.7 nm², 0.8 nm², 0.9 nm², 1.0 nm², 2.0 nm², 3.0 nm², 4.0 nm², 5.0 nm², 6.0 nm², 7.0 nm², 8.0 nm², 9.0 nm², to about 10.0 nm². In some cases, a headgroup area can be from about 0.40 nm² to about 5 nm². In some cases, a cationic lipid can have a headgroup area from about 1.66 nm² approximately.

In some cases, a hydrocarbon tail of a lipid can be about 8 to 18 carbons in length. In some cases, a hydrocarbon tail can be 8, 9, 10, 11, 12, 13, 14, 15, 16, 17, 18, or over 18 carbons in length. A hydrocarbon tail can also be exactly 8 or exactly 18 carbons in length. A hydrocarbon tail can be saturated. In some cases, a hydrocarbon tail may not be saturated. In other cases, a saturated hydrocarbon tail may have a single double bond. In some cases, a combination of hydrocarbon chains can be symmetric, asymmetric, or any combination. In some cases, symmetry of a hydrocarbon tail can be modulated to improve transfection efficiency. For example, an asymmetric hydrocarbon tail with both shorter saturated carbon chains and long unsaturated carbon chains may produce high transfection efficiencies as compared to mixed formulations of symmetric hydrocarbon tails.

A liposome can be formed by any means. In some cases, a liposome can be formed by self-assembly of dissolved lipid molecules. In some cases, a hydrophilic lipid head group and a hydrophobic lipid tail may be involved in self-assembly. Hydrophilic lipids may take on associations which can yield entropically favorable states of low free energy, in some cases forming bimolecular lipid leaflets. A leaflet can be characterized by hydrophobic lipid hydrocarbon tails facing each other and hydrophilic lipid head groups facing outward to associate with aqueous solution. At this point, a bilayer formation may still be energetically unfavorable because hydrophobic parts of a lipid molecules may still be in contact with water, this may be overcome through curvature of forming of a bilayer membrane upon itself to form a vesicle with closed edges. In some cases, a liposome may form a vesicle. A liposome can include an exterior surface and an interior compartment. A lipid molecule used to generate a liposome may be a conserved entity with a head group and hydrophobic hydrocarbon tails connected via a backbone linker. A backbone linker can be glycerol. Examples of classes of linkers that may be used include but are not limited to amide, alkylamine, thiolether, alkyl, cycloalkyl, and/or aryl linkages.

In certain applications, it may be desirable to release a moiety once a drug such as a polynucleic acid has entered a cell. A moiety can be utilized to identify a number of cells that have received a polynucleic acid. A moiety can be an antibody, dye, scFv, peptide, glycoprotein, carbohydrate, ligand, polymer, to name a few. A moiety can be in contact with a linker. A linker can be non-cleavable. Accordingly, in some cases, a linker can be a cleavable linker. This may enable a moiety to be released from a liposomal structure once contact to a target cell has been made. This may be desirable when a moiety has a greater therapeutic effect when separated from a liposomal structure. In some cases, a moiety may have a better ability to be absorbed by an intracellular component of a cell, such as an intestinal crypt cell or intestinal crypt stem cell, when separated from a liposomal structure. In some cases, a linker may comprise a disulfide bond, acyl hydrazone, vinyl ether, orthoester, or a N—PO3.

Accordingly, it may be necessary or desirable to separate a moiety from a liposomal structure so that a moiety can enter an intracellular compartment. Cleavage of a linker releasing a moiety may be as a result of a change in conditions within a cell as compared to outside cells, for example, due to a change in pH within a cell. Cleavage of a linker may occur due to the presence of an enzyme within a cell which cleaves a linker once a drug, such as a polynucleic acid, enters a cell. Alternatively, cleavage of a linker may occur in response to energy or a chemical being applied to the cell. Examples of types of energies that may be used to effect cleavage of a linker include, but are not limited to light, ultrasound, microwave and radiofrequency energy. In some cases, a linker may be a photolabile linker. A linker used to link a complex may also be an acid labile linker. Examples of acid labile linkers include linkers formed by using cis-aconitic acid, cis-carboxylic alkatriene, polymaleic anhydride, and other acidlabile linkers.

In some cases, a cationic lipid may attain a positive charge through one or more amines present in a polar head group. In some cases, a liposome can be a cationic liposome. In some cases, a liposome may be a cationic liposome used to carry negatively charged polynucleic acid, such as DNA. The presence of positively charged amines may facilitate binding with anions such as those found in DNA. A liposome thus formed may be a result of energetic contributions by Van der Waals forces and electrostatic binding to a DNA cargo which may partially contribute to liposome shape. In some cases, a cationic (and neutral) lipid may be used for gene delivery. In other cases, an anionic liposome may be used to deliver other therapeutic agents.

In some cases, an anionic liposome structure may be utilized to deliver a polynucleic acid such as DNA. Formation of a DNA-containing liposome using anionic lipids can be brought about through the use of divalent cations to negate the mutual electrostatic repulsion and facilitate lipoplex assembly. Anionic lipoplexes can be composed of physiologically safe components including anionic lipids, cations, and DNA. Commonly used lipids in this category are phospholipids that can be found naturally in cellular membranes such as phosphatidic acid, phosphatidylglycerol, and phosphatidylserine.

Anionic lipids can contain any of a wide range of fatty acid chains in the hydrophobic region. The specific fatty acids incorporated are responsible for the fluidic characteristics of the liposome in terms of phase behavior and elasticity. Perhaps due to the natural presence of specific phospholipids in the host cell membrane, gene delivery via lipoplexes with net negative surface potentials has been associated with lower clearance and phagocytosis by macrophages, which is consistent with favorable biocompatibility. Divalent cations can be incorporated into an anionic liposome system to enable the condensation of nucleic acids prior to envelopment by anionic lipids. Several divalent cations can be used in anionic lipoplexes such as Ca²⁺, Mg²⁺, Mn²⁺, and Ba²⁺. In some cases, Ca²⁺ can be utilized in an anionic liposome system.

A cationic lipid can be used to form a liposome. Cationic lipids may commonly attain a positive charge through one or more amines present in the polar head group. The presence of positively charged amines facilitates binding with anions such as those found in DNA. The liposome that formed can be a result of energetic contributions by Van der Waals forces and electrostatic binding to the DNA which may partially dictate liposome shape. Because of the polyanionic nature of DNA, cationic (and neutral) lipids are typically used for gene delivery, while the use of anionic liposomes has been fairly restricted to the delivery of other therapeutic macromolecules.

A solution of cationic lipids, often formed with neutral helper lipids, can be mixed with DNA to form a positively charged complex termed a lipoplex. Reagents for cationic lipid transfection can include N-[1-(2,3-dioleyloxy)propyl]-N,N,N-trimethylammonium chloride (DOTMA), [1,2-bis(oleoyloxy)-3-(trimethylammonio)propane] (DOTAP), 3β[N—(N′, N′-dimethylaminoethane)-carbamoyl] cholesterol (DC-Chol), and dioctadecylamidoglycylspermine (DOGS). Dioleoylphosphatidylethanolamine (DOPE), a neutral lipid, may often be used in conjunction with cationic lipids because of its membrane destabilizing effects at low pH, which can aide in endolysosomal escape.

A liposome may be formed with neutral helper lipids. A liposome may be generated using cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), Dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-Distearoyl-sn-glycero-3-phosphoethanolamine (DSPE), 1,2-distearoyl-sn-glycero-3-phosphocholine (DSPC), Dimethyldioctadecylammonium (DDAB), a salt thereof, and any combination thereof.

In some cases, a liposomal structure can be composed of MVL5 and GMO. A molar concentration of MVL5 and GMO can range from about 10:1 to 1:25. A molar ratio of MVL5 and GMO can range from about 10:1 to about 1:10. In other cases, a molar ratio of MVL5 and GMO can range from about 50:1 to 1:1. For example a molar ratio can be from about 50:1 to 1:1, 40:1 to 1:1, 30:1 to 1:1, 20:1 to 1:1, 10:1 to 1:1 or about 5:1 to 1:1. A molar ratio of MVL5 to GMO can range from about 10:1 to about 1:1, or 10:1 to about 1:25.

In some cases, Dioleoylphosphatidylethanolamine (DOPE), a neutral lipid, can be used in conjunction with a cationic lipid because of its membrane destabilizing effects at low pH, which can aide in endolysosomal escape. In some cases, a cationic liposome can be generated using other reagents. In some cases a combination of DOGS and DOPE can be used. DOGS and DOPE can be combined with at least one polymer, such as PEG, in a manufacturing process. DOGS can be cationic and can hydrogen bond with an anionic polynucleic acid, such as DNA. In some cases, a polynucelic acid DNA can form a salt with a branched-PEI and can be encapsulated by a neutral liposome.

DOTMA, can consist of two unsaturated oleoyl chains (C18:Δ9), bound by an ether bond to a three-carbon skeleton of a glycerol, with a quaternary amine as the cationic head group. In some cases, modifications can be made. For example, a modification to DOTMA can be different combinations of side chains and alkyl attachments to a head group, replacement of a methyl group on a quaternary amine of DOTMA with a hydroxyl, or any combination thereof. {2, 3-dioleyloxy-N-[2(sperminecarboxamido) ethyl]-N, N-dimethyl-1-propanaminium trifluoroacetate}, or DOSPA, can be another cationic lipid synthesized as a derivative of DOTMA. Its structure can be similar to DOTMA except for a spermine group which can be bound via a peptide bond to its hydrophobic chains. In general, the addition of the spermine functional group can allow for a more efficient packing of DNA in terms of liposome size.

DOTAP can consist of a quaternary amine head group coupled to a glycerol backbone with two oleoyl chains. DC-Chol can consist of a cholesterol moiety attached by an ester bond to a hydrolysable dimethylethylenediamine. DOGS can have a structure similar to DOSPA. In some cases both molecules can have a multivalent spermine head group and two 18-carbon alkyl chains. However, the chains in DOGS can be saturated, can be linked to a head group through a peptide bond, and can lack a quaternary amine.

In certain cases, a lipid bilayer can be generated of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5 rapeseed sterol. In certain embodiments, the phytosterol comprises a mixture of at least two of the following compositions: sistosterol, camposterol and stigmasterol. In still other embodiments, a lipid layer can be comprised of one or more phosphatidyl groups selected from the group comprising phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol or lyso-phosphatidyl-inositol. In other cases, a lipid bilayer can be comprised of phospholipid selected from a monoacyl or diacylphosphoglyceride. In still other cases, a lipid bilayer can be comprised of one or more phosphoinositides selected from the group comprising phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), phosphatidyl-inositol (PI), or lysophosphatidyl-inositol (LPI).

Lipids or liposomes of the present disclosure may be modified. A modification can be a surface modification. A surface modification can enhance an average rate at which a liposomal structure moves in mucus compared to a comparable liposomal structure. A comparable liposomal structure may not be surface modified or a comparable liposomal structure may be modified with a polyethylene gycol (PEG) polymer. A modification can facilitate protection from degradation in vivo. A modification may also assist in trafficking of a liposome. For example, a modification may allow a liposome to traffic within a gastrointestinal (GI) track with an acidic pH due to pH sensitive modifications. A surface modification can also improve an average rate at which a liposome moves in mucous. For example, a modification may enhance a rate by 1×, 2×, 3×, 4×, 5×, 6×, 7×, 8×, 9×, 10×, 20×, 30×, 40×, 50×, 60×, 70×, 80×, 90×, 100×, 300×, 500×, 700×, 900×, or up to about 1000× when compared to a comparable liposomal structure without a modification or a liposomal structure with a modification comprising PEG. In some cases, a modification to a liposomal occurs via a bond. A bond can be covalent, noncovalent, polar, ionic, hydrogen, or any combination thereof. A bond can be considered an association of two groups or portions of groups. For example, a liposomal structure can be bonded to a PEG via a linker comprising a covalent bond. In some cases, a bond can occur between two adjacent groups. Bonds can be dynamic. A dynamic bond can occur when one group temporarily associates with a second group. For example, a polynucleic acid in suspension within a liposome may bond with portions of a lipid bilayer during its suspension.

In some cases, a modification can be a polyethylene glycol (PEG) addition. Methods of modifying liposomal surfaces with PEG can include its physical adsorption onto a liposomal surface, its covalent attachment onto liposomes, its coating onto a liposome, or any combination thereof. In some cases, PEG can be covalently attached to a lipid particle before a liposome can be formed.

A variety of molecular weights of PEG may be used. PEG can range from about 10 to about 100 units of an ethylene PEG component which may be conjugated to phospholipid through an amine group comprising or comprising about 1% to about 20%, preferably about 5% to about 15%, about 10% by weight of the lipids which are included in a lipid bilayer.

In certain cases, a nanostructure can further comprise at least one targeting agent. The term targeting agent can refer to a moiety, compound, antibody, etc. that specifically binds a particular type or category of cell and/or other particular type compounds, (e.g., a moiety that targets a specific cell or type of cell). A targeting agent can be specific (e.g., have an affinity) for the surface of certain target cells, a target cell surface antigen, a target cell receptor, or a combination thereof. In some cases a targeting agent can refer to an agent that has a particular action (e.g., cleaves) when exposed to a particular type or category of substances and/or cells, and this action can drive the nanostructure to target a particular type or category of cell. Thus, the term targeting agent can refer to an agent that can be part of a nanostructure and plays a role in the nanostructure's targeting mechanism, although the agent itself may or may not be specific for the particular type or category of cell itself. In certain instances, the efficiency of the cellular uptake of a polynucleic acid delivered by a nanostructure can be enhanced and/or made more specific by incorporation of targeting agents into the present nanostructures. In certain embodiments, nanostructures described herein can comprise one or more small molecule targeting agents (e.g., carbohydrate moieties). Suitable targeting agents also include, by way of non-limiting example, antibodies, antibody-like molecules, or peptides, such as an integrin-binding peptides such as RGD-containing peptides, or small molecules, such as vitamins, e.g., folate, sugars such as lactose and galactose, or other small molecules. Cell surface antigens include a cell surface molecule such as a protein, sugar, lipid or other antigen on the cell surface. In specific embodiments, the cell surface antigen undergoes internalization. Examples of cell surface antigens targeted by the targeting agents of embodiments of the present nanoparticles include, but are not limited, to the transferrin receptor type 1 and 2, the EGF receptor, HER2/Neu, VEGF receptors, integrins, NGF, CD2, CD3, CD4, CDS, CDI9, CD20, CD22, CD33, CD43, i 1)38. CD56, CD69, and the leucine-rich repeat-containing G-protein coupled receptor 5 (LGR5). A targeting agent can also comprise an artificial affinity molecule, e.g., a peptidomimetic or an aptamer. Peptidomimetics can refer to compounds in which at least a portion of a peptide, such as a therapeutic peptide, is modified, and the three-dimensional structure of the peptidomimetic remains substantially the same as that of the peptide. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry.

In some embodiments, the targeting agent can be a proteinaceous targeting agent (e.g., a peptide, and antibody, an antibody fragment). In some specific embodiments, a nanostructure can comprise a plurality of different targeting agents.

In some embodiments, one or more targeting agents can be coupled to the polymers that form the nanostructure. In some cases, the targeting agents can be bound to a polymer that coats a nanostructure. In some instances, a targeting agent can be covalently coupled to a polymer. In some cases a targeting agent can be bound to a polymer such that a targeting agent can be substantially at or near the surface of the resulting nanostructure. In certain embodiments, a monomer comprising a targeting agent residue (e.g, a polymerizable derivative of a targeting agent such as an (alkyl) acrylic acid derivative of a peptide) can be co-polymerized to form the copolymer forming the nanostructure provided herein. In certain embodiments, one or more targeting agents can be coupled to the polymer of the present nanoparticles through a linking moiety. In some embodiments, the linking moiety coupling the targeting agent to the membrane-destabilizing polymer can be a cleavable linking moiety (e.g., comprises a cleavable bond). In some embodiments, the linking moiety can be cleavable and/or comprises a bond that can be cleavable in endosomal conditions. In some embodiments, the linking moiety can be cleavable and/or comprise a bond that can be cleaved by a specific enzyme (e.g., a phosphatase, or a protease). In some embodiments, the linking moiety can be cleavable and/or comprise a bond that may be cleavable upon a change in an intracellular parameter (e.g., pH, redox potential), in some embodiments, a linking moiety can be cleavable and/or comprise a bond that can be cleaved upon exposure to a matrix metalloproteinase (MMP) (e.g., MMP-cleavable peptide linking moiety).

In certain cases a targeting mechanism of a nanoparticle can depend on a cleavage of a cleavable segment in a polymer. For instance, the present polymers can comprise a cleavable segment that, when cleaved, exposes the nanoparticle and/or the core of a nanoparticle. The cleavable segment can be located at either or both terminal ends of the present polymers in some embodiments. In some embodiments the cleavable segment is located along a length of a polymer, and optionally can be located between blocks of a polymer. For example, in certain embodiments the cleavable segment can be located between a first block and a second block of a polymer, and when a nanoparticle can be exposed to a particular cleaving substance the first block can be cleaved from a second block. In specific embodiments a cleavable segment can be an MMP-cleavable peptide that can be cleaved upon exposure to MMP.

Attachment of a targeting agent, such as an antibody, to a polymer can be achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl tinkers, sulfhydryl-sulfhydryl linkers, carbohydrate-hydroxyl linkers, carbohydrate-carbohydrate linkers, and hydroxyl-hydroxyl linkers. In specific embodiments, “click” chemistry can be used to attach the targeting agent to the polymers of the nanoparticles provided herein. A large variety of conjugation chemistries are optionally utilized, in some embodiments, targeting agents can be attached to a monomer and the resulting compound can then be used in a polymerization synthesis of a polymer (e.g., copolymer) utilized in a nanoparticle described herein. In some embodiments, a targeting agent can be attached to the sense or antisense strand of siRNA bound to a polymer of a nanoparticle. In certain embodiments, a targeting agent can be attached to a 5′ or a 3′ end of the sense or the antisense strand.

Methods for linking compounds can include but are not limited to proteins, labels, and other chemical entities, to nucleotides. Cross-linking reagents such as n-maleimidobutyryloxy-succinimide ester (GMBS) and sulfo-GMBS, have reduced immunogenicity. Substituents have been attached to the 5′ end of preconstructed oligonucleotides using amidite or H-phosphonate chemistry. Substituents can also be attached to the 3′ end of oligomers. This last method utilizes 2,2′-dithioethanol attached to a solid support to displace diisopropylamine from a 3′ phosphonate bearing the acridine moiety and is subsequently deleted after oxidation of the phosphorus. Alternatively, an oligonucleotide may include one or more modified nucleotides having a group attached via a linker arm to the base. For example, the attachment of biotin to the C-5 position of dUTP by an allylamine linker arm may be utilized. The attachment of biotin and other groups to the 5-position of pyrimidines via a linker arm may also be performed.

Chemical cross-linking may include the use of spacer arms, i.e., linkers or tethers. Spacer arms provide intramolecular flexibility or adjust intramolecular distances between conjugated moieties and thereby may help preserve biological activity. A spacer arm may be in the form of a peptide moiety comprising spacer amino acids. Alternatively, a spacer arm may be part of the cross-linking reagent, such as in “long-chain SPDP”.

A variety of coupling or crosslinking agents such as protein A, carbodiimide, dimaleimide, dithio-bis-nitrobenzoic acid (DTNB), N-succinimidyl-5-acetyl-thioacetate (SATA), and N-succinimidyl-3-(2-pyrid-yldithio)propionate (SPDP), 6-hydrazinonicotimide (HYNIC), N₃S and N₂S₂ can be used in well-known procedures to synthesize targeted constructs. For example, biotin can be conjugated to an oligonucleotide via DTPA using a bicyclic anhydride method. In addition, sulfosuccinimidyl 6-(biotinamido)hexanoate (NHS-LC-biotin, which can be purchased from Pierce Chemical Co. Rockford, Ill.), “biocytin,” a lysine conjugate of biotin, can be useful for making biotin compounds due to the availability of a primary amine. In addition, corresponding biotin acid chloride or acid precursors can be coupled with an amino derivative of the therapeutic agent by known methods. By coupling a biotin moiety to the surface of a particle, another moiety may be coupled to avidin and then coupled to the particle by the strong avidin-biotin affinity, or vice versa. In certain embodiments where a polymeric particle comprises PEG moieties on the surface of the particle, the free hydroxyl group of PEG may be used for linkage or attachment (e.g., covalent attachment) of additional molecules or moieties to the particle.

In embodiments, a liposome modification can provide biocompatibility and can be modified to possess targeting species including, for example, targeting peptides including antibodies, aptamers, polyethylene, or combinations thereof. A targeting species can also be a receptor. In some cases, a T cell receptor (TCR), B cell receptor (BCR), single chain variable fragment (scFv), chimeric antigen receptor (CAR), or combinations thereof are used.

Liposome can be of any size and morphology. A morphology may be a unilamellar vesicle. A unilamellar vesicle may be characterized by a single bilayer membrane which can encapsulate an internal aqueous solution. Both cationic amine head groups and anionic phospholipid head groups can form single-walled vesicles. A liposome may also be multilamellar. A multilamellar liposome may contain multiple concentric bilayers. An oligolamellar liposome may contain two concentric bilayers. A multivesicular liposome may contain multiple smaller unilamellar vesicles inside of one giant vesicle.

Vesicle sizes fall into the nanometer to micrometer range: small unilamellar vesicles are 20-200 nm, large unilamellar vesicles are 200 nm-1 μm, and giant unilamellar vesicles are larger than 1 μm. In some cases, a vesicle can range in size from 1 μm to more than 100 μm. In some cases, a polynucleic acid may be condensed to be properly encapsulated by a liposomal structure. Condensation of DNA may be performed by divalent metal ions such as Mn²⁺, Ni²⁺, Co²⁺, and Cu²⁺ that can condense DNA through neutralization of phosphate groups of the DNA backbone and distortion of the B-DNA structure through hydrogen bonding with bases, permitting both local bending of the DNA and inter-helical associations. In some cases, the concentration of metal ions utilized for condensation can be dependent on the dielectric constant of a medium used in the condensation. The addition of ethanol or methanol may also reduce the concentration of metal ion required for condensation. In some cases, ethanol can be used to condense DNA at concentrations from about 0.5% up to about 60% by volume. In some cases, ethanol can be used to condense DNA at concentrations from about 0.5%, 5%, 10%, 15%, 20%, 25%, 30%, 35%, 40%, 45%, 50%, 55% or up to 60% by volume. In some cases, Ca²⁺ may also be used for condensation. Ca²⁺ not only binds to DNA phosphates, but can also form a complex with the nitrogen₍₇₎ and oxygen₍₆₎ of guanine, disrupting base pairing.

An exterior surface of a liposome can be coated with a polymer in some cases. In other cases, an exterior surface may not be coated. A liposome can carry a therapeutic gene. A liposome can be a form of nano-container, such as nanoparticles or liposomes that can be used for encapsulation of therapeutic agents. A liposome can be made with neutral phospholipids such as 1-palmitoyl-2-oleoyl-sn-glycerol-3-phosphocholine (POPC), diphosphatidy phosphocholine, distearoylphosphatidylethanolamine (DSPE), or cholesterol, along with a small amount (1%) of cationic lipid, such as didodecyldimethylammonium bromide (DDAB). In some cases, a material that can be in a lipid bilayer can be a lipid with a net positive charge or a lipid with a neutral charge. In some cases, a cationic lipid can be used to stabilize a therapeutic agent encapsulated within a liposome, such as DNA.

In some cases, a polynucleic acid such as a minicircle can be fully encapsulated in a liposomal structure. Full encapsulation can indicate that a polynucleic acid in a liposomal structure may not be significantly degraded after exposure to serum or a nuclease or protease assay that would significantly degrade free DNA, RNA, or protein. In a fully encapsulated system, preferably less than about 25% of a polynucleic acid in a liposomal structure can be degraded in a treatment that would normally degrade 100% of free polynucleic acid, more preferably less than about 10%, and most preferably less than about 5% of a polynucleic acid in a liposomal structure can be degraded. In the context of polynucleic acids, full encapsulation may be determined by an Oligreen® assay. Oligreen® is an ultra-sensitive fluorescent nucleic acid stain for quantitating oligonucleotides and single-stranded DNA or RNA in solution (available from Invitrogen Corporation; Carlsbad, Calif.). “Fully encapsulated” can also indicate that a liposomal structure may be serum-stable, that is, that they do not rapidly decompose into their component parts upon in vivo administration.

Mucus-penetrating particle or MPP as used herein, can refer to particles which have been coated with a mucosal penetration enhancing coating. In some cases, a particle can be or can deliver a particle of an active agent, such as a therapeutic, diagnostic, prophylactic, and/or nutraceutical agent (i.e., drug particle) that can be coated with a mucosal penetrating enhancing coating. In other cases, particles can be formed of a matrix material, such as a polymeric material, in which a therapeutic, diagnostic, prophylactic, and/or nutraceutical agent can be encapsulated, dispersed, and/or associated. Coating material can be covalently or non-covalently associated with a drug particle or polymeric particle II.

Further, provided herein can be a liposomal structure that can pass through a mucosal barrier at a greater rate than other liposomal structures, e.g., unmodified liposomal structures. A liposomal structure may pass through a mucosal barrier at a rate that is at least 2, 5, 10, 20, 30, 50, 100, 200, 500, 1000- or greater fold higher than, e.g., an unmodified liposomal structure of a similar size. In some cases, a non-PEG modified liposomal structure can penetrate a mucosal barrier more efficiently than a PEG-modified liposomal structure as measured by a transwell migration assay.

Mucus-penetrating nanoparticles (MPPs) or nanoparticles can contain polymers. A polymer can be any polymeric particle. Any number of biocompatible polymers can be used to prepare nanoparticles. In one embodiment, a biocompatible polymer can be biodegradable. In another embodiment, a particle may not be non-degradable. In other embodiments, particles can be a mixture of degradable and non-degradable particles.

An MPP can have a near-neutral zeta potential from about −100 mV to about 100 mV. An MPP can have a zeta potential from about −50 mV to about 50 mV, from about −30 mV to about 30 mV, from about −20 mV to about 20 mV, from about −10 mV to about 10 mV, from about −5 mV to about 5 mV.

Biodegradable polymers typically differ from non-biodegradable polymers in that the former may degrade during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to sidechain or that connects a side chain to the polymer backbone. For example, a therapeutic agent or other chemical moiety attached as a side chain to the polymer backbone may be released by biodegradation. In certain embodiments, one or the other or both general types of biodegradation may occur during use of a polymer. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of the implant, and the mode and location of administration. For example, the greater the molecular weight, the higher the degree of crystallinity, and/or the greater the biostability, the biodegradation of any biodegradable polymer is usually slower.

In certain embodiments a biodegradable polymer may also have a therapeutic agent or other material associated with it, the biodegradation rate of such polymer may be characterized by a release rate of such materials. For example, the biodegradation rate may depend on not only the chemical identity and physical characteristics of the polymer, but also on the identity of material(s) incorporated therein. In some cases, polymeric formulations of the present invention biodegrade within a period that is acceptable in a desired application. In certain embodiments, such as in vivo therapy, such degradation occurs in a period usually less than about five years, one year, six months, three months, one month, fifteen days, five days, three days, or even one day or less (e.g., 4-8 hours) on exposure to a physiological solution with a pH between 6 and 8 having a temperature of between 25 and 37° C. In other embodiments, the polymer degrades in a period of between about one hour and several weeks, depending on the desired application.

Polymers

Polymers can include, but may not be limited to, cyclodextrin-containing polymers, in particular cationic cyclodextrin-containing polymers, such as those described in U.S. Pat. No. 6,509,323; polymers prepared from lactones, such as poly(caprolactone) (PCL); polyhydroxy acids and copolymers thereof such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,Wactide) (PDLA), poly(2-methyl-2-oxazoline), poly(2-ethyl-2-oxazoline), and poly(2-n-propyl-2-oxazoline), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), and blends thereof, polyalkyl cyanoacralate, polyurethanes, polyamino acids such as poly-L-lysine (PLL), poly(valeric acid), and poly-L-glutamic acid; hydroxypropyl methacrylate (HPMA); polyanhydrides; polyesters; polyorthoesters; poly(ester amides); polyamides; poly(ester ethers); polycarbonates; polyalkylenes such as polyethylene and polypropylene; polyalkylene glycols such as polyethylene glycol) (PEG) and polyalkylene oxides (PEO), and block copolymers thereof such as polyoxyalkylene oxide (“PLURONICS®”); polyalkylene terephthalates such as polyethylene terephthalate); ethylene vinyl acetate polymer (EVA); polyvinyl alcohols (PVA); polyvinyl ethers; polyvinyl esters such as poly(vinyl acetate); polyvinyl halides such as polyvinyl chloride) (PVC), polyvinylpyrrolidone; polysiloxanes; polystyrene (PS; celluloses including derivatized celluloses such as alkyl celluloses, hydroxyalkyl celluloses, cellulose ethers, cellulose esters, nitro celluloses, hydroxypropylcellulose, and carboxymethylcellulose; polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (jointly referred to herein as “polyacrylic acids”); polydioxanone and its copolymers; polyhydroxyalkanoates; polypropylene fumarate; polyoxymethylene; poloxamers; poly(butyric acid); trimethylene carbonate; polyphosphazenes, or any combination thereof. Examples of natural polymers can include proteins such as albumin, collagen, gelatin and prolamines, for example, zein, and polysaccharides such as alginate. Copolymers of the above, such as random, block, or graft copolymers, or blends of a polymer listed above can also be used. A polymer can be PEG. A polymer can be PEG 2000. A polymer can also be a non-PEG polymer such as poly (2-alkyl-2-oxazoline). In some cases, a side chain variation in a polymer may contribute to diffusion of a structure through a mucosal barrier. In some cases, a non-PEG can comprise an L-amino-acid-modified complex.

A polymer can be poly (ethylene glycol), also known as PEG. PEG may be employed to reduce adhesion in mucous in certain configurations, e.g., wherein the length of PEG chains extending from the surface is controlled (such that long, unbranched chains that interpenetrate into the ECM are reduced or eliminated). For example, linear high MW PEG may be employed in the preparation of particles such that only portions of the linear strands extend from the surface of the particles (e.g., portions equivalent in length to lower MW PEG molecules). Alternatively, branched high MW PEG may be employed. In such embodiments, although the molecular weight of a PEG molecule may be high, the linear length of any individual strand of the molecule that extends from the surface of a particle would correspond to a linear chain of a lower MW PEG molecule. In some aspects, an average molecular weight of a PEG can be at least 200 Da, 500 Da, 950 Da, 1000 Da, 1500 Da, 2000 Da, 2500 Da, 3000 Da, 3500 Da, 4000 Da, 4500 Da, 5000 Da, 5500 Da, 6000 Da, 6500 Da, 7000 Da, 7500 Da, 8000 Da, 8500 Da, 9000 Da, 9500 Da, or 10000 Da

In alternative embodiments, polymer can be a poloxamer such as the polyethylene glycol-polyethylene oxide block copolymers marketed as PLUORONICs®. PEG alternative polymers can be soluble, hydrophilic, have highly flexible main chain, and high biocompatibility. Synthetic polymers, such as poly (vinyl pyrrolidone) (PVP) and poly (acryl amide) (PAA), are the most prominent examples of other potentially protective polymers. In some cases, a liposome containing DSPE covalently linked to poly (2-methyl-2-oxazoline) or to poly (2-ethyl-2-oxazoline) can also exhibit extended blood circulation time. In some cases, a liposome containing DSPE covalently linked to poly (2-methyl-2-oxazoline) or to poly (2-ethyl-2-oxazoline) can also exhibit decreased uptake by the liver and/or spleen. A polymer can also be poly [N-(2-hydroxypropyl) methacrylamide], amphiphilic poly-N-vinylpyrrolidones, L-amino-acid-based biodegradable polymer-lipid conjugates, and polyvinyl alcohol. L-amino-acid-based polymers may also be used.

In some cases, a thiolated nanoparticle can show little penetration into the gastric mucosa when compared to a non-thiolated nanoparticle. In some cases, PEGylated and POZylated particles can have greater penetration into, and permeation through, a mucosa. In some cases, thiolation can be inferior to PEGylation and POZylation in terms of nanoparticle mucosal penetration. In other cases, PEGylation can be inferior to POZylation in terms of nanoparticle mucosal penetration. In some cases, a nanoparticle comprising a POZylation can have from about 2×, 3×, 4×, 5×, 6×, 7×, 8×, 9×, 10×, 11×, 12×, 13×, 14×, 15×, or more than 20× increased mucosal penetration when compared to a non-POZylated particle. Mucosal penetration can be measured by one or more assays. In some cases, mucosal penetration is measured by a transwell migration assay. In other cases, mucosal penetration can be measured by nanoparticle tracking analysis (NTA). In other cases, mucosal penetration can be measured by multiparticle tracking (MPT).

A POZylation can comprise Formula I. For example, a POZylation (POZ) polymer can comprise Formula I:

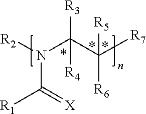

R₁ can be independently selected from a group consisting of hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof.

In some cases, R₂ can be independently selected from a group consisting of a coupling group capable of coupling to a linker, or a substrate; hydrogen; deuterium; C₁₋₆ alkyl; C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; XCY₂X or any combinations thereof.

In some cases, R₃ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof.

R₄ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof.

R₅ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof.

R₆ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof.

R₇ can be independently selected from a group consisting of hydrogen; deuterium; C₁₋₆ alkyl; C₂₋₆ alkynyl; C₂₋₆ alkynyl, C₃₋₈cycloalkyl; heteroaryl, cycloalkyl; C₁₋₆alkylheteroaryl; C₁₋₆alkylaryl; and alkylcycloalkyl; each of which may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combinations thereof; wherein * can independently be R, S or achiral; wherein ** can independently be R, S or achiral; wherein X is independently selected from oxygen or sulfur; Y is independently selected from deuterium or hydrogen; A is hydrogen, deuterium, aryl, or heteroaryl and n can be from about 1 to about 100. In other cases, n can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or up to 100.

In some cases, a polymer can comprise at least a portion of Formula I. For example, in some cases, a polymer can comprise at least 50% of Formula I. In other cases, a polymer can comprise from 50% to about 100% of Formula I. A polymer have a percent similarity to Formula I from 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or up to 100% of Formula I.

Phosphate salts of poly-2-oxazolines can have a weight average molecular weight of at least about 1000, as determined by the intrinsic viscosity-universal calibration curve. In other cases, a poly-2-oxazoline can have a weight average molecular weight of at least about 10,000. In other cases, a poly-2-oxazoline can have a weight average molecular weight of at least about 250,000. In other cases, a poly-2-oxazoline can have a weight average molecular weight of at least about 1,000,000 is preferred. In other cases, a poly-2-oxazoline can have a weight average molecular weight of at least about 500,000. A poly-2-oxazoline can have an average molecular weight from about 1000, 2000, 3000, 4000, 5000, 6000, 7000, 8000, 9000, 10000, 20000, 30000, 40000, 50000, 60000, 70000, 80000, 90000, 1000000, 200000, 300000, 400000, or up to about 500000 Da or greater. In certain instances, a polymer can be substituted by an alkyl, alkoxy, acyl, or aryl group. In some cases, a polymer such as POZ or PEG, can be conjugated directly to a lipid of a liposome or may be linked to a lipid of a liposome via a linker moiety. Any linker moiety suitable for coupling a polymer to a lipid can be used including, e.g., non-ester containing linker moieties and ester-containing linker moieties. A linker moiety can be a non-ester containing linker moiety. As used herein, the term “non-ester containing linker moiety” can refer to a linker moiety that does not contain a carboxylic ester bond (—OC(O)—). Suitable non-ester containing linker moieties include, but are not limited to, amino (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amino linker moiety). A carbamate linker can be used to couple the PEG to the lipid. In other embodiments, an ester containing linker moiety can be used to couple the PEG to the lipid. Suitable ester containing linker moieties include but is not limited, e.g., carbonate (—OC(O)O—), succinoyl, phosphate esters (—O—(O)POH—O—), sulfonate esters, and combinations thereof.

In some cases, Formula I can have an average molecular weight from about 1000 Da to about 8000 Da. Formula I can have an average molecular weight from about 500 Da to about 10000 Da. Formula I can have an average molecular weight from about 500 Da, 550 Da, 600 Da, 650 Da, 700 Da, 750 Da, 800 Da, 850 Da, 900 Da, 950 Da, 1000 Da, 1500 Da, 2000 Da, 2500 Da, 3000 Da, 3500 Da, 4000 Da, 4500 Da, 5000 Da, 5500 Da, 6000 Da, 6500 Da, 7000 Da, 7500 Da, 8000 Da, 8500 Da, 9000 Da, 9500 Da, or up to 10000 Da or greater.

In some cases, masking a thiol group can lead to increased mucosal penetration. For example, a POZylation can mask a thiol group. In some cases, the use of a poly 2-oxazoline compound can lead to increased mucosal penetration over a PEGylated liposomal structure. POZ can also be more readily excreted via the renal route compared to PEG. For example, a POZ group can have from 1% to 100% higher renal excretion when compared to PEG. POZ can have from about 1%, 10%, 20%, 30%, 40%, 50%, 60%, 70%, 80%, 90%, or up to about 100% higher renal excretion than PEG. In some cases, a POZ group may be more biodegradable compared to a PEG group. Biodegradation can be measured by oxidative degradation in some cases.

In some cases, POZ can also be more readily excreted via the renal route compared to PEG. For example, a POZ group can have from 1% to 100% higher renal excretion when compared to PEG. POZ can have from about 1%, 10%, 20%, 30%, 40%, 50%, 60%, 70%, 80%, 90%, or up to about 100% higher renal excretion than PEG. In some cases, a POZ group may be more biodegradable compared to a PEG group. Biodegradation can be measured by oxidative degradation in some cases.

In some cases, a polymer may be modified. Functional groups on a polymer can be capped to alter properties of a polymer and/or modify (e.g., decrease or increase) reactivity of a functional group. For example, a carboxyl termini of carboxylic acid contain polymers, such as lactide- and glycolide-containing polymers, may optionally be capped, e.g., by esterification, and a hydroxyl termini may optionally be capped, e.g. by etherification or esterification. Copolymers of PEG or derivatives thereof with any polymer described above may be used to make polymeric particles. In certain embodiments, PEG or derivatives may be located in interior positions of a copolymer. Alternatively, PEG or derivatives may locate near or at a terminal position of a copolymer. For example, one or more polymers above can be terminated with a block of polyethylene glycol. In some embodiments, a core polymer can be a blend of PEGylated polymer and non-PEGylated polymer, wherein a base polymer can be the same (e.g., PLGA and PLGA-PEG) or different (e.g., PLGA-PEG and PLA). In certain cases, a nanoparticle can be formed under conditions that can allow regions of PEG to phase separate or otherwise locate to a surface of a nanoparticle. A surface-localized PEG region alone may perform the function of, or include, a surface-altering agent. In some cases, a nanoparticle can be prepared from one or more polymers terminated with blocks of polyethylene glycol as a surface-altering material. In some cases, a nanoparticle can be coated with PEG. In some cases, a liposome can be coated with PEG. In some cases, PEG can be associated to a lipid before a liposome may be formed.

A polymer can be of any size and weight. In some cases a polymer's weight can vary for a given polymer but can be from about 25 Dalton, 50 Daltons, 100 Daltons, 200 Daltons, 300 Daltons, 400 Daltons, 500 Daltons, 600 Daltons, 700 Daltons, 800 Daltons, 900 Daltons, 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

In some cases, a nanoparticle may be used as gene carrier. In some embodiments, a nanoparticle can be formed of one or more polycationic polymers which complex with one or more nucleic acids which can be negatively charged. A cationic polymer can be any synthetic or natural polymer bearing at least two positive charges per molecule and having sufficient charge density and molecular size to bind to nucleic acid under physiological conditions (i.e., pH and salt conditions encountered within a body or within cells). In certain embodiments, a polycationic polymer contains one or more amine residues.

In some cases, a nanoparticle containing a therapeutic, diagnostic, prophylactic, and/or nutraceutical agent can be coated with a mucosal penetration enhancing coating. A nanoparticle can be a microparticle or a nanoparticle. A coating can be applied using any means, techniques, supplies, or combinations thereof. A mucosal penetration enhancing coating can be covalently or non-covalently associated with a lipid, polymer, or any combination. In some embodiments, it may be non-covalently associated. In other embodiments, a lipid or polymer can contain a reactive functional group or one can be incorporated to which a mucosal penetration enhancing coating can be covalently bound.

Nanoparticles may be coated with or contain one or more surface altering agents. In some cases, a surface-alternating agent can provide a direct therapeutic effect, such as reducing inflammation. A nanoparticle can be coated such as a coating provides a nanoparticle with a near-neutral zeta potential. A coating can be PEGylation. A coating can be a partial coating or a full coating. Examples of surface-altering agents include, but are not limited to, proteins, including anionic proteins (e.g., albumin), surfactants, sugars or sugar derivatives (e.g., cyclodextrin), therapeutics agents, and polymers. Polymers may also include heparin, polyethylene glycol (“PEG”) and poloxomers (polyethylene oxide block copolymers). A polymer may be PEG, PLURONIC F127®, PEG2000, or any derivative, modified version thereof, or combination thereof.

A surface-altering agent may increase charge or hydrophilicity of the liposomal structure or liposomal particle, or otherwise decrease interactions between the particle and mucus, thereby promoting motility through mucus. A surface-altering agent may enhance the average rate at which the polymeric or liposomal particles, or a fraction of the particles, move in or through mucus. Examples of suitable surface-altering agents include but are not limited to anionic protein (e.g., serum albumin), nucleic acids, surfactants such as cationic surfactants (e.g., dimethyldioctadecyl-ammonium bromide), sugars or sugar derivatives (e.g., cyclodextrin), polyethylene glycol, mucolytic agents, or other non-mucoadhesive agents. Certain agents, e.g., cyclodextrin, may form inclusion complexes with other molecules and can be used to form attachments to additional moieties and facilitate the functionalization of the particle surface and/or the attached molecules or moieties. In some cases, a surface altering agent can cause a surface modification. A surface altering agent can be PEG, PEG can be a polymer used in a liposomal structure. A surface modification can be interchanged with modification. In some cases, a modification can refer to a surface modification. In other cases, a modification may not refer to a surface modification.

In some cases, mucus disruptive agents can be delivered or can be found on a particle. Mucus can be a biological gel that coats tissue surfaces generally exposed to the external environment such as the airways, GI tract, eyes and reproductive tract. It can form a defensive barrier that captures or blocks foreign bodies and pathogenic bacteria from reaching the underlying cells and causing damage or disease. Mucus is predominantly comprised of water (around 95%), glycoproteins (2-5%), lipids, and salts. Glycosylated proteins can be from a MUC family. In some routes of drug administration, such as oral, nasal, pulmonary or vaginal, mucus may act as a barrier. Liposomal structures carrying a polynucleic acid or other cargo may need to be specifically designed to penetrate a mucosal layer before they are removed via mucus clearance. Enhancing mucosal penetration and permeation is therefore essential to avoid capture and excretion from a mucosal barrier, and to fully exploit the benefits of nanoparticle-based drug delivery.

Mucus disruptive agents can be an NSAID, a miRNA against B-catenin or an agent that may be known to disrupt mucus. Mucus disruptive agents can be surface altering agents. In some cases, disrupting mucous can be eliminating production of mucous. In other cases, disrupting mucous can be reducing the production of mucous. For example, reducing mucous may mean reducing the production of mucous by targeting a cell that generates mucous. Mucous disruption may also mean adjusting the consistency of mucous. For example, mucous disruption may mean loosening the consistency of mucous.

In some cases, a liposomal structure can comprise an NSAID. An NSAID can be ibuprofen, aspirin, ketoprofen, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, salsalate, or a combination thereof.

In some cases, CEQ508, an RNAi therapeutic for the treatment of Familial Adenomatous Polyposis (FAP) can be delivered to a subject in need thereof enclosed within a liposome complex. CEQ508 can act by utilizing an RNA interference (tkRNAi) platform. CEQ508 can comprise attenuated bacteria that are engineered to enter into dysplastic tissue and release a payload of short-hairpin RNA (shRNA), a mediator in an RNAi pathway. The shRNA targets the mRNA of beta-catenin, which is known to be dysregulated in classical FAP. CEQ508 can be an administered treatment to reduce the levels of beta-catenin protein in the epithelial cells of the small and large intestine.

In some cases, a nanoparticle can be coated with or contain polyethylene glycol (PEG). Alternatively, a PEG can be in the form of blocks covalently bound (e.g., in the interior or at one or both terminals) to a lipid used to form a nanoparticles. In particular embodiments, a nanoparticle can be formed from block copolymers containing PEG. A nanoparticle can also be prepared from block copolymers containing PEG, wherein PEG may be covalently bound to a terminal of a base lipid. Representative PEG molecular weights can include 300 Da, 600 Da, 1 kDa, 2 kDa, 3 kDa, 4 kDa, 6 kDa, 8 kDa, 10 kDa, 15 kDa, 20 kDa, 30 kDa, 50 kDa, 100 kDa, 200 kDa, 500 kDa, and 1 MDa and all values within the range of 300 Daltons to 1 MDa. A PEG can be about 2 kDa in some cases. PEG of any given molecular weight may vary in other characteristics such as length, density, and branching.

A PEG coating can be applied at any concentration. In some cases, a concentration between lipid to PEG can be 5 to 10%. A concentration can be at least 5% or at most 10%. In some cases, a concentration can be over 10%. A concentration can be or can be about 1%, 2%, 3%, 4%, 5%, 6%, 7%, 8%, 9%, 10%, or over 10%. In some embodiments, PEG surface density can be controlled by preparing a nanoparticle from a mixture of PEGylated and non-PEGylated particles. For example, a surface density of PEG on nanoparticles can be precisely controlled by preparing particles from a mixture of poly (lactic-co-glycolic acid) and poly (ethylene glycol) (PLGA-PEG).

In some cases, a PEG coating can be measured for density on a nanoparticle. Quantitative ¹H nuclear magnetic resonance (NMR) can be used to measure surface PEG density on nanoparticles. In some cases, a density can be or can be about 10 to 16 PEG chains/100 nm². In some cases a density can be over 10 to 16 PEG chains/100 nm². This density threshold may vary depending on a variety of factors including a liposome of a nanoparticle, particle size, and/or molecular weight of PEG. Density of a coating that can be applied to a liposome can be varied based on a variety of factors including a surface altering material and a composition of a particle. In one embodiment, density of a surface altering material, such as PEG, as measured by ¹H NMR can be or can be about, 0.1, 0.2, 0.5, 0.8, 1, 2, 5, 8, 10, 15, 20, 25, 40, 50, 60, 75, 80, 90, or 100 chains per nm². The range above can be inclusive of all values from 0.1 to 100 units per nm². In some cases, a density of a surface altering material, such as PEG, can be or can be about 1 to about 25 chains/nm², can be or can be about 1 to about 20 chains/nm², can be or can be about 5 to about 20 chains/nm², can be or can be about 5 to about 18 chains/nm², can be or can be about 5 to about 15 chains/nm², or can be or can be about 10 to about 15 chains/nm². In other cases a density can be or can be about 0.05 to about 0.5 PEG chains/nm². PEG can be 10 to 20 chains per 100 nm².

A concentration of a surface altering material, such as PEG, can also be varied. In particular embodiments, a density of a surface-altering material (e.g., PEG) can be such that a surface-altering material (e.g. PEG) adopted an extended brush configuration. In other embodiments, a mass of a surface-altering moiety can be at least or can be at least about 1/10,000, 1/7500, 1/5000, 1/4000, 1/3400, 1/2500, 1/2000, 1/1500, 1/1000, 1/750, 1/500, 1/250, 1/200, 1/150, 1/100, 1/75, 1/50, 1/25, 1/20, ⅕, ½, or 9/10 of a mass of a nanoparticle. The range above can be inclusive of all values from 1/10,000 to 9/10.

A polymer such as PEG or POZ, can be at a density from about 0.05 μg/nm² to about 0.25 μg/nm². A polymer can also be at a density from about 0.01 μg/nm 2, 0.02 μg/nm 2, 0.03 μg/nm² 0.04 μg/nm², 0.05 μg/nm², 0.06 μg/nm², 0.07 μg/nm², 0.08 μg/nm², 0.09 μg/nm², 0.1 μg/nm², 0.15 μg/nm 2, 0.2 μg/nm 2, 0.25 μg/nm 2, 0.3 μg/nm 2, 0.35 μg/nm 2, 0.4 μg/nm 2, 0.45 μg/nm 2, 0.5 μg/nm 2, 0.55 μg/nm 2, 0.6 μg/nm 2, 0.65 μg/nm 2, 0.7 μg/nm 2, 0.75 μg/nm 2, 0.8 μg/nm 2 μg/nm 2, 0.85 μg/nm², 0.9 μg/nm², 0.95 μg/nm², or up to 1 μg/nm². In some embodiments μg/nm² with regard to density can refer to μg polymer per nm² liposomal structure or liposomal structure surface. In some embodiments μg refers to microgram. In some embodiments, nm refers to nanometer.

In some cases, a polymer can be a poly (2-alkyl-2-oxazoline) addition. Similar to PEG, poly (2-alkyl-2-oxazoline) has “stealth” properties, is non-toxic and biocompatible, has a pendent group for further functionalization, and a high degree of renal clearance with low bioaccumulation. Poly (2-alkyl-2-oxazoline) can increase mucosal penetration of a structure. In some cases, non-PEG coated structures may have increased mucosal penetration to structures coated with PEG. Increased mucosal penetration can be measured by a transwell migration assay. Additional assays that can be utilized to measure mucosal penetration can comprise multiple particle tracking (MPT), Using chamber, or a combination thereof. In some cases, a mucosal penetration assay can record a liposomal structure's dynamic transit in a mucus using fluorescence microscopy, such as fluorescence recovery after photobleaching (FRAP) and multiple particle tracking (MPT). FRAP can be the fluorescently labeled liposomal structure's exposure to a laser beam to form a floating white spot. The diffusion coefficient can be obtained by recovery of a fluorescence intensity, which may result following diffusion of a fluorescently labeled molecule into an area with a flow of liposomal structures.

To better understand a fate of a particles and how results might translate in humans, a mucosal penetration study can adopt an animal model to investigate a therapeutic effect or pharmacokinetics of a liposomal structure, which mainly include isolated intestinal experiments, in situ experiments and in vivo experiments. For example, in an in situ experiment of mucosal penetration a portion of a small intestine can be excised from an abdominal cavity, subsequently ligated at both ends to make an isolated “loop”, and a liposomal structure can be directly injected into a loop. After a chosen time period, an animal can be sacrificed and the intestinal loop can be removed from a body cavity for further morphology or quantitative analysis.

In some cases a coating can be an enteric coating. Enteric coatings can be utilized to prevent or minimize dissolution in the stomach but allow dissolution in the small intestine. In some embodiments, a coating can include an enteric coating. An enteric coating can be a barrier applied to oral medication that prevents release of medication before it reaches the small intestine. Delayed-release formulations, such as enteric coatings, can an irritant effect on the stomach from administration of a medicament from dissolving in the stomach. Such coatings are also used to protect acid-unstable drugs from the stomach's acidic exposure, delivering them instead to a basic pH environment (intestine's pH 5.5 and above) where they may not degrade.

Dissolution can occur in an organ. For example, dissolution can occur within a duodenum, jejunum, ilium, and/or colon, or any combination thereof. In some cases, dissolution can occur in proximity to a duodenum, jejunum, ilium, and/or colon. Some enteric coatings work by presenting a surface that is stable at a highly acidic pH found in the stomach, but break down rapidly at a less acidic (relatively more basic) pH. Therefore, an enteric coated pill may not dissolve in the acidic environment of the stomach, but can dissolve in an alkaline environment present in a small intestine. Examples of enteric coating materials include, but are not limited to, methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid.

An enteric coating can be applied at a functional concentration. An enteric coating can be cellulose acetate phthalate, Polyvinyl acetate phthalate, Hydroxypropylmethylcellulose acetate succinate, Poly(methacylic acid-co-ethyl acrylate) 1:1, Poly(methacrylic acid-co-ethyl acrylate) 1:1, Poly(methacylic acid-co-methyl methacrylate) 1:1, Poly(methacylic acid-co-methyl methacrylate) 1:1, Poly(methacylic acid-co-methyl methacrylate) 1:2, Poly(methacylic acid-co-methyl methacrylate) 1:2, Poly(methyl acrylate-co-methyl methacrylate-co-methacrylic acid) 7:3:1, or any combination thereof. An enteric coating can be applied from about 6 mg/(cm²) to about 12 mg/(cm²). An enteric coating can also be applied to a structure from about 1 mg/(cm²), 2 mg/(cm²), 3 mg/(cm²), 4 mg/(cm²), 5 mg/(cm²), 6 mg/(cm²), 7 mg/(cm²), 8 mg/(cm²), 9 mg/(cm²), 10 mg/(cm²), 11 mg/(cm²), 12 mg/(cm²), 13 mg/(cm²), 14 mg/(cm²), 15 mg/(cm²), 16 mg/(cm²), 17 mg/(cm²), 18 mg/(cm²), 19 mg/(cm²), to about 20 mg/(cm²).

In some embodiments, a pharmaceutical composition can be orally administered from a variety of drug formulations designed to provide delayed-release. Delayed oral dosage forms include, for example, tablets, capsules, caplets, and may also comprise a plurality of granules, beads, powders or pellets that may or may not be encapsulated. Tablets and capsules can represent oral dosage forms, in which case solid pharmaceutical carriers can be employed. In a delayed-release formulation, one or more barrier coatings may be applied to pellets, tablets, or capsules to facilitate slow dissolution and concomitant release of drugs into the intestine. Typically, a barrier coating can contain one or more polymers encasing, surrounding, or forming a layer, or membrane around a therapeutic composition or active core. In some embodiments, active agents, such as a polynucleic acid, can be delivered in a formulation to provide delayed-release at a pre-determined time following administration. The delay may be up to about 10 minutes, about 20 minutes, about 30 minutes, about 1 hour, about 2 hours, about 3 hours, about 4 hours, about 5 hours, about 6 hours, or up to 1 week in length. In some cases, an enteric coating may not be used to coat a particle.

Polymers or coatings that can be used to achieve enteric release can be anionic polymethacrylates (copoly-merisate of methacrylic acid and either methyl-methacrylate or ethylacrylate (Eudragit®), cellulose based polymers, e.g. cellulose acetatephthalate (Aquateric®) or polyvinyl derivatives, e.g. polyvinyl acetate phthalate (Coateric®) in some cases.

Depending upon the ratio of polynucleic acid to polymer and the nature of the particular polymer employed, the rate of polynucleic acid, such as minicircle DNA, release can be controlled. In some cases, a depot injectable formulation can be prepared by entrapping a polynucleic acid in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

A nanoparticle may have a variety of shapes and cross-sectional geometries that may depend, in part, upon the process used to produce it. In one case, a nanoparticle may have a shape that can be a sphere, a rod, a tube, a flake, a fiber, a plate, a wire, a cube, or a whisker. A nanoparticle may include particles having two or more of the aforementioned shapes. In another case, a cross-sectional geometry of the particle may be one or more of circular, ellipsoidal, triangular, rectangular, or polygonal. In one embodiment, a nanoparticle may be a non-spherical particle. For example, a nanoparticle may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis can be substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles. Cell Penetrating Peptides

TABLE 1
Cell Penetrating Peptides: One letter code used.

| SEQ | |
|---|---|
| ID | |
| NO | Sequence |
| 1 | RQIKIWFQNRRMKWKK |
| 2 | YGRKKRRQRRRPPQ |
| 3 | PLSSIFSRIGDP |
| 4 | RRRRRRRRR |
| 5 | AAVALLPAVLLALLAP |
| 6 | RGGRLSYSRRRFSTSTGR |
| 7 | LLIILRRRIRKQAHAHSK |
| 8 | PKKKRKV |
| 9 | RKKRRQRRR |
| 10 | GRKKRRQRRRPPQ |
| 11 | LGTYTQDFNKFHTFPQTAIGVGAP |
| 12 | VQRKRQKLMP |
| 13 | SKKKKTKV |
| 14 | GRKRKKRT |
| 15 | RRRERRAEK |
| 16 | KCPSRRPKR |
| 17 | (VRLPPP)n |
| 18 | (VHLPPP)n |
| 19 | (VKLPPP)n |
| 20 | AVGAIGALFLGFLGAAG |
| 21 | VTVLALGALAGVGVG |
| 22 | IAARIKLRSRQHIKLRHL |
| 23 | DTWPGVEALIRILQQLLFIHFRIGCQH |
| 24 | DAATATRGRSAASRPTERPRAPARSASRPRRPVD |
| 25 | GALFLGWLGAAGSTMGA |
| 26 | MGLGLHLLVLAAALQGA |
| 27 | RQGAARVTSWLGRQLRIAGKRLEGRSK |
| 28 | MANLGYWLLALFVTMWTDVGLCKKRPKP |
| 29 | TRRNKRNRIQEQLNRK |
| 30 | TAKTRYKARRAELIAERR |
| 31 | MDAQTRRRERRAEKQAQWKAAN |
| 32 | RRRRNRTRRNRRRVR |
| 33 | KMTRAQRRAAARRNRWTAR |
| 34 | TRRQRTRRARRNR |
| 35 | RQARRNRRRRWR |
| 36 | DRRRRGSRPSGAERRRRRAAAA |
| 37 | QTRRRERRAEKQAQW |
| 38 | KRPAAIKKAGQAKKKK |
| 39 | TRRSKRRSHRKF |
| 40 | RAGLQFPVGRVHRLLRK |
| 41 | TRSSRAGLQFPVGRVHRLLRK |
| 42 | RHIKIWFQNRRMKWKK |
| 43 | YKQCHKKGGHCFPKEKICLPPSSDFGKMDCRWRWKCCKKGSG |
| 44 | RVIRVWFQNKRCKDKK |
| 45 | SKRTRQTYTRYQTLELEKEFHFNRYITRRRRIDIANALSLSERQ |
| | IKIWFQNRRMKSKKDR |
| 46 | EKRPRTAFSSEQLARLKREFNENRYLTERRRQQLSSELGLNEAQ |
| | IKIWFQNKRAKIKKST |
| 47 | GWTLNSAGYLLGKINLKALAALAKKIL |
| 48 | RRRRRRRR |
| 49 | rrrrrrrrr |
| 50 | KLALKLALKALKAALKLA |
| 51 | WEAKLAKALAKALAKHLAKALAKALKACEA |
| 52 | YARLAARQARA |
| 53 | DPKGDPPKGVTVTVTVTVTGKGDPKPD |
| 54 | KKWKMRRNQFWVRVQR |
| 55 | RRWRRWWWRRWWRRWRR |
| 56 | GALFLGFLGAAGSTMGAWSQPKSKRKVC |
| 57 | KETWWETWWTEWSQPKKKRKV |
| 58 | GALFLGWLGAAGSTMGAWSQPKKKRKV |
| 59 | RRQRRTSKLMKR |
| 60 | RRIPNRRPRR |
| 61 | YGRRARRRRRR |
| 62 | SQMTRQARRLYV |
| 63 | SIPPEVKFNKPFVYLI |
| 64 | KKWKMRRNQFWVKVQRG |
| 65 | AAVALLPAVLLALLAVTDQLGEDFFAVDLEAFLQEFGLLPEKE |
| 66 | KKAAAVLLPVLLAAP |
| 67 | INLKALAALAKKIL |
| 68 | MPKKKPTPIQLNP |
| 69 | AAVALLPAVLLALLAK |
| 70 | MNLLRKIVKNRRDEDTQKSSPASAPLDDG |
| 71 | ACSSSPSKHCG |
| 72 | RRLSYSRRRF |
| 73 | PIRRRKKLRRLK |
| 74 | MGLGLHLLVLAAALQGAWSQPKKKRKV |
| 75 | KETWEETWFTEWSQPKKKRKV |
| 76 | (R)n (n ≥ 3) |
| 77 | (K)n (n ≥ 3) |
| 78 | KIAAKSIAKIWKSILKIA |
| 79 | KALAKALAKLWKALAKAA |
| 80 | KLALKLALKWAKLALKAA |
| 81 | KLLAKAAKKWLLLALKAA |
| 82 | KLLAKAALKWLLKALKAA |
| 83 | KALKKLLAKWLAAAKALL |
| 84 | KLAAALLKKWKKLAAALL |
| 85 | KALAALLKKWAKLLAALK |
| 86 | KALAALLKKLAKLLAALK |
| 87 | KLALKLALKALKLALK |
| 88 | KLALKALKAALKLA |
| 89 | KLALKLALKALKAA |
| 90 | KLGLKLGLKGLKGGLKLG |
| 91 | KLALKLALKALQAALQLA |
| 92 | KLALQLALQALQAALQLA |
| 93 | QLALQLALQALQAALQLA |
| 94 | ELALELALEALEAALELA |
| 95 | LKTLATALTKLAKTLTTL |
| 96 | LLKTTALLKTTALLKTTA |
| 97 | LKTLTETLKELTKTLTEL |
| 98 | LLKTTELLKTTELLKTTE |
| 99 | klalklalkalkaalkla |
| 100 | KALKLKLALALLAKLKLA |
| 101 | KKWKMRRNQFWIKIQR |
| 102 | rqikiwfqnrrmkwkk |
| 103 | RQIKIWFPNRRMKWKK |
| 104 | RQPKIWFPNRRKPWKK |
| 105 | ROIKIWFQNRRMKWK |
| 106 | RQIKIWFQNRRMKW |
| 107 | RQIKIWFQNRRMK |
| 108 | RQIKIWFQNRRM |
| 109 | RQIKTWFQNRR |
| 110 | RQIKIWFQNR |
| 111 | RQIKIWFQN |
| 112 | RQIKIWFQ |
| 113 | RQIKIW |
| 114 | QIKIWFQNRRMKWKK |
| 115 | IKIWFQNRRMKWKK |
| 116 | KIWFQNRRMKWKK |
| 117 | IWFQNRRMKWKK |
| 118 | WFQNRRMKWKK |
| 119 | FQNRRMKWKK |
| 120 | QNRRMKWKK |
| 121 | NRRMKWKK |
| 122 | RRMKWKK |
| 123 | RMKWKK |
| 124 | AQIKIWFQNRRMKWKK |
| 125 | RAIKIWFQNRRMKWKK |
| 126 | RQAKIWFQNRRMKWKK |
| 127 | RQIAIWFQNRRMKWKK |
| 128 | RQIKAWFQNRRMKWKK |
| 129 | RQIKIWAQNRRMKWKK |
| 130 | RQIKIWAQNRRMKWKK |
| 131 | RQIKIWFANRRMKWKK |
| 132 | RQIKIWFQARRMKWKK |
| 133 | RQIKIWFQNARMKWKK |
| 134 | KIKIWFQNRAMKWKK |
| 135 | RQIKIWFQNRRAKWKK |
| 136 | RQTKIWFQNRRMAWKK |
| 137 | RQIKIWFQNRRMKAKK |
| 138 | RQIKIWFQNRRMKWAK |
| 139 | RQIKIWFQNRRMKWKA |
| 140 | CRQIKIWFPNRRMKWKKC |
| 141 | RQIKIWFPNRRMKWKK |
| 142 | RQIKIWFQNRRMKAKK |
| 143 | RQIRIWFQNRRMRWRR |
| 144 | RRRRRRRW |
| 145 | GRKKRRQRRRPWQ |
| 146 | GRKKRRQRRRPWQ |
| 147 | RQIRIWFQNRRMRWRR |
| 148 | RRWRRWWRRWWRRWRR |
| 149 | RQIKIWFQNMRRKWKK |
| 150 | KMDCRWRWKCCKK |
| 151 | MDCRWRWKCCKK |
| 152 | DCRWRWKCCKK |
| 153 | CRWRWKCCKK |
| 154 | RWRWKCCKK |
| 155 | KMDCRWRWKCKK |
| 156 | KMDCRWRWKKK |
| 157 | KMDRWRWKKK |
| 158 | KDCRWRWKCCKK |
| 159 | KCRWRWKCCKK |
| 160 | KRWRWKCCKK |
| 161 | MDCRWRWKXCKK |
| 162 | DCRWRWKXCKK |
| 163 | DCRWRWKCXKK |
| 164 | CRWRWKXCKK |
| 165 | CRWRWKCXKK |
| 166 | RWRWKXCKK |
| 167 | MDCRWRWKXXKK |
| 168 | DCRWRWKXXKK |
| 169 | CRWRWKXXKK |
| 170 | RWRWKXXKK |
| 171 | CRWRWKCSKK |
| 172 | SRWRWKCCKK |
| 173 | SRWRWKCSKK |
| 174 | SRWRWKSCKK |
| 175 | CRWRWKSSKK |
| 176 | SRWRWKSSKK |
| 177 | CRFRWKCCKK |
| 178 | CRWRFKCCKK |
| 179 | CRFRFKCCKK |
| 180 | crwrwkcckk |
| 181 | KCCKWRWRCK |
| 182 | kcckwrwrck |
| 183 | CrWRWKCCKK |
| 184 | CRwRWKCCKK |
| 185 | CRWrWKCCKK |
| 186 | CRWRwKCCKK |
| 187 | CrwrwKCCKK |
| 188 | CRWRWKCGCKK |
| 189 | KCGCRWRWKCGCKK |
| 190 | CRWRWKCG |
| 191 | KMDXRWRWKCCKK |
| 192 | KMDXRWRWKXCKK |
| 193 | KMDXRWRWKXXKK |
| 194 | KMDXRWRWKCXKK |
| 195 | MDCRWRWKCXKK |
| 196 | KMDCRWRWKCSKK |
| 197 | KMDCRWRWKSCKK |
| 198 | KMDXRWRWKCCKK |
| 199 | KMDCRWRWKSSKK |
| 200 | KMDSRWRWKSSKK |
| 201 | KMDSRWRWKSCKK |
| 202 | KMDSRWRWKCSKK |
| 203 | KMDCRWRPKCCKK |
| 204 | KMDCRPRPKCCKK |
| 205 | KMDXRPRPKCCKK |
| 206 | KMDXRPRPKXCKK |
| 207 | KMDXRPRPKCXKK |
| 208 | KMDCRPRPKXCKK |
| 209 | KMDCRPRPKCXKK |
| 210 | rkkrrqrrr |
| 211 | rrrcrrkkr |
| 212 | RKKRRRESRKKRRRES |
| 213 | GRPRESGKKRKRKRLKP |
| 214 | GKRKKKGKLGKKRDP |
| 215 | GKRKKKGKLGKKRPRSR |
| 216 | RKKRRRESRRARRSPRHL |
| 217 | SRRARRSPRESGKKRKRKR |
| 218 | VKRGLKLRHVRPRVTRMDV |
| 219 | SRRARRSPRHLGSG |
| 220 | LRRERQSRLRRERQSR |
| 221 | GAYDLRRRERQSRLRRRERQSR |
| 222 | VPMLK |
| 223 | VPMLK |
| 224 | VPALR |
| 225 | VSALK |
| 226 | PMLKE |
| 227 | VPALK |
| 228 | VSLKK |
| 229 | VSGKK |
| 230 | KLPVM |
| 231 | IPMIK |
| 232 | KLGVM |
| 233 | KLPVT |
| 234 | VPMIK |
| 235 | IPALK |
| 236 | IPMLK |
| 237 | VPTLQ |
| 238 | QLPVM |
| 239 | ELPVM |
| 240 | VPTLE |
| 241 | vpt1k |
| 242 | AYRIKPTFRRLKWKYKGKFW |
| 243 | HARIKPTFRRLKWKYKGKFW |
| 244 | HYRIKPTARRLKWKYKGKFW |
| 245 | HYRIKPTF.RRLAWKYKGKFW |
| 246 | HYRIKPTFRRLKWKYKGKFA |
| 247 | VNADIKATTVFGGKYVSLTTP |
| 248 | GKYVSLTTPKNPTKRRITPKDV |
| 249 | TKRRITPKDVIDVRSVTTEINT |
| 250 | RSVTTEINTLFQTLTSIAEKVDP |
| 251 | AEKVDPVKLNLTLSAAAEALTGLGDK |
| 252 | GLGDKFGESIVNANTVLDDLNSRMPQSRHDIQQL |
| 253 | GDVYADAAPDLFDFLDSSVTTARTINA |
| 254 | ARTINAQQAELDSALLAAAGFGNTTADVFDRG |
| 255 | ADVFDRGGPYLQRGVADLVPTATLLDTYSP |
| 256 | LDTYSPELFCTIRNFYDADRPDRGAAA |
| 257 | TKRRITPKDVIDVRSVTTEINT |
| 258 | TKRRITPDDVIDVRSVTTEINT |
| 259 | TKRRITPKKVIDVRSVTTEINT |
| 260 | TKRRITPKDVIDVRSVTTKINT |
| 261 | TKRRITPKDVIDV |
| 262 | TKRRITPKDVIDVESVTTEINT |
| 263 | TARRITPKDVIDVRSVTTEINT |
| 264 | TKAARITPKDVIDVRSVTTEINT |
| 265 | HHHHHHTKRRITPKDVIDVRSVTTEINT |
| 266 | KLWMRWYSPTTRRYG |
| 267 | DSLKSYWYLQKFSWR |
| 268 | RTLVNEYKNTLKFSK |
| 269 | IPSRWKDQFWKRWHY |
| 270 | GYGNCRHFKQKPRRD |
| 271 | KNAWKHSSCHHRHQI |
| 272 | RVREWWYTITLKQES |
| 273 | QQHLLIAINGYPRYN |
| 274 | WKCRRQCFRVLHHWN |
| 275 | RLWMRWYSPTTRRYG |
| 276 | KLWMRWYSATTRRYG |
| 277 | KLWMRWYSPWTRRYG |
| 278 | RLWMRWYSPWTRRYG |
| 279 | RLWMRWYSPWTRRWG |
| 280 | ALWMRWYSPTTRRYG |
| 281 | RAWMRWYSPTTRRYG |
| 282 | RLWMRWYSPTTRRYG |
| 283 | RLWARWYSPTTRRYG |
| 284 | RLWMAWYSPTIRRYG |
| 285 | RLWMRWYAPTTRRYG |
| 286 | RLWMRWYAPTTRRYG |
| 287 | RLWMRWYAPTTRRYG |
| 288 | RLWMRWYSPATRRYG |
| 289 | RLWMRWYSPTARRYG |
| 290 | RLWMRWYSPTARRYG |
| 291 | RLWMRWYSPTTRAYG |
| 292 | RLWMRWYSPTTRRAG |
| 293 | RLWMRWYSPTTRRYA |
| 294 | RLIMRIYSPTTRRYG |
| 295 | RLIMRIYSPTTRRYG |
| 296 | RLIMRIYSPTTRRYG |
| 297 | RLVMRVYSPTTRRYG |
| 298 | RLYMRYYSPTTRRYG |
| 299 | YGRKKKRRQRRR |
| 300 | ALIILRRRIRKQAHAHSK |
| 301 | LAIILRRRIRKQAHAHSK |
| 302 | LLAILRRRIRKQAHAHSK |
| 303 | LLIALRRRIRKQAHAHSK |
| 304 | LLIIARRRIRKQAHAHSK |
| 305 | LLIILARRIRKQAHAHSK |
| 306 | LLIILRARIRKQAHAHSK |
| 307 | LLIILRRAIRKQAHAHSK |
| 308 | LLIILRRRARKQAHAHSK |
| 309 | LLIILRRRIARKQAHAHSK |
| 310 | LLIILRRRIRAQAHAHSK |
| 311 | LLIILRRRIRKAAHAHSK |
| 312 | LLIILRRRIRKQaHAHSK |
| 313 | LLIILRRRIRKQAAAHSK |
| 314 | LLIILRRRIRKQAHaHSK |
| 315 | LLIILRRRIRKQAHAASK |
| 316 | LLIILRRRIRKQAHAHAK |
| 317 | LLIILRRRIRKQAHAHSA |
| 318 | KSHAHAQKRIRRRLIILL |
| 319 | lliilrrrirkqahahsk |
| 320 | RRIRPRP |
| 321 | RRIRPRPPRLPRPRP |
| 322 | RRIRPRPPRLPRPRPRPLPFPRPG |
| 323 | RRIRPRPPRLPRPRPRP |
| 324 | PRPPRLPRPRPRPLPFPRPG |
| 325 | PPRLPRPRPRPLPFPRPG |
| 326 | RLPRPRPRPLPFPRPG |
| 327 | PRPRPRPLPFPRPG |
| 328 | PRPRPLPFPRPG |
| 329 | PRPLPFPRPG |
| 330 | RVTSWLGRQLRIAGKRLEGRSK |
| 331 | GRQLRIAGKRLEGRSK |
| 332 | RRVTSWLGRQLRIAGKRLEGRSK |
| 333 | RVRSWLGRQLRIAGKRLEGRSK |
| 334 | GRQLRIAGKRLRGRSK |
| 335 | GRQLRIAGRRLRGRSR |
| 336 | GRQLRRAGRRLRGRSR |
| 337 | GRQLRIAGRRLRRRSR |
| 338 | GRQLRRAGRRLRRRSR |
| 339 | RQLRIAGRRLRGRSR |
| 340 | rsrgrlrrgairlqrg |
| 341 | KLIKGRTPIKFGKADCDRPPKHSQNGMGK |
| 342 | KLIKGRTPIKFGKADCDRPPKHSQNGM |
| 343 | KLIKGRTPIKFGKADCDRPPKHSQNGK |
| 344 | KGRTPIKFGKADCDRPPKHSQNGMGK |
| 345 | KLIKGRTPIKFGKADCDRPPKHSGK |
| 346 | KLIKGRTPIKFGKARCRRPPKHSGK |
| 347 | KLIKGRTPIKFGK |
| 348 | KRIPNKKPGKKTTTKPTKKPTIKTTKKDLKPQTTKPK |
| 349 | KRIPNKKPGKKTTTKPTKKPTIKTTKKDLK |
| 350 | KRTPNKKPGKKTTTKPTKKPTIKTTKK |
| 351 | KRIPNKKPGKKTTTKPTKKPTIK |
| 352 | KRIPNKKPGKKTTTKPTKK |
| 353 | KRIPNKKPGKKT |
| 354 | KRIPNKKPGKK |
| 355 | KRIPNKKPKK |
| 356 | KKPGKKTTTKPTKKPTIKTTKK |
| 357 | KKPGKKTTTKPTKK |
| 358 | KKPTIKTTKK |
| 359 | KKTTTKPTKK |
| 360 | KSICKTIPSNKPKKK |
| 361 | KTIPSNKPKKK |
| 362 | KPRSKNPPKKPK |
| 363 | DRDDRDDRDDRDDRDDR |
| 364 | ERERERERERERER |
| 365 | WRWRWRWRWRWRWR |
| 366 | DRDRDRDRDR |
| 367 | GALFLGFLGAAGSTMGAWSQPKKKRKV |
| 368 | DRRRRGSRPSGAERRRR |
| 369 | NRARRNRRRVR |
| 370 | RTRRNRRRVR |
| 371 | RNRSRHRR |
| 372 | MVRRFLVTLRIRRACGPPRVRV |
| 373 | FVTRGCPRRLVARLIRVMVPRR |
| 374 | VRRFLVTLRIRRA |
| 375 | RVRILARFLRTRV |
| 376 | RVRVIEVVHIPRLT |
| 377 | VIRVHFRLPVRTV |
| 378 | MVRRELVTLRIRRACGPPRVRVFVVHIPRLTGEWAAP |
| 379 | FRVPLRIRPCVVAPRLVMVRHTFGRIARWVAGPLETR |
| 380 | AGYLLGKINLKALAALAKKIL |
| 381 | GTKMIFVGIKKKEERADLIAYLKKA |
| 382 | KKKEERADLIAYLKKA |
| 383 | KMIFVGIKKKEERA |
| 384 | KMIFVGIKKK |
| 385 | EKGKKIFIMK |
| 386 | KGKKIFIMK |
| 387 | RRRRNRTRRNRRRVRGC |
| 388 | TRRQRTRRARRNRGC |
| 389 | KMTRAQRRAAARRNRWTARGC |
| 390 | KLTRAQRRAAARKNKRNTRGC |
| 391 | NAKTRRHERRRKLAIERGC |
| 392 | MDAQTRRRERRAEKQAQWKAANGC |
| 393 | TAKTRYKARRAELIAERRGC |
| 394 | SQMTRQARRLYBGC |
| 395 | KRRIRRERNKMAAAKSRNRRRELTDTGC |
| 396 | RIKAERKRMRNRIAASKSRKRKLERIARGC |
| 397 | KRARNTEAARRSRARKLQRMKQGC |
| 398 | KCFQWQRNMRKVRGPPVSCIKR |
| 399 | KCFQWQRNMRKVRGPPVSC |
| 400 | KCFQWQRNMRKVRGPPVSSIKR |
| 401 | KCFQWQRNMRKVR |
| 402 | FQWQRNMRKVRGPPVS |
| 403 | QWQRNMRKVRGPPVSCIKR |
| 404 | QWQRNMRKVR |
| 405 | KCFMWQEMLNKAGVPKLRCARK |
| 406 | KWFETWFTEWPKKRK |
| 407 | GLWRALWRLLRSLWRLLWRA |
| 408 | GLWWRLWWRLRSWFRLWFRA |
| 409 | DAATATRGRSAASRPTQRPRAPARSASRPRRPVE |
| 410 | GALFLGFLGAAGSTMGAWSQPKKKRKV |
| 411 | GALFLGFLGAAGSTMGAWSQPKSKRKV |
| 412 | AKVKDEPQRRSARLSAKPAPPKPEPKPKKAPAKK |
| 413 | akykdepgrrsarlsakpappkpepkpkkapakk |
| 414 | PSSSSSSRIGDP |
| 415 | vrlpppyripppvrlppp |
| 416 | VELPPPVELPPPVELPPP |
| 417 | ALWMTLLKKVLKAAAKAALNAVLVGANA |
| 418 | ALWKTLLKKVLKA |
| 419 | ALWKTLLKKVLKAPKKKRKV |
| 420 | PKKKRKVALWKTLLKKVLKA |
| 421 | VKRKKKPALWKTLLKKVLKA |
| 422 | RQARRNRRRALWKTLLKKVLKA |
| 423 | RQARRNRRRC |
| 424 | EEEAAGRKRKKRT |
| 425 | EEE |
| 426 | EEEAA |
| 427 | EEEAAKKK |
| 428 | FFFAAGRKRKKRT |
| 429 | AAGRKRKKRT |
| 430 | YYYAAGRKRKKRT |
| 431 | MVTVLFRRLRIRRACGPPRVRV |
| 432 | AGYLLGKINLKALAALAKKIL |
| 433 | GKKKKRKREKL |
| 434 | ERKKRRRE |
| 435 | FKKFRKF |
| 436 | YTQDFNKFHTFPQTAIGVGAP |
| 437 | DFNKFHTFPQTAIGVGAP |
| 438 | KFHTFPQTAIGVGAP |
| 439 | TFPQTAIGVGAP |
| 440 | GYGRKKRRQRRRG |
| 441 | FLGKKFKKYFLQLLK |
| 442 | FLIFIRVICIVIAKLKANLMCKT |
| 443 | KKAAQIRSQVMTHLRVI |
| 444 | YIVLRRRRKRVNTKRS |
| 445 | RRKLSQQKEKK |
| 446 | VQAILRRNWNQYKIQ |
| 447 | KTVLLRKLLKLLVRKI |
| 448 | LLKKRKVVRLIKFLLK |
| 449 | KLPCRSNTFLNIFRRKKPG |
| 450 | KKICTRKPRFMSAWAQ |
| 451 | rggrlsysrrrfststgr |
| 452 | rr1sysrrrf |
| 453 | RGGRLAYLRRRWAVLGR |
| 454 | MANLGCWMLVLEVATWSDLGLCKKRPKP |
| 455 | MVKSKIGSWILVLFVAMWSDVGLCKKRPKP |
| 456 | LLGDFFRKSKEKIGKEFKRIVQRIKDFLRNLVPRTESC |
| 457 | GIGKFLHSAKKWGKAFVGQIMNC |
| 458 | TRSSRAGLQWPVGRVHRLLRKGGC |
| 459 | YGRKKRRQRRR |
| 460 | EKRPRTAFSSEQLARLKREFNENRYLTTERRRQQLSSELGLNEA |
| | QIKIWFQNKRAKIKKST |
| 461 | GRRRRRRRRRPPQ |
| 462 | GALFLGFLGAAGSTMGAWSQPKKKRKV |
| 463 | GALFLAFLAAALSLMGLWSQPKKKRRV |
| 464 | MLLLTRRRST |
| 465 | CGNKRTRGC |
| 466 | TSPLNIHNGQKL |
| 467 | GLRKRLRKFRNKIKEK |
| 468 | GLLEALAELLEGLRKRLRKFRNKIKEK |
| 469 | CVQWSLLRGYQPC |
| 470 | RQIKIFFQNRRMKWKK |
| 471 | ASMWERVKSIIKSSLAAASNI |
| 472 | ASMWERVKSIIKSSLAAASNI |
| 473 | DPKGDPKGVTVTVTVIVTGKGDPKPD |
| 474 | CSIPPEVKFNPFVYLI |
| 475 | csippevkfnpfvyli |
| 476 | PFVYLI |
| 477 | NKPILVFY |
| 478 | YKQCHKKGGKKGSG |
| 479 | YKQCHKKGGXKKGSG |
| 480 | GSGKKGGKKHCQKY |
| 481 | GSGKKGGKKICQKY |
| 482 | YTAIAWVKAFIRKLRK |
| 483 | IAWVKAFIRKLRKGPLG |
| 484 | LIRLWSHLIHIWFQNRRLKWKKK |
| 485 | KKKKKKGGFLGEWRGENGRKTRSAYERMCILKGK |
| 486 | RLSGMNEVLSFRWL |
| 487 | GPFHEYQFLEPPV |
| 488 | GSPWGLQHHPPRT |
| 489 | AAVALLPAVLLALLAPEILLPNNYNAYESYKYPGMFIALSK |
| 490 | AAVALLPAVLLALLAPVQRKRQKLMP |
| 491 | MGLGLHLLVLAAALQGAKKKRKV |
| 492 | WEAALAEALAEALAEHLAEALAEALEALAA |
| 493 | GLFEALLELLESLWELLLEA |
| 494 | GLFKALLKLLKSLWKLLLKA |
| 495 | GLFRALLRLLRSLWRLLLRA |
| 496 | CGAYDLRRRERQSRLRRRERQSR |
| 497 | RKKRRRESRKKRRRESC |
| 498 | CVKRGLKLRHVRPRVTRDV |
| 499 | CRQIKIWFQNRRMKWKK |
| 500 | PPKKSAQCLRYKKPE |
| 501 | DPVDTPNPTRRKPGK |
| 502 | KRVSRNKSEKKRR |
| 503 | GRRHHCRSKAKRSRHH |
| 504 | SARHHCRSKAKRSRHH |
| 505 | SRAHHCRSKAKRSRHH |
| 506 | SRRAHCRSKAKRSRHH |
| 507 | SRRHACRSKAKRSRHH |
| 508 | SRRHHARSKAKRSRHH |
| 509 | SRRHHCRAKAKRSRHH |
| 510 | SRRHHCRSAAKRSRHH |
| 511 | SRRHHCRSKAARSRRH |
| 512 | SRRHHCRSKAKASRHH |
| 513 | SRRHHCRSKAKRARHH |
| 514 | SRRHHCRSKAKRSAHH |
| 515 | RRHHCRSKAKRSR |
| 516 | GRKGKHKRKKLP |
| 517 | GKKKKKKKKK |
| 518 | GKRVAKRKLIEQNRERRR |
| 519 | GRKLKKKKNEKEDKRPRT |
| 520 | GKKTNLFSALIKKKKTA |
| 521 | GRRERNKMAAAKCRNRRR |
| 522 | GKRARNTEAARRSRARKL |
| 523 | GRRRRATAKYRTAH |
| 524 | GKRRRRATAKYRSAH |
| 525 | GRRRRKRLSHRT |
| 526 | GRRRRRERNK |
| 527 | GKHRHERGHHRDRRER |
| 528 | GKKKRKLSNRESAKRSR |
| 529 | MIIYRDLISH |
| 530 | MIIYRDLIS |
| 531 | MIIYRDLI |
| 532 | IIYRDLISH |
| 533 | MIIYRDL |
| 534 | MIIYRD |
| 535 | IYRDLISH |
| 536 | AIIYRDLIS |
| 537 | MAIYRDLIS |
| 538 | MIAYRDLIS |
| 539 | MIIARDLIS |
| 540 | MIIYADLIS |
| 541 | MIIYRDLIS |
| 542 | MIIYRDAIS |
| 543 | MIIYRDLAS |
| 544 | MIIYRDLIA |
| 545 | MIIYRDLISKK |
| 546 | MITYRDKKSH |
| 547 | MIIFRDLISH |
| 548 | MIISRDLISH |
| 549 | QIISRDLISH |
| 550 | CIISRDLISH |
| 551 | MIIYRALISHKK |
| 552 | MIIYRIAASHKK |
| 553 | MIIRRDLISE |
| 554 | MIIYRAEISH |
| 555 | MIIYARRAEE |
| 556 | MIIFRIAASHKK |
| 557 | MIIFRALISHKK |
| 558 | MIIFRAAASHKK |
| 559 | FIIFRIAASHKK |
| 560 | LIIFRIAASHKK |
| 561 | WIIFRIAASHKK |
| 562 | WIIFRAAASHKK |
| 563 | WIIFRALISHKK |
| 564 | MIIFRIAAYHKK |
| 565 | WIIFRIAAYHKK |
| 566 | MIIFRIAATHKK |
| 567 | WIIFRIAATHKK |
| 568 | MIIFKIAASHKK |
| 569 | WIIFKIAASHKK |
| 570 | MIIFRIAASHKK |
| 571 | LIIFRILISHKK |
| 572 | MIIFRILISHKK |
| 573 | LIIFRILISHRR |
| 574 | LIIFRILISHHH |
| 575 | LIIFRILISHK |
| 576 | LIIFRILISHR |
| 577 | LIIFRILISH |
| 578 | LIIFAIAASHKK |
| 579 | LIIFAILISHKK |
| 580 | RILQQLLFIHFRIGCRHSRI |
| 581 | RILQQLLFIHFRIGCRH |
| 582 | RILQQLLFIHFRIGC |
| 583 | RIFIHFRIGC |
| 584 | RIFIRIGC |
| 585 | RILQQLLFTHF |
| 586 | RIFIGC |
| 587 | FIRIGC |
| 588 | DTWAGVEATIRILQQLLFTHFR |
| 589 | ICCRH |
| 590 | GYGRKKRRGRRRTHRLPRRRRRR |
| 591 | KRIIQRILSRNS |
| 592 | KRIHPRLTRSIR |
| 593 | PPRLRKRRQLNM |
| 594 | MHKRPTTPSRKM |
| 595 | RQRSRRRPLNIR |
| 596 | RIRMIQNLIKKT |
| 597 | SRRKRQRSNMRI |
| 598 | QRIRKSKISRTL |
| 599 | PSKRLLHNNLRR |
| 600 | HRHIRRQSLIML |
| 601 | PQNRLQIRRHSK |
| 602 | PPHNRIQRRLNM |
| 603 | SMLKRNHSTSNR |
| 604 | GSRHPSLIIPRQ |
| 605 | SPMQKTMNLPPM |
| 606 | NKRILIRIMTRP |
| 607 | HGWZIHGLLHRA |
| 608 | AVPAKKRZKSV |
| 609 | PNTRVRPDVSF |
| 610 | LTRNYEAWVPTP |
| 611 | SAETVESCLAKSH |
| 612 | YSHIATLPFTPT |
| 613 | SYIQRTPSTTLP |
| 614 | AVPAENALNNPF |
| 615 | SFHQFARATLAS |
| 616 | QSPTDFTFPNPL |
| 617 | HFAAWGGWSLVH |
| 618 | HIQLSPFSQSWR |
| 619 | LTMPSDLQPVLW |
| 620 | FQPYDHPAEVSY |
| 621 | FDPFFWKYSPRD |
| 622 | FAPWDTASFMLG |
| 623 | FTYKNFFWLPEL |
| 624 | SATGAPWKMWVR |
| 625 | SLGWMLPFSPPF |
| 626 | SHAFTWPTYLQL |
| 627 | SHNWLPLWPLRP |
| 628 | SWLPYPWHVPSS |
| 629 | SWWTPWHVHSES |
| 630 | SWAQHLSLPPVL |
| 631 | SSSIFPPWLSFF |
| 632 | LNVPPSWFLSQR |
| 633 | LDITPFLSLTLP |
| 634 | LPHPVLHMGPLR |
| 635 | VSKQPYYMWNGN |
| 636 | NYTTYKSHFQDR |
| 637 | AIPNNQLGFPFK |
| 638 | NIENSTLATPLS |
| 639 | YPYDANHTRSPT |
| 640 | DPATNPGPHFPR |
| 641 | TLPSPLALLTVH |
| 642 | HPGSPFPPEHRP |
| 643 | TSHTDAPPARSP |
| 644 | MTPSSLSTLPWP |
| 645 | VLGQSGYLMPMR |
| 646 | QPIIITSPYLPS |
| 647 | TPKTMTQTYDFS |
| 648 | NSGTMQSASRAT |
| 649 | QAASRVENYMHR |
| 650 | HQHKPPPLTNNW |
| 651 | SNPWDSLLSVST |
| 652 | KTIEAHPPYYAS |
| 653 | EPDNWSLDFPRR |
| 654 | HQHKPPPLTNNW |
| 655 | GLWRALWRLLRSLWRLLWKA |
| 656 | GLWRALWRALWRSLWKLKRKV |
| 657 | GLWRALWRALRSLWKLKRKV |
| 658 | GLWRALWRGLRSLWKLKRKV |
| 659 | GLWRALWRGLRSLWKKKRKV |
| 660 | GLWRALWRALWRSLWKLKWKV |
| 661 | GLWRALWRALWRSLWKSKRKV |
| 662 | GLWRALWRALWRSLWKKKRKV |
| 663 | GLWRALWRALWRSLWKLKRKV |
| 664 | GLWRALWRLLRSLWRLLWSQPKKKRKV |
| 665 | YARAARRAARR |
| 666 | PARAARRAARR |
| 667 | YPRAARRAARR |
| 668 | YRRAARRAARA |
| 669 | YGRRARRAARR |
| 670 | YAREARRAARR |
| 671 | YGRRARRAARR |
| 672 | YKRAARRAARR |
| 673 | YARKARRAARR |
| 674 | YKRKARRAARR |
| 675 | YGRRARRAARR |
| 676 | YGRRARRRARR |
| 677 | YGRRRRRRRRR |
| 678 | YRRRRRRRRRR |
| 679 | GKINLKALAALAKKIL |
| 680 | GRKKRRQRRRPPQGRKKRRQRRRPPQGRKKRRQRRRPPQ |
| 681 | GEQIAQLIAGYIDIILKKKKSK |
| 682 | AAVALLPAVLLALLAPRKKRRQRRRPPQ |
| 683 | AAVALLPAVLLALLAPRKKRRQRRRPPQC |
| 684 | RKKRRQRRRPPQCAAVALLPAVLLALLAP |
| 685 | RRRQRRKRGGDIMGEWGNEIFGAIAGFLG |
| 686 | RRRQRRKRGGDIMGEWGNEIFGAIAGFLG |
| 687 | YGRKKRRQRRRGCYGRKKRRQRRRG |
| 688 | AAVALLPAVLLALLAPRRRRRR |
| 689 | RLWRALPRVLRRLLRP |
| 690 | AAVALLPAVLLALLAPSGASGLDKRDYV |
| 691 | LLETLLKPFQCRICMRNFSTRQARRNHRRRHRR |
| 692 | AAVACRICMRNFSTRQARRNHRRRHRR |
| 693 | RQIKIWFQNRRMKWKKDIMGEWGNEIFGAIAGFLG |
| 694 | SGRGKQGGKARAKAKTRSSRAGLQFPVGRVHRLLRKG |
| 695 | SGRGKQGGKARAKAKTRSSRAGLQFPVGRVHRLLRKGC |
| 696 | KKDGKKRKRSRKESYSVYVYKVLKQ |
| 697 | KGSKKAVTKAQKKDGKKRKRSRKESYSVYVYKVLKQ |
| 698 | KETWWETWWTEWSQPGRKKRRQRRRPPQ |
| 699 | RVIRWFQNKRCKDKK |
| 700 | LGLLLRHLRFIHSNLLANI |
| 701 | KLWSAWPSLWSSLWKP |
| 702 | GLGSLLKKAGKKLKQPKSKRKV |
| 703 | FKQqQqQqQqQq |
| 704 | YRFK |
| 705 | YRFKYRFKYRLFK |
| 706 | WRFKKSKRKV |
| 707 | WRFKAAVALLPAVLLALLAP |
| 708 | WRFKWRFK |
| 709 | WRFKWRFKWRFK |
| 710 | KGSKKAVTKAQKKDGKKRKRSRKESYSVYVYKVLKQ |
| 711 | RGSRRAVTRAQRRDGRRRRRSRRESYSVYVYRVLRQ |
| 712 | RVIRWFQNKRSKDKK |
| 713 | AAVALLPAVLLALLAPRKKRRQRRRPPQ |
| 714 | CWKKK |
| 715 | CWKKKKKKKK |
| 716 | CWKKKKKKKKKKKKK |
| 717 | CWKKKKKKKKKKKKKKKKKK |
| 718 | KKKKKKKKKKKKKKKKKKK |
| 719 | kkwkmrrGaGrrrrrrrrr |
| 720 | APWHLSSQYSRT |
| 721 | AAVALLPAVLLALLAKKNNLKDCGLF |
| 722 | AAVALLPAVLLALLAKKNNLKECGLY |
| 723 | AHALCLTERQIKIWFQNRRMKWKKEN |
| 724 | AHALCPPERQIKIWFQNRRMKWKKEN |
| 725 | AYALCLTERQIKIWFANRRMKWKKEN |
| 726 | GGVCPKILKKGRRDSDCPGACICRGNGYCGSGSD |
| 727 | GGVCPKILAACRRDSDCPGACICRGNGYCGSGSD |
| 728 | GGVCPAILKKCRRDSDCPGACICRGNGYCGSGSD |
| 729 | GGVCPKILAKCRRDSDCPGACICRGNGYCGSGSD |
| 730 | GGVCPKILKACRRDSDCPGACICRGNGYCGSGSD |
| 731 | GLPVCGETCVGGICNIPGCKCSWPVCIRN |
| 732 | GLPVCGETCVGGTCNTPGCTCSWPKCTRN |
| 733 | GRCTKSIPPICFPD |
| 734 | RQIKIWFQNRRMKWKKTYADFIASGRTGRRNAI |
| 735 | GRKKRRQRRRPPQTYADFIASGRTGRRNAI |
| 736 | AGYLLGKINLKALAALAKKIL |
| 737 | AGYLLGKINLKALAALAKKILTYADFIASGRTGRRNAI |
| 738 | RRRRRRRRRRR |
| 739 | RRRRRRRRRRRTYADFIASGRTGRRNAI |
| 740 | rrrrrrrrrk |
| 741 | rRRRRRRRr |
| 742 | rRrRrRrRr |
| 743 | KCFQWQRNMRKVRGPPVSCIKR |
| 744 | kcfqwqrnmrkyrqppyscikr |
| 745 | KLALKLALKALKAALKLAGC |
| 746 | KLULKLULKULKAULKLUGC Wherein U is Aib (α- |
| | aminoisobutyricacid) |
| 747 | GGGARKKAAKAARKKAAKAARKKAAKAARKKAAKA |
| 748 | GRKKRRQRRRPPQC |
| 749 | TRQARRNRRRRWRERQRGC |
| 750 | RRRRNRTRRNRRRVRGC |
| 751 | KMTRAQRRAAARRNRWTARGC |
| 752 | TRRQRTRRARRNRGC |
| 753 | RIKAERKRMRNRIAASKSRKRKLERIARGC |
| 754 | KRRIRRERNKMAAAKSRNRRRELTDTGC |
| 755 | WLRRIKAWLRRIKALNRQLGVAA |
| 756 | crkkrrqrrr |
| 757 | crrrrrrrrr |
| 758 | ckkkkkkkkk |
| 759 | GRKKRRQRRRPP |
| 760 | rrrrrrrr |

L-amino acids are in upper case,
D-amino acids in lower case.
Repetitions are written in parenthesis.

A cell penetrating peptide described herein can comprise one or more sequences described in Table 1. In some cases, a cell penetrating peptide can have an amino acid composition that can contain a high relative abundance of positively charged amino acids, such as lysine or arginine. In other cases, a cell penetrating peptide can have sequences that contain an alternating pattern of polar/charged amino acids and non-polar, hydrophobic amino acids. A cell penetrating peptide can be polycationic or cationic. A cell penetrating peptide can be amphipathic. A cell penetrating peptide may have an ability to translocate the plasma membrane and facilitate the delivery of various molecular cargoes to the cytoplasm or an organelle of a target cell. A cell penetrating peptide can directly penetrate a cellular membrane. A cell penetrating peptide can use endocytosis-mediated entry into a cell. In some cases, a cell penetrating peptide can use translocation through the formation of a transitory structure. A cell-penetrating peptide can have an amino acid sequence having from about 5 to about 10 amino acids, from about 10 amino acids to about 20 amino acids, from about 20 amino acids to about 30 amino acids, from about 30 amino acids to about 40 amino acids, from about 40 amino acids to about 60 amino acids. In some cases, a cell penetrating peptide can have from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or up to about 99 amino acids or greater. Preferably, a cell penetrating peptide can comprise natural amino acids, amino acid derivatives, D-amino acids, modified amino acids, β-amino acid derivatives, α,α-disubstituted amino acid derivatives, N-substituted a-amino acid derivatives, aliphatic or cyclic amines, amino- and carboxy-substituted cycloalkyl derivatives, amino- and carboxy-substituted aromatic derivatives, γ-amino acid derivatives, aliphatic a-amino acid derivatives, diamines and polyamines. Further modified amino acids are known to the skilled artisan

In some cases, a cell penetrating peptide can have in its respective primary amino acid sequence at least 25%, at least 30% positively charged amino acid residues. The term “positively charged amino acids” as used herein, can denote the entirety of lysine (K), histidine (H), and arginine (R) residue present in a particular peptide. In certain cases, a peptide used can comprise in its amino acid sequence from about 25%, 26%, 27%, 28%, 29%, 30%, 31%, 32%, 33%, 34% or up to about 35% positively charged amino acid residues. In other cases, peptides used herein can comprise in amino acid sequences at least about 35%, at least 40%, at least 45%, at least 50%, at least 55%, or at least 60% positively charged amino acid residues.

Polynucleic Acid Cargo

Disclosed herein can be a liposome carrying a polynucleic acid. A polynucleic acid can be a vector. Polynucleic acid can be DNA- or RNA-based. DNA-based vectors can be non-viral, and include molecules such as plasmids, minicircles, closed linear DNA (doggybone), linear DNA, and single-stranded DNA. A nucleic acid that can be present in a lipid-nucleic acid particle includes any form of nucleic acid that is known. The nucleic acids used herein can be single-stranded DNA or RNA, or double-stranded DNA or RNA, or DNA-RNA hybrids. Examples of double-stranded DNA include structural genes, genes including control and termination regions, and self-replicating systems such as viral or plasmid DNA. Examples of double-stranded RNA include siRNA and other RNA interference reagents. Single-stranded nucleic acids include antisense oligonucleotides, ribozymes, microRNA, and triplex-forming oligonucleotides. The nucleic acid that is present in a lipid-nucleic acid particle may include one or more of the oligonucleotide modifications described below. Nucleic acids may be of various lengths, generally dependent upon the particular form of nucleic acid. For example, in particular embodiments, plasmids or genes may be from about 1,000 to 100,000 nucleotide residues in length. In particular embodiments, oligonucleotides may range from about 10 to 100 nucleotides in length. In various related embodiments, oligonucleotides, single-stranded, double-stranded, and triple-stranded, may range in length from about 10 to about 50 nucleotides, from about 20 to about 50 nucleotides, from about 15 to about 30 nucleotides, from about 20 to about 30 nucleotides in length. In particular embodiments, oligonucleotides may range from about 2 nucleotides to 10 nucleotides in length.

DNA-based vectors can also be viral, and include adeno-associated virus, lentivirus, adenovirus, and others. Vectors can also be RNA. RNA vectors can be linear or circular forms of unmodified RNA. They can also include various nucleotide modifications designed to increase half-life, decrease immunogenicity, and/or increase level of translation. A vector as used herein can be composed of either DNA or RNA. In some embodiments, a vector can be composed of DNA. Vectors can be capable of autonomous replication in a prokaryote such as E. coli, used for growth. In some embodiments a vector may be stably integrated into a genome of an organism. In other cases, a vector can remain separate, either in a cytoplasm or a nucleus. In some embodiments, a vector can contain a targeting sequence. In some embodiments, a vector can contain an antibiotic resistance gene. A vector can contain regulatory elements for regulating gene expression. In some cases, a mini-circle can be enclosed within a liposome.

In certain embodiments, described herein can be methods and compositions for the delivery of a liposomal structure. A liposomal structure can contain a therapeutic polynucleic acid. A polynucleic acid can be a gene, high molecular weight DNA, plasmid DNA, an antisense oligonucleotide, peptides, peptidomimetics, ribozymes, peptide nucleic acids, a chemical agent such as a chemotherapeutic molecule, or any large molecule including, but not limited to, DNA, RNA, viral particles, growth factors cytokines, immunomodulating agents and other proteins, including proteins which when expressed present an antigen which stimulates or suppresses the immune system. In some cases, a portion of a gene can be expressed by a polynucleic acid. A portion of a gene can be from three nucleotides up to the entire whole genomic sequence. For example, a portion of a gene can be from about 1% up to about 100% of an endogenous genomic sequence. A portion of a gene can be from about 1%, 10%, 20%, 30%, 40%, 50%, 60%, 70%, 80%, 90%, or up to about 100% of a whole genomic sequence of a gene.

Transgene expression duration from plasmid vectors can be reduced due to promoter inactivation mediated by the bacterial region (i.e. region encoding bacterial replication origin and selectable marker which may be encoded in the spacer region) of the vector. This can result in short duration of transgene expression. A strategy to improve transgene expression duration may involve removal of bacterial regions of a plasmid. For example, minicircle and ‘linear Minimalistic immunogenic defined gene expression’ (MIDGE) vectors have been developed which do not contain a bacterial region. Removal of the bacterial region in minicircle vectors improved transgene expression duration. In minicircle vectors, the eukaryotic region polyadenylation signal can be covalently linked to the eukaryotic region promoter. This linkage (spacer region) can tolerate a spacer sequence of at least 500 bp since in vivo expression duration can be improved with plasmid vectors in which the bacterial region can be removed or replaced with a spacer sequence (spacer region) up to 500 bp in length. In some cases, a polynucleic acid can be a minicircle vector, Table 3.

Minicircle (MC) DNA can be similar to plasmid DNA as both may contain expression cassettes that may permit transgene products to be made at high levels shortly after delivery. In some cases, a MC can differ in that MC DNA can be devoid of prokaryotic sequence elements (e.g., bacterial origin of replication and antibiotic-resistance genes). Removal of prokaryotic sequence elements from a backbone plasmid DNA can be achieved via site-specific recombination in Escherichia coli before episomal DNA isolation. The lack of prokaryotic sequence elements may reduce MC size relative to its parental full-length (FL) plasmid DNA, which may lead to enhanced transfection efficiencies. The result may be that when compared with their FL plasmid DNA counterparts, MCs can transfect more cells and may permit sustained high level transgene expression upon delivery.

In some cases, a minicircle DNA can be free of a bacterial origin of replication. For example, a minicircle DNA or closed linear DNA, can be free of a bacterial origin of replication from about 50% of a bacterial origin of replication sequence or up to 100% of a bacterial origin of replication. In some cases a bacterial origin of replication is truncated or inactive. A polynucleic acid can be derived from a vector that initially encoded a bacterial origin of replication. A method can be utilized to remove the entirety of a bacterial origin of replication or a portion thereof, leaving a polynucleic acid free of a bacterial origin of replication. In some cases, a bacterial origin of replication can be identified by its high adenine and thymine content.

Minicircle DNA vectors can be supercoiled minimal expression cassettes, derived from conventional plasmid DNA by site-specific recombination in vivo in Escherichia coli for the use in non-viral gene therapy and vaccination. Minicircle DNA may lack or have reduced bacterial backbone sequences such as an antibiotic resistance gene, an origin of replication, and/or inflammatory sequences intrinsic to bacterial DNA. In addition to their improved safety profile, minicircles can greatly increase efficiency of transgene expression.

A liposome can carry to a capacity up to over 100% weight: defined as (cargo weight/weight of liposome)×100. The optimal loading of cargo can be or can be from about 1% to 100% weight of a liposome structure. For example, a liposomal structure can contain a polynucleic acid cargo from about 1% weight of a structure to about 10%, from about 10% to about 20%, from about 20% to about 30%, from about 30% to about 40%, from about 40% to about 50%, from about 50%, to about 60%, from about 60% to about 70%, from about 70% to about 80%, from about 80% to about 90%, from about 90% to about 100%, from about 100% to about 200%, from about 200% to about 300%, from about 300% to about 400%, from about 400% to about 500% or greater weight of a structure.

Cargo can include, for example, small molecule drugs, peptides, proteins, antibodies, DNA (minicircle DNA for example), double stranded DNA, single stranded DNA, double stranded RNA, single stranded RNA, RNAs (including shRNA and siRNA (which may also be expressed by the plasmid DNA incorporated as cargo within a liposome) fluorescent dyes, including fluorescent dye peptides which may be expressed by a DNA incorporated within a liposome, or any combination thereof.

In some cases a polynucleic acid can encode for a heterologous sequence. A heterologous sequence can provide for subcellular localization (e.g., a nuclear localization signal (NLS) for targeting to a nucleus; a mitochondrial localization signal for targeting to a mitochondria; a chloroplast localization signal for targeting to a chloroplast; an ER retention signal; and the like). In some case, a polynucleic acid, such as minicircle DNA or closed linear DNA, can comprise a nuclear localization sequence (NLS).

In some embodiments, a vector encodes a protein such as APC. A vector can comprise one or more nuclear localization sequences (NLSs). A number of NLS sequences can be from about 1, 2, 3, 4, 5, 6, 7, 8, 9, 10, or more NLSs. In some embodiments, a vector comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies.

Non-limiting examples of NLSs can include an NLS sequence derived from: the NLS of the SV40 virus large T-antigen, having the amino acid sequence PKKKRKV; the NLS from nucleoplasmin (e.g. the nucleoplasmin bipartite NLS with the sequence KRPAATKKAGQAKKKK); the c-myc NLS having the amino acid sequence PAAKRVKLD or RQRRNELKRSP; the hRNPA1 M9 NLS having the sequence NQSSNFGPMKGGNFGGRSSGPYGGGGQYFAKPRNQGGY; the sequence RMRIZFKNKGKDTAELRRRRVEVSVELRKAKKDEQILKRRNV of the IBB domain from importin-alpha; the sequences VSRKRPRP and PPKKARED of the myoma T protein; the sequence POPKKKPL of human p53; the sequence SALIKKKKKMAP of mouse c-abl IV; the sequences DRLRR and PKQKKRK of the influenza virus NS 1; the sequence RKLKKKIKKL of the Hepatitis virus delta antigen; the sequence REKKKFLKRR of the mouse Mxl protein; the sequence KRKGDEVDGVDEVAKKKSKK of the human poly(ADP-ribose) polymerase; and the sequence RKCLQAGMNLEARKTKK of the steroid hormone receptors (human) glucocorticoid. In general, the one or more NLSs can be of sufficient strength to drive accumulation of the minicircle DNA vector or short linear DNA vector in a detectable amount in the nucleus of a eukaryotic cell. A eukaryotic cell can be a human intestinal crypt cell.

Detection of accumulation in the nucleus may be performed by any suitable technique. For example, a detectable marker may be fused to a vector, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. An embodiment herein can exhibit time dependent pH triggered release of a liposome cargo into a target site. An embodiment herein can contain and provide cellular delivery of complex multiple cargoes. An additional cargo can be a small molecule, an antibody, an inhibitor such as a DNAse inhibitor or RNAse inhibitor.

In some cases, a particle may contain a DNAse inhibitor. A DNAse inhibitor may be localized within a particle or on a particle. In other cases, a polynucleic acid encoding for an inhibitor can be enclosed within a particle. In other cases, an inhibitor can be a DNA methyltransferase inhibitor such as DNA methyltransferase inhibitors-2 (DMI-2). DMI-2 can be produced by Streptomyces sp. strain No. 560. A structure of DMI-2 can be 4′″R,6aR,10S,10aS-8-acetyl-6a, 10a-dihydroxy-2-methoxy-12-methyl-10-[4′-[3″-hydroxy-3″,5″-dimethyl-4″ (Z-2′″,4′″-dimethyl-2′″-heptenoyloxy) tetrahydropyran-1″-yloxy]-5′-methylcyclohexan-1′-yloxy]-1,4,6,7,9-pentaoxo-1,4,6,6a,7,8,9,10,10a,11-decahydronaphthacene. Other inhibitors, such as chloroquine, can also be enclosed within a particle or on a particle, such as on a surface of a particle.

Polynucleic acids can be delivered to cells of the intestinal tract. For example, a polynucleic acid can be delivered by a liposome to an intestinal crypt stem cell. For example, a delivered polynucleic acid can be: (1) not normally found in intestinal epithelial stem cells; (2) normally found in intestinal epithelial stem cells, but not expressed at physiological significant levels; (3) normally found in intestinal epithelial stem cells and normally expressed at physiological desired levels in the stem cells or their progeny; (4) any other DNA which can be modified for expression in intestinal epithelial stem cells; and (5) any combination of the above.

A variety of protein and polypeptides can be delivered to an intestinal crypt stem cell, including proteins for treating metabolic disorders and endocrine disorders. Examples of proteins are phenylalanine hydroxylase, insulin, anti-diuretic hormone and growth hormone. Disorders include phenylketonuria, diabetes, organic acidurias, tyrosinemia, urea cycle disorders, familial hypercholesteremia. Genes for any of the proteins or peptides which can correct the defects in phenylketonuria, diabetes, organic acidurias, tyrosinemia, urea cycle disorders, familial hypercholesteremia can be introduced into stem cells such that the protein or peptide products are expressed by the intestinal epithelium. Coagulation factors such as antihemophilic factor (factor 8), Christmas factor (factor 9) and factor 7 can likewise be produced in the intestinal epithelium. Proteins which can be used to treat deficiency of a circulatory protein can also be expressed in the intestinal epithelium. Proteins which can be used to treat deficiency of a circulatory protein can be, for example, albumin for the treatment of an albuminemia, alpha-1-antitrypsin, hormone binding protein. Additionally, the intestinal symptoms of cystic fibrosis can be treated by inserting the gene for the normal cystic fibrosis transmembrane conductance regulator into the stem cells of intestinal epithelium. Abetalipoproteinemia can be treated by the insertion of the apolipoprotein B. Disaccharidase intolerance can be treated by the insertion of sucrase-isomaltose, lactase-phlorizin hydrolase and maltase-glucoamylase. The insertion of the intrinsic factor for the absorption of vitamin B₁₂ or the receptor for the intrinsic factor/cobalamin complex for absorption of vitamin B₁₂, as well as the transporter for bile acids can be inserted into the intestinal epithelium. Further, any drug which can be encoded by nucleic acid can be inserted into the stem cell of the intestinal epithelium to be secreted in localized, high concentrations for the treatment of cancer. In this respect, one skilled in the art will readily recognize that antisense RNA can be encoded into the stem cells after production of antisense it can incorporate into the cancerous cells for the treatment of cancer.

In some cases, a protein that is encoded by a polynucleic acid comprised within a liposomal structure can be measured and quantified. In some cases, modified cells can be isolated and a western blot performed on modified cells to determine a presence and a relative amount of protein production as compared to unmodified cells. In other cases, intracellular staining of a protein utilizing flow cytometry can be performed to determine a presence and a relative amount of protein production. Additional assays can also be performed to determine if a protein, such as APC, is functional. For example, modified cells expressing an APC transgene, can be measured for cytosolic β-catenin expression and compared to unmodified cells. Reduced expression of β-catenin in the cytosol of modified cells as compared to unmodified cells can be indicative of a functional APC transgene. In other cases, a murine model of FAP can be utilized to determine functionality of a transgene encoding an APC protein. For example, mice with FAP can be treated with modified cells, encoding for APC, and a reduction of FAP disease measured versus untreated mice.

In some cases, liposomal cargo may not be limited to polynucleic acids. Disclosed herein can be a nanoparticle having encapsulated therein, dispersed therein, and/or covalently or non-covalently associate with a surface one or more therapeutic agents or drugs. A therapeutic agent or drug can be a small molecule, protein, polysaccharide or saccharide, nucleic acid molecule, lipid, peptidomimetic, or a combination thereof. A liposomal structure can include any molecule or compound capable of exerting a desired effect on a cell, tissue, organ, or subject. Such effects may be biological, physiological, or cosmetic, for example. Molecules or compounds may include e.g., nucleic acids, peptides and polypeptides, including, e.g., antibodies, such as, e.g., polyclonal antibodies, monoclonal antibodies, antibody fragments; humanized antibodies, recombinant antibodies, recombinant human antibodies, and Primatized™ antibodies, cytokines, growth factors, apoptotic factors, differentiation-inducing factors, cell surface receptors and their ligands; hormones; and small molecules, including small organic molecules or compounds. In one embodiment, a molecules or compound can be a therapeutic agent, or a salt or derivative thereof. Therapeutic agent derivatives may be therapeutically active themselves or they may be prodrugs, which become active upon further modification. Thus, in one embodiment, a molecules or compound derivative may retains some or all of the therapeutic activity as compared to the unmodified agent, while in another embodiment, a therapeutic derivative lacks therapeutic activity.

In various embodiments, therapeutic agents include any therapeutically effective agent or drug, such as anti-inflammatory compounds, anti-depressants, stimulants, analgesics, antibiotics, birth control medication, antipyretics, vasodilators, anti-angiogenics, cytovascular agents, signal transduction inhibitors, cardiovascular drugs, e.g., anti-arrhythmic agents, vasoconstrictors, hormones, and steroids. In certain embodiments, a molecule or compound can be an oncology drug, which may also be referred to as an anti-tumor drug, an anti-cancer drug, a tumor drug, an antineoplastic agent, or the like. Examples of oncology drugs that may be used include, but are not limited to, adriamycin, alkeran, allopurinal, altretamine, amifostine, anastrozole, araC, arsenic trioxide, azathioprine, bexarotene, biCNU, bleomycin, busulfan intravenous, busulfan oral, capecitabine (Xeloda), carboplatin, carmustine, CCNU, celecoxib, chlorambucil, cisplatin, cladribine, cyclosporin A, cytarabine, cytosine arabinoside, daunorubicin, cytoxan, daunorubicin, dexamethasone, dexrazoxane, dodetaxel, doxorubicin, doxorubicin, DTIC, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, idarubicin, ifosfamide, imatinib mesylate, interferon, irinotecan (Camptostar, CPT-111), letrozole, leucovorin, leustatin, leuprolide, levamisole, litretinoin, megastrol, melphalan, L-PAM, mesna, methotrexate, methoxsalen, mithramycin, mitomycin, mitoxantrone, nitrogen mustard, paclitaxel, pamidronate, Pegademase, pentostatin, porfimer sodium, prednisone, rituxan, streptozocin, STI-571, tamoxifen, taxotere, temozolamide, teniposide, VM-26, topotecan (Hycarntin), toremifene, tretinoin, ATRA, valrubicin, velban, vinblastine, vincristine, VP 16, and vinorelbine. Other examples of oncology drugs that may be used are ellipticin and ellipticin analogs or derivatives, epothilones, intracellular kinase inhibitors and camptothecins.

In certain embodiments, a liposomal structure can comprise an imaging agent that may be further attached to a detectable label (e.g., the label can be a radioisotope, fluorescent compound, enzyme or enzyme co-factor). The active moiety may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. A liposomal structure including such a moiety may be used as an imaging agent and be administered in an amount effective for diagnostic use in a mammal such as a human. In this manner, the localization and accumulation of the imaging agent can be detected. The localization and accumulation of the imaging agent may be detected by radioscintiography, nuclear magnetic resonance imaging, computed tomography, or positron emission tomography. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically 0.1-100 millicuries per dose of imaging agent, 1-10 millicuries, and 2-5 millicuries can be administered. Thus, compositions useful as imaging agents can comprise a targeting moiety conjugated to a radioactive moiety that can comprise 0.1-100 millicuries, in some embodiments preferably 1-10 millicuries, in some embodiments preferably 2-5 millicuries, in some embodiments more preferably 1-5 millicuries. The means of detection used to detect the label is dependent of the nature of the label used and the nature of the biological sample used, and may also include fluorescence polarization, high performance liquid chromatography, antibody capture, gel electrophoresis, differential precipitation, organic extraction, size exclusion chromatography, fluorescence microscopy, or fluorescence activated cell sorting (FACS) assay. A targeting moiety can also refer to a protein, nucleic acid, nucleic acid analog, carbohydrate, or small molecule. The entity may be, for example, a therapeutic compound such as a small molecule, or a diagnostic entity such as a detectable label. A locale may be a tissue, a particular cell type, or a subcellular compartment. In one embodiment, the targeting moiety can direct the localization of an active entity. The active entity may be a small molecule, protein, polymer, or metal. The active entity, such as a liposome comprising a nucleic acid, may be useful for therapeutic, prophylactic, or diagnostic purposes. In some cases, a moiety may allow a liposomal structure to penetrate a blood brain barrier.

In other cases, a computerized tomography scan (CT) can or magnetic resonance imaging (MRI) can be taken. A CT can be taken on a slice thickness of 5 mm or less. If CT scans have slice thickness greater than 5 mm, the minimum size for a measurable lesion should be twice the slice thickness. In some cases, an FDG-PET scan can be used. FDG-PET can be used to evaluate new lesions. A negative FDG-PET at baseline, with a positive FDG-PET at follow up is a sign of progressive disease (PD) based on a new lesion. No FDG-PET at baseline and a positive FDG-PET at follow up: if a positive FDG-PET at follow-up corresponds to a new site of disease confirmed by CT, this is PD. If a positive PDG-PET at follow up corresponds to a pre-existing site of disease on CT that may not be progressing on a basis of anatomic imagines, this may not be PD. In some cases, FDG-PET may be used to upgrade a response to a CR in a manner similar to biopsy in cases where a residual radiographic abnormality is thought to represent fibrosis or scarring. A positive FDG-PET scan lesion means one which is FDG avid with an uptake greater than twice that of the surrounding tissue on an attenuation corrected image.

In some cases an evaluation of a lesion can be performed. A complete response (CR) can be a disappearance of all target lesions. Any pathological lymph nodes (target or non-target) may have reduction in short axis to less than 10 mm. A partial response (PR) can be at least a 30% decrease in a sum of the diameters of target lesions, taking as reference the baseline sum of diameters. Progressive disease (PD) can be at least a 20% increase in the sum of the diameters of target lesions, taking as reference the smallest sum. In addition to the relative increase of 20%, the sum must also demonstrate an absolute increase of at least 5 mm. Stable disease (SD) can be neither sufficient shrinkage to quality for PR nor sufficient increase to quality for PD, taking as reference the smallest sum of diameters.

In some cases, non-target lesions can be evaluated. A complete response of a non-target lesion can be a disappearance and normalization of tumor marker level. All lymph nodes must be non-pathological in size (less than 10 mm short axis). If tumor markers are initially above the upper normal limit, they must normalize for a patient to be considered a complete clinical response. Non-CR/Non-PD is persistence of one or more non-target lesions and or maintenance of tumor marker level above the normal limit. Progressive disease can be appearance of one or more new lesions and or unequivocal progression of existing non-target lesions. Unequivocal progression should not normally trump target lesion status.

In some cases, a best overall response can be the best response recorded from the start of treatment until disease progression/recurrence.

Pharmaceutical Compositions and Formulations

The compositions described throughout can be formulation into a pharmaceutical medicament and be used to treat a human or mammal, in need thereof, diagnosed with a disease, e.g., familial adenomatous polyposis (FAP). Medicaments can be co-administered with any additional therapy.

A disease that can be treated with a liposomal structure can be cancerous or non-cancerous. A disease can be familial adenomatous polyposis (FAP), attenuated FAP, cancer, chronic inflammatory bowel disease, chronic inflammatory bowel disease, ileal Crohn's or any combination thereof. In some cases, a disease can be identified by genetic screening. For example, a genetic screen can identify a BCRA mutation in a subject that can predispose them to breast cancer. In other cases, a genetic screen can identify a mutation in an APC gene that can result in FAP. A disease can also be a cardiovascular disease, a neurodegenerative disease, an ocular disease, a reproductive disease, a gastrointestinal disease, a brain disease, a skin disease, a skeletal disease, a muscoskeletal disease, a pulmonary disease, a thoracic disease, to name a few. A disease can be a genetic disease such as cystic fibrosis, tay-sachs, fragile X, Huntington's, neurofibromatosis, sickle cell, thalassemias, Duchenne's muscular dystrophy, or a combination thereof. A disease can produce polyps in a gastrointestinal tract. In some cases, a disease is FAP. FAP can progress to cancer. A gastrointestinal disease can be hereditary. For example, a hereditary gastrointestinal disease can be Gilbert's syndrome, telangiectasia, mucopolysaccaride, Osler-Weber-Rendu syndrome, pancreatitis, keratoacanthoma, biliary atresia, Morquio's syndrome, Hurler's syndrome, Hunter's syndrome, Crigler-Najjar, Rotor's, Peutz-Jeghers' syndrome, Dubin-Johnson, Osteochondroses, Osteochondrodysplasias, polyposis, or a combination thereof.

For oral administration, an excipient may include pharmaceutical grades of mannitol, lactose, starch, magnesium stearate, sodium saccharine, talcum, cellulose, glucose, gelatin, sucrose, magnesium carbonate, and the like. If desired, a liposomal composition may also contain minor amounts of non-toxic auxiliary substances such as wetting agents, emulsifying agents, or buffers.

A composition can be administered orally, by subcutaneous or other injection, intravenously, intracerebrally, intramuscularly, parenternally, transdermally, nasally or rectally. The form in which the compound or composition is administered depends at least in part on the route by which the compound is administered. In some cases, a liposomalcomposition can be employed in the form of solid preparations for oral administration; preparations may be tablets, granules, powders, capsules or the like. In a tablet formulation, a composition is typically formulated with additives, e.g. an excipient such as a saccharide or cellulose preparation, a binder such as starch paste or methyl cellulose, a filler, a disintegrator, and other additives typically used in the manufacture of medical preparations. Methods for preparing such dosage forms may be apparent to those skilled in the art. A liposomal composition to be administered may contain a quantity of a nanoparticle in a pharmaceutically effective amount for therapeutic use in a biological system, including a patient or subject. A pharmaceutical composition may be administered daily or administered on an as needed basis. In certain embodiments, a pharmaceutical composition can be administered to a subject prior to bedtime. In some embodiments, a pharmaceutical composition can be administered immediately before bedtime. In some embodiments, a pharmaceutical composition can be administered within about two hours before bedtime, preferably within about one hour before bedtime. In another embodiment, a pharmaceutical composition can be administered about two hours before bedtime. In a further embodiment, a pharmaceutical composition can be administered at least two hours before bedtime. In another embodiment, a pharmaceutical composition can be administered about one hour before bedtime. In a further embodiment, a pharmaceutical composition can be administered at least one hour before bedtime. In a still further embodiment, a pharmaceutical composition can be administered less than one hour before bedtime. In still another embodiment, the pharmaceutical composition can be administered immediately before bedtime. A pharmaceutical composition is administered orally or rectally.

An appropriate dosage (“therapeutically effective amount”) of an active agent(s) in a composition may depend, for example, on the severity and course of a condition, a mode of administration, a bioavailability of a particular agent(s), the age and weight of a subject, a subject's clinical history and response to an active agent(s), discretion of a physician, or any combination thereof. A therapeutically effective amount of an active agent(s) in a composition to be administered to a subject can be in the range of about 100 μg/kg body weight/day to about 1000 mg/kg body weight/day whether by one or more administrations. In some embodiments, the range of each active agent administered daily can be from about 100 μg/kg body weight/day to about 50 mg/kg body weight/day, 100 μg/kg body weight/day to about 10 mg/kg body weight/day, 100 μg/kg body weight/day to about 1 mg/kg body weight/day, 100 μg/kg body weight/day to about 10 mg/kg body weight/day, 500 μg/kg body weight/day to about 100 mg/kg body weight/day, 500 μg/kg body weight/day to about 50 mg/kg body weight/day, 500 μg/kg body weight/day to about 5 mg/kg body weight/day, 1 mg/kg body weight/day to about 100 mg/kg body weight/day, 1 mg/kg body weight/day to about 50 mg/kg body weight/day, 1 mg/kg body weight/day to about 10 mg/kg body weight/day, 5 mg/kg body weight/dose to about 100 mg/kg body weight/day, 5 mg/kg body weight/dose to about 50 mg/kg body weight/day, 10 mg/kg body weight/day to about 100 mg/kg body weight/day, and 10 mg/kg body weight/day to about 50 mg/kg body weight/day.

As used herein, “pharmaceutically acceptable carrier” includes any and all solvents, dispersion media, coatings, antibacterial and antifungal agents, isotonic and absorption delaying agents, sweeteners, salts, buffers, and the like. The pharmaceutically acceptable carriers may be prepared from a wide range of materials including, but not limited to, flavoring agents, sweetening agents and miscellaneous materials such as buffers and absorbents that may be needed in order to prepare a particular therapeutic composition.

A liposome complex can be formulated under sterile conditions within a reasonable time prior to administration. In some cases, a secondary therapy can also be administered. For example, another therapy such as chemotherapy or radiation therapy may be administered before or subsequent to the administration of the complex, for example within 12 hr. to 7 days. A combination of therapies, such as both chemotherapy and radiation therapy may be employed in addition to the administration of the complex.

The chemotherapeutic agents that can be used in combination with the disclosed structures include, but are not limited to, mitotic inhibitors (vinca alkaloids). Chemotherapeutic agents can also include vincristine, vinblastine, vindesine and Navelbine™ (vinorelbine, 5′-noranhydroblastine). In yet other cases, chemotherapeutic agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic agents that can be used in the methods and compositions disclosed herein can be podophyllotoxin derivatives, such as etoposide, teniposide and mitopodozide. The present disclosure further encompasses other chemotherapeutic agents known as alkylating agents, which alkylate the genetic material in tumor cells. chemotherapeutic agents include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of chemotherapeutic agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. The present disclosure further encompasses other chemotherapeutic cancer agents or agents for the treatment of a disease including without limitation anti-tumor antibodies, dacarbazine, azacytidine, amsacrine, melphalan, ifosfamide and mitoxantrone. A chemotherapeutic agent can be used to treat a disease, such as cancer, or a non-cancer disease. A non-cancer disease can be FAP. A cancer can be colorectal cancer.

The disclosed structures herein can be administered in combination with other chemotherapeutic agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other agents that can be used in combination with the disclosed structures include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug can be 5-fluorouracil, taxol, or leucovorin. In further embodiments, structures be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation, These drugs can inhibit either the calcium dependent phosphatase calcineurin (cyclosporine and F 506) or inhibit the p70S6 kinase that can be important for growth factor induced signaling (rapamycin).

### METHOD OF USE

An alternative of the diagnostic method can be used to monitor a therapy for familial adenomatous polyposis (FAP) or other disease state in a patient. A patient may be administered an effective amount of nanoparticles and a diagnostic method may include determining a level of APC incorporated into a cell genome whereupon a difference in APC levels before the start of therapy in a patient and during and/or after therapy will evidence the effectiveness of therapy in a patient, including whether a patient has completed therapy or whether the disease state has been inhibited or eliminated. In other cases, a gene for delivery by a liposome may be administered to a subject as a preventive measure. For example, a subject may not have diagnosed disease and may appear to be predisposed to a disease such as cancer. In some cases, a cancer can be a colon cancer.

In some cases, a cell that can be targeted with a structure comprising a polynucleic acid can be epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, granulosa cells, cumulus cells, epidermal cells, endothelial cells, pancreatic islet cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, non-keratinized epithelial cells, keratinized epithelial cells, lung cells, goblet cells, columnar epithelial cells, dopaminergic cells, squamous epithelial cells, osteocytes, osteoblasts, osteoclasts, dopaminergic cells, embryonic stem cells, fibroblasts and fetal fibroblasts. Further, the one or more cells can be pancreatic islet cells and/or cell clusters or the like, including, but not limited to pancreatic α cells, pancreatic β cells, pancreatic δ cells, pancreatic F cells (e.g., PP cells), or pancreatic ε cells. A cell can be pancreatic α cells. In another instance, a cell can be an intestinal crypt cell.

In some cases a cell that contacts a polynucleic acid that can be delivered by a structure can be genetically modified. For example, a polynucleic acid may transduce a cell that it contacts. An efficiency of transduction with a polynucleic acid described herein, for example, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% of the total number of cells that are contacted.

For example, a structure, such as a liposome, can be functional for at least or at least about 1, 2, 3, 4, 5, 6, 7, 8, 9, 10, 11, 12, 13, 14, 15, 16, 17, 18, 19, 20, 21, 22, 23, 24, 25, 6, 27, 28, 29, 30, 40, 50, 60, 70, 80, 90, or 100 days after introduction to a subject in need thereof. Structures can be functional for at least or at least about 1, 2, 3, 4, 5, 6, 7, 8, 9, 10, 11, or 12 months after introduction into a subject. A structure, such as a liposome, can be functional for at least or at least about 1, 2, 3, 4, 5, 6, 7, 8, 9, 10, 15, 20, 25, or 30 years after introduction to a subject. In some cases, a liposome can be functional for up to the lifetime of a recipient. Further, a structure such as a liposome can function at 100% of its normal intended operation. Liposomes can also function 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of their normal intended operation. Function of a liposome may refer to the efficiency of delivery, persistence of a liposome, stability of a liposome, or any combination thereof.

Liposomes or liposomal structures can also function over 100% of their normal intended operation. For example, liposomes can function 110, 120, 130, 140, 150, 160, 170, 180, 190, 200, 250, 300, 400, 500, 600, 700, 800, 900, 1000 or more % of their normal intended operation. Function can include an intended use. For example, a functional liposome can deliver a cargo, such as a minicircle DNA vector, to a target cell. In some cases, function can include a percent of cells that received a minicircle DNA vector from a liposome. In other cases, function can refer to a frequency or efficiency of protein generation from a polynucleic acid. For example, a liposome may deliver a vector to a cell that encodes for at least a portion of a gene, such as APC. A frequency of efficiency of APC generation from a vector may describe a functionality of a vector or liposome.

A minicircle vector concentration can be from 0.5 nanograms to 50 micrograms. A minicircle vector concentration can be from about 0.5 ng, 1 ng, 2 ng, 5 ng, 10 ng, 50 ng, 100 ng, 150 ng, 200 ng, 300 ng, 400 ng, 500 ng, 600 ng, 700 ng, 800 ng, 900 ng, 1000 ng, 1 μg, 2 μg, 5 μg, 10 μg, 20 μg, 30 μg, 40 μg, 50 μg, 60 μg, or up to 50 μg or greater. In some cases, the amount of nucleic acid (e.g., ssDNA, dsDNA, RNA) that may be introduced to a cell by a structure may be varied to optimize transfection efficiency and/or cell viability. In some cases, less than about 100 picograms of nucleic acid may be introduced to a subject. In some cases, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid may be added to each cell sample (e.g., one or more cells being electroporated). In some cases, the amount of nucleic acid (e.g., dsDNA) required for optimal transfection efficiency and/or cell viability may be specific to the cell type.

In some cases, an effective amount of a structure can mean an amount sufficient to increase the expression level of at least one gene which can be decreased in a subject prior to the treatment or an amount sufficient to alleviate one or more symptoms of cancer. For example, an effective amount can be an amount sufficient to increase the expression level of at least one gene selected from the group consisting of gastrointestinal differentiation genes, cell cycle inhibition genes, and tumor suppressor genes by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

In some embodiments, an effective amount means an amount sufficient to decrease the expression level of at least one gene which may be increased in the subject prior to the treatment or an amount sufficient to alleviate one or more symptoms of cancer. For example, an effective amount can be an amount sufficient to decrease the expression level of at least one gene selected from the group consisting of hedgehog pathway genes, MYC and EZH2 by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

Disclosed herein can be a method of determining efficacy of a cancer treatment in a subject in need thereof by (a) measuring the expression level of at least one gene in a sample obtained from the subject, (b) comparing the expression level of at least one gene in step (a) to a reference value or a prior measurement, and (c) determining the efficacy of the cancer treatment based on the comparison step. For example, the treatment can be effective when the tested subject has an increased expression of at least one gene 1) compared to a reference value or a prior measurement; or 2) over the period of time being monitored, such as 1, 2, 3, 4, 5, 6, or 7 days, or 1, 2, 3, 4, 5, 6, 7, 8, 9, 10 weeks, or 1, 2, 3, 4, 5, 6, 7, 8, 9, 10, 11, 12 months or longer. When the existing treatment may not be effective, a new treatment or an increased dosage of the existing treatment (for example, increasing the dosage of the structure administered to the subject) should be sought for the tested subject. In other cases, more frequent administration may be performed.

An effective amount for a subject will depend upon the subject's body weight, size, and health; the nature and extent of the condition; and the therapeutic selected for administration. An effective amount for a given situation can be determined by routine experimentation that may be within the skill and judgment of a clinician. An effective amount, as used herein, can refer to an amount of nanostructures sufficient to produce a measurable biological response (e.g., presence of APC in a cell). Actual dosage levels of the nanostructures can be varied so as to administer an amount of antioxidant molecules that may be effective to achieve the desired response for a particular subject and/or application. The selected dosage level will depend upon a variety of factors including the type of tissue being addressed, the types of cells and gel beads used, combination with other drugs or treatments, severity of the condition being treated, and the physical condition and prior medical history of the subject being treated. Preferably, a minimal dose can be administered, and a dose can be escalated in the absence of dose-limiting toxicity to a minimally effective amount.

A structure can be administered routinely in some cases. Routine administration can encompass hourly, daily, monthly, or yearly administration of a structure to a subject. For example, in some cases, a subject may be administered a structure daily for the entirety of the subject's life. In other cases, a structure may be administered daily for the duration of the presence of disease in a subject. A subject may be administered a structure comprising a polynucleic acid to treat a disease or disorder until the disease or disorder is reduced, controlled, or eliminated. Disease control may encompass the stabilization of a disease. For example, a cancer that is controlled may have stopped growing or spreading as measured by CT scan. A cancer may be a colon cancer. In other cases, a structure may be administered prophylactically. In some cases, a subject may have undergone a genetic screen that identifies the subject as being predisposed to a cancer, such as colon cancer. In this case, a predisposed subject may begin prophylactic treatment by receiving a structure comprising a polynucleic acid. In the case, that a subject contains a genetic mutation that predisposes the subject to colon cancer, that subject may begin prophylactic treatment with a structure comprising a polynucleic acid that encodes for at least a portion of an APC gene.

In some cases, prophylactic treatment can prevent a disease, such as cancer. When prevention can be used in relation to a condition, such as a local recurrence (e.g., pain), a disease such as cancer, a syndrome complex such as heart failure or any other medical condition prevention can include administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

A polynucleic acid may encode for a tumor-suppressor gene. A tumor-suppressor gene can generally encode for a protein that in one way or another can inhibit cell proliferation. Loss of one or more of these “brakes” may contribute to the development of a cancer. Five broad classes of proteins can be generally recognized as being encoded by tumor-suppressor genes: Intracellular proteins, such as the p16 cyclin-kinase inhibitor, that can regulate or inhibit progression through a specific stage of the cell cycle, receptors for secreted hormones (e.g., tumor derived growth factor 13) that may function to inhibit cell proliferation, checkpoint-control proteins that arrest the cell cycle if DNA may be damaged or chromosomes are abnormal, proteins that can promote apoptosis, enzymes that participate in DNA repair, or a combination thereof. Although DNA-repair enzymes may not directly function to inhibit cell proliferation, cells that have lost the ability to repair errors, gaps, or broken ends in DNA accumulate mutations in many genes, including those that are critical in controlling cell growth and proliferation. Thus loss-of-function mutations in the genes encoding DNA-repair enzymes may promote inactivation of other tumor-suppressor genes as well as activation of oncogenes. Since generally one copy of a tumor-suppressor gene suffices to control cell proliferation, both alleles of a tumor-suppressor gene must be lost or inactivated in order to promote tumor development. Thus oncogenic loss-of-function mutations in tumor-suppressor genes act recessively. Tumor-suppressor genes in many cancers have deletions or point mutations that prevent production of any protein or lead to production of a nonfunctional protein. In some cases, introducing a tumor suppressor gene encoding for a protein may ameliorate disease, prevent disease, or treat disease in a subject.

In some cases, a subject who inherits a mutant allele of APC, a tumor-suppressor gene, may have a high risk of developing colon cancer. Inheriting one mutant allele of another tumor-suppressor gene increase to almost 100 percent the probability that a subject will develop a specific tumor. In some cases, a subject that has inherited a mutant allele of APC, or a tumor-suppressor gene, may receive a structure described herein. In some cases, a structure may contain a polynucleic acid encoding for a protein produced by a mutant allele inherited in a subject. A mutant allele can be a tumor-suppressor protein such as APC. A protein can also be GLB1, DEFA5, WAC, DEFA6, or a combination thereof. Additional tumor-suppressor genes can be delivered. In some cases, a tumor suppressor can be a WW domain-containing adaptor with coiled-coil (WAC) gene.

The list of genes with, loss-of-function mutation is large—in the hundreds. In some cases, a website such as the Bioinformatics and Systems Medicine Laboratory of Vanderbilt University Medical Center, the TS GENE TUMOR. SUPPRESSOR GENE DATABASE, http://bioinfb.mc.vanderbiit.edu/TSGene/index.html, may be used. The database that includes 716 human genes (637 coding and 79 non-coding genes), 628 mouse genes, and 567 rat genes may be used to identify a gene to be introduced by a structure described herein. The structure of the disclosure can comprise the described genes. In some cases, a structure can be utilized to transport a gene for which loss of function mutation(s) can lead to neoplasm or tumor growth or cancer; and the skilled person well appreciates loss of function mutation(s) in cancer (as well as gain of function mutations in cancer and the disclosure can be analogously applicable to such mutations). The term “tumor suppressor” is herein used as understood in the art, namely to include loss of function mutation(s) in or involved in or that leads to or contributes to or is a factor in neoplasm or tumor growth or cancer, including tumor formation and/or spread and/or hyperplasia (altered cell divides in an uncontrolled manner leading to an excess of cells in that region of the tissue) and/or dysplasia (additional genetic changes in the hyperplastic cells lead to increasingly abnormal growth; cells and the tissue no longer look normal; cells and the tissue may become disorganized) and/or Carcinoma in situ (additional changes make the cells and tissues appear even more abnormal cells are spread over a larger area and the region of the tissue involved primarily contains altered cells; cells often ‘regress’ or become more primitive in their capabilities, e.g., a liver cell that no longer makes liver-specific proteins; cells may be de-differentiated or anaplastic and/or ‘benign tumor(s)’ and/or Cancer (including Malignant tumor(s)). Examples of genes to be targeted include the genes encoding the proteins involved in tumor suppression or any tumor suppressor gene(s), as that term is used herein, including, for example, any gene of the TS GENE TUMOR SUPPRESSOR GENE DATABASE, involved in “loss-of-function” or may be a tumor suppressor gene consistent with the use of the term in this disclosure.

A tumor suppressor gene that can be delivered by a structure, such as a liposome, can be APC, ARHGEF12, ATM, BCL11B, BLM, BMPR1A, BRCA1, BRCA2, CARS, CBFA2T3, CDH1, CDH11, CDK6, CDKN2C, CEBPA, CHEK2, CREB1, CREBBP, CYLD, DDX5, EXT1, EXT2, FBXW7, FH, FLT3, FOXP1, GPC3, IDH1, IL2, JAK2, MAP2K4, MDM4, MEN1, MLH1, MSH2, NF1, NF2, NOTCH1, NPM1, NR4A3, NUP98, PALB2, PML, PTEN, RB1, RUNX1, SDHB, SDHD, SMARCA4, SMARCB1, SOCS1, STK11, SUFU, SUZ12, SYK, TCF3, TNFAIP3, TP53, TSC1, TSC2, VHL, WRN, WT1, and any combination thereof. A gene that can be delivered can be or can be from about 50%, 60%, 70%, 80%, 90%, 95%, 96%, 97%, 98%, 99% or up to about 100% homologous to any one of SEQ ID NO: 761 to SEQ ID NO: 764.

TABLE 2
Summary of genes for delivery

| | | | | NCBI Gene | | | | |
|---|---|---|---|---|---|---|---|---|
| | | | | number | | | | Location |
| SEQ | Gene | | | (GRCh38.p2)* | Accession | Original | Original | in |
| ID | Symbol | Abbreviation | Name | GRCh38.p7 | Number | Start | Stop | genome |
| | APC | GS; DP2; DP3; | adenomatous | 324 | M74088 | 112707505 | 112846239 | 5q21-q22 |
| | | BTPS2; DP2.5; | polyposis | | | | | |
| | | PPP1R46; APC | coli | | | | | |
| 761 | APC | gtccaagggtagccaaggatggctgcagcttcatatgatcagttgttaaagcaagttgaggcactgaagatggagaactcaaatcttcgac | | | | | | |
| | | aagagctagaagataattccaatcatcttacaaaactggaaactgaggcatctaatatgaaggaagtacttaaacaactacaaggaagtat | | | | | | |
| | | tgaagatgaagctatggcttcttctggacagattgatttattagagcgtcttaaagagcttaacttagatagcagtaatttccctggagta | | | | | | |
| | | aaactgcggtcaaaaatgtccctccgttcttatggaagccgggaaggatctgtatcaagccgttctggagagtgcagtcctgttcctatgg | | | | | | |
| | | gttcatttccaagaagagggtttgtaaatggaagcagagaaagtactggatatttagaagaacttgagaaagagaggtcattgcttcttgc | | | | | | |
| | | tgatcttgacaaagaagaaaaggaaaaagactggtattacgctcaacttcagaatctcactaaaagaatagatagtcttcctttaactgaa | | | | | | |
| | | aatttttccttacaaacagatatgaccagaaggcaattggaatatgaagcaaggcaaatcagagttgcgatggaagaacaactaggtacct | | | | | | |
| | | gccaggatatggaaaaacgagcacagcgaagaatagccagaattcagcaaatcgaaaaggacatacttcgtatacgacagcttttacagtc | | | | | | |
| | | ccaagcaacagaagcagagaggtcatctcagaacaagcatgaaaccggctcacatgatgctgagcggcagaatgaaggtcaaggagtggga | | | | | | |
| | | gaaatcaacatggcaacttctggtaatggtcagggttcaactacacgaatggaccatgaaacagccagtgttttgagttctagtagcacac | | | | | | |
| | | actctgcacctcgaaggctgacaagtcatctgggaaccaaggtggaaatggtgtattcattgttgtcaatgcttggtactcatgataagga | | | | | | |
| | | tgatatgtcgcgaactttgctagctatgtctagctcccaagacagctgtatatccatgcgacagtctggatgtcttcctctcctcatccag | | | | | | |
| | | cttttacatggcaatgacaaagactctgtattgttgggaaattcccggggcagtaaagaggctcgggccagggccagtgcagcactccaca | | | | | | |
| | | acatcattcactcacagcctgatgacaagagaggcaggcgtgaaatccgagtccttcatcttttggaacagatacgcgcttactgtgaaac | | | | | | |
| | | ctgttgggagtggcaggaagctcatgaaccaggcatggaccaggacaaaaatccaatgccagctcctgttgaacatcagatctgtcctgct | | | | | | |
| | | gtgtgtgttctaatgaaactttcatttgatgaagagcatagacatgcaatgaatgaactagggggactacaggccattgcagaattattgc | | | | | | |
| | | aagtggactgtgaaatgtacgggcttactaatgaccactacagtattacactaagacgatatgctggaatggctttgacaaacttgacttt | | | | | | |
| | | tggagatgtagccaacaaggctacgctatgctctatgaaaggctgcatgagagcacttgtggcccaactaaaatctgaaagtgaagactta | | | | | | |
| | | cagcaggttattgcaagtgttttgaggaatttgtcttggcgagcagatgtaaatagtaaaaagacgttgcgagaagttggaagtgtgaaag | | | | | | |
| | | cattgatggaatgtgctttagaagttaaaaaggaatcaaccctcaaaagcgtattgagtgccttatggaatttgtcagcacattgcactga | | | | | | |
| | | gaataaagctgatatatgtgctgtagatggtgcacttgcatttttggttggcactcttacttaccggagccagacaaacactttagccatt | | | | | | |
| | | attgaaagtggaggtgggatattacggaatgtgtccagcttgatagctacaaatgaggaccacaggcaaatcctaagagagaacaactgtc | | | | | | |
| | | tacaaactttattacaacacttaaaatctcatagtttgacaatagtcagtaatgcatgtggaactttgtggaatctctcagcaagaaatcc | | | | | | |
| | | taaagaccaggaagcattatgggacatgggggcagttagcatgctcaagaacctcattcattcaaagcacaaaatgattgctatgggaagt | | | | | | |
| | | gctgcagctttaaggaatctcatggcaaataggcctgcgaagtacaaggatgccaatattatgtctcctggctcaagcttgccatctcttc | | | | | | |
| | | atgttaggaaacaaaaagccctagaagcagaattagatgctcagcacttatcagaaacttttgacaatatagacaatttaagtcccaaggc | | | | | | |
| | | atctcatcgtagtaagcagagacacaagcaaagtctctatggtgattatgtttttgacaccaatcgacatgatgataataggtcagacaat | | | | | | |
| | | tttaatactggcaacatgactgtcctttcaccatatttgaatactacagtgttacccagctcctcttcatcaagaggaagcttagatagtt | | | | | | |
| | | ctcgttctgaaaaagatagaagtttggagagagaacgcggaattggtctaggcaactaccatccagcaacagaaaatccaggaacttcttc | | | | | | |
| | | aaagcgaggtttgcagatctccaccactgcagcccagattgccaaagtcatggaagaagtgtcagccattcatacctctcaggaagacaga | | | | | | |
| | | agttctgggtctaccactgaattacattgtgtgacagatgagagaaatgcacttagaagaagctctgctgcccatacacattcaaacactt | | | | | | |
| | | acaatttcactaagtcggaaaattcaaataggacatgttctatgccttatgccaaattagaatacaagagatcttcaaatgatagtttaaa | | | | | | |
| | | tagtgtcagtagtagtgatggttatggtaaaagaggtcaaatgaaaccctcgattgaatcctattctgaagatgatgaaagtaagttttgc | | | | | | |
| | | agttatggtcaatacccagccgacctagcccataaaatacatagtgcaaatcatatggatgataatgatggagaactagatacaccaataa | | | | | | |
| | | attatagtcttaaatattcagatgagcagttgaactctggaaggcaaagtccttcacagaatgaaagatgggcaagacccaaacacataat | | | | | | |
| | | agaagatgaaataaaacaaagtgagcaaagacaatcaaggaatcaaagtacaacttatcctgtttatactgagagcactgatgataaacac | | | | | | |
| | | ctcaagttccaaccacattttggacagcaggaatgtgtttctccatacaggtcacggggagccaatggttcagaaacaaatcgagtgggtt | | | | | | |
| | | ctaatcatggaattaatcaaaatgtaagccagtctttgtgtcaagaagatgactatgaagatgataagcctaccaattatagtgaacgtta | | | | | | |
| | | ctctgaagaagaacagcatgaagaagaagagagaccaacaaattatagcataaaatataatgaagagaaacgtcatgtggatcagcctatt | | | | | | |
| | | gattatagtttaaaatatgccacagatattccttcatcacagaaacagtcattttcattctcaaagagttcatctggacaaagcagtaaaa | | | | | | |
| | | ccgaacatatgtcttcaagcagtgagaatacgtccacaccttcatctaatgccaagaggcagaatcagctccatccaagttctgcacagag | | | | | | |
| | | tagaagtggtcagcctcaaaaggctgccacttgcaaagtttcttctattaaccaagaaacaatacagacttattgtgtagaagatactcca | | | | | | |
| | | atatgtttttcaagatgtagttcattatcatctttgtcatcagctgaagatgaaataggatgtaatcagacgacacaggaagcagattctg | | | | | | |
| | | ctaataccctgcaaatagcagaaataaaagaaaagattggaactaggtcagctgaagatcctgtgagcgaagttccagcagtgtcacagca | | | | | | |
| | | ccctagaaccaaatccagcagactgcagggttctagtttatcttcagaatcagccaggcacaaagctgttgaattttcttcaggagcgaaa | | | | | | |
| | | tctccctccaaaagtggtgctcagacacccaaaagtccacctgaacactatgttcaggagaccccactcatgtttagcagatgtacttctg | | | | | | |
| | | tcagttcacttgatagttttgagagtcgttcgattgccagctccgttcagagtgaaccatgcagtggaatggtaagtggcattataagccc | | | | | | |
| | | cagtgatcttccagatagccctggacaaaccatgccaccaagcagaagtaaaacacctccaccacctcctcaaacagctcaaaccaagcga | | | | | | |
| | | gaagtacctaaaaataaagcacctactgctgaaaagagagagagtggacctaagcaagctgcagtaaatgctgcagttcagagggtccagg | | | | | | |
| | | ttcttccagatgctgatactttattacattttgccacggaaagtactccagatggattttcttgttcatccagcctgagtgctctgagcct | | | | | | |
| | | cgatgagccatttatacagaaagatgtggaattaagaataatgcctccagttcaggaaaatgacaatgggaatgaaacagaatcagagcag | | | | | | |
| | | cctaaagaatcaaatgaaaaccaagagaaagaggcagaaaaaactattgattctgaaaaggacctattagatgattcagatgatgatgata | | | | | | |
| | | ttgaaatactagaagaatgtattatttctgccatgccaacaaagtcatcacgtaaagcaaaaaagccagcccagactgcttcaaaattacc | | | | | | |
| | | tccacctgtggcaaggaaaccaagtcagctgcctgtgtacaaacttctaccatcacaaaacaggttgcaaccccaaaagcatgttagtttt | | | | | | |
| | | acaccgggggatgatatgccacgggtgtattgtgttgaagggacacctataaacttttccacagctacatctctaagtgatctaacaatcg | | | | | | |
| | | aatcccctccaaatgagttagctgctggagaaggagttagaggaggagcacagtcaggtgaatttgaaaaacgagataccattcctacaga | | | | | | |
| | | aggcagaagtacagatgaggctcaaggaggaaaaacctcatctgtaaccatacctgaattggatgacaataaagcagaggaaggtgatatt | | | | | | |
| | | cttgcagaatgcattaattctgctatgcccaaagggaaaagtcacaagcctttccgtgtgaaaaagataatggaccaggtccagcaagcat | | | | | | |
| | | ctgcgtcgtcttctgcacccaacaaaaatcagttagatggtaagaaaaagaaaccaacttcaccagtaaaacctataccacaaaatactga | | | | | | |
| | | atataggacacgtgtaagaaaaaatgcagactcaaaaaataatttaaatgctgagagagttttctcagacaacaaagattcaaagaaacag | | | | | | |
| | | aatttgaaaaataattccaaggacttcaatgataagctcccaaataatgaagatagagtcagaggaagttttgcttttgattcacctcatc | | | | | | |
| | | attacacgcctattgaaggaactccttactgatttcacgaaatgattctttgagttctctagattttgatgatgatgatgttgacctttcc | | | | | | |
| | | agggaaaaggctgaattaagaaaggcaaaagaaaataaggaatcagaggctaaagttaccagccacacagaactaacctccaaccaacaat | | | | | | |
| | | cagctaataagacacaagctattgcaaagcagccaataaatcgaggtcagcctaaacccatacttcagaaacaatccacttttccccagtc | | | | | | |
| | | atccaaagacataccagacagaggggcagcaactgatgaaaagttacagaattttgctattgaaaatactccagtttgcttttctcataat | | | | | | |
| | | tcctctctgagttctctcagtgacattgaccaagaaaacaacaataaagaaaatgaacctatcaaagagactgagccccctgactcacagg | | | | | | |
| | | gagaaccaagtaaacctcaagcatcaggctatgctcctaaatcatttcatgttgaagataccccagtttgtttctcaagaaacagttctct | | | | | | |
| | | cagttctcttagtattgactctgaagatgacctgttgcaggaatgtataagctccgcaatgccaaaaaagaaaaagccttcaagactcaag | | | | | | |
| | | ggtgataatgaaaaacatagtcccagaaatatgggtggcatattaggtgaagatctgacacttgatttgaaagatatacagagaccagatt | | | | | | |
| | | cagaacatggtctatcccctgattcagaaaattttgattggaaagctattcaggaaggtgcaaattccatagtaagtagtttacatcaagc | | | | | | |
| | | tgctgctgctgcatgtttatctagacaagcttcgtctgattcagattccatcctttccctgaaatcaggaatctctctgggatcaccattt | | | | | | |
| | | catcttacacctgatcaagaagaaaaaccctttacaagtaataaaggcccacgaattctaaaaccaggggagaaaagtacattggaaacta | | | | | | |
| | | aaaagatagaatctgaaagtaaaggaatcaaaggaggaaaaaaagtttataaaagtttgattactggaaaagttcgatctaattcagaaat | | | | | | |
| | | ttcaggccaaatgaaacagccccttcaagcaaacatgccttcaatctctcgaggcaggacaatgattcatattccaggagttcgaaatagc | | | | | | |
| | | tcctcaagtacaagtcctgtttctaaaaaaggcccaccccttaagactccagcctccaaaagccctagtgaaggtcaaacagccaccactt | | | | | | |
| | | ctcctagaggagccaagccatctgtgaaatcagaattaagccctgttgccaggcagacatcccaaataggtgggtcaagtaaagcaccttc | | | | | | |
| | | tagatcaggatctagagattcgaccccttcaagacctgcccagcaaccattaagtagacctatacagtctcctggccgaaactcaatttcc | | | | | | |
| | | cctggtagaaatggaataagtcctcctaacaaattatctcaacttccaaggacatcatcccctagtactgcttcaactaagtcctcaggtt | | | | | | |
| | | ctggaaaaatgtcatatacatctccaggtagacagatgagccaacagaaccttaccaaacaaacaggtttatccaagaatgccagtagtat | | | | | | |
| | | tccaagaagtgagtctgcctccaaaggactaaatcagatgaataatggtaatggagccaataaaaaggtagaactttctagaatgtcttca | | | | | | |
| | | actaaatcaagtggaagtgaatctgatagatcagaaagacctgtattagtacgccagtcaactttcatcaaagaagctccaagcccaacct | | | | | | |
| | | taagaagaaaattggaggaatctgcttcatttgaatctctttctccatcatctagaccagcttctcccactaggtcccaggcacaaactcc | | | | | | |
| | | agttttaagtccttcccttcctgatatgtctctatccacacattcgtctgttcaggctggtggatggcgaaaactcccacctaatctcagt | | | | | | |
| | | cccactatagagtataatgatggaagaccagcaaagcgccatgatattgcacggtctcattctgaaagtccttctagacttccaatcaata | | | | | | |
| | | ggtcaggaacctggaaacgtgagcacagcaaacattcatcatcccttcctcgagtaagcacttggagaagaactggaagttcatcttcaat | | | | | | |
| | | tctttctgcttcatcagaatccagtgaaaaagcaaaaagtgaggatgaaaaacatgtgaactctatttcaggaaccaaacaaagtaaagaa | | | | | | |
| | | aaccaagtatccgcaaaaggaacatggagaaaaataaaagaaaatgaattttctcccacaaatagtacttctcagaccgtttcctcaggtg | | | | | | |
| | | ctacaaatggtgctgaatcaaagactctaatttatcaaatggcacctgctgtttctaaaacagaggatgtttgggtgagaattgaggactg | | | | | | |
| | | tcccattaacaatcctagatctggaagatctcccacaggtaatactcccccggtgattgacagtgtttcagaaaaggcaaatccaaacatt | | | | | | |
| | | aaagattcaaaagataatcaggcaaaacaaaatgtgggtaatggcagtgttcccatgcgtaccgtgggtttggaaaatcgcctgaactcct | | | | | | |
| | | ttattcaggtggatgcccctgaccaaaaaggaactgagataaaaccaggacaaaataatcctgtccctgtatcagagactaatgaaagttc | | | | | | |
| | | tatagtggaacgtaccccattcagttctagcagctcaagcaaacacagttcacctagtgggactgttgctgccagagtgactccttttaat | | | | | | |
| | | tacaacccaagccctaggaaaagcagcgcagatagcacttcagctcggccatctcagatcccaactccagtgaataacaacacaaagaagc | | | | | | |
| | | gagattccaaaactgacagcacagaatccagtggaacccaaagtcctaagcgccattctgggtcttaccttgtgacatctgtttaaaagag | | | | | | |
| | | aggaagaatgaaactaagaaaattctatgttaattacaactgctatatagacattttgtttcaaatgaaactttaaaagactgaaaaattt | | | | | | |
| | | tgtaaataggtttgattcttgttagagggtttttgttctggaagccatatttgatagtatactttgtcttcactggtcttattttgggagg | | | | | | |
| | | cactcttgatggttaggaaaaaaatagtaaagccaagtatgtttgtacagtatgttttacatgtatttaaagtagcacccatcccaacttc | | | | | | |
| | | ctttaattattgcttgtcttaaaataatgaacactacagatagaaaatatgatatattgctgttatcaatcatttctagattataaactga | | | | | | |
| | | ctaaacttacatcagggaaaaattggtatttatgcaaaaaaaaatgtttttgt | | | | | | |
| | GLB1 | EBP; ELNR1; | galactosidase | 2720 | NM_001317040.1 | 32996608 | 33097202 | 3p21.33 |
| | | MPS4B; GLB1 | beta 1 | | | | | |
| 762 | GLB1 | cacagcggggcggggcgcggggctgagcaggcgcgagggctggctgctgggccgacgaggggcggtgccaggccgtgggtccttagtcaag | | | | | | |
| | | tgacgcgaagcggccggcctgggcgccgactgcagagccgggaggctggtggtcatgccggggttcctggttcgcatcctccctctgttgc | | | | | | |
| | | tggttctgctgcttctgggccctacgcgcggcttgcgctctcgattccttccttgggcttttcatctgccacggcaagcgcctaagtctca | | | | | | |
| | | gctgccgcttcacaaaagaggtaccaagactgcaccgaatgagcacgcaagcagcaacaggagtgggagaagaaggaggcggcagcagtgg | | | | | | |
| | | aatgccacccagaggatgtttgaaattgactatagccgggactccttcctcaaggatggccagccatttcgctacatctcaggaagcattc | | | | | | |
| | | actactcccgtgtgccccgcttctactggaaggaccggctgctgaagatgaagatggctgggctgaacgccatccagacgtatgtgccctg | | | | | | |
| | | gaactttcatgagccctggccaggacagtaccagttttctgaggaccatgatgtggaatattttcttcggctggctcatgagctgggactg | | | | | | |
| | | ctggttatcctgaggcccgggccctacatctgtgcagagtgggaaatgggaggattacctgcttggctgctagagaaagagtctattcttc | | | | | | |
| | | tccgctcctccgacccagattacctggcagctgtggacaagtggttgggagtccttctgcccaagatgaagcctctcctctatcagaatgg | | | | | | |
| | | agggccagttataacagtgcaggttgaaaatgaatatggcagctactttgcctgtgattttgactacctgcgcttcctgcagaagcgcttt | | | | | | |
| | | cgccaccatctgggggatgatgtggttctgtttaccactgatggagcacataaaacattcctgaaatgtggggccctgcagggcctctaca | | | | | | |
| | | ccacggtggactttggaacaggcagcaacatcacagatgctttcctaagccagaggaagtgtgagcccaaaggacccttgatcaattctga | | | | | | |
| | | attctatactggctggctagatcactggggccaacctcactccacaatcaagaccgaagcagtggcttcctccctctatgatatacttgcc | | | | | | |
| | | cgtggggcgagtgtgaacttgtacatgtttataggtgggaccaattttgcctattggaatggggccaactcaccctatgcagcacagccca | | | | | | |
| | | ccagctacgactatgatgccccactgagtgaggctggggacctcactgagaagtattttgctctgcgaaacatcatccagaagtttgaaaa | | | | | | |
| | | agtaccagaaggtcctatccctccatctacaccaaagtttgcatatggaaaggtcactttggaaaagttaaagacagtgggagcagctctg | | | | | | |
| | | gacattctgtgtccctctgggcccatcaaaagcctttatcccttgacatttatccaggtgaaacagcattatgggtttgtgctgtaccgga | | | | | | |
| | | caacacttcctcaagattgcagcaacccagcacctctctcttcacccctcaatggagtccacgatcgagcatatgttgctgtggatgggat | | | | | | |
| | | cccccagggagtccttgagcgaaacaatgtgatcactctgaacataacagggaaagctggagccactctggaccttctggtagagaacatg | | | | | | |
| | | ggacgtgtgaactatggtgcatatatcaacgattttaagggtttggtttctaacctgactctcagttccaatatcctcacggactggacga | | | | | | |
| | | tctttccactggacactgaggatgcagtgcgcagccacctggggggctggggacaccgtgacagtggccaccatgatgaagcctgggccca | | | | | | |
| | | caactcatccaactacacgctcccggccttttatatggggaacttctccattcccagtgggatcccagacttgccccaggacacctttatc | | | | | | |
| | | cagtttcctggatggaccaagggccaggtctggattaatggctttaaccttggccgctattggccagcccggggccctcagttgaccttgt | | | | | | |
| | | ttgtgccccagcacatcctgatgacctcggccccaaacaccatcaccgtgctggaactggagtgggcaccctgcagcagtgatgatccaga | | | | | | |
| | | actatgtgctgtgacgttcgtggacaggccagttattggctcatctgtgacctacgatcatccctccaaacctgttgaaaaaagactcatg | | | | | | |
| | | cccccacccccgcaaaaaaacaaagattcatggctggaccatgtatgatgatgaaagcctgtgtctttgagggattctaccctgaacatac | | | | | | |
| | | ctcacagatcctccctgtcatgccacatttcactgattggaatgtggaaatggaaaaggaatttaggatgtgcattttcacctgaggtttc | | | | | | |
| | | cctgcatccctgcagtgccaaagccccaccttcagggaccacctggaatgtgtgaggggctgacagcacagtaacgtgcatacatatctgc | | | | | | |
| | | agggctggaatggaagctttaaaggtggtagtgatttttattttggaagaatcatgttacctttttgttaaataaaatttgtactcaaatg | | | | | | |
| | | atgatgtcactgtttttaatgtgcaggtattgaattatatggtctgacttaaatcataactagacttgagtgggctgaataaaccacttca | | | | | | |
| | | ctaacttgaagttcaaaaggatggaaaataaaaaaaaaaaaaaaaaaaa | | | | | | |
| | DEF | DEF5; HD-5 | defensin | 1670 | NM_021010 | 7055300 | 7056739 | 8p23.1 |
| | A5* | | alpha 5 | | | | | |
| 763 | DEF | acatatccactcctgctctccctcctgcaggtgaccccagccatgaggaccatcgccatccttgctgccattctcctggtggccctgcagg | | | | | | |
| | A5* | cccaggctgagtcactccaggaaagagctgatgaggctacaacccagaagcagtctggggaagacaaccaggaccttgctatctcctttgc | | | | | | |
| | | aggaaatggactctctgctcttagaacctcaggttctcaggcaagagccacctgctattgccgaaccggccgttgtgctacccgtgagtcc | | | | | | |
| | | ctctccggggtgtgtgaaatcagtggccgcctctacagactctgctgtcgctgagcttcctagatagaaaccaaagcagtgcaagattcag | | | | | | |
| | | ttcaaggtcctgaaaaaagaaaaacattttactctgtgtaccttgtgtctttctaaatttctctctccaaaataaagttcaagcattaaac | | | | | | |
| | | ttaaaaaaaaaaa | | | | | | |
| | DEF | DEF6; HD-6 | defensin | 1671 | U33317 | 6924694 | 6926076 | 8p23.1 |
| | A6* | | alpha 6 | | | | | |

| | | |
|---|---|---|
| 764 | DEF | attcagcacaattcctatcaaaatccccatggcattctttacagaaataaaaaaaaaatcctaaaatttgtatgaaatggtgaaagaaccc |
| | A6* | aaatagacaaaacaattctgagaaaaaaaaacaaagttgaagacatcacaattcctggttttaaattatattacaaagctacggtaatcaa |
| | | aacaatatggtattggcataaaaccagacacataccagtgaaataaaattgagagtccagaaacacatcgaaacacatatggtcaagtaac |
| | | ttttgacaatggcatgaagaagacacaatagggacaggagagtctcttcaataaatggtggtgggaatactatttccatatgcaaaacaat |
| | | aaaactggacccttctcttttggcatatacaaaacttaactcaaaatggataaaatacctgatagaagaccagaaacaagagactatgaaa |
| | | ataatggtagagttctatatgttttgagctacaaagtattcaaattacagcattagggagaaaaaacaaagcacaaaccaatgtttaattt |
| | | ttaagtggagatttcctggagcagagctgcagggctggggcaaggatggaggctcattactccttttaccctttgaatggtccttgtgggc |
| | | aattcaacattttctaaaattaaaaataaaataaaatttcagagggggattgtgggcatgtcaattgtttcgatatttctcaaagtggaga |
| | | gaaatacctagtacatattttacttgatacattcagatcacatgggtttcttgaaataaatcttttggctttagttttactaactctttac |
| | | ctagtatcccactgagttatttcccttataggctgataaggtcattatcttctccacactgtgccccacaagagcctattcaccccataat |
| | | ctcaaagcctccttcatgagggcagaaatgccccctgaatcctgcaatgaattaactctctactctagcgggatccagctctggcctcaag |
| | | gtctacacctccagagagtggccagccccaccttcagaaaataagaggcatttgattcctgaaattattcattgaaagcactgttcttttc |
| | | ttttttgaatattaacaagtaaatattccagcagatggaaaacaggacaatgtaacactgttcttatcatcactatcagctgggaccagaa |
| | | cagacactcaataaacagcctcacactacaatgaagcttggagaacaaaggagcatcaaagggacatggagggcaagggtagctcttctgc |
| | | tccccaatcacatgcactccccgtctcaccgcaacatctgtccctgagccttctccaagcagacctataaatccaggctggctcctcactc |
| | | cccacacatctgctcctgctctctctcctccagcgaccctagccatgagaaccctcaccatcctcactgctgttctcctcgtggccctcca |
| | | ggccaaggctgagccactccaagctgaggatgatccactgcaggcaaaagcttatgaggctgatgcccaggagcagcgtggggcaaatgac |
| | | caggactttgccgtctcctttgcagaggatgcaagctcaagtcttagagctttgggtaagagacaccagcattgcagagctaggagtgtag |
| | | agaggaaaaccaagcacttctagaattagatccaacagctggctctttctcttaggtgatcacctccccaggcctcaattactttatgttt |
| | | gtactgaaaaggaagaatcagtgatattcaaggcatgacttttctctaaagattttttaattctatgataagtctatgaaattttctaatt |
| | | ttttgccatgtagaaatgtattgaggagtctctacttcaaggaagagagcctaattttaaaggaatgttttgttttgctttgttttgtttt |
| | | gttttgtttgatggagtctcgctctgtcacccaggctggagtgcagtggcactatctcagatcactgcaacctctgcctcctgggttcaag |
| | | caattctctccctcagcctcccaagtcactaggattacaggcaccagacaccaagcctggctaaattttgtattttttttttttcagtaga |
| | | gacggggtttcaccatcttggccaggctagtcttgaactcctgaccttgtgataaacccaccttggcctcccaaagtgctgggattacagg |
| | | cgtaagccaccacatccagctgctaaaggaatgttttttaatctgacttttataggaaccgttggaaactggagacagtcatatgggtgca |
| | | ttcagatgtgtgtgtgacagggaaggagcagataagtacagcatatcagaatgggtctctaatcctgtgtgtgaccaacactgctctgcgt |
| | | atttattcctattgatggtgtgatcatgctattggctgtaatgcagccagcattacatgtcagcaagcatgcaacttcctgaagattctct |
| | | ttactgcccgctgctgaccctggtgctcaatttctgatgctctctctctctgtccccaggctcaacaagggctttcacttgccattgcaga |
| | | aggtcctgttattcaacagaatattcctatgggacctgcactgtcatgggtattaaccacagattctgctgcctctgagggatgagaacag |
| | | agagaaatatattcataatttactttatgacctagaaggaaactgtcgtgtgtcctatacattgccatcaactttgtttcctcatctcaaa |
| | | taaagtcctttcagcaagttcttttgtgtttgtgcttttctggtgtttgataattcaggattcttcagatgcaaaaacaaaaacccaagtc |
| | | gtatctcagaacactagctcttcgaaagagttttctatgtagaccagagaagtggtagagaggatgttgagagaagagaggatttgggttt |
| | | tttgtttttttgttttttgctttctgagatggagtctcgctctgttgcccaggctgcagtgcagtggcacaatcttggctcactgaaacct |
| | | ttacctcctgggttcaagtgattctcctgcctcagcctcccaagtagctgggatta |

Suitable formulations can include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. Suitable inert carriers can include sugars such as lactose. In some cases, the compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

A carrier can be a solvent or dispersion medium containing, for example, water, ethanol, one or more polyols (e.g., glycerol, propylene glycol, and liquid polyethylene glycol), oils, such as vegetable oils (e.g, peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride. Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof. Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimoniuni bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine. The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s). The formulation is typically buffered to a pH of 3-8 for parenteral administration upon reconstitution. Suitable buffers include, but are not limited to, phosphate buffers, acetate buffers, and citrate buffers. Water soluble polymers can be often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions can be prepared by incorporating the various sterilized active ingredients into a sterile vehicle which contains the basic dispersion medium and the required other ingredients from those listed above. In the case of sterile powders for the preparation of sterile injectable solutions, a method of preparation can be vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

A formulation can be an ocular formulation or a topical formation. Pharmaceutical formulations for ocular administration can be in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol. In still other embodiments, the liposomes can be formulated for topical administration to mucosa. Suitable dosage forms for topical administration include creams, ointments, salves, sprays, gels, lotions, emulsions, liquids, and transdermal patches. The formulation may be formulated for transmucosal, transepithelial, transendothelial, or transdermal administration. The compositions contain one or more chemical penetration enhancers, membrane permeability agents, membrane transport agents, emollients, surfactants, stabilizers, and combination thereof. In some embodiments, the liposomes can be administered as a liquid formulation, such as a solution or suspension, a semi-solid formulation, such as a lotion or ointment, or a solid formulation. In some embodiments, the liposomes can be formulated as liquids, including solutions and suspensions, such as eye drops or as a semi-solid formulation, such as ointment or lotion for topical application to mucosa, such as the eye or vaginally or rectally. The formulation may contain one or more excipients, such as emollients, surfactants, emulsifiers, and penetration enhancers.

In some cases, formulations can be presented in unit-dose or multi-dose containers, for example sealed ampoules and vials, and can be stored in a frozen or freeze-dried (lyophilized) condition requiring only the addition of sterile liquid carrier immediately prior to use. For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated in some cases. Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner. In some cases, compositions can also be formulated as a preparation for implantation or injection. Thus, for example, a structure can be formulated with suitable polymeric, aqueous, and/or hydrophilic materials, or resins, or as sparingly soluble derivatives (e.g., as a sparingly soluble salt). The compounds can also be formulated in rectal compositions, creams or lotions, or transdermal patches.

In some cases, a pharmaceutical composition may include a salt. A salt can be relatively non-toxic. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like.

In some cases, nanostructures can have a circulation half-life in a subject of about 6 hours, 12 hours, 18 hours, 24 hours, 30 hours, 36 hours, 42 hours, or 48 hours. In some embodiments the nanoparticles can comprise a circulation half-life of more than about 48 hours. In some embodiments circulation half-life can be enhanced by increasing the concentration of a hydrophobic monomer of the polymer, thereby increasing the forces necessary to disassemble the nanostructures.

In some embodiments delivery of the nanostructures to an environment having a relatively lower pH can cause at least the partial disassembly of the nanostructures. In particular, some nanostructures have a pH-responsive character, and when exposed to a lower pHs the nanostructures at least partially dissemble to expose the core of the nanostructures. Without being bound by theory or mechanism, in some embodiments a lower pH can increase the cationic charges on the cationic monomers present in the core of a nanoparticle, and the repulsive forces due to the increased cationic charges can cause the nanostructures to at least partially disassemble. The at least partially disassembly of the nanostructures can expose the polynucleic acid that are bound to in the core of the nanostructures to the surrounding environment. Thus, at least partial disassembly of the nanostructures can allow the polynucleic acid to be delivered to their final target. At least partial disassembly can also expose the cationic monomer and hydrophobic monomer to the surrounding environment, and the cationic monomer and/or hydrophobic monomer can have a membrane disruptive character. More specifically, exposure of the monomers in the second block of the polymers that comprise the nanostructures can induce the disruption of any membranes that contain the nanostructures. Thus, in some embodiments, after the uptake of the nanostructures into a cell, the nanostructures can at least partially disassemble to deliver the polynucleic acid in a pH-responsive manner. In some embodiments the nanostructures at least partially disassemble at about or below an endosomal H, and therefore after uptake of the nanostructures into the endosome, endosomal pH's can drive the nanostructures to disassemble. The at least partially disassembled nanostructures can then disrupt the endosomal or liposomal membranes so that the polynucleotide can be delivered to the cytosol of a particular cell.

In some cases, a level of disease can be determined in sequence or concurrent with a liposomal treatment regime. A level of disease on target lesions can be measured as a Complete Response (CR): Disappearance of all target lesions, Partial Response (PR): At least a 30% decrease in the sum of the longest diameter (LD) of target lesions taking as reference the baseline sum LD, Progression (PD): At least a 20% increase in the sum of LD of target lesions taking as reference the smallest sum LD recorded since the treatment started or the appearance of one or more new lesions, Stable Disease (SD): Neither sufficient shrinkage to qualify for PR nor sufficient increase to qualify for PD taking as references the smallest sum LD. In other cases, a non-target lesion can be measured. A level of disease of a non-target lesion can be Complete Response (CR): Disappearance of all non-target lesions and normalization of tumor marker level, Non-Complete Response: Persistence of one or more non-target lesions, Progression (PD): Appearance of one or more new lesions. Unequivocal progression of existing non-target lesions.

### Kits

Disclosed herein can be kits comprising liposomal compositions. Disclosed herein can also be kits for the treatment or prevention of a cancer, pathogen infection, immune disorder or a disease or condition disclosed herein. In some cases, a kit can include a therapeutic or prophylactic liposomal composition containing an effective amount of a liposome containing a nucleic acid in unit dosage form. In some cases, a kit comprises a sterile container which can contain a therapeutic composition of liposomes; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

### EXAMPLES

### Example 1: Liposome Preparation

To generate the mucus penetrating particles (MPP), DOGS (dioctadecylamidoglycylspermine)/DOPE (dioleoylphosphatidylethanolamine)/PEG2000 can be combined in chloroform at a 80/20/8% ratio. After mixing the lipid solutions in chloroform, the mixture can be dried, first by a stream of nitrogen and then in a vacuum for 12 hours. The appropriate amount of sterile, high resistivity (18.2MΩcm) water can be used to achieve a final concentration of 1 mM of lipid. The resulting mixture can be incubated at 37 degrees Celsius for 16 hours to form liposomes. Following the incubation, the liposome solution can be sonicated using a tip sonicator to form small unilamellar vesicles.

Using dynamic light scattering, nanoparticle size can be determined and the ideal near neutral zeta potential, which indicates that the surface can be sufficiently PEGylated, can be measured by laser Doppler anemometry.

### Example 2: Parent Vector Construction and Transfection

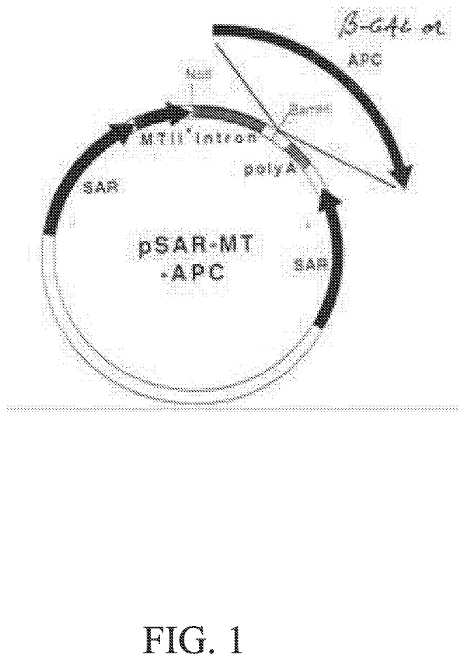

The pSAR-MT inducible expression plasmid can be constructed as follows. Two oligonucleotides (5′-GATCTCGAGCTCCCTGCA-3′ and 5′-GGGAGCTCGA-3′) can be annealed, and the resulting fragment can be cloned into the BamHI and PstI sites of the scaffold attachment region (SAR)-containing plasmid. The resulting plasmid, pJM7, can consist of two SAR sequences flanking a short polylinker containing restriction sites for Xho1 and PstI. The mutant metallothionein (MT) promoter can be amplified by PCR and cloned into pCEP4 (Invitrogen) linearized with BglII and NotI, yielding pCEP-MT. A SalI-BamHI fragment containing an intron from the globin gene can be then inserted into the XhoI and BamHI sites of pCEP-MT, yielding pCEP-MTi. The MT promoter/intron/poly (A) region can be removed from pCEP-MTi by digestion with Sal1 and cloned into the XhoI site of pJM7, yielding pSAR-MT, as shown in FIG. 1.

### Example 3: NTC9385R-APC Vector Construction

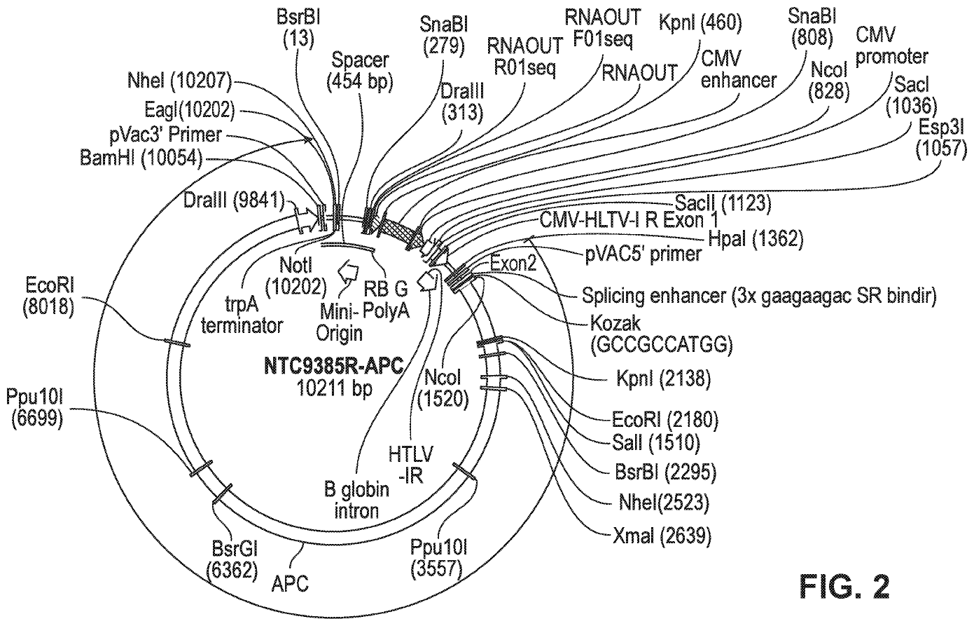

A PstI BamHI compatible vector can be generated by cloning the following oligos into SalI/BglII digested NTC9385R parent vector, “TCGACGCCGCCATGGCTGCAGAAAAAAGGATCCA” and “GATCTGGATCCTTTTTTCTGCAGCCATGGCGGCG.” Subsequently, APC fragments can be cloned into the vector (PstI-BamHI, as PstI-BglII (1537 bp) and BglI BamHI (6987 bp). The vector is shown in FIG. 2.

### Example 4: Liposome Transfection

APC-Liposome complexes can be formed by diluting minicircle-DNA, encoding for APC, and the liposome solution at a charge ratio of 4 to 1 and incubated for 6 hours. The cationic lipids spontaneously associate with the mini-circle DNA.

### Example 5: Dynamic Light Scattering and Zeta Potential

The size and effective charge measurement of APC-PEG2K nanoparticles can be measured using a Malvern Nanosizer ZS (Malvern Instruments). The nanoparticles can be prepared in light-scattering vials at a charge ratio of 10 suspended in 1 mL of the appropriate buffer and incubated at room temperature for 20 minutes. Dynamic light scattering can be performed in high resistivity water. Plots show the z-average diameter. The data points for dynamic light scatting and zeta potential are the average of two measurements performed on the same sample.

### Example 6: In Vitro Transfection of Caco-2 Cells

Human Colorectal adenocarcinoma, Caco-2 cells (ATCC number: HTB-37) can be cultured in ATCC-formulated Eagle's Minimum Essential Medium supplemented with 20% fetal bovine serum (HyClone) and 1% Penicillin/Streptomyocin (Invitrogen). Cells can be kept at 37° C. in a humidified atmosphere containing 5% CO2 and can be reseeded every 72 h to maintain subconfluency. For transfection studies, cells can be seeded in 24 well-plates such that confluency at transfection can be 60-80%. APC-Luciferase-DNA nanostructures can be formed by diluting 1 μg of DNA and the appropriate amount of liposome solution to 250 μL each with Optimem (Invitrogen) and mixing. Nanostructures can be incubated for 20 minutes at room temperature before addition to cells. Cells can be subsequently washed once with PBS and then incubated with 200 μL of complex suspension (0.4 μg of DNA per well) for 6 h. After 6 h, the transfection medium can be removed, and the cells can be rinsed once with PBS and then incubated in supplemented DMEM for 18 h. Cells can be harvested in 150 μL of Passive Lysis Buffer (Promega) and subjected to one freeze-thaw cycle. Luciferase expression can be measured using a Perkin-Elmer 1420 Victor3 V multilabel counter following the assay manufacturer's (Promega) instructions. TE results can be normalized to total cellular protein as measured by Bradford Assay (Bio-Rad). Data points represent an average of two measurements with error bars showing the standard deviation.

### Example 7: In Vivo Murine Model

A murine study can be performed in the C57BL/6J-Apc^{Min}/J (The Jackson Laboratory) mutant mouse model to determine if the APC vector (Table 3) may be capable of rescuing the disease. The APC gene in this mouse model maintains 90% homology with the human gene and results in over 100 intestinal polyps. A C57BL/6J-Apc^{Min}/J mouse undergoes gavage trans-orally with one cohort receiving the APC nanostructures and one cohort receiving GFP negative control nanostructures. The nanostructures can be given weekly for a total of 6 weeks. Mice can be subsequently sacrificed and polyp count can be performed and used to assess tumor regression. Immunohistochemistry can also be performed to determine APC expression in LGR5⁺ cells. Immunohistochemistry can be used to determine if there is an increase in the CD3⁺ or CD11b⁺ cell populations to measure immune responses to either the nanostructure or APC protein.

TABLE 3
APC minicircle vector sequence

| SEQ | |
|---|---|
| ID No. | APC Vector Sequence 5′ to 3′ |
| 765 | ccgcctaatgagcgggcttttttttggcttgttgtccacaaccgttaaaccttaaaagctttaaaagccttatatattcttttttttcttataaaa |
| | cttaaaaccttagaggctatttaagttgctgatttatattaattttattgttcaaacatgagagcttagtacgtgaaacatgagagcttagtacgt |
| | tagccatgagagcttagtacgttagccatgagggtttagttcgttaaacatgagagcttagtacgttaaacatgagagcttagtacgtactatcaa |
| | caggttgaactgctgatccacgttgtggtagaattggtaaagagagtcgtgtaaaatatcgagttcgcacatcttgttgtctgattattgattttt |
| | ggcgaaaccatttgatcatatgacaagatgtgtatctaccttaacttaatgattttgataaaaatcattaggtaccccggctctagttattaatag |
| | taatcaattacggggtcattagttcatagcccatatatggagttccgcgttacataacttacggtaaatggcccgcctggctgaccgcccaacgac |
| | ccccgcccattgacgtcaataatgacgtatgttcccatagtaacgccaatagggactttccattgacgtcaatgggtggagtatttacggtaaact |
| | gcccacttggcagtacatcaagtgtatcatatgccaagtacgccccctattgacgtcaatgacggtaaatggcccgcctggcattatgcccagtac |
| | atgaccttatgggactttcctacttggcagtacatctacgtattagtcatcgctattaccatggtgatgcggttttggcagtacatcaatgggcgt |
| | ggatagcggtttgactcacggggatttccaagtctccaccccattgacgtcaatgggagtttgttttggcaccaaaatcaacgggactttccaaaa |
| | tgtcgtaacaactccgccccattgacgcaaatgggcggtaggcgtgtacggtgggaggtctatataagcagagctcgtttagtgaaccgtcagatc |
| | gcctggagacgccatccacgctgttttgacctccatagaagacaccgggaccgatccagcctccgcggctcgcatctctccttcacgcgcccgccg |
| | ccctacctgaggccgccatccacgccggttgagtcgcgttctgccgcctcccgcctgtggtgcctcctgaactgcgtccgccgtctaggtaagttt |
| | aaagctcaggtcgagaccgggcctttgtccggcgctcccttggagcctacctagactcagccggctctccacgctttgcctgaccctgcttgctca |
| | actctagttctctcgttaacttaatgagacagatagaaactggtcttgtagaaacagagtagtcgcctgcttttctgccaggtgctgacttctctc |
| | ccctgggcttttttctttttctcaggttgaaaagaagaagacgaagaagacgaagaagacaaaccgtcgtcgacgccgccatggctgcagcttcat |
| | atgatcagttgttaaagcaagttgaggcactgaagatggagaactcaaatcttcgacaagagctagaagataattccaatcatcttacaaaactgg |
| | aaactgaggcatctaatatgaaggaagtacttaaacaactacaaggaagtattgaagatgaagctatggcttcttctggacagattgatttattag |
| | agcgtcttaaagagcttaacttagatagcagtaatttccctggagtaaaactgcggtcaaaaatgtccctccgttcttatggaagccgggaaggat |
| | ctgtatcaagccgttctggagagtgcagtcctgttcctatgggttcatttccaagaagagggtttgtaaatggaagcagagaaagtactggatatt |
| | tagaagaacttgagaaagagaggtcattgcttcttgctgatcttgacaaagaagaaaaggaaaaagactggtattacgctcaacttcagaatctca |
| | ctaaaagaatagatagtcttcctttaactgaaaatttttccttacaaacagatatgaccagaaggcaattggaatatgaagcaaggcaaatcagag |
| | ttgcgatggaagaacaactaggtacctgccaggatatggaaaaacgagcacagcgaagaatagccagaattcagcaaatcgaaaaggacatacttc |
| | gtatacgacagcttttacagtcccaagcaacagaagcagagaggtcatctcagaacaagcatgaaaccggctcacatgatgctgagcggcagaatg |
| | aaggtcaaggagtgggagaaatcaacatggcaacttctggtaatggtcagggttcaactacacgaatggaccatgaaacagccagtgttttgagtt |
| | ctagtagcacacactctgcacctcgaaggctgacaagtcatctgggaaccaaggtggaaatggtgtattcattgttgtcaatgcttggtactcatg |
| | ataaggatgatatgtcgcgaactttgctagctatgtctagctcccaagacagctgtatatccatgcgacagtctggatgtcttcctctcctcatcc |
| | agcttttacatggcaatgacaaagactctgtattgttgggaaattcccggggcagtaaagaggctcgggccagggccagtgcagcactccacaaca |
| | tcattcactcacagcctgatgacaagagaggcaggcgtgaaatccgagtccttcatcttttggaacagatacgcgcttactgtgaaacctgttggg |
| | agtggcaggaagctcatgaaccaggcatggaccaggacaaaaatccaatgccagctcctgttgaacatcagatctgtcctgctgtgtgtgttctaa |
| | tgaaactttcatttgatgaagagcatagacatgcaatgaatgaactagggggactacaggccattgcagaattattgcaagtggactgtgaaatgt |
| | atgggcttactaatgaccactacagtattacactaagacgatatgctggaatggctttgacaaacttgacttttggagatgtagccaacaaggcta |
| | cgctatgctctatgaaaggctgcatgagagcacttgtggcccaactaaaatctgaaagtgaagacttacagcaggttattgcgagtgttttgagga |
| | atttgtcttggcgagcagatgtaaatagtaaaaagacgttgcgagaagttggaagtgtgaaagcattgatggaatgtgctttagaagttaaaaagg |
| | aatcaaccctcaaaagcgtattgagtgccttatggaatttgtcagcacattgcactgagaataaagctgatatatgtgctgtagatggtgcacttg |
| | catttttggttggcactcttacttaccggagccagacaaacactttagccattattgaaagtggaggtgggatattacggaatgtgtccagcttga |
| | tagctacaaatgaggaccacaggcaaatcctaagagagaacaactgtctacaaactttattacaacacttaaaatctcatagtttgacaatagtca |
| | gtaatgcatgtggaactttgtggaatctctcagcaagaaatcctaaagaccaggaagcattatgggacatgggggcagttagcatgctcaagaacc |
| | tcattcattcaaagcacaaaatgattgctatgggaagtgctgcagctttaaggaatctcatggcaaataggcctgcgaagtacaaggatgccaata |
| | ttatgtctcctggctcaagcttgccatctcttcatgttaggaaacaaaaagccctagaagcagaattagatgctcagcacttatcagaaacttttg |
| | acaatatagacaatttaagtcccaaggcatctcatcgtagtaagcagagacacaagcaaagtctctatggtgattatgtttttgacaccaatcgac |
| | atgatgataataggtcagacaattttaatactggcaacatgactgtcctttcaccatatttgaatactacagtgttacccagctcctcttcatcaa |
| | gaggaagcttagatagttctcgttctgaaaaagatagaagtttggagagagaacgcggaattggtctaggcaactaccatccagcaacagaaaatc |
| | caggaacttcttcaaagcgaggtttgcagatctccaccactgcagcccagattgccaaagtcatggaagaagtgtcagccattcatacctctcagg |
| | aagacagaagttctgggtctaccactgaattacattgtgtgacagatgagagaaatgcacttagaagaagctctgctgcccatacacattcaaaca |
| | cttacaatttcactaagtcggaaaattcaaataggacatgttctatgccttatgccaaattagaatacaagagatcttcaaatgatagtttaaata |
| | gtgtcagtagtagtgatggttatggtaaaagaggtcaaatgaaaccctcgattgaatcctattctgaagatgatgaaagtaagttttcagttatgg |
| | tcaatacccagccgacctagcccataaaatacatagtgcaaatcatatggatgataatgatggagaactagatacaccaataaattatagtcttaa |
| | atattcagatgagcagttgaactctggaaggcaaagtccttcacagaatgaaagatgggcaagacccaaacacataatagaagatgaaataaaaca |
| | aagtgagcaaagacaatcaaggaatcaaagtacaacttatcctgtttatactgagagcactgatgataaacacctcaagttccaaccacattttgg |
| | acagcaggaatgtgtttctccatacaggtcacggggagccaatggttcagaaacaaatcgagtgggttctaatcatggaattaatcaaaatgtaag |
| | ccagtctttgtgtcaagaagatgactatgaagatgataagcctaccaattatagtgaacgttactctgaagaagaacagcatgaagaagaagagag |
| | accaacaaattatagcataaaatataatgaagagaaacgtcatgtggatcagcctattgattatagtttaaaatatgccacagatattccttcatc |
| | acagaaacagtcattttcattctcaaagagttcatctggacaaagcagtaaaaccgaacatatgtcttcaagcagtgagaatacgtccacaccttc |
| | atctaatgccaagaggcagaatcagctccatccaagttctgcacagagtagaagtggtcagcctcaaaaggctgccacttgcaaagtttcttctat |
| | taaccaagaaacaatacagacttattgtgtagaagatactccaatatgtttttcaagatgtagttcattatcatctttgtcatcagctgaagatga |
| | aataggatgtaatcagacgacacaggaagcagattctgctaataccctgcaaatagcagaaataaaagaaaagattggaactaggtcagctgaaga |
| | tcctgtgagcgaagttccagcagtgtcacagcaccctagaaccaaatccagcagactgcagggttctagtttatcttcagaatcagccaggcacaa |
| | agctgttgaattttcttcaggagcgaaatctccctccaaaagtggtgctcagacacccaaaagtccacctgaacactatgttcaggagaccccact |
| | catgtttagcagatgtacttctgtcagttcacttgatagttttgagagtcgttcgattgccagctccgttcagagtgaaccatgcagtggaatggt |
| | aagtggcattataagccccagtgatcttccagatagccctggacaaaccatgccaccaagcagaagtaaaacacctccaccacctcctcaaacagc |
| | tcaaaccaagcgagaagtacctaaaaataaagcacctactgctgaaaagagagagagtggacctaagcaagctgcagtaaatgctgcagttcagag |
| | ggtccaggttcttccagatgctgatactttattacattttgccacggaaagtactccagatggattttcttgttcatccagcctgagtgctctgag |
| | cctcgatgagccatttatacagaaagatgtggaattaagaataatgcctccagttcaggaaaatgacaatgggaatgaaacagaatcagagcagcc |
| | taaagaatcaaatgaaaaccaagagaaagaggcagaaaaaactattgattctgaaaaggacctattagatgattcagatgatgatgatattgaaat |
| | actagaagaatgtattatttctgccatgccaacaaagtcatcacgtaaagcaaaaaagccagcccagactgcttcaaaattacctccacctgtggc |
| | aaggaaaccaagtcagctgcctgtgtacaaacttctaccatcacaaaacaggttgcaaccccaaaagcatgttagttttacaccgggggatgatat |
| | gccacgggtgtattgtgttgaagggacacctataaacttttccacagctacatctctaagtgatctaacaatcgaatcccctccaaatgagttagc |
| | tgctggagaaggagttagaggaggggcacagtcaggtgaatttgaaaaacgagataccattcctacagaaggcagaagtacagatgaggctcaagg |
| | aggaaaaacctcatctgtaaccatacctgaattggatgacaataaagcagaggaaggtgatattcttgcagaatgcattaattctgctatgcccaa |
| | agggaaaagtcacaagcctttccgtgtgaaaaagataatggaccaggtccagcaagcatctgcgtcttcttctgcacccaacaaaaatcagttaga |
| | tggtaagaaaaagaaaccaacttcaccagtaaaacctataccacaaaatactgaatataggacacgtgtaagaaaaaatgcagactcaaaaaataa |
| | tttaaatgctgagagagttttctcagacaacaaagattcaaagaaacagaatttgaaaaataattccaaggtcttcaatgataagctcccaaataa |
| | tgaagatagagtcagaggaagttttgcttttgattcacctcatcattacacgcctattgaaggaactccttactgtttttcacgaaatgattcttt |
| | gagttctctagattttgatgatgatgatgttgacctttccagggaaaaggctgaattaagaaaggcaaaagaaaataaggaatcagaggctaaagt |
| | taccagccacacagaactaacctccaaccaacaatcagctaataagacacaagctattgcaaagcagccaataaatcgaggtcagcctaaacccat |
| | acttcagaaacaatccacttttccccagtcatccaaagacataccagacagaggggcagcaactgatgaaaagttacagaattttgctattgaaaa |
| | tactccggtttgcttttctcataattcctctctgagttctctcagtgacattgaccaagaaaacaacaataaagaaaatgaacctatcaaagagac |
| | tgagccccctgactcacagggagaaccaagtaaacctcaagcatcaggctatgctcctaaatcatttcatgttgaagataccccagtttgtttctc |
| | aagaaacagttctctcagttctcttagtattgactctgaagatgacctgttgcaggaatgtataagctccgcaatgccaaaaaagaaaaagccttc |
| | aagactcaagggtgataatgaaaaacatagtcccagaaatatgggtggcatattaggtgaagatctgacacttgatttgaaagatatacagagacc |
| | agattcagaacatggtctatcccctgattcagaaaattttgattggaaagctattcaggaaggtgcaaattccatagtaagtagtttacatcaagc |
| | tgctgctgctgcatgtttatctagacaagcttcgtctgattcagattccatcattccctgaaatcaggaatctctctgggatcaccatttcatctt |
| | acacctgatcaagaagaaaaaccctttacaagtaataaaggcccacgaattctaaaaccaggggagaaaagtacattggaaactaaaaagatagaa |
| | tctgaaagtaaaggaatcaaaggaggaaaaaaagtttataaaagtttgattactggaaaagttcgatctaattcagaaatttcaggccaaatgaaa |
| | cagccccttcaagcaaacatgccttcaatctctcgaggcaggacaatgattcatattccaggagttcgaaatagctcctcaagtacaagtcctgtt |
| | tctaaaaaaggcccaccccttaagactccagcctccaaaagccctagtgaaggtcaaacagccaccacttctcctagaggagccaagccatctgtg |
| | aaatcagaattaagccctgttgccaggcagacatcccaaataggtgggtcaagtaaagcaccttctagatcaggatctagagattcgaccccttca |
| | agacctgcccagcaaccattaagtagacctatacagtctcctggccgaaactcaatttcccctggtagaaatggaataagtcctcctaacaaatta |
| | tctcaacttccaaggacatcatcccctagtactgcttcaactaagtcctcaggttctggaaaaatgtcatatacatctccaggtagacagatgagc |
| | caacagaaccttaccaaacaaacaggtttatccaagaatgccagtagtattccaagaagtgagtctgcctccaaaggactaaatcagatgaataat |
| | ggtaatggagccaataaaaaggtagaactttctagaatgtcttcaactaaatcaagtggaagtgaatctgatagatcagaaagacctgtattagta |
| | cgccagtcaactttcatcaaagaagctccaagcccaaccttaagaagaaaattggaggaatctgcttcatttgaatctctttctccatcatctaga |
| | ccagcttctcccactaggtcccaggcacaaactccagttttaagtccttcccttcctgatatgtctctatccacacattcgtctgttcaggctggt |
| | ggatggcgaaaactcccacctaatctcagtcccactatagagtataatgatggaagaccagcaaagcgccatgatattgcacggtctcattctgaa |
| | agtccttctagacttccaatcaataggtcaggaacctggaaacgtgagcacagcaaacattcatcatcccttcctcgagtaagcacttggagaaga |
| | actggaagttcatcttcaattctttctgcttcatcagaatccagtgaaaaagcaaaaagtgaggatgaaaaacatgtgaactctatttcaggaacc |
| | aaacaaagtaaagaaaaccaagtatccgcaaaaggaacatggagaaaaataaaagaaaatgaattttctcccacaaatagtacttctcagaccgtt |
| | tcctcaggtgctacaaatggtgctgaatcaaagactctaatttatcaaatggcacctgctgtttctaaaacagaggatgtttgggtgagaattgag |
| | gactgtcccattaacaatcctagatctggaagatctcccacaggtaatactcccccggtgattgacagtgtttcagaaaaggcaaatccaaacatt |
| | aaagattcaaaagataatcaggcaaaacaaaatgtgggtaatggcagtgttcccatgcgtaccgtgggtttggaaaatcgcctgaactcctttatt |
| | caggtggatgcccctgaccaaaaaggaactgagataaaaccaggacaaaataatcctgtccctgtatcagagactaatgaaagttctatagtggaa |
| | cgtaccccattcagttctagcagctcaagcaaacacagttcacctagtgggactgttgctgccagagtgactccttttaattacaacccaagccct |
| | aggaaaagcagcgcagatagcacttcagctcggccatctcagatcccaactccagtgaataacaacacaaagaagcgagattccaaaactgacagc |
| | acagaatccagtggaacccaaagtcctaagcgccattctgggtcttaccttgtgacatctgtttaaggatccagatctttttccctctgccaaaaa |
| | ttatggggacatcatgaagccccttgagcatctgacttctggctaataaaggaaatttattttcattgcaatagtgtgttggaattttttgtgtct |
| | ctcactcggaaggacataagggcggccgctagc |

### Example 8: Synthesis of an HPEG2K-Lipid

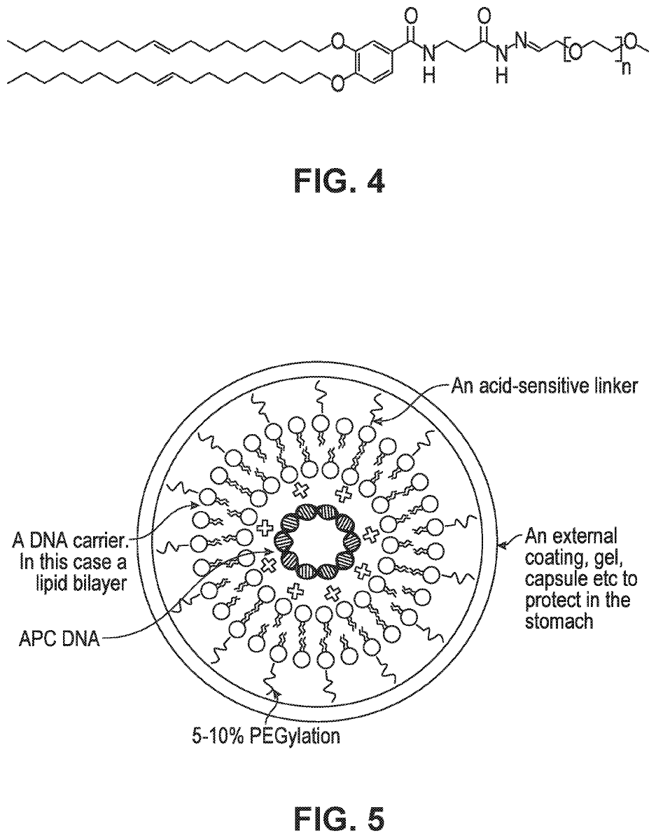

A scheme that can be used to synthesize the acid-labile PEG-lipid is provided below (FIG. 4). The oxidation of PEG can be performed as described in (Masson C, Scherman D, Bessodes M. 2, 2, 6, 6-Tetramethyl-1-piperidinyl-oxyl/[bis (acetoxy)-iodo] benzene-mediated oxidation: a versatile and convenient route to poly (ethylene glycol) aldehyde or carboxylic acid derivatives. J Polym Sci A 2001; 39:4022-4.). Coupling of hydrophobic building block DOB (3, 4-Di (oleyloxy) Benzoic Acid) to β-Alanine ethyl ester proceeds in high yield to yields a product which can be treated with a large excess of hydrazine hydrate in a minimum amount of chloroform/methanol solvent to yield DOB-O-Ala-hydrazide. Monomethylated PEG of MW 2000 (n≈44) can be oxidized to the corresponding aldehyde with (bis (acetoxy)-iodo] benzene and catalytic TEMPO (2, 2, 6, 6-Tetramethyl-1-piperidinyl-oxyl). The two components can be coupled in anhydrous dichloromethane/methanol mixture with added molecular sieves. The resulting PEG-lipid can be purified by preparative thin-layer chromatography.

### Example 9: TLC Analysis of the Stability of HPEG2K-Lipid at Varied pH

Buffer solutions can be prepared at pH 4, 5, 6 (citric acid (0.1 M)/phosphate (0.2 M)) and pH 7.4 (0.1 M HEPES). For each pH tested, a sample can be prepared in a small glass vial by dissolving about 1 mg HPEG2K-lipid in dichloromethane/methanol 1:1 and evaporating the solvent, first under a stream of nitrogen and then in a vacuum for 14 hours. The vial can be warmed to 37° C. for 30 minutes and 100 μl of warm (37° C.) buffer of the desired pH can be added. The resulting mixture can be incubated for 5 minutes, sonicated for 2.5 minutes and incubated further (all incubations can be performed at 37° C.). At designated time points, an aliquot of 10 μl can be removed, mixed with 20 μl of methanol and immediately analyzed by TLC. A Chloroform/methanol/concentration of NH₄OH (100:15:1) can be used as the mobile phase. TLC plates (Merck, silica 60, glass-backed) can be pre-run without sample in the same solvent and used after brief air-drying. Spots can be detected by UV absorption (specific for DOB derivatives in this context), absorption of iodine vapor (unspecific stain), and using a spray reagent (modified Dragendorff reagent) specific for PEG.

### Example 10: Neutral Liposome Complex Preparation

Chloroform solutions of lipids can be mixed and dried to a thin film by rotary evaporation. Residual solvent can be removed under high vacuum for at least 4 hrs. or overnight. Dried lipids can be dispersed with 10 mM Tris buffer, pH 7.4 (TB7.4), to a concentration of 80 mM total lipid in volumes ranging from 1 to 5 ml. Small unilamellar vesicles (SUVs) can be formed by probe sonication (Fisher Scientific). A sonication program (five cycles of 3-min pulsing, 1-min off) and immersion of the sample tube in an ice bath throughout the process can be used to minimize sample heating. Liposomes can be then centrifuged at 12,000 rpm in an Eppendorf 5415C centrifuge for 5 min to remove debris, and filtered through 0.2-μm sterilizing membranes.

Plasmid (pCMV-APC) can be isolated and purified from Escherichia coli. The purity of plasmid preparations can be determined by 1% agarose gel electrophoresis followed by SYBR Green fluorescent staining. DNA concentration can be measured by UV absorption at 260 nm. The percentage of supercoiled pDNA can be in the range of 80-95%, and the OD_{260/280} ratio can be between 1.85 and 1.9. Endotoxin levels of pDNA can be determined using a chromogenic limulus amebocyte lysate assay (LAL BioWhitaker, Walkersville, Md.). Values can be less than 20 EU/mg.

Neutral liposome complexes (NLCs) can be formed by first mixing SUVs (250 μl, 20 μmol), plasmid (0.1 mg), and TB7.4 to give a total volume of 400 μl. To this can be added 600 μl of a given mixture of absolute ethanol, calcium chloride (from a 500 mM stock) and TB7.4. Addition can be performed drop-wise over approximately 30 s with maximum vortex mixing. The resulting aggregated complexes can be dialyzed against 500 vols. of TB7.4 for 24 h with two changes of buffer. For experiments requiring physiological tonicity, samples can be further dialyzed against 500 vols. of PBS for 24 h. For a given lipid composition, plasmid entrapment and particle size can be optimized using central composite experimental designs with two factors (ethanol and calcium concentrations) and four centerpoints. Design and analysis can be performed using Essential Experiment Design, an add-in macro for Microsoft Excel. Factor ranges can be estimated from preliminary trapping experiments, and these can be different for each lipid composition: DOPC, 35-50% ethanol, 0-5 mM Ca²⁺; DOPC/DOPE, 20-40% ethanol, 0-10 mM Ca²⁺; DOPC/DOPE/Chol, 35-45% ethanol, 5-15 mM Ca²⁺. Quadratic models with up to six terms can be fit to the measured entrapment data from each design, but only those terms that contributed significantly to the fit (P<0.05) can be used in predicting optimal formulations. The criteria for optimization can be maximized entrapment and average particle size less than 200 nm. A single design can be required to optimize each lipid formulation, but each can be repeated to validate the resulting model and terms.

### Example 11: Neutral Liposome Complex Size Analysis

Average particle diameters can be determined by quasi-elastic light scattering (QELS) using a BI-9000AT correlator (Brookhaven Instruments, Holtsville, N.Y.). Samples can be diluted to 1.0 mM lipid in water or PBS, as appropriate, to give sufficient scattering intensity with a minimum pinhole (100 μm). Averages of three autocorrelations, each collected over 1 min with a minimum scattering intensity of 5×10⁴ counts/s, can be converted to Gaussian distributions, from which mean diameters and standard deviations can be derived using software supplied by the manufacturer. Polydispersities can be also noted.

### Example 12: Entrapment Assay

The fraction of plasmid entrapped by the complexation procedure can be determined using the fluorescent DNA probe TO-PRO-1. Complexes can be diluted 100-fold into a total 2.0 ml volume with TB7.4, and 1 μl of 1 mM TO-PRO-1 can be added from a DMSO stock solution. Fluorescence can be measured on a Perkin-Elmer LS 50B spectrofluorometer, with excitation and emission wavelengths of 514 and 531 nm, respectively, 2.5 nm slit-widths, and the photo-multiplier voltage set to 900 V. The signal due to scatter prior to the addition of TO-PRO-1 can be set to zero fluorescence. Percent entrapment can be calculated as the fluorescence signal of TO-PRO-1 divided by the fluorescence following the addition of 20 μl of 100 mM Triton X-100 to release DNA from the lipid, correcting for the 1% increase in volume. At the concentrations specified, the neutral lipids and Triton X-100 can be determined to have no effect on TO-PRO-1 fluorescence in the presence of free DNA plasmids. Increasing the concentration of TO-PRO-1 does not increase fluorescence, indicating that it may be present in sufficient excess.

### Example 13: POZylation of Nanoparticles

Alkyne terminated POZ (100 mg) can be added to a 5 mL suspension of fluorescent nanoparticles (19±3 mg mL⁻¹), which had been diluted with 5 mL DMSO prior to the reaction. Following this, 200 μL Triethylamine (TEA) can be added, and the reaction left for 24 hours under constant stirring in the dark.

### Example 14: Assessing the Diffusion of Nanoparticles in Porcine Gastric Mucin Dispersions Using NTA

All diffusion measurements can be carried out using the Nano-Sight LM10, with LM14 top-plate and syringe pump. Fluorescent nanoparticles can be diluted down by a factor of 10,000 in deionized water. 10 μL of this dilution can be then added to a 990 μL suspension of 1% w/v gastric mucus, forming a final dilution of 1:1,000,000. Samples can be injected into the NanoSight system and the flow-rate can be set at 70 AU in order to minimize fluorescent bleaching of the nanoparticles during analysis. All videos can be recorded through a long pass filter, with a wavelength cut-on of 550 nm (Thorlabs, UK). 6×60 second videos can be recorded at 25 and 37° C. Each independent stock dispersion of mucin can be analyzed three times with each nanoparticle type, resulting in a total of 9×660 second videos for each temperature, with a viscosity of 25 cP at 25° C. and 28 cP at 37° C. (as determined from rheological analyses).

### Example 15: Particle-Tracking Analysis

Movies of particles penetrating mucous can be analyzed using automated particle-tracking software custom-written in MATLAB (MathWorks, Natick, Mass.). To determine the x and y positions of particles over time. Images can be first processed by convolving them with a spatial bandpass filter to reduce noise and non-uniform background. Local maxima of pixel intensity can be identified as candidate particle positions. These positions can be refined by calculating the intensity-weighted centroid of the bright spots, to yield subpixel resolution. By examining particle brightness, size, and eccentricity, true particles can be retained and spurious ones (noise) discarded. Trajectories can be constructed by linking particle positions identified in subsequent frames via a nearest neighbor method. Trajectories shorter than 1 second can be discarded. The time-averaged mean squared displacement (MSD) of each trajectory can be calculated as MSD (τ)= [symbol] [x(t+τ)−x(t)]² [symbol] + [symbol] [y(t+τ)−y(t)]² [symbol] , where r can be the time scale and the angled brackets denote the average over many starting times t.

Tracking resolution can be estimated by first calculating the signal-to-noise ratio from the experimental movies. These can be compared with a standard curve of static error as a function of signal-to-noise ratio, to estimate the static error in the experimental movies. The standard curve can be generated by affixing particles to a glass slide and tracking them under different illumination intensities; the apparent motion of these fixed particles can be due to static error.

### Example 16: Precursor Synthesis

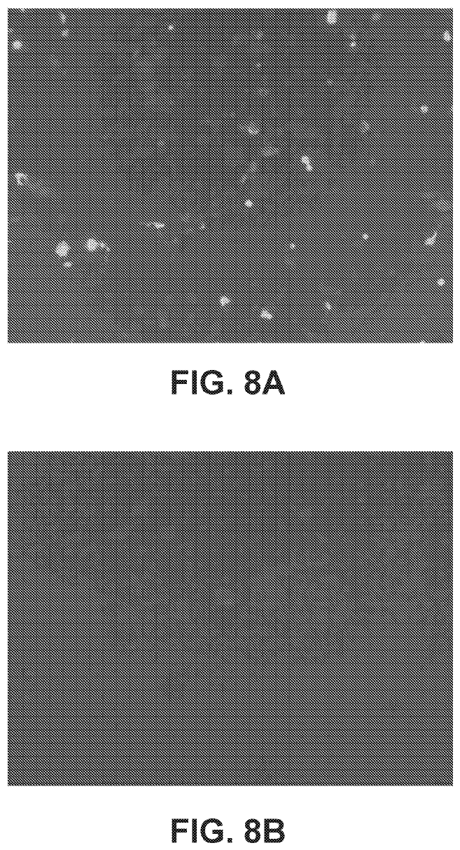

An acylhydrazone-based PEG-2000 lipid (HPEG2K-Lipid) can be synthesized. Briefly, 3, 4-Di (oleyloxy) Benzoic acid (DOB) can be synthesized by reacting oleyl bromide (in excess) and protocatechuic acid ethyl ester (limited) in cyclohexanone in the presence of potassium carbonate and potassium iodide and stirred at 100 C for under nitrogen. The reaction mixture can be filtered and the residue dissolved and refluxed in ethanol containing potassium hydroxide. Acidifying the reaction mixture resulted in a white precipitate (DOB) which can be collected as residue upon filtering. DOB can be coupled to B-alanine ethyl ester in the presence of O-(Benzotriazol-1-yl)-N,N,N′,N′-tetramethyluronium tetrafluoroborate (TBTU) and N,N-Diisopropylethylamine (DIEA). Hydrazine hydrate in chloroform/methanol solvent can be added to the product resulting in DOB-B-Ala-Hydrazide. DOB-B-Ala-Hydrazide can be coupled with oxidized monomethylated PEG (MW: 2000) in anhydrous dichloromethane/methanol mixture in the presence of molecular sieves. Product can be purified using preparative TLC (yield of ˜50% from DOB coupling) and its acid-sensitive properties confirmed using TLC after incubation of the product at pH 4 and neutral pH. Data shows in FIG. 7.

### Example 17: Nanoparticle Formulation

Liposomes were formed with the composition MVL5 (Avanti Polar Lipids)/Glyceryl Mono-Oleate (MP Biomedicals LLC)/HPEG-2K-LIPD at a ratio of 50/43/7% mol. Individual lipid stocks were made in CHCl3/MeOH (9:1) and appreciate volumes of the stocks were mixed according to the composition in a small round-bottom flask. Lipid thin-film can be made by rota-vap and further dried under vacuum for 5 hours. The lipids were then hydrated with 5 ml Milli-Q water by incubation on shaker at 37 deg C. for 4-5 hours. The liposornes were then sonicated multiple times using a small probe sonicator for 5-10 min intervals. Particle size can be monitored at the end of each sonication. The particle size can be 150 nm after 10-15 min of sonication. The liposome can be then diluted to 1 mM and filtered through a 0.45 um filter in a BSC hood. Liposome-DNA complexes were formulated at a charge ratio of 5 with MVL5 assumed to have full protonation (i.e headgroup charge+5e). 20 uL of 1 μg/mL DNA can be diluted to 1 mL and added into a 1 mL of 0.125 mM (total lipid) solution and mixed promptly by pipetting up-down. The particle size and zeta potential were then measured after 10-20 min incubation using a Brookhaven ZetaPALS particle sizer. Decrease in both particle size and Zeta potential values were observed for the DNA complexes compared to the starting liposomes as shown in Table 4.

TABLE 4
Nanoparticle size and zeta potential

| | Liposomes alone | Liposomes + DNA |
|---|---|---|
| Particle Size | 134.7 ± 2.2 nm | 82.0 ± 1.9 nm |
| Zeta Potential (Milli-Q | 31.8 ± 3.1 mV | 8.3 ± 1.3 mV |
| water) | | |

### Example 18: In-Vitro Transfection

In-vitro transfection studies were carried out using Caco-2 and HT29 cells with an eGFP expression plasmid (NTC9385-eGFP vector) driven by a cytomegalovirus (CMV) promoter. The NTC9385 plasmid can be designed to have a reduced bacterial backbone and antibiotic free selection (to not confer antibiotic resistance to the gut bacteria).

### Liposome-DNA Formulation

The liposome-DNA can be formulated at either a +5 or +10 charge ratios (assuming a +5 charge on MVL5). A stock of 1 mM of MVL5/GMO/HPEG liposome can be added to the plasmid diluted in OPTI-MEM media, mixed via pipetting, left to couple at room temperature for 20 minutes, and then applied to cells.

### Transfecting

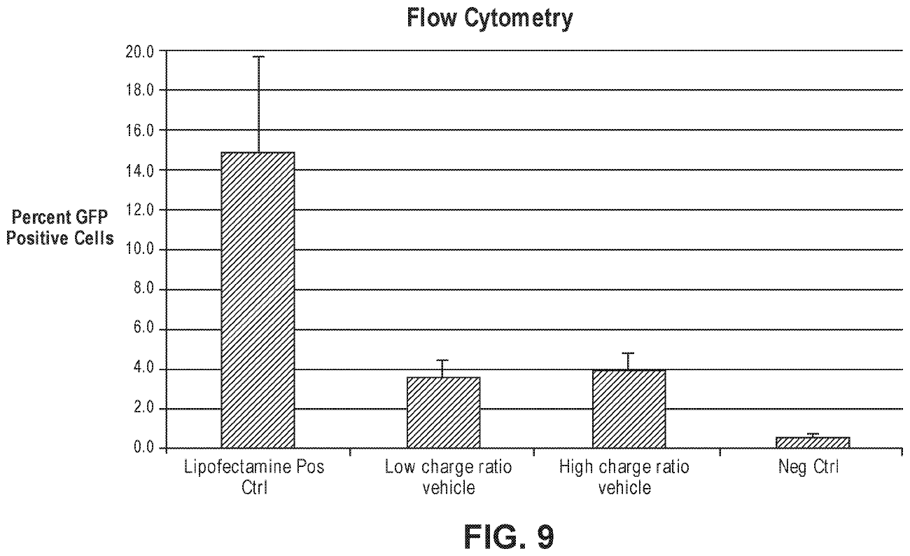

DNA concentrations ranging from 100 ng to 2 ug were transfected into 12-well plates with cells at 80% confluence. The plates were then imaged using a Zeiss AxioObserver epi-fluorescent microscope 48 hours after transfection for fluorescence, FIG. 8A and FIG. 8B.

### Flow Cytometry

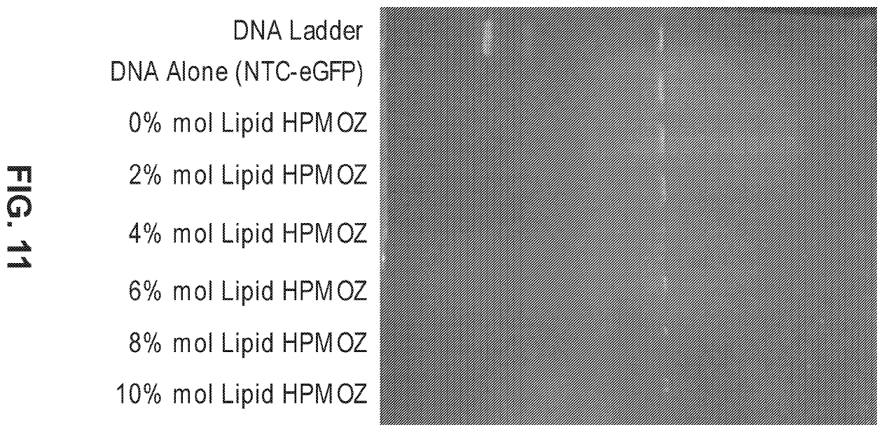

Following 48 hours of transfection, ˜4×10⁵ cells were washed once with PBS and then cells were removed from each well in 500 uL of PBS. Samples were assayed for fluorescence using a Millipore Guava HT6 Benchtop Flow Cytometer, utilizing a laser excitation wavelength of 488 nm. For each assay sample, 10,000 events were collected. A negative control, with cells only, can be used to set gating thresholds and a positive lipofectamine control can be also evaluated, FIG. 10.

### Example 19: PMOZ Formulation

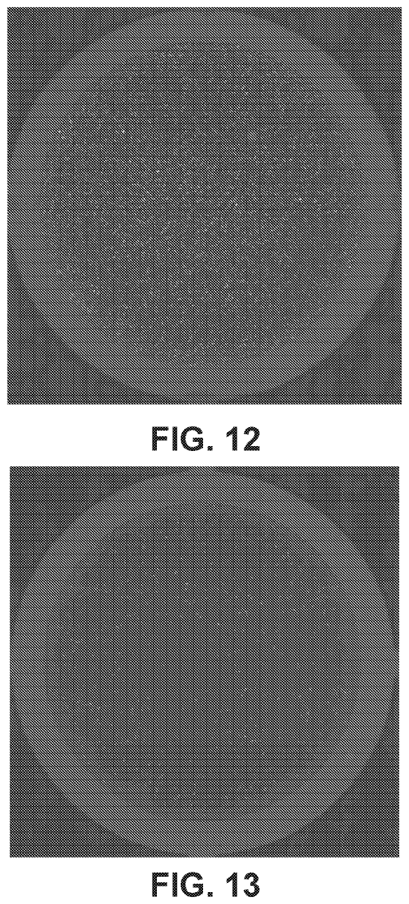

MVL5/GMO/lipid-HPMOZ were formulated at different ratios (50/50−x/x % mol). Briefly, the solutions of each lipid in Chloroform: Methanol (9:1) were mixed together in the appropriate ratios. Thin-film hydration method was used by vacuuming the solution till a thin-film formed and Milli-Q water added to suspend the lipids. Lipids were then shaken overnight at room temperature. Lipids were, then, sonicated for 10-15 minutes with 30 second breaks using a probe sonicator. Lipids were found to complex with DNA with almost 100% efficiency (FIG. 2). Briefly, lipids were added to NTC-eGFP DNA at a charge ratio of 5 (assuming MVL5 has +5 charge) and mixed by pipetting up and down. Complexes were left to rest for 20 minutes at room temperature. DNA was pre-coupled to Ethidium Bromide at a final concentration of 0.5 μg/ml. Complexes were loaded onto a 1% agarose gel and run at 80 mV (FIG. 11).

### Example 20: PMOZ Transfection

On day one, 24-well plates were seeded with Caco-2 cells to 80% confluence. After 24 hours, 6 μL each of the samples below were coupled with 1 μg of the NTC9385R-GFP plasmid each, gently pipetted to mix, and allowed to sit at room temperature for 15 min before being applied to cells. This was performed in triplicate for each sample. After 48 hours, the wells were imaged for GFP expression with a Keyence BZ-X700 at 4× magnification. Using image J the average pixel intensity of each well was determined and plotted. PMOZ 4% demonstrated the strongest GFP expression of the PMOZ samples tested.

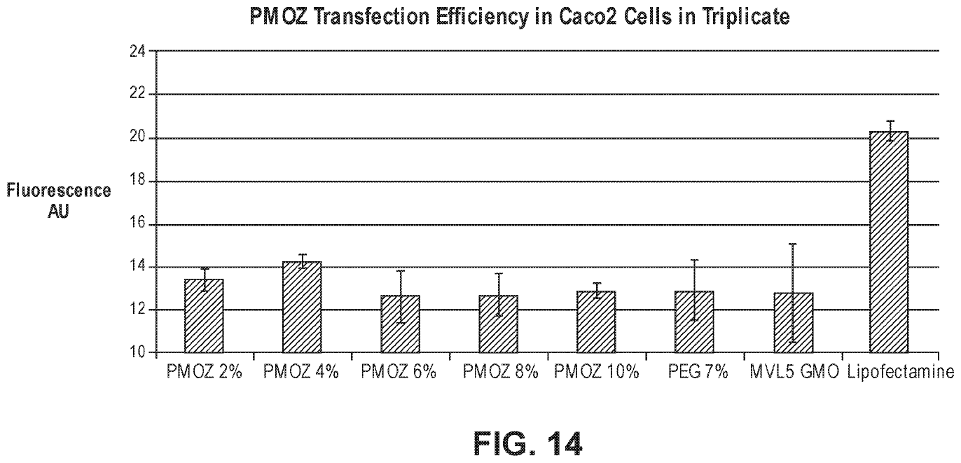

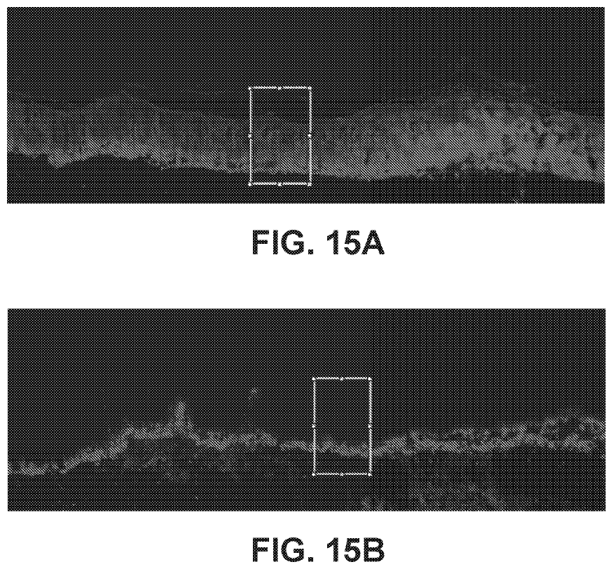

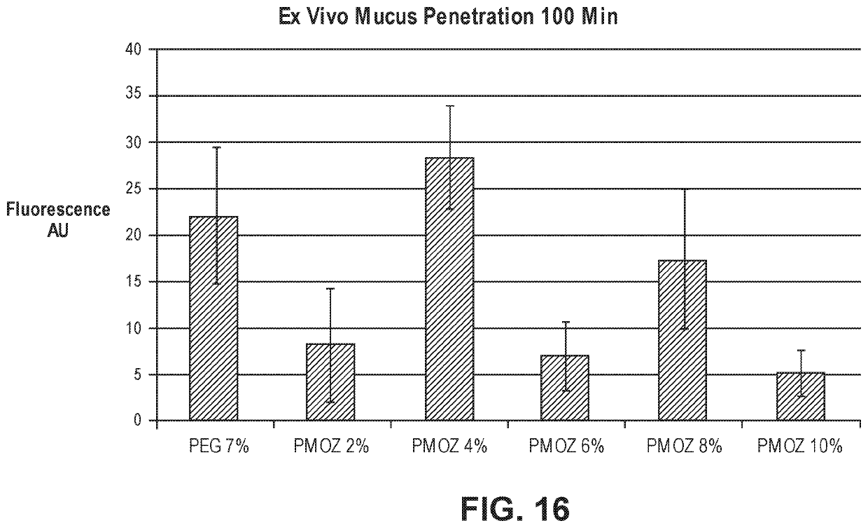

Samples: (a) MVL5/GMO/Lipid-HPMOZ (50:50−x:x). PMOZ was analyzed between 2-10%, FIG. 13 and FIG. 14. (b) MVL5/GMO/Lipid-HPEG (50:43:7) (c) MVL5:GMO (d) Lipofectamine2000 control, FIG. 12.

### Example 21: Ex Vivo Mucus Penetration

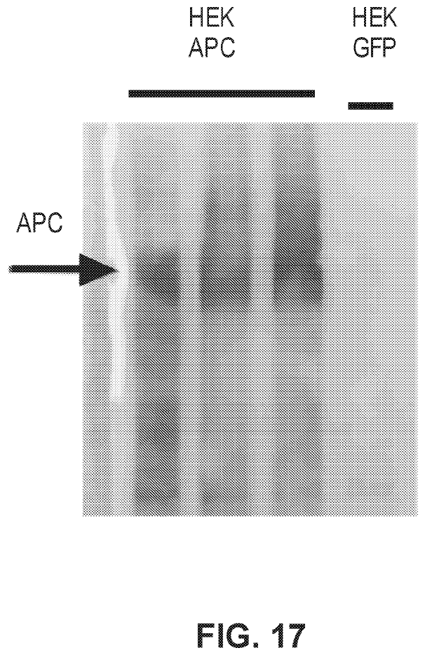

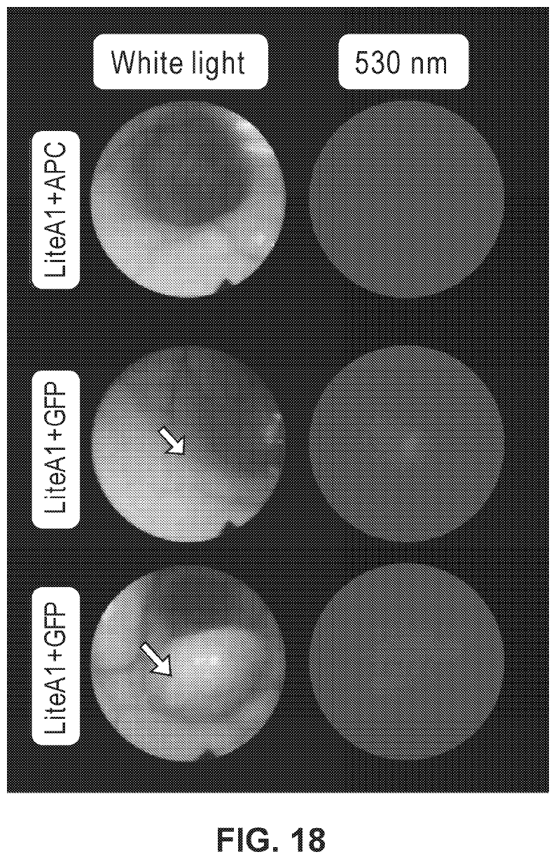

Fresh porcine colons obtained from a local abattoir was collected and kept on ice. The colon was cut longitudinally and then laid flat. Sections 2 mm×4 mm in size were cut and placed into a 6-well plate with the mucus layer facing up. The day before, 50 uL of 1 mM vehicle (MVL5, PMOZ 2%, PMOZ 4%, PMOZ 6%, PMOZ 8%, or PMOZ 10%) was coupled with 8 μg of a Cy5 labeled 60 bp oligonucleotide for 30 min and then stored at 4 C overnight. These vehicles were then applied directly to the mucus layer and incubated at 37 C with 5% CO2 for 100 min. This was performed in triplicate for each sample. The tissue was embedded in OCT media and frozen in a dry ice/ethanol slurry. The samples were then cryosectioned into 30 μm sections and placed on glass slides. The slides were then imaged with a Keyence BZ-X700 with ⅓ s exposure at 4× magnification. ImageJ was then used to analyze the images and the average pixel intensity for a 160×260 pixel rectangle placed directly over the epithelial layer was used to determine the relative level of dye that penetrates through after 100 min. PMOZ 4% was found to have the greatest mucus penetrance, FIG. 15A, FIG. 15B, and FIG. 16.

### Example 22: Pirc Rat Colorectal Cancer Model

The F344-Apc^{am1137/+} PIRC rat (4-7 months old) was used as an animal model of Familial Adenomatous Polyposis (FAP). The therapeutic transgene that was delivered was a wild-type copy of the human Adenomatous Polyposis Coli (APC) with GFP given as a control, both were driven by a CMV promoter. Three different cohorts with 5 animals (3 females and 2 males) in each were tested. Cohort 1 was treated with Lipofectamine2000+APC, Cohort 2 was treated with LiteA1+APC, Cohort 3 was treated with LiteA1+GFP to serve as a negative control as well as allow for vehicle localization. Each animal was dosed intrarectally with 30 μg of DNA three times a week for a total of 7 weeks. The delivery vehicle and DNA were coupled as follows immediately before administration. For the LiteA1 vehicle, 30 μL of 1 μg/L DNA (either CMV-APC or CMV-GFP) was coupled with 187.5 μL of 1 mM LiteA1 with OPTI-MEM up to a volume of 750 μL. For the Lipofectamine group, 30 μL of 1 μg/L DNA (CMV-APC) was coupled with 30 μL of Lipofectamine 2000. Once the components were added, the tube was gently inverted 20 times and the coupling was allowed to take place for 30 min at room temperature.

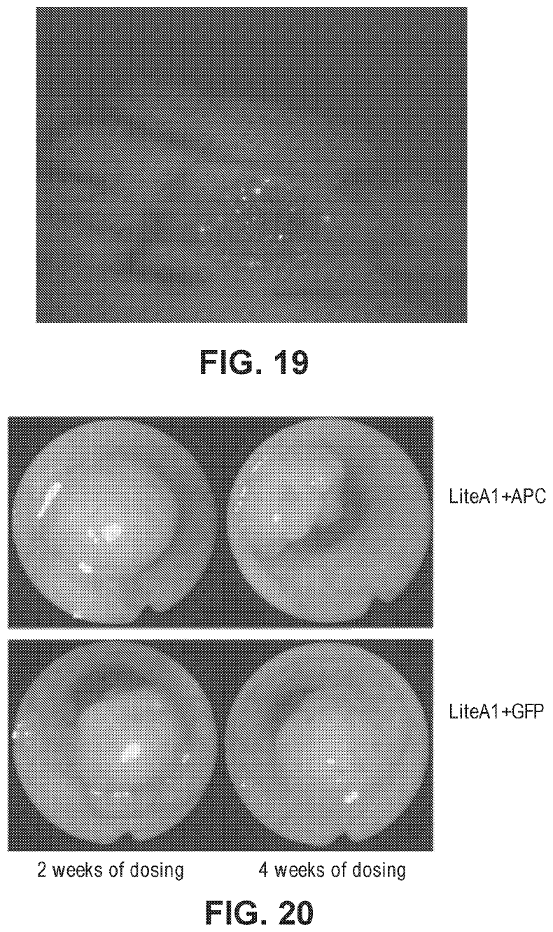

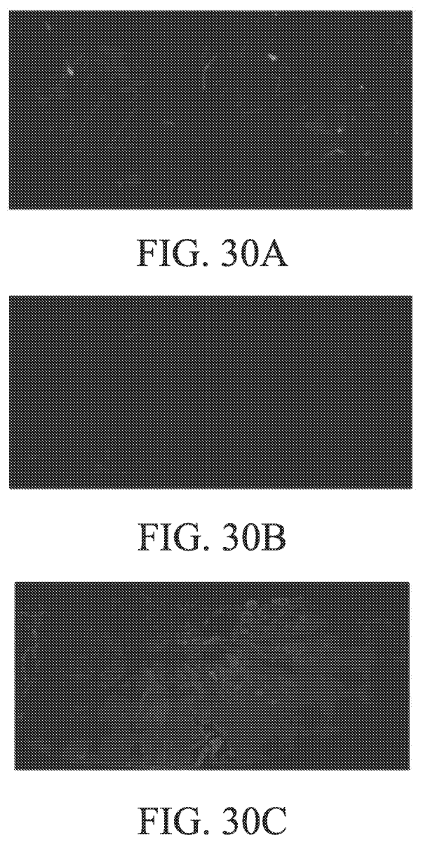

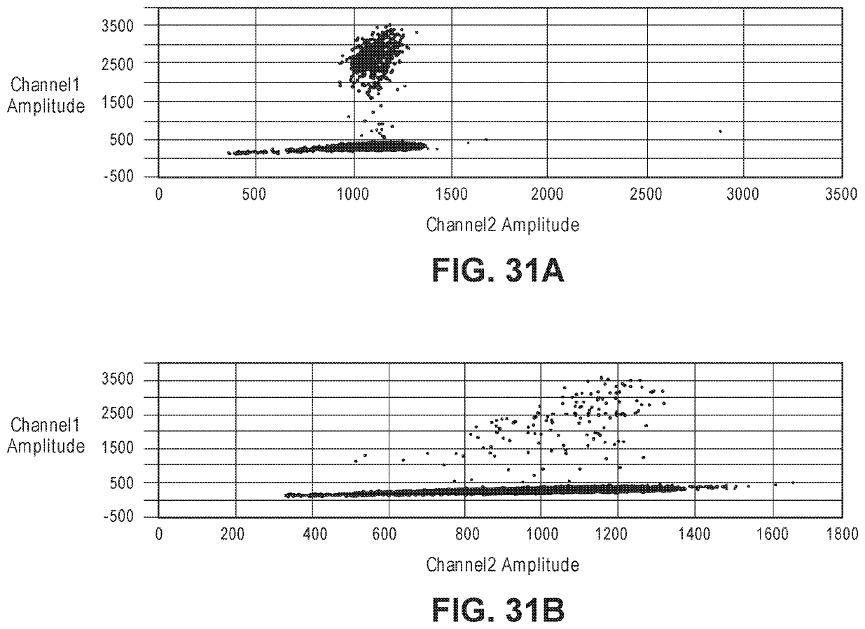

Animals were anesthetized with isoflurane and dosed intrarectally. Prior to dosing a PBS enema was administered and manual peristalsis was performed to remove any blockage and clear the colon. The drug (30 μg DNA coupled to vehicle) was then delivered with equal distribution along the lower two thirds of the colon. The anus was physically pinched and held for 2 min to prevent leakage. Routine endoscopic surveillance was performed prior to dosing, at 2 weeks, 4 weeks, 6 weeks, and before necropsy at 7 weeks. In vitro testing of the NTC9385R-APC plasmid was performed to validate protein expression. 4 μg of DNA (NTC9385R-APC or NTC9385R-GFP as control) was coupled with 25 μL of 1 mM LiteA1 and transfected into a single well of a 6 well plate. This was performed in triplicate. This was allowed to express for 48 hours. Cells were then suspended in RIPA buffer and lysed via shaking for 1 hour. The lysate was pelleted via centrifugation and the protein concentration in the supernatant was measured. 40 μg of the supernatant was then added to 4× NuPAGE LDS sample buffer. Protein was then denatured in a 95 C water bath for 5 min. The sample was then run at 150 V for 2.5 hours on an 4-20% Mini-PROTEAN TG Precast gel. The protein was then transferred to a PVDF membrane. This was then incubated with 1:2000 primary Anti-APC antibody (ab15270) from Abcam overnight at 4 C. The membrane was washed and then incubated with the secondary Goat Anti-Rabbit IgG H&L (HRP) (ab205718) from Abcam for 1 hour at room temperature. The membrane was then washed and visualization was accomplished using the 1-Step Ultra TMB-Blotting Solution for 30 min, FIG. 17. Intestinal crypt staining was performed post-mortem on the intestinal epitheliam, FIG. 28A and FIG. 28B. GFP expression was detected within the intestinal crypt, FIG. 29.

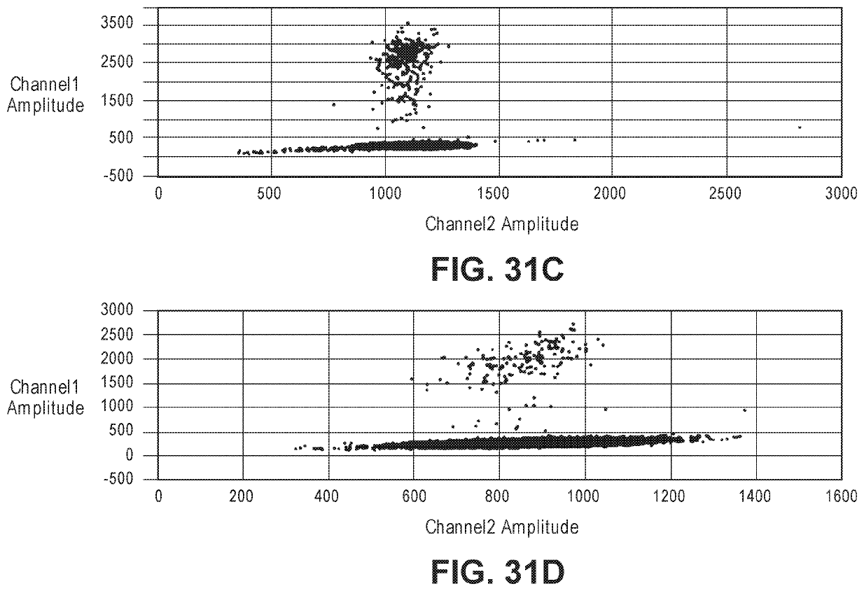

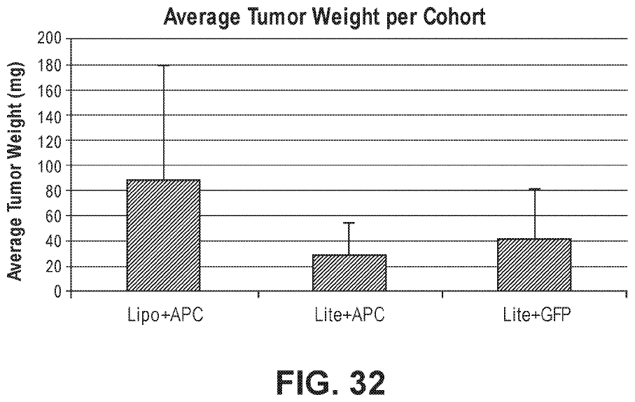

In order to both treat and prevent FAP, the ability to transfect tumors was measured. Pirc rats with colorectal tumors were treated with LiteA1+GFP or LiteA1+APC (control). Post mortem, tumors were collected and GFP was measured via microscopy. Lite+GFP was detected in tumors of Pirc rats, FIG. 30B and FIG. 30C whereas little to no expression of GFP was measured in the GFP negative control, FIG. 30A. Tumor weight was measured post-mortem, FIG. 32. Largest tumors for Lite-GFP were removed for histology and could not be weighed so Lite-GFP average is showing a lower value than it should be.

### Digital Droplet PCR

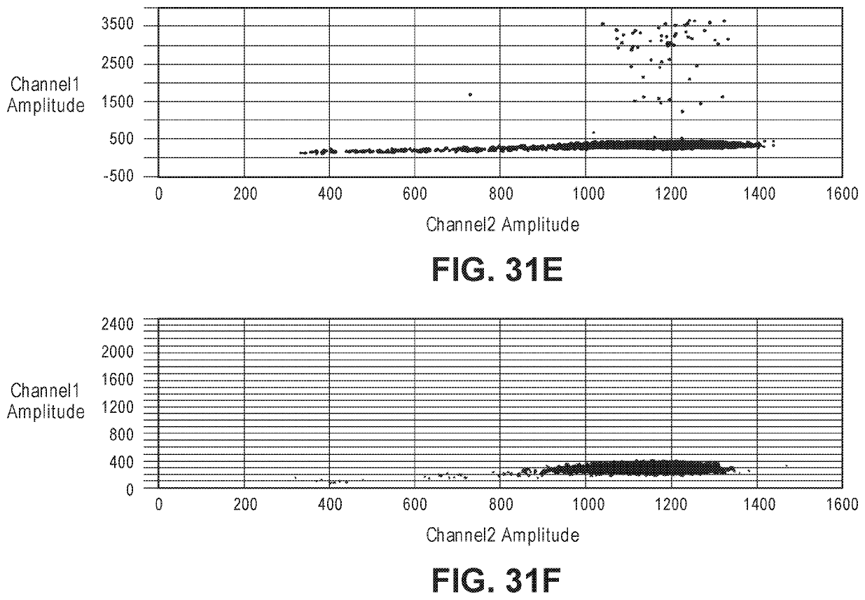

Digital droplet PCR was performed on animals from the LiteA1+GFP cohort. DNA added to each ddPCR ranged between animals and tissues: Liver (200 ng), Spleen (˜800 ng-1 ug), Serum (<ng), Normal Epithelium (500 ng), Tumor (100 ng-1 ug). Graphs depicts 4 separate animals overlayed, FIG. 31A, FIG. 31B, FIG. 31C, FIG. 31D, FIG. 31E, and FIG. 31F. All animals had roughly the same representation of vector probe. Channel 1 was HTLV primers and probe labeled with FAM. The Channel 2 was HPRT housekeeping gene (labeled with HEX) to show DNA addition.

### Example 23: In Vitro Mucus Penetration Analysis

### I. Transfection Efficiency

An in-vitro mucus assay was used to measure the mucus penetration of the Lite delivery system (MVL5/GMO/Lipid-HPEG 50:43:7) complexed with DNA at different charge ratios (+5, +3, +2 and +1). Lipofectamine 2000 was used as a mucoadhesive control. The ratios were decided based on DNA-binding efficiency, transfection efficiency and mucus penetration. In order to assess the binding efficiencies and overall charge of the complex, the Lite delivery system was complexed with DNA and agarose gel electrophoresis was performed. Briefly, DNA was complexed with Ethidium Bromide at 0.5 μg/ml and then mixed with the appropriate ratio of Lite delivery system. Agarose gel (1%) electrophoresis was performed at a constant voltage of 80 mV. No free DNA was detected for charge ratios of +5, +3 and +2. Charge ratio of +1 showed little binding of DNA. Transfection efficiency was determined by transfecting HEK cells using Lite at different charge ratios complexed with the NTC-eGFP plasmid, Table 5.

### II. Mucus Collection

To assess penetration of mucus, physiologically relevant adherent mucus was collected from porcine colons. Fresh porcine colons were obtained from a local abattoir were collected and kept on ice. Chyme was physically removed and the intestine was rinsed carefully with physiological saline (NaCl 0.9%) to remove residual matter. A lateral incision was made and the intestine was laid flat. The mucus collection was done by scrapping off the mucus layer from the membrane with a glass slide. To 1 g of mucus, 5 mL of 0.1 M sodium chloride was added and agitated for 1 h (shaking at 50 rpm) after which the suspension was centrifuged for 2 h (at 13000 rpm). Large debris was physically removed and only the clean portion of the pellet was retained. Mucus was stored at −20° C. and was warmed to 37 C before use.

III. In vitro Mucus Assay

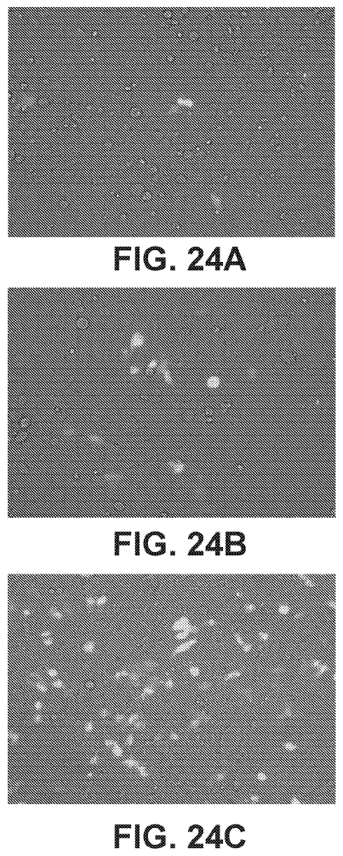

Non-diluted clean mucus as prepared above was used for this assay. A thin cylindrical polypropylene tube (˜4 mm) with one closed end was filled with mucus up to 10 mm. Each charge ratio and type of lipid was analyzed in triplicates. 4 μg of Cy5-labelled DNA was complexed with the appropriate ratio of lipids. Triplicates of a negative control were also performed with just MVL5/GMO/Lipid-HPEG and OPTI-MEM. Lipoplex-DNA complexes were diluted with OPTI-MEM to an end-volume of 50 ul and pipetted on top of the mucus. Samples were incubated at 37 C at 50 rpm for 4 hours after which they were frozen at −80 C overnight. Each tube was cut at 2 mm lengths with the 0 mm point being at the top of the mucus meniscus. Tubes were cut from the end to the top to prevent artificial penetration of the mucus caused by the physical force of cutting. The aqueous portion was also stored and analyzed. Polypropylene tube pieces were removed from the collected samples and 120 uL of extraction buffer (0.1M Sodium Acetate pH 5.4, 20% DMSO and 1% SDS) was added to the tubes to disrupt the mucus and release the Cy5 tagged DNA. Samples were vortexed for 30 s and shaken at 300 rpm at 37 C for 1 h. Mucus was pelleted by centrifugation at 13,200 g for 5 min and the supernatants were analyzed using a microplate reader (ex: 633 nm, em: 670 nm, cutoff: 665 nm). Each 2 mm piece was adjusted for background by subtraction of the average fluorescence intensity of the negative controls. Percentage fluorescent contribution of each piece for each tube was calculated and analyzed, FIG. 23.

Alternatively, the observed heterogeneity in mucus even from the same colon could introduce variation. However, the charge ratio of 2 was found to exhibit a higher mucosal penetration than other particles. Cy5-DNA alone was found dominantly in the aqueous layer and did not display similar characteristics as the particle shown.

TABLE 5
Transfection

| | μL Lite Delivery system | μL pCMV-GFP |
|---|---|---|
| Charge Ratio | (1 mM) | (1 μg/uL) |
| 5+ | 12.5 | 2 |
| 3+ | 7.5 | 2 |
| 2+ | 5 | 2 |

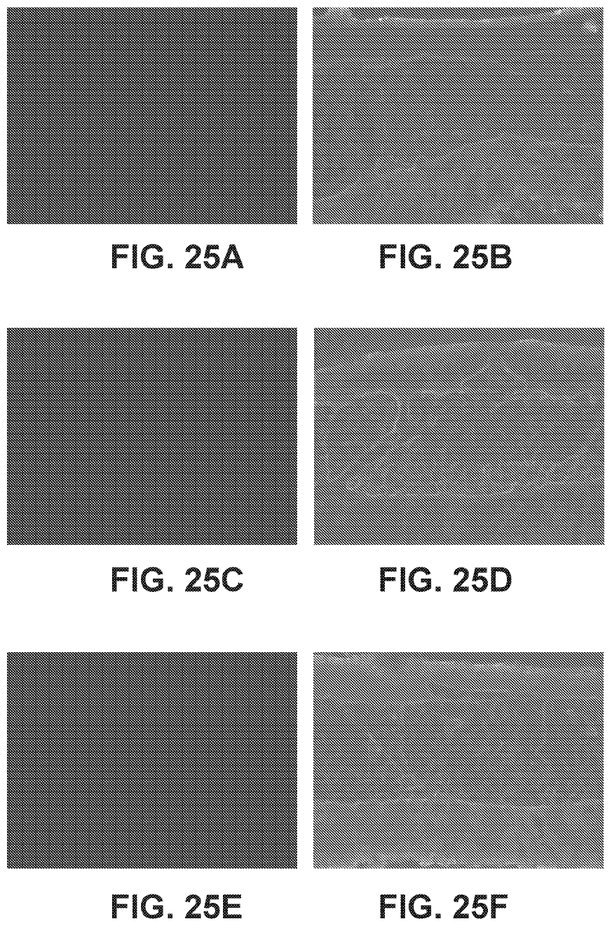

Lite delivery system was coupled as follows to produce a vector delivering 2 μg pCMV-GFP and incubated at room temperature for 10 min. Each vector was then transfected into a 6-well plate of HEK 293T cells at 80% confluency. Each well was then imaged 48 hrs later using an EVOS FLoid cell imaging station (Life Technologies) with green light at 20%, FIG. 24A, FIG. 24B, and FIG. 24C.

### Example 24: Ex Vivo Porcine Mucus Assay

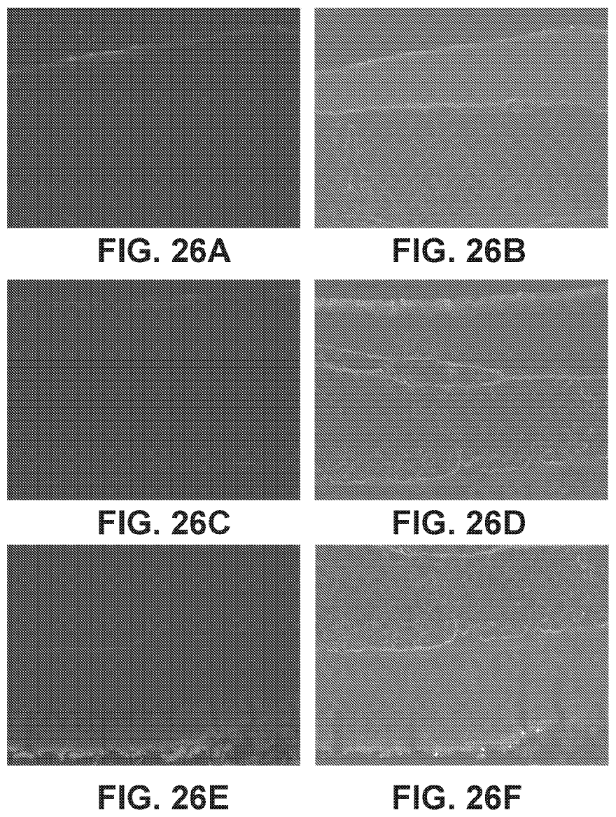

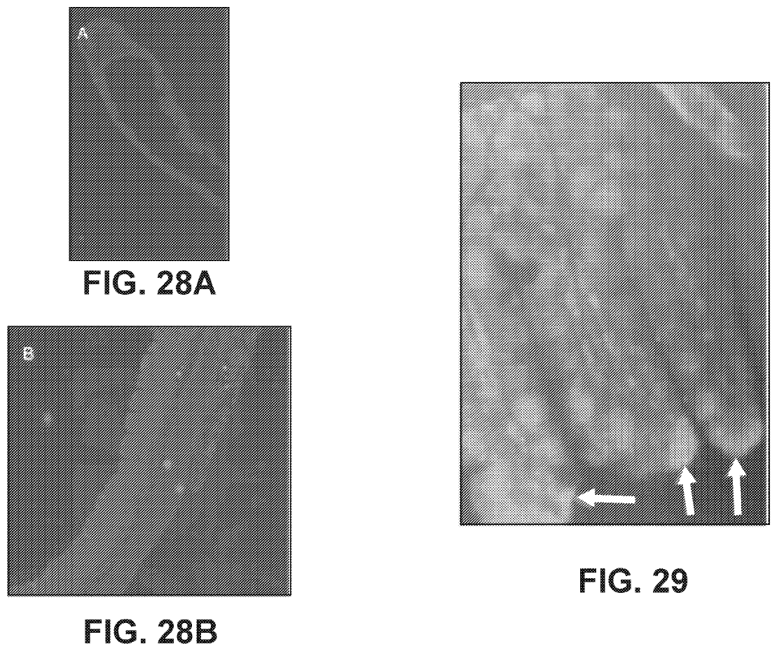

Vehicle was formulated as MVL5/GMO/Lipid-HPEG (50:43:7) using the thin film-hydration method. Approximately 4 feet of large pig intestine was collected from the abattoir Marin Sun Farms in Petaluma, Calif. A longitudinal incision was made along the intestine and 2 mm×4 mm sections were cut in regions with minimal chyme. Two conditions were tested; a 60 bp ssDNA oligonucleotide linked to Cy5 was coupled with the delivery vehicle or the vehicle alone. 100 μL of each condition was pipetted on a colon section and incubated at 37° C. with 5% CO₂ for different time points: 5 min, 60 min, 100 min. This was performed in triplicate for each condition and time point. The sections were then embedded in OCT embedding medium and flash frozen in dry ice slurry. The samples were then stored at −80° C. until they were cryosectioned. Several approximately 40 μm sections were taken from the center of each sample, FIG. 25A, FIG. 25B, FIG. 25C, FIG. 25D, FIG. 25E, FIG. 25F, FIG. 27A, FIG. 27B, FIG. 27A, FIG. 27B, FIG. 27C, FIG. 27D, and FIG. 27E.

While some embodiments have been shown and described herein, such embodiments are provided by way of example only. Numerous variations, changes, and substitutions will now occur to those skilled in the art without departing from the invention. It should be understood that various alternatives to the embodiments of the invention described herein can be employed in practicing the invention.

What is claimed is:

1. A structure comprising:
a) an isolated and purified circular polynucleic acid encoding at least a fragment of a protein that is active in a gastrointestinal tract; and
b) a liposome comprising a lipid bilayer wherein an outer surface of the liposome contacts a polymer wherein the isolated and purified circular polynucleic acid is at least partially encapsulated in the liposome.

2. The structure of claim 1, wherein said structure is a nanostructure.

3. The structure of any one of claims 1 to 2, wherein said structure has a diameter selected from a group consisting of: from about 10 nm to about 100 nm, from about 100 nm to about 200 nm, from about 200 nm to about 300 nm, from about 300 nm to about 400 nm, and from about 400 nm to about 500 nm as measured by dynamic light scattering.

4. The structure of claim 3, wherein said structure has a diameter from about 100 nm to about 200 nm as measured by dynamic light scattering.

5. The structure of any one of claims 1 to 4, further comprising an external coating.

6. The structure of claim 5, wherein said external coating is an enteric coating.

7. The structure of any one of claims 5 to 6, wherein said external coating fully coats a surface of said structure.

8. The structure of any one of claims 5 to 7, wherein said external coating comprises a material selected from a group consisting of: cellulose acetate phthalate, polyvinyl acetate phthalate, hydroxypropylmethylcellulose acetate succinate, poly(methacylic acid-co-ethyl acrylate), poly(methacrylic acid-co-ethyl acrylate), poly(methacylic acid-co-methyl methacrylate), poly(methacylic acid-co-methyl methacrylate), poly(methacylic acid-co-methyl methacrylate), poly(methacylic acid-co-methyl methacrylate), poly(methyl acrylate-co-methyl methacrylate-co-methacrylic acid), hydroxyethyl Cellulose (HEC), polyacrylates (carbomer), alginates, chitosan, cellulosic derivatives (hydroxyethylcellulose, hydroxypropylmethylcellulose, carboxymethylcellulose, and any combination thereof.

9. The structure of claim 8, wherein said external coating comprises poly (methacylic acid-co-ethyl acrylate).

10. The structure of any one of claims 5 to 9, wherein said external coating is a mucoadhesive hydrogel.

11. The structure of any one of claims 5 to 10, wherein said external coating is pH sensitive.

12. The structure of claim 11, wherein when said pH sensitive external coating when placed in 1 L of water with a stirring rod rotating at 200 revolutions per minute at a pH from about 5.5 to about 14 as measured at 37 degrees Celsius with a pH meter at least partially dissolves.

13. The structure of claim 11, wherein when said pH sensitive external coating when placed in 1 L of water with a stirring rod rotating at 200 revolutions per minute at a pH from about 6 to about 14 as measured at 37 degrees Celsius with a pH meter at least partially dissolves.

14. The structure of claim 11, wherein when said pH sensitive external coating when placed in 1 L of water with a stirring rod rotating at 200 revolutions per minute at a pH from about 7 to about 14 as measured at 37 degrees Celsius with a pH meter at least partially dissolves.

15. The structure of any one of claims 1 to 14, wherein said structure when orally administered to a primate at least partially dissolves in a duodenum, jejunum, ilium, colon, or any combination thereof.

16. The structure of claim 15, wherein said structure when orally administered to a primate at least partially dissolves adjacent to an intestinal crypt cell.

17. The structure of any one of claims 1 to 16, wherein said at least one polymer allows said structure to traverse mucous more so than an otherwise comparable structure that does not comprise said at least one polymer as measured by a transwell migration assay.

18. The structure of any one of claims 1 to 17, wherein said polymer is selected from a group consisting of a polyethylene glycol (PEG) containing polymer, a triblock copolymer of PEG-polypropyelene oxide, poly(2-methyl-2-oxazoline), poly(vinyl alcohol), poly(vinyl ethers), poly(N-[2-hydroxypropyl)methylacrylamide), polyethyleneimine (PEI), poly(2-dimethylaminoethyl methacrylate) (pDMAEMA), and any combination thereof.

19. The structure of claim 18, wherein said polymer comprises poly(2-methyl-2-oxazoline).

20. The structure of claim 18, wherein said polymer comprises PEG.

21. The structure of any one of claims 1 to 20, wherein said polymer is associated with said lipid bilayer via a linker.

22. The structure of claim 21, wherein said linker is an acid-labile linker.

23. The structure of any one of claims 21 to 22, wherein said linker further comprises at least one of a disulfide bond, acyl hydrazone, vinyl ether, orthoester, or a N—PO3 group.

24. The structure of any one of claims 1 to 23, wherein said lipid bilayer forms a liposome.

25. The structure of claim 24, wherein said polymer is substantially uniformly dispersed on at least part of a surface of said liposome.

26. The structure of claim 25, wherein said liposome has an exterior surface and an interior surface and wherein said polymer is substantially uniformly dispersed on said exterior surface.

27. The structure of any one of claims 1 to 24, wherein said polymer is not uniformly dispersed on said liposome.

28. The structure of claim 27, wherein said liposome has an exterior surface and an interior surface and wherein said polymer is not uniformly dispersed on said exterior surface.

29. The structure of any one of claims 1 to 28, wherein when said polymer is a PEG containing polymer that comprises a weight average molecular weight ranging from about 1900 g/mol to about 2200 g/mol.

30. The structure of claim 29, wherein when said polymer is a PEG containing polymer having a weight average molecular weight from about 1900 g/mol to about 2200 g/mol said polymer is associated with said lipid bilayer at a ratio from about 10 chains per 100 nm² of said lipid bilayer to about 20 chains per 100 nm² of said lipid bilayer as measured by a relative comparison of the actual molar ratio of lipid PEG and a calculated weight average surface area of said liposome.

31. The structure of any one of claims 1 to 30, wherein said polymer is at least in part in a mushroom configuration.

32. The structure of any one of claims 1 to 30, wherein said polymer is at least in part in a brush configuration.

33. The structure of any one of claims 1 to 32, wherein said lipid bilayer comprises a material selected from the group consisting of: cholesterol, N-[1-(2,3-dioleyloxy)propyl]-N,N,N-trimethylammonium chloride (DOTMA), [1,2-bis(oleoyloxy)-3 (trimethylammonio)propane](DOTAP), 3β[N—(N′, N′-dimethylaminoethane)-carbamoyl] cholesterol (DC-Chol), dioctadecylamidoglycylspermine (DOGS), Dioleoylphosphatidylethanolamine (DOPE), N 1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-Distearoyl-sn-glycero-3-phosphoethanolamine (DSPE), 1,2-distearoyl-sn-glycero-3-phosphocholine (DSPC), Dimethyldioctadecylammonium (DDAB), a salt of any of these, and any combination thereof.

34. The structure of claim 33, wherein said material comprises a lipid with a net positive charge or a lipid with a neutral charge.

35. The structure of claim 33 or 34, wherein said material comprises MVL5 and GMO.

36. The structure of claim 35, wherein a molar ratio of MVL5 and GMO ranges from about 1:10 to about 1:1.

37. The structure of any one of claims 35 to 36, wherein the molar ratio of MVL5/GMO/lipid-HPEG is selected from group consisting of about: 50 mol/45 mol/5 mol, 50 mol/44 mol/6 mol, 50 mol/43 mol/7 mol, 50 mol/42 mol/8 mol, 50 mol/41 mol/9 mol, and to about 50 mol/40 mol/10 mol.

38. The structure of any one of claims 35 to 37, wherein when said lipid bilayer comprises said MVL5, said MVL5 hydrogen bonds said isolated and purified circular polynucleic acid.

39. The structure of any one of claims 18 to 38, further comprising a PEG complex.

40. The structure of any one of claims 24 to 39, wherein said circular polynucleic acid is fully encapsulated in said liposome.

41. The structure of any one of claims 1 to 40, further comprising a linker.

42. The structure of claim 41, wherein said linker is covalently associated with said polymer.

43. The structure of claims 41 to 42, wherein said linker is an acid sensitive linker.

44. The structure of any one of claims 1 to 43, wherein said structure further comprises a peptide, antibody or fragment thereof, carbohydrate, single chain variable fragment (scFv), cellular receptor, or any combination thereof.

45. The structure of claim 44, wherein said structure further comprises a peptide, antibody or fragment thereof, single chain variable fragment (scFv), or cellular receptor in contact with said polymer.

46. The structure of claim 45, wherein when said structure comprises said peptide it is a cell-penetrating peptide.

47. The structure of claim 44, wherein when said structure comprises said antibody or fragment thereof it targets a leucine-rich repeat-containing G-protein coupled receptor 5 (LGR5).

48. The structure of any one of claims 5 to 47, wherein said external coating is cationic.

49. The structure of any one of claims 5 to 47, wherein said external coating is anionic.

50. The structure of any one of claims 5 to 47, wherein said external coating is neutral.

51. The structure of any one of claims 5 to 50, wherein said external coating's charge is measured by laser doppler anemometry.

52. The structure of claim 51, wherein said charge is from about −100 mV to about 100 mV for structures at a DNA charge ratio from about 5 to about 15 in 1 mL of high-resistivity water as measured by a two angle particle and molecular size analyzer.

53. The structure of any one of claims 1 to 52, wherein said isolated and purified circular polynucleic acid is DNA or RNA.

54. The structure of any one of claims 1 to 53, wherein said isolated and purified circular polynucleic acid is single stranded.

55. The structure of any one of claims 1 to 53, wherein said isolated and purified circular polynucleic acid is double stranded.

56. The structure of any one of claims 53 to 55, wherein said isolated and purified circular polynucleic acid is DNA.

57. The structure of claim 56, wherein said DNA is minicircle DNA.

58. The structure of any one of claims 1 to 57, wherein said isolated and purified circular polynucleic acid is at least partially water soluble.

59. The structure of claim 58, wherein said isolated and purified circular polynucleic acid is present in an aqueous solution enclosed in said lipid bilayer.

60. The structure of any one of claims 1 to 59, wherein said at least a fragment of a protein that is active in a gastrointestinal tract is at least a portion of adenomatous polyposis coli (APC), at least a portion of B-galactosidase (B-Gal), or any combination thereof.

61. The structure of claim 60, wherein said at least a fragment of a protein that is active in a gastrointestinal tract is at least a portion of adenomatous polyposis coli (APC).

62. The structure of any one of claims 1 to 61, wherein said at least a fragment of a protein that is active in a gastrointestinal tract comprises at least a portion of defensin alpha 5 (HD-5), at least a portion of defensin alpha 6 (HD-6), or any combination thereof.

63. The structure of any one of claims 1 to 62, wherein said isolated and purified circular polynucleic acid comprises at least one promoter.

64. The structure of claim 63, wherein said promoter is selected from a list comprising a cytomegalovirus (CMV) derived promoter, chicken 3-actin (CBM) derived promoter, adenomatous polyposis coli (APC) derived promoter, leucine-rich repeat containing G protein-coupled receptor 5 (LGR5), CAG promoter, Beta actin promoter, elongation factor-1 (EF1) promoter, early growth response 1 (EGR-1) promoter, eukaryotic initiation factor 4A (EIF4A1) promoter, or any combination thereof.

65. The structure of any one of claims 1 to 64, wherein said isolated and purified circular polynucleic acid is at least partially in contact with said structure, is in contact with at least one component of said structure, or a combination thereof.

66. The structure of claim 65, wherein said isolated and purified circular polynucleic acid is in contact with a cationic lipid.

67. The structure of any one of claims 1 to 66, further comprising a protein or peptide.

68. The structure of claim 67, wherein said protein or peptide comprises a nuclear localization signal (NLS).

69. The structure of any one of claims 67 to 68, wherein said protein or peptide contacts said isolated and purified circular polynucleic acid.

70. The structure of any one of claims 67 to 69, wherein said protein or peptide does not contact said isolated and purified circular polynucleic acid.

71. The structure of any one of claims 1 to 70, further comprising a nuclease inhibitor.

72. The structure of claim 71, wherein said nuclease inhibitor is selected from the group consisting of aurintricarboxylic acid (ATA), Zn²⁺, DMI-2, or a combination thereof.

73. The structure of any one of claim 1 to 72, further comprising an effector of RNA interference (RNAi) enclosed in said structure.

74. The structure of claim 73, wherein said effector of RNA interference (RNAi) is a CEQ508 or salt thereof.

75. The structure of any one of claims 1 to 74, wherein when said salt is a cationic metal.

76. The structure of any one of claims 1 to 75, wherein when said buffering agent is a buffer selected from the group consisting of phosphate buffered saline (PBS) tris-(hydroxymethyl)-aminomethane hydrochloride (TRIS) buffer, N-2-hydroxyethyl piperazine-N′-2-ethane sulfonic acid (HEPES), glycine buffer, glutamic acid, and any combination thereof.

77. The structure of claim 76, wherein said buffering agent is PBS.

78. The structure of any one of claims 1 to 77, wherein said structure is a mucus penetrating particle (MPP).

79. The structure of claim 78, wherein said MPP has a near neutral zeta potential from about −20 mV to about 20 mV as measured by laser doppler anemometry.

80. The structure of claim 78 to 79, wherein said MPP is able to penetrate mucus from 1 μm to 200 μm in thickness as measured by a transwell migration assay.

81. The structure of any one of claims 1 to 80, wherein said structure is spherical.

82. The structure of any one of claims 1 to 81, wherein said structure is at least partially biodegradable.

83. The structure of any one of claims 1 to 82, wherein said structure is freeze-dried.

84. The structure of any one of claims 1 to 83, wherein said structure is comprised in a pill, a hydrogel, or any combination thereof.

85. The structure of any one of claims 1 to 84, wherein said isolated and purified circular polynucleic acid encodes a tumor suppressor protein or precursor thereof.

86. A pharmaceutical composition comprising the structure of any one of claims 1 to 85.

87. The pharmaceutical composition of claim 86, wherein said pharmaceutical composition is in unit dosage form.

88. The pharmaceutical composition of any one of claims 86 to 87, wherein said pharmaceutical composition comprises a pharmaceutically acceptable excipient.

89. A method comprising administering a structure or pharmaceutical composition of any one of claims 1 to 88 to a subject in need thereof.

90. The method of claim 89, wherein said method treats a disease or condition in said subject and wherein the structure or pharmaceutical composition is administered in a therapeutically effective amount.

91. The method of claim 90, wherein said disease or condition is familial adenomatous polyposis (FAP), attenuated FAP, cancer, chronic inflammatory bowel disease, chronic inflammatory bowel disease, ileal Crohn's or any combination thereof.

92. The method of any one of claims 89 to 91, wherein said structure or pharmaceutical composition is used to treat FAP.

93. The method of any one of claims 89 to 92, wherein said subject has a polyp in a gastrointestinal tract.

94. The method of any one of claims 89 to 93, wherein said subject has a polyp surgically removed prior to, after, or concurrent to said administration of said structure or said pharmaceutical composition.

95. The method of any one of claims 89 to 94, wherein said structure or pharmaceutical composition is administered orally, rectally, or orally and rectally.

96. The method of any one of claims 89 to 95, wherein said structure or pharmaceutical composition is administered routinely.

97. The method of any one of claims 89 to 96, wherein said structure or pharmaceutical composition is administered prophylactically.

98. The method of any one of claims 89 to 97, wherein said structure or pharmaceutical composition is administered 1 time per day, 2 times per day, 3 times per day, daily, weekly, yearly or any combination thereof.

99. The method of any one of claims 89 to 98, wherein said subject is administered an additional therapy in a therapeutically effective amount.

100. The method of claim 99, wherein said additional therapy comprises a non-steroidal anti-inflammatory drug (N SAID), guaifenesin, a miRNA against B-catenin, a mucus disrupting agent, or a salt, or any combination thereof.

101. The method of claim 100, wherein said additional therapy comprises said NSAID and said NSAID is Celecoxib.

102. The method of any one of claims 89 to 101, wherein said subject is genetically screened for disease.

103. A method comprising administering the structure of any one of claims 1 to 85 with delivery efficiency of said structure of at least 30% to a gastrointestinal cell as measured by Transwell-Snapwell diffusion chamber assay.

104. A polynucleic acid with at least 50% homology to SEQ ID 5.

105. The polynucleic acid of claim 104, wherein said polynucleic acid consists of at least 55%, 60%, 65%, 70%, 75%, 80%, 85%, 90%, 95%, 96%, 97%, 98%, or up to 99% homology to SEQ ID 5.

106. A polynucleic acid comprising SEQ ID 5.

107. The polynucleic acid of any one of claims 104 to 106, wherein said polynucleic acid is isolated and purified.

108. A method of making a structure comprising forming a liposome around a circular polynucleic acid encoding a tumor suppressor protein or portion thereof.

109. A method of making a structure comprising forming a liposome around a circular polynucleic acid encoding a protein or portion thereof active in a gastrointestinal tract.

110. The method of any one of claims 108 to 109, further comprising introducing a solvent.

111. The method of claim 110, wherein said solvent comprises chloroform.

112. The method of any one of claims 108 to 111, further comprising drying of said solvent.

113. The method of claim 112, wherein said drying of said solvent is performed by a method comprising dry nitrogen stream, argon stream, rotary evaporation, vacuum, or any combination thereof.

114. The method of claim 113, wherein said method comprises dry nitrogen stream.

115. The method of claim 113, wherein said method comprises vacuuming.

116. The method of any one of claims 113 to 115, wherein said drying is performed by dry nitrogen stream followed by vacuuming.

117. The method of any one of claims 112 to 116, wherein said drying forms a lipid film that is hydrated by addition of an aqueous solution.

118. The method of any one of claims 108 to 117, further comprising an aqueous solution.

119. The method of any one of claims 108 to 118, wherein said circular polynucleic acid comprises DNA or RNA.

120. The method of claim 119, wherein said circular polynucleic acid comprises DNA.

121. The method of claim 120, wherein said circular polynucleic acid comprises mini-circle DNA.

122. A kit comprising the structure of any one of claims 1 to 85 and instructions for use thereof.

123. A kit comprising the polynucleic acid of claims 106 to 107 and instructions for use thereof.

124. A method of making the kit of claim 122 or claim 123.

125. A method of making a pharmaceutical composition comprising contacting the structure of any one of claims 1 to 85 and a pharmaceutically acceptable excipient.

126. A liposomal structure comprising:
a) an isolated and purified circular polynucleic acid, wherein the liposomal structure is surface modified with a polymer, wherein the polymer enhances an average rate at which the liposomal structure moves in mucus compared to a comparable liposomal structure, wherein the comparable liposomal structure is surface modified with polyethylene glycol (PEG) at an average molecular weight ranging from about 2000 Da to about 3000 Da.

127. The liposomal structure of claim 126, wherein the liposomal structure has increased hydrophilicity compared to the comparable liposomal structure.

128. A liposomal structure comprising:
an isolated and purified polynucleic acid, wherein the polynucleic acid is free of a bacterial origin of replication, wherein the liposomal structure is surface modified with a polymer.

129. The liposomal structure of claim 128, wherein the polynucleic acid is circular.

130. The liposomal structure of any one of claims 126 to 129, wherein the liposomal structure is selected from a group comprising a liposome, lipoplex, or lipopolyplex.

131. The liposomal structure of any one of claims 126 to 130, wherein the liposomal structure is surface modified with a polymer of Formula I: 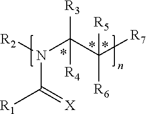
wherein R₁ is independently selected from a group consisting of a bond; hydrogen; deuterium; C₁₋₆ alkyl; C₃₋₈ cycloalkyl; heteroaryl; C₁₋₆ alkylheteroaryl; C₁₋₆ alkylaryl; and alkylcycloalkyl; each of which except hydrogen and deuterium may be individually and independently substituted one or more times with XA; halogen; NY₂; CXXY; XCY₃; alkyl; hydrogen; deuterium; carboxylic acid; ether; amine; XX₂NY₂; XCY₂X or any combination thereof;
R₂ is independently selected from a group consisting of a bond; hydrogen; deuterium; C₁₋₆ alkyl; C₃₋₈ cycloalkyl; heteroaryl; C₁₋₆ alkylheteroaryl; C₁₋₆ alkylaryl; and alkylcycloalkyl; each of which except hydrogen and deuterium may be individually and independently substituted one or more times with XA; halogen; NY₂; CXXY; XCY₃; alkyl; hydrogen; deuterium; carboxylic acid; ether; amine; XX₂NY₂; XCY₂X or any combination thereof;
R₃ is independently selected from a group consisting of a bond; hydrogen; deuterium; C₁₋₆ alkyl; C₂₋₆ alkenyl; C₂₋₆ alkynyl; C₃₋₈ cycloalkyl; heteroaryl; C₁₋₆ alkylheteroaryl; C₁₋₆ alkylaryl; and alkylcycloalkyl; each of which except hydrogen and deuterium may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combination thereof;
R₄ is independently selected from a group consisting of a bond; hydrogen; deuterium; C₁₋₆ alkyl; C₂₋₆ alkenyl; C₂₋₆ alkynyl; C₃₋₈ cycloalkyl; heteroaryl; C₁₋₆ alkylheteroaryl; C₁₋₆ alkylaryl; and alkylcycloalkyl; each of which except hydrogen and deuterium may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combination thereof;
R₅ is independently selected from a group consisting of a bond; hydrogen; deuterium; C₁₋₆ alkyl; C₂₋₆ alkenyl; C₂₋₆ alkynyl; C₃₋₈ cycloalkyl; heteroaryl; C₁₋₆ alkylheteroaryl; C₁₋₆ alkylaryl; and alkylcycloalkyl; each of which except hydrogen and deuterium may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combination thereof;
R₆ is independently selected from a group consisting of a bond; hydrogen; deuterium; C₁₋₆ alkyl; C₂₋₆ alkenyl; C₂₋₆ alkynyl; C₃₋₈ cycloalkyl; heteroaryl; C₁₋₆ alkylheteroaryl; C₁₋₆ alkylaryl; and alkylcycloalkyl; each of which except hydrogen and deuterium may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combination thereof;
R₇ is independently selected from a group consisting of a bond; hydrogen; deuterium; C₁₋₆ alkyl; C₂₋₆ alkenyl; C₂₋₆ alkynyl; C₃₋₈ cycloalkyl; heteroaryl; C₁₋₆ alkylheteroaryl; C₁₋₆ alkylaryl; and alkylcycloalkyl; each of which except hydrogen and deuterium may be individually and independently substituted one or more times with XA; halogen; NY₂; CXXY; XCY₃; alkyl; hydrogen; deuterium; carboxylic acid; ether; amine; XX₂NY₂; ═X; XCY₂X or any combination thereof; wherein * can independently be R, S or achiral; wherein ** can independently be R, S or achiral; wherein X is independently selected from oxygen or sulfur; Y is independently selected from deuterium or hydrogen; A is hydrogen, deuterium, aryl, or heteroaryl and n is from about 1 to about 100.

132. The liposomal structure of claim 131, wherein R₁ is a C₁₋₆ alkyl.

133. The liposomal structure of any one of claims 131 to 132, wherein any one of R₃, R₄, R₅, or R₆ is selected from the group consisting of deuterium and hydrogen.

134. The liposomal structure of any one of claims 131 to 133, wherein X is oxygen.

135. The liposomal structure of claim any one of claims 131 to 134, wherein Formula I has an average molecular weight from about 1000 Da to about 8000 Da.

136. The liposomal structure of claim 126 or 128, wherein the polymer comprises poly(2-methyl-2-oxazoline), poly(2-ethyl-2-oxazoline), a salt thereof, a di block polymer thereof, a tri block polymer thereof, or a combination thereof.

137. The liposomal structure of any one of claims 126 to 136, wherein the polymer is at a density from about 0.05 ug/nm² to about 0.25 ug/nm².

138. The liposomal structure of any one of claims 126 to 137, wherein the average rate at which the liposomal structure moves in mucus is from about 2 fold to about 5 fold greater than the average rate of a comparable liposomal structure as measured by a transwell migration assay.

139. The liposomal structure of claim 138, wherein the polynucleic acid comprises minicircle DNA or closed-linear DNA.

140. The liposomal structure of any one of claims 126 to 139, wherein the liposomal structure further comprises a peptide, antibody or fragment thereof, carbohydrate, single chain variable fragment (scFv), cellular receptor, or any combination thereof.

141. The liposomal structure of any one of claims 126 to 140, further comprising an exterior coating.

142. The liposomal structure of claim 141, wherein the exterior coating comprises ethyl acrylate in polymerized form.

143. The liposomal structure of any one of claims 141 to 142, wherein the exterior coating has a near-neutral zeta potential as measured by laser doppler anemometry.

144. The liposomal structure of any one of claims 126 to 143, wherein the liposomal structure comprises a lipid bilayer.

145. The liposomal structure of claim 144, wherein the lipid bilayer comprises one or more of cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N—(N′, N′-dimethylaminoethane)-carbamoyl] cholesterol (DC-Chol), dioctadecylamidoglycylspermine (DOGS), dioleoylphosphatidylethanolamine (DOPE), N1-[2-((1 S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-distearoyl-sn-glycero-3-phosphoethanolamine (DSPE), 1,2-distearoyl-sn-glycero-3-phosphocholine (DSPC), dimethyldioctadecylammonium (DDAB), a salt thereof, or any combination thereof.

146. The liposomal structure of claim 145, wherein the lipid bilayer comprises MVL5 and GMO.

147. The liposomal structure of claim 146, wherein a molar ratio of MVL5 to GMO ranges from about 10:1 to about 1:10, or 10:1 to about 1:25.

148. The liposomal structure of any one of claims 144 to 147, wherein the liposomal structure further comprises a second lipid bilayer.

149. The liposomal structure of any one of claims 126 to 148, further comprising an acid sensitive linker.

150. The liposomal structure of claim 149, wherein the acid sensitive linker associates with the polymer of the liposomal structure.

151. The liposomal structure of any one of claims 126 to 150, wherein the polynucleic acid encodes for at least a biologically active fragment of a protein.

152. The liposomal structure of claim 151, wherein the biologically active fragment of the protein is active in a bodily area comprising a mucosal membrane.

153. The liposomal structure of claim 126 to 152, wherein the polynucleic acid encodes for at least a biologically active fragment of adenomatous polyposis coli (APC), defensin alpha 5 (HD-5), defensin alpha 6 (HD-6), or any combination thereof.

154. The liposomal structure of any one of claims 126 to 153, wherein the liposomal structure has a diameter selected from the group consisting of: from about 10 nm to about 100 nm, from about 100 nm to about 200 nm, from about 200 nm to about 300 nm, from about 300 nm to about 400 nm, and from about 400 nm to about 500 nm as measured by dynamic light scattering.

155. The liposomal structure of any one of claims 126 to 154, wherein the liposomal structure comprises at least two polynucleic acids.

156. The liposomal structure of any one of claims 126 to 155, wherein the polynucleic acid comprises at least one promoter.

157. The liposomal structure of claim 156, wherein the at least one promoter is selected from cytomegalovirus (CMV) derived promoter, chicken 3-actin (CBM) derived promoter, adenomatous polyposis coli (APC) derived promoter, leucine-rich repeat containing G protein-coupled receptor 5 (LGR5), CAG promoter, Beta actin promoter, elongation factor-1 (EF1) promoter, early growth response 1 (EGR-1) promoter, eukaryotic initiation factor 4A (EIF4A1) promoter, or any combination thereof.

158. The liposomal structure of any one of claims 126 to 157, wherein the liposomal structure further comprises a peptide, antibody or fragment thereof, carbohydrate, single chain variable fragment (scFv), cellular receptor, or any combination thereof.

159. A pharmaceutical composition comprising:
a. the liposomal structure of any one of claims 126 to 158; and
b. at least one of: an excipient, a diluent, or a carrier.

160. The pharmaceutical composition of claim 159, wherein the pharmaceutical composition is in unit dosage form.

161. The pharmaceutical composition of any one of claims 159 to 160, wherein the pharmaceutical composition is in the form of a tablet, a liquid, a syrup, an oral formulation, an intravenous formulation, an intranasal formulation, a subcutaneous formulation, an inhalable respiratory formulation, a suppository, and any combination thereof.

162. A method of treating a subject in need thereof comprising administering to the subject in need thereof a therapeutically effective amount of the liposomal structure of any one of claims 126 to 158 or the pharmaceutical composition of any one of claims 159 to 161.

163. The method of claim 162, wherein the administering of the liposomal structure or the pharmaceutical composition at least partially ameliorates a disease or condition in the subject in need thereof.

164. The method of claim 163, wherein the disease or condition comprises familial adenomatous polyposis (FAP), attenuated FAP, colorectal cancer, chronic inflammatory bowel disease, chronic inflammatory bowel disease, ileal Crohn's or any combination thereof.

165. The method of any one of claims 162 to 164, wherein the liposomal structure or the pharmaceutical composition is administered orally, rectally, or orally and rectally.

166. The method of any one of claims 162 to 165, wherein the liposomal structure or the pharmaceutical composition is administered routinely, prophylactically, or a combination thereof.

167. The method of any one of claims 162 to 166, wherein the liposomal structure or the pharmaceutical composition is administered 1 time per day, 2 times per day, 3 times per day, daily, weekly, yearly or any combination thereof.

168. The method of any one of claims 162 to 167, wherein the subject is administered an additional therapy in a therapeutically effective amount comprising a non-steroidal anti-inflammatory drug (NSAID) or a salt thereof, a miRNA against (3-catenin, a mucus disrupting agent or a salt a salt thereof, or any combination thereof.

169. A kit comprising:
a. the liposomal structure of any one of claims 126 to 158 or the pharmaceutical composition of any one of claims 159 to 161;
b. and instructions for use thereof.

170. The kit of claim 169, further comprising a container.
